SEQUENCE LISTING (150 sequences)

SEQ ID NO: 1 - protein, 125 aa, synthetic construct
  REGION 1..125: note = Anti-TIGIT antibody Clone 13 VH Protein
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 2 - protein, 125 aa, synthetic construct
  REGION 1..125: note = Anti-TIGIT antibody Clone 13A VH
QVQLVQSGAEVKKPGSSVKVSCKASGGTFLSSAISWVRQAPGQGLEWMGSLIPYFGTANYAQKFQGRVTITADESTSTAYMELSSLRSEDTAVYYCARGPSEVGAILGYVWFDPWGQGTLVTVSS

SEQ ID NO: 3 - protein, 125 aa, synthetic construct
  REGION 1..125: note = Anti-TIGIT antibody Clone 13B VH
QVQLVQSGAEVKKPGSSVKVSCKASGGTFSAWAISWVRQAPGQGLEWMGSIIPYFGKANYAQKFQGRVTITADESTSTAYMELSSLRSEDTAVYYCARGPSEVSGILGYVWFDPWGQGTLVTVSS

SEQ ID NO: 4 - protein, 125 aa, synthetic construct
  REGION 1..125: note = Anti-TIGIT antibody Clone 13C VH
QVQLVQSGAEVKKPGSSVKVSCKASGGTFLSSAISWVRQAPGQGLEWMGSIIPLFGKANYAQKFQGRVTITADESTSTAYMELSSLRSEDTAVYYCARGPSEVKGILGYVWFDPWGQGTLVTVSS

SEQ ID NO: 5 - protein, 125 aa, synthetic construct
  REGION 1..125: note = Anti-TIGIT antibody Clone 13D VH
QVQLVQSGAEVKKPGSSVKVSCKASGGTFLSSAISWVRQAPGQGLEWMGSIIPYFGKANYAQKFQGRVTITADESTSTAYMELSSLRSEDTAVYYCARGPSEVKGILGYVWFDPWGQGTLVTVSS

SEQ ID NO: 6 - protein, 112 aa, synthetic construct
  REGION 1..112: note = Clones 13, 13A, 13B, 13C, and 13D VL Protein
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 7 - protein, 9 aa, synthetic construct
  REGION 1..9: note = Clone 13 VH CDR1
GTFSSYAIS

SEQ ID NO: 8 - protein, 9 aa, synthetic construct
  REGION 1..9: note = Clones 13A, 13C, and 13D VH CDR1
GTFLSSAIS

SEQ ID NO: 9 - protein, 9 aa, synthetic construct
  REGION 1..9: note = Clone 13B VH CDR1
GTFSAWAIS

SEQ ID NO: 10 - protein, 17 aa, synthetic construct
  REGION 1..17: note = Clone 13 VH CDR2
SIIPIFGTANYAQKFQG

SEQ ID NO: 11 - protein, 17 aa, synthetic construct
  REGION 1..17: note = Clone 13A VH CDR2
SLIPYFGTANYAQKFQG

SEQ ID NO: 12 - protein, 17 aa, synthetic construct
  REGION 1..17: note = Clones 13B and 13D VH CDR2
SIIPYFGKANYAQKFQG

SEQ ID NO: 13 - protein, 17 aa, synthetic construct
  REGION 1..17: note = Clone 13C VH CDR2
SIIPLFGKANYAQKFQG

SEQ ID NO: 14 - protein, 18 aa, synthetic construct
  REGION 1..18: note = Clones 13 and 13A VH CDR3
ARGPSEVGAILGYVWFDP

SEQ ID NO: 15 - protein, 18 aa, synthetic construct
  REGION 1..18: note = Clone 13B VH CDR3
ARGPSEVSGILGYVWFDP

SEQ ID NO: 16 - protein, 18 aa, synthetic construct
  REGION 1..18: note = Clones 13C and 13D VH CDR3
ARGPSEVKGILGYVWFDP

SEQ ID NO: 17 - protein, 16 aa, synthetic construct
  REGION 1..16: note = Clones 13, 13A, 13B, 13C, and 13D VL CDR1
RSSQSLLHSNGYNYLD

SEQ ID NO: 18 - protein, 7 aa, synthetic construct
  REGION 1..7: note = Clones 13, 13A, 13B, 13C, and 13D VL CDR2
LGSNRAS

SEQ ID NO: 19 - protein, 9 aa, synthetic construct
  REGION 1..9: note = Clones 13, 13A, 13B, 13C, and 13D VL CDR3
MQARRIPIT

SEQ ID NO: 20 - protein, 455 aa, synthetic construct
  REGION 1..455: note = Clone 13 heavy chain hIgG1 (and hIgG1 nonfucosylated) amino acid sequence
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 455 aa, synthetic construct
  REGION 1..455: note = Clone 13A heavy chain hIgG1 (and hIgG1 nonfucosylated) amino acid sequence
QVQLVQSGAEVKKPGSSVKVSCKASGGTFLSSAISWVRQAPGQGLEWMGSLIPYFGTANYAQKFQGRVTITADESTSTAYMELSSLRSEDTAVYYCARGPSEVGAILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 455 aa, synthetic construct
  REGION 1..455: note = Clone 13B heavy chain hIgG1 (and hIgG1 nonfucosylated) amino acid sequence
QVQLVQSGAEVKKPGSSVKVSCKASGGTFSAWAISWVRQAPGQGLEWMGSIIPYFGKANYAQKFQGRVTITADESTSTAYMELSSLRSEDTAVYYCARGPSEVSGILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 455 aa, synthetic construct
  REGION 1..455: note = Clone 13C heavy chain hIgG1 (and hIgG1 nonfucosylated) amino acid sequence
QVQLVQSGAEVKKPGSSVKVSCKASGGTFLSSAISWVRQAPGQGLEWMGSIIPLFGKANYAQKFQGRVTITADESTSTAYMELSSLRSEDTAVYYCARGPSEVKGILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 455 aa, synthetic construct
  REGION 1..455: note = Clone 13D heavy chain hIgG1 (and hIgG1 nonfucosylated) amino acid sequence
QVQLVQSGAEVKKPGSSVKVSCKASGGTFLSSAISWVRQAPGQGLEWMGSIIPYFGKANYAQKFQGRVTITADESTSTAYMELSSLRSEDTAVYYCARGPSEVKGILGYVWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 219 aa, synthetic construct
  REGION 1..219: note = Clone 13, 13A, 13B, 13C, and 13D light chain hkappa (and nonfucosylated) amino acid sequence
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 444 aa, synthetic construct
  REGION 1..444: note = SEA-CD40 (nonfucosylated hS2C6) heavy chain
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 219 aa, synthetic construct
  REGION 1..219: note = SEA-CD40 (nonfucosylated hS2C6) light chain
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 114 aa, synthetic construct [sequence omitted]

SEQ ID NO: 29 - protein, 112 aa, synthetic construct [sequence omitted]

SEQ ID NO: 30 - protein, 5 aa, synthetic construct
  REGION 1..5: note = SEA-CD40 VH CDR1
GYYIH

SEQ ID NO: 31 - protein, 17 aa, synthetic construct
  REGION 1..17: note = SEA-CD40 VH CDR2
RVIPNAGGTSYNQKFKG

SEQ ID NO: 32 - protein, 5 aa, synthetic construct
  REGION 1..5: note = SEA-CD40 VH CDR3
EGIYW

SEQ ID NO: 33 - protein, 16 aa, synthetic construct
  REGION 1..16: note = SEA-CD40 VL CDR1
RSSQSLVHSNGNTFLH

SEQ ID NO: 34 - protein, 7 aa, synthetic construct
  REGION 1..7: note = SEA-CD40 VL CDR2
TVSNRFS

SEQ ID NO: 35 - protein, 9 aa, synthetic construct
  REGION 1..9: note = SEA-CD40 VL CDR3
SQTTHVPWT

SEQ ID NO: 36 - protein, 17 aa, synthetic construct
  REGION 1..17: note = Alternative anti-CD40 antibody CDR2
RVIPQAGGTSYNQKFKG

SEQ ID NO: 37 - protein, 116 aa, synthetic construct
  REGION 1..116: note = SGN-B6A heavy chain variable region
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 38 - protein, 107 aa, synthetic construct
  REGION 1..107: note = SGN-B6A light chain variable region
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 39 - protein, 115 aa, synthetic construct
  REGION 1..115: note = SGN-STNV heavy chain variable region
EVQLVQSGAEVKKPGASVKVSCKASGYTFTDHAIHWVRQAPGQGLEWMGYFSPGNDDIKYNEKFRGRVTMTADKSSSTAYMELRSLRSDDTAVYFCKRSLSTPYWGQGTLVTVSS

SEQ ID NO: 40 - protein, 113 aa, synthetic construct
  REGION 1..113: note = SGN-STNV heavy chain variable region
DIVMTQSPDSLAVSLGERATINCKSSQSLLNRGNHKNYLTWYQQKPGQPPKLLIYWASTRESGVPDRFSGSGSGTDFTLTISSLQAEDVAVYYCQNDYTYPYTFGQGTKVEIK

SEQ ID NO: 41 - protein, 118 aa, synthetic construct
  REGION 1..118: note = SEA-CD70 heavy chain variable region
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 42 - protein, 111 aa, synthetic construct
  REGION 1..111: note = SGN-CD70 light chain variable region
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 43 - protein, 119 aa, synthetic construct
  REGION 1..119: note = SGN-CD228A heavy chain variable region
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSS

SEQ ID NO: 44 - protein, 112 aa, synthetic construct
  REGION 1..112: note = SGN-CD228A light chain variable region
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIK

SEQ ID NO: 45 - protein, 121 aa, synthetic construct
  REGION 1..121: note = SGN-BCMA heavy chain variable region
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 46 - protein, 107 aa, synthetic construct
  REGION 1..107: note = SGN-BCMA light chain variable region
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIK

SEQ ID NO: 47 - protein, 5 aa, synthetic construct
  REGION 1..5: note = SGN-BCMA VH CDR1
DYYIH

SEQ ID NO: 48 - protein, 17 aa, synthetic construct
  REGION 1..17: note = SGN-BCMA VH CDR2
YINPNSGYTNYAQKFQG

SEQ ID NO: 49 - protein, 12 aa, synthetic construct
  REGION 1..12: note = SGN-BCMA VH CDR3
YMWERVTGFFDF

SEQ ID NO: 50 - protein, 11 aa, synthetic construct
  REGION 1..11: note = SGN-BCMA VL CDR1
LASEDISDDLA

SEQ ID NO: 51 - protein, 7 aa, synthetic construct
  REGION 1..7: note = SGN-BCMA VL CDR2
TTSSLQS

SEQ ID NO: 52 - protein, 9 aa, synthetic construct
  REGION 1..9: note = SGN-BCMA VL CDR3
QQTYKFPPT

SEQ ID NO: 53 - protein, 5 aa, synthetic construct
  REGION 1..5: note = SEA-CD70 VH CDR1
NYGMN

SEQ ID NO: 54 - protein, 17 aa, synthetic construct
  REGION 1..17: note = SEA-CD70 VH CDR2
WINTYTGEPTYADAFKG

SEQ ID NO: 55 - protein, 9 aa, synthetic construct
  REGION 1..9: note = SEA-CD70 VH CDR3
DYGDYGMDY

SEQ ID NO: 56 - protein, 15 aa, synthetic construct
  REGION 1..15: note = SEA-CD70 VL CDR1
RASKSVSTSGYSFMH

SEQ ID NO: 57 - protein, 7 aa, synthetic construct
  REGION 1..7: note = SEA-CD70 VL CDR2
LASNLES

SEQ ID NO: 58 - protein, 9 aa, synthetic construct
  REGION 1..9: note = SEA-CD70 VL CDR3
QHSREVPWT

SEQ ID NO: 59 - protein, 118 aa, synthetic construct
  REGION 1..118: note = Zolbetuximab (175D10) heavy chain variable region
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 60 - protein, 109 aa, synthetic construct
  REGION 1..109: note = Zolbetuximab (175D10) light chain variable region
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTK

SEQ ID NO: 61 - protein, 5 aa, synthetic construct
  REGION 1..5: note = Zolbetuximab (175D10) VH CDR1
SYWIN

SEQ ID NO: 62 - protein, 17 aa, synthetic construct
  REGION 1..17: note = Zolbetuximab (175D10) VH CDR2
NIYPSDSYTNYNQKFKD

SEQ ID NO: 63 - protein, 9 aa, synthetic construct
  REGION 1..9: note = Zolbetuximab (175D10) VH CDR3
SWRGNSFDY

SEQ ID NO: 64 - protein, 17 aa, synthetic construct
  REGION 1..17: note = Zolbetuximab (175D10) VL CDR1
KSSQSLLNSGNQKNYLT

SEQ ID NO: 65 - protein, 7 aa, synthetic construct
  REGION 1..7: note = Zolbetuximab (175D10) VL CDR2
WASTRES

SEQ ID NO: 66 - protein, 9 aa, synthetic construct
  REGION 1..9: note = Zolbetuximab (175D10) VL CDR3
QNDYSYPFT

SEQ ID NO: 67 - protein, 118 aa, synthetic construct
  REGION 1..118: note = 163E12 heavy chain variable region
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 68 - protein, 113 aa, synthetic construct
  REGION 1..113: note = 163E12 light chain variable region
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 69 - protein, 5 aa, synthetic construct
  REGION 1..5: note = 163E12 VH CDR1
NYGMN

SEQ ID NO: 70 - protein, 17 aa, synthetic construct
  REGION 1..17: note = 163E12 VH CDR2
WINTNTGEPTYAEEFKG

SEQ ID NO: 71 - protein, 9 aa, synthetic construct
  REGION 1..9: note = 163E12 VH CDR3
LGFGNAMDY

SEQ ID NO: 72 - protein, 17 aa, synthetic construct
  REGION 1..17: note = 163E12 VL CDR1
KSSQSLLNSGNQKNYLT

SEQ ID NO: 73 - protein, 7 aa, synthetic construct
  REGION 1..7: note = 163E12 VL CDR2
WASTRES

SEQ ID NO: 74 - protein, 9 aa, synthetic construct
  REGION 1..9: note = 163E12 VL CDR3
QNDYSYPLT

SEQ ID NO: 75 - protein, 123 aa, synthetic construct
  REGION 1..123: note = SGN-PDL1V heavy chain variable region
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 76 - protein, 106 aa, synthetic construct
  REGION 1..106: note = SGN-PDL1V light chain variable region
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 77 - protein, 5 aa, synthetic construct
  REGION 1..5: note = SGN-PDL1V VH CDR1
TAAIS

SEQ ID NO: 78 - protein, 17 aa, synthetic construct
  REGION 1..17: note = SGN-PDL1V VH CDR2
GIIPIFGKAHYAQKFQG

SEQ ID NO: 79 - protein, 14 aa, synthetic construct
  REGION 1..14: note = SGN-PDL1V VH CDR3
KFHFVSGSPFGMDV

SEQ ID NO: 80 - protein, 11 aa, synthetic construct
  REGION 1..11: note = SGN-PDL1V VL CDR1
RASQSVSSYLA

SEQ ID NO: 81 - protein, 7 aa, synthetic construct
  REGION 1..7: note = SGN-PDL1V VL CDR2
DASNRAT

SEQ ID NO: 82 - protein, 8 aa, synthetic construct
  REGION 1..8: note = SGN-PDL1V VL CDR3
QQRSNWPT

SEQ ID NO: 83 - protein, 123 aa, synthetic construct
  REGION 1..123: note = SGN-ALPV heavy chain variable region
EVQLVESGGGLVQPGRSLRLSCTASGFTFTDYYMSWVRQAPGKGLEWLALIRNKATGYTTEYTASVKGRFTISRDNSKSILYLQMNSLKTEDTAVYYCARASFYYDGKVLAYWGQGTLVTVSS

SEQ ID NO: 84 - protein, 106 aa, synthetic construct
  REGION 1..106: note = SGN-ALPV light chain variable region
DTQMTQSPSSLSASVGDRVTITCQASQDINKYLAWYQYKPGKAPKLLIHYTSSLQSGVPSRFSGSGSGRDYTFTISSLQPEDIATYYCLQYDNLYTFGQGTKLEIK

SEQ ID NO: 85 - protein, 5 aa, synthetic construct
  REGION 1..5: note = SGN-ALPV VH CDR1
DYYMS

SEQ ID NO: 86 - protein, 19 aa, synthetic construct
  REGION 1..19: note = SGN-ALPV VH CDR2
LIRNKATGYTTEYTASVKG

SEQ ID NO: 87 - protein, 12 aa, synthetic construct
  REGION 1..12: note = SGN-ALPV VH CDR3
ASFYYDGKVLAY

SEQ ID NO: 88 - protein, 11 aa, synthetic construct
  REGION 1..11: note = SGN-ALPV VL CDR1
QASQDINKYLA

SEQ ID NO: 89 - protein, 7 aa, synthetic construct
  REGION 1..7: note = SGN-ALPV VL CDR2
YTSSLQS

SEQ ID NO: 90 - protein, 8 aa, synthetic construct
  REGION 1..8: note = SGN-ALPV VL CDR3
LQYDNLYT

SEQ ID NO: 91 - protein, 120 aa, synthetic construct
  REGION 1..120: note = SGN-B7H4V heavy chain variable region
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 92 - protein, 107 aa, synthetic construct
  REGION 1..107: note = SGN-B7H4V light chain variable region
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 93 - protein, 11 aa, synthetic construct
  REGION 1..11: note = SGN-B7H4V VH CDR1
GSIKSGSYYWG

SEQ ID NO: 94 - protein, 16 aa, synthetic construct
  REGION 1..16: note = SGN-B7H4V VH CDR2
NIYYSGSTYYNPSLRS

SEQ ID NO: 95 - protein, 12 aa, synthetic construct
  REGION 1..12: note = SGN-B7H4V VH CDR3
AREGSYPNQFDP

SEQ ID NO: 96 - protein, 11 aa, synthetic construct
  REGION 1..11: note = SGN-B7H4V VL CDR1
RASQSVSSNLA

SEQ ID NO: 97 - protein, 7 aa, synthetic construct
  REGION 1..7: note = SGN-B7H4V VL CDR2
GASTRAT

SEQ ID NO: 98 - protein, 9 aa, synthetic construct
  REGION 1..9: note = SGN-B7H4V VL CDR3
QQYHSFPFT

SEQ ID NO: 99 - protein, 445 aa, synthetic construct
  REGION 1..445: note = Disitamab vedotin heavy chain
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 212 aa, synthetic construct
  REGION 1..212: note = Disitamab vedotin light chain
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 450 aa, synthetic construct
  REGION 1..450: note = Lifastuzumab vedotin heavy chain
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 219 aa, synthetic construct
  REGION 1..219: note = Lifastuzumab vedotin light chain
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 117 aa, synthetic construct
  REGION 1..117: note = PADCEV (Enfortumab Vedotin) (EV) heavy chain variable region
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 104 - protein, 107 aa, synthetic construct
  REGION 1..107: note = PADCEV (Enfortumab Vedotin) (EV) light chain variable region
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 105 - protein, 5 aa, synthetic construct
  REGION 1..5: note = PADCEV (Enfortumab Vedotin) (EV) VH CDR1
SYNMN

SEQ ID NO: 106 - protein, 17 aa, synthetic construct
  REGION 1..17: note = PADCEV (Enfortumab Vedotin) (EV) VH CDR2
YISSSSSTIYYADSVKG

SEQ ID NO: 107 - protein, 8 aa, synthetic construct
  REGION 1..8: note = PADCEV (Enfortumab Vedotin) (EV) VH CDR3
AYYYGMDV

SEQ ID NO: 108 - protein, 11 aa, synthetic construct
  REGION 1..11: note = PADCEV (Enfortumab Vedotin) (EV) VL CDR1
RASQGISGWLA

SEQ ID NO: 109 - protein, 7 aa, synthetic construct
  REGION 1..7: note = PADCEV (Enfortumab Vedotin) (EV) VL CDR2
AASTLQS

SEQ ID NO: 110 - protein, 9 aa, synthetic construct
  REGION 1..9: note = PADCEV (Enfortumab Vedotin) (EV) VL CDR3
QQANSFPPT

SEQ ID NO: 111 - protein, 116 aa, synthetic construct; identical to SEQ ID NO: 37
  REGION 1..116: note = h2A2 heavy chain variable region

SEQ ID NO: 112 - protein, 107 aa, synthetic construct; identical to SEQ ID NO: 38
  REGION 1..107: note = h2A2 light chain variable region

SEQ ID NO: 113 - protein, 5 aa, synthetic construct
  REGION 1..5: note = h2A2 VH CDR1
DYNVN

SEQ ID NO: 114 - protein, 17 aa, synthetic construct
  REGION 1..17: note = h2A2 VH CDR2
VINPKYGTTRYNQKFKG

SEQ ID NO: 115 - protein, 7 aa, synthetic construct
  REGION 1..7: note = h2A2 VH CDR3
GLNAWDY

SEQ ID NO: 116 - protein, 11 aa, synthetic construct
  REGION 1..11: note = h2A2 VL CDR1
GASENIYGALN

SEQ ID NO: 117 - protein, 7 aa, synthetic construct
  REGION 1..7: note = h2A2 VL CDR2
GATNLED

SEQ ID NO: 118 - protein, 9 aa, synthetic construct
  REGION 1..9: note = h2A2 VL CDR3
QNVLTTPYT

SEQ ID NO: 119 - protein, 117 aa, synthetic construct
  REGION 1..117: note = h15H3 heavy chain variable region
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSS

SEQ ID NO: 120 - protein, 112 aa, synthetic construct
  REGION 1..112: note = h15H3 light chain variable region
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIK

SEQ ID NO: 121 - protein, 5 aa, synthetic construct
  REGION 1..5: note = h15H3 VH CDR1
GYFMN

SEQ ID NO: 122 - protein, 17 aa, synthetic construct
  REGION 1..17: note = h15H3 VH CDR2
LINPYNGDSFYNQKFKG

SEQ ID NO: 123 - protein, 8 aa, synthetic construct
  REGION 1..8: note = h15H3 VH CDR3
GLRRDFDY

SEQ ID NO: 124 - protein, 16 aa, synthetic construct
  REGION 1..16: note = h15H3 VL CDR1
KSSQSLLDSDGKTYLN

SEQ ID NO: 125 - protein, 7 aa, synthetic construct
  REGION 1..7: note = h15H3 VL CDR2
LVSELDS

SEQ ID NO: 126 - protein, 9 aa, synthetic construct
  REGION 1..9: note = h15H3 VL CDR3
WQGTHFPRT

SEQ ID NO: 127 - protein, 119 aa, synthetic construct; identical to SEQ ID NO: 43
  REGION 1..119: note = SGN-CD228A heavy chain variable region

SEQ ID NO: 128 - protein, 112 aa, synthetic construct; identical to SEQ ID NO: 44
  REGION 1..112: note = SGN-CD228A light chain variable region

SEQ ID NO: 129 - protein, 5 aa, synthetic construct
  REGION 1..5: note = SGN-CD228A VH CDR1
SGYWN

SEQ ID NO: 130 - protein, 16 aa, synthetic construct
  REGION 1..16: note = SGN-CD228A VH CDR2
YISDSGITYYNPSLKS

SEQ ID NO: 131 - protein, 11 aa, synthetic construct
  REGION 1..11: note = SGN-CD228A VH CDR3
RTLATYYAMDY

SEQ ID NO: 132 - protein, 16 aa, synthetic construct
  REGION 1..16: note = SGN-CD228A VL CDR1
RASQSLVHSDGNTYLH

SEQ ID NO: 133 - protein, 7 aa, synthetic construct
  REGION 1..7: note = SGN-CD228A VL CDR2
RVSNRFS

SEQ ID NO: 134 - protein, 9 aa, synthetic construct
  REGION 1..9: note = SGN-CD228A VL CDR3
SQSTHVPPT

SEQ ID NO: 135 - protein, 120 aa, synthetic construct
  REGION 1..120: note = SGN-LIV1A Ladiratuzumab Vedotin (LV) heavy chain variable region
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 136 - protein, 112 aa, synthetic construct
  REGION 1..112: note = SGN-LIV1A Ladiratuzumab Vedotin (LV) light chain variable region
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIK

SEQ ID NO: 137 - protein, 5 aa, synthetic construct
  REGION 1..5: note = SGN-LIV1A Ladiratuzumab Vedotin (LV) VH CDR1
DYYMH

SEQ ID NO: 138 - protein, 17 aa, synthetic construct
  REGION 1..17: note = SGN-LIV1A Ladiratuzumab Vedotin (LV) VH CDR2
WIDPENGDTEYGPKFQG

SEQ ID NO: 139 - protein, 11 aa, synthetic construct
  REGION 1..11: note = SGN-LIV1A Ladiratuzumab Vedotin (LV) VH CDR3
HNAHYGTWFAY

SEQ ID NO: 140 - protein, 16 aa, synthetic construct
  REGION 1..16: note = SGN-LIV1A Ladiratuzumab Vedotin (LV) VL CDR1
RSSQSLLHSSGNTYLE

SEQ ID NO: 141 - protein, 7 aa, synthetic construct
  REGION 1..7: note = SGN-LIV1A Ladiratuzumab Vedotin (LV) VL CDR2
KISTRFS

SEQ ID NO: 142 - protein, 9 aa, synthetic construct
  REGION 1..9: note = SGN-LIV1A Ladiratuzumab Vedotin (LV) VL CDR3
FQGSHVPYT

SEQ ID NO: 143 - protein, 118 aa, synthetic construct
  REGION 1..118: note = Tisotumab Vedotin (TV) heavy chain variable region
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 144 - protein, 107 aa, synthetic construct
  REGION 1..107: note = Tisotumab Vedotin (TV) light chain variable region
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 145 - protein, 8 aa, synthetic construct
  REGION 1..8: note = Tisotumab Vedotin (TV) VH CDR1
GFTFSNYA

SEQ ID NO: 146 - protein, 8 aa, synthetic construct
  REGION 1..8: note = Tisotumab Vedotin (TV) VH CDR2
ISGSGDYT

SEQ ID NO: 147 - protein, 11 aa, synthetic construct
  REGION 1..11: note = Tisotumab Vedotin (TV) VH CDR3
ARSPWGYYLDS

SEQ ID NO: 148 - protein, 6 aa, synthetic construct
  REGION 1..6: note = Tisotumab Vedotin (TV) VL CDR1
QGISSR

SEQ ID NO: 149 - skipped

SEQ ID NO: 150 - protein, 9 aa, synthetic construct
  REGION 1..9: note = Tisotumab Vedotin (TV) VL CDR3
QQYNSYPYT

(19) United States
(12) Patent Application Publication
(10) Pub. No.: US 2023/0270877 A1
(43) Pub. Date: Aug. 31, 2023

(54) Combination Therapy

(71) Applicant: Seagen Inc., Bothell, WA (US)

(72) Inventors: Shyra Gardai, Monroe, WA (US); Alyson Smith, Monroe, WA (US); Kerry Klussman, Bothell, WA (US); Bernard Liu, Bothell, WA (US); Heather Van Epps, Bothell, WA (US)

(73) Assignee: Seagen Inc., Bothell, WA (US)

(21) Appl. No.: 18/309,388

(22) Filed: Apr. 28, 2023

Related U.S. Application Data
(63) Continuation of application No. PCT/US2021/058208, filed on Nov. 5, 2021.
(60) Provisional application No. 63/208,179, filed on Jun. 8, 2021.
(60) Provisional application No. 63/174,411, filed on Apr. 13, 2021.
(60) Provisional application No. 63/111,045, filed on Nov. 8, 2020.

(51) Int. Cl. A61K 47/68; A61K 39/395; A61P 35/00

(52) U.S. Cl. CPC A61K 47/6851; A61K 39/3955; A61K 47/6817; A61K 47/6889; A61K 47/6829; A61P 35/00

(57) ABSTRACT

Provided herein are methods of treating cancer with an antibody that binds an immune cell engager in combination with an antibody-drug conjugate.

### CROSS-REFERENCE TO RELATED APPLICATIONS

This application is a continuation of International Application No. PCT/US2021/058208, filed Nov. 5, 2021, which claims the benefit of priority of U.S. Provisional Application No. 63/111,045, filed Nov. 8, 2020, U.S. Provisional Application No. 63/172,411, filed Apr. 8, 2021, and U.S. Provisional Application No. 63/208,179, filed Jun. 8, 2021, each of which is incorporated by reference herein in its entirety for any purpose.

### SEQUENCE LISTING

The present application contains a Sequence Listing, which has been submitted electronically in XML format. Said XML copy, created on Mar. 29, 2023, is named “2023-03-29_01218-0027-00PCT_ST26.xml” and is 196,984 bytes in size. The information in the Sequence Listing is incorporated herein by reference in its entirety.

### FIELD

Provided herein are methods of treating cancer with an antibody that binds an immune cell engager in combination with an antibody-drug conjugate.

### BACKGROUND

Immuno-oncology therapeutics and antibody-drug conjugates (ADCs) have been used to treat cancer in patients. Neither class of therapeutics has been able to treat the full complement of patients in targeted indications. This disclosure solves this and other problems.

### BRIEF SUMMARY

Embodiment 1. A method of treating cancer, comprising administering to a subject with cancer (1) an antibody-drug conjugate (ADC) that comprises a first antibody that binds a tumor-associated antigen and a cytotoxic agent, wherein the cytotoxic agent is a tubulin disrupter; and (2) a second antibody that binds to an immune cell engager, wherein the second antibody comprises an Fc with enhanced binding to one or more activating FcγRs, wherein the activating FcγRs include one or more of FcγRIIIa, FcγRIIa, and/or FcγRI.

Embodiment 2. The method of embodiment 1, wherein the second antibody comprises an Fc with enhanced binding to at least FcγRIIIa.

Embodiment 3. The method of embodiment 1, wherein second antibody comprises an Fc with enhanced binding to at least FcγRIIIa and FcγRIIa.

Embodiment 4. The method of embodiment 1, wherein the second antibody comprises an Fc with enhanced binding to at least FcγRIIIa and FcγRI.

Embodiment 5. The method of embodiment 1, wherein the second antibody comprises an Fc with enhanced binding to FcγRIIIa, FcγRIIa, and FcγRI.

Embodiment 6. The method of any one of embodiments 1-5, wherein the Fc of the second antibody has reduced binding to one or more inhibitory FcγRs.

Embodiment 7. The method of embodiment 6, wherein the Fc of the second antibody has reduced binding to FcγRIIb.

Embodiment 8. The method of any one of embodiments 1-7, wherein the Fc of the second antibody has reduced fucose levels and/or has been engineered to comprise one or more mutations such that the Fc has enhanced binding to the one or more activating FcγRs.

Embodiment 9. The method of embodiment 8, wherein the second antibody is nonfucosylated.

Embodiment 10. The method of embodiment 8, wherein the second antibody comprises substitutions S293D, A330L, and 1332E in the heavy chain constant region.

Embodiment 11. A method of treating cancer, comprising administering to a subject with cancer an antibody-drug conjugate, wherein the antibody-drug conjugate comprises a first antibody conjugated to a cytotoxic agent, wherein the cytotoxic agent is a tubulin disrupter; and a second antibody that binds an immune cell engager, wherein the second antibody is nonfucosylated.

Embodiment 12. The method of any one of embodiments 1-11, wherein the first antibody binds a tumor-associated antigen.

Embodiment 13. A method of treating cancer, comprising administering to a subject with cancer (1) an antibody-drug conjugate (ADC), wherein the ADC comprises a first antibody that binds a tumor-associated antigen and a cytotoxic agent, wherein the cytotoxic agent is a tubulin disrupter, and (2) a second antibody that binds an immune cell engager, wherein the second antibody comprises an Fc with enhanced ADCC activity relative to a corresponding wild-type Fc of the same isotype.

Embodiment 14. The method of embodiment 13, wherein the second antibody comprises an Fc with enhanced ADCC and ADCP activity relative to a corresponding wild-type Fc of the same isotype.

Embodiment 15. The method of embodiment 13 or 14, wherein the second antibody is nonfucosylated.

Embodiment 16. The method of any one of embodiments 13-15, wherein the second antibody comprises an Fc with enhanced binding to one or more activating FcγRs, wherein the activating FcγRs include one or more of FcγRIIIa, FcγRIIa, and/or FcγRI.

Embodiment 17. The method of embodiment 16, wherein the second antibody comprise an Fc with enhanced binding to at least FcγRIIIa.

Embodiment 18. The method of embodiment 16, wherein second antibody comprises an Fc with enhanced binding to at least FcγRIIIa and FcγRIIa.

Embodiment 19. The method of embodiment 16, wherein the second antibody comprises an Fc with enhanced binding to at least FcγRIIIa and FcγRI.

Embodiment 20. The method of embodiment 16, wherein the second antibody comprises an Fc with enhanced binding to FcγRIIIa, FcγRIIa, and FcγRI.

Embodiment 21. The method of any one of embodiments 13-20, wherein the Fc of the second antibody has reduced binding to one or more inhibitory FcγRs.

Embodiment 22. The method of embodiment 21, wherein the Fc of the second antibody has reduced binding to FcγRIIb.

Embodiment 23. The method of any one of embodiments 1-22, wherein the first antibody binds an antigen selected from 5T4 (TPBG), ADAM-9, AG-7, ALK, ALP, AM4HRII, APLP2, ASCT2, AVB6, AXL (UFO), B7-H3 (CD276), B7-H4, BCMA, C3a, C3b, C4.4a (LYPD3), C5, C5a, CA6, CA9, CanAg, carbonic anhydrase IX (CAIX), Cathepsin D, CCR7, CD1, CD10, CD100, CD101, CD102, CD103, CD104, CD105, CD106, CD107a, CD107b, CD108, CD109, CD111, CD112, CD113, CD116, CD117, CD118, CD119, CD11A, CD11b, CD11c, CD120a, CD121a, CD121b, CD122, CD123, CD124, CD125, CD126, CD127, CD13, CD130, CD131, CD132, CD133, CD135, CD136, CD137, CD138, CD14, CD140a, CD140b, CD141, CD142, CD143, CD144, CD146, CD147, CD148, CD15, CD150, CD151, CD154, CD155, CD156a, CD156b, CD156c, CD157, CD158b2, CD158e, CD158f1, CD158h, CD158i, CD159a, CD16, CD160, CD161, CD162, CD163, CD164, CD166, CD167b, CD169, CD16a, CD16b, CD170, CD171, CD172a, CD172b, CD172g, CD18, CD180, CD181, CD183, CD184, CD185, CD19, CD194, CD197, CD1a, CD1b, CD1c, CD1d, CD2, CD20, CD200, CD201, CD202b, CD203c, CD204, CD205, CD206, CD208, CD21, CD213al, CD213a2, CD217, CD218a, CD22, CD220, CD221, CD222, CD224, CD226, CD228, CD229, CD23, CD230, CD232, CD239, CD243, CD244, CD248, CD249, CD25, CD26, CD265, CD267, CD269, CD27, CD272, CD273, CD274, CD275, CD279, CD28, CD280, CD281, CD282, CD283, CD284, CD289, CD29, CD294, CD295, CD298, CD3, CD3 epsilon, CD30, CD300f, CD302, CD304, CD305, CD307, CD31, CD312, CD315, CD316, CD317, CD318, CD319, CD32, CD321, CD322, CD324, CD325, CD326, CD327, CD328, CD32b, CD33, CD331, CD332, CD333, CD334, CD337, CD339, CD34, CD340, CD344, CD35, CD352, CD36, CD37, CD38, CD39, CD3d, CD3g, CD4, CD41, CD42d, CD44, CD44v6, CD45, CD46, CD47, CD48, CD49a, CD49b, CD49c, CD49d, CD49e, CD49f, CD5, CD50, CD51, CD51 (integrin alpha-V), CD52, CD53, CD54, CD55, CD56, CD58, CD59, CD6, CD61, CD62L, CD62P, CD63, CD64, CD66a-e, CD67, CD68, CD69, CD7, CD70, CD70L, CD71, CD71 (TfR), CD72, CD73, CD74, CD79a, CD79b, CD8, CD80, CD82, CD83, CD84, CD85f, CD85i, CD85j, CD86, CD87, CD89, CD90, CD91, CD92, CD95, CD96, CD97, CD98, CDH6, CDH6 (cadherin 6), CDw210a, CDw210b, CEA, CEACAM5, CEACAM6, CFC1B, cKIT, CLDN18.2 (claudin 18.2), CLDN6, CLDN9, CLL-1, c-MET, complement factors C3, Cripto, CSP-1, CXCR5, DCLK1, DLK-1, DLL3, DPEP3, DR5 (Death receptor 5), Dysadherin, EFNA4, EGFR, EGFR wild type, EGFRviii, EGP-1 (TROP-2), EGP-2, EMP2, ENPP3, EpCAM, EphA2, EphA3, Ephrin-A4 (EFNA4), ETBR, FAP, FcRH5, FGFR2, FGFR3, FLT3, FOLR, FOLR1, FOLR-alpha, FSH, GCC, GD2, GD3, globo H, GPC1, GPC-1, GPC3, GPNMB, GPR20, HER2, HER-2, HER3, HER-3, HGFR (c-Met), HLA-DR, HM1.24, HSP90, Ia, IGF-1R, IL-13R, IL-15, IL1RAP, IL-2, IL-3, IL-4, IL7R, integrin alphaVbeta3 (integrin αVPβ3), integrin beta-6, Interleukin-4 Receptor (IL4R), KAAG-1, KLK2, LAMP-1, Le(y), Lewis Y antigen, LGALS3BP, LGR5, LH/hCG, LHRH, Lipid raft, LIV-1 (SLC39A6 or ZIP6), LRP-1, LRRC15, LY6E, Macrophage mannose receptor 1, MAGE, Mesothelin (MSLN), MET, MHC class I chain-related protein A and B (MICA and MICB), MN/CA IX, MRC2, MT1-MMP, MTX3, MTX5, MUC1, MUC16, MUC2, MUC3, MUC4, MUC5, MUC5ac, NaPi2b, NCA-90, NCA-95, Nectin-4, Notch3, Nucleolin, OAcGD2, OT-MUC1 (onco-tethered-MUC1), OX001L, P1GF, PAM4 antigen, p-cadherin (cadherin 3), PD-L1, Phosphatidyl Serine(PS), PRLR, Prolactin Receptor (PRLR), Pseudomonas, PSMA, PTK4, PTK7, Receptor tyrosine kinase (RTK), RNF43, ROR1, ROR2, SAIL, SEZ6, SLAMF7, SLC44A4, SLITRK6, SLMAMF7 (CS1), SLTRK6, Sortilin (SORT1), SSEA-4, SSTR2, Staphylococcus aureus (antibiotic agent), STEAP-1, STING, STn, T101, TAA, TAC, TDGF1, tenascin, TENB2, TGF-B, Thomson-Friedenreich antigens, Thy1.1, TIM-1, tissue factor (TF; CD142), TM4SF1, Tn antigen, TNF-alpha (TNFα), TRA-1-60, TRAIL receptor (R1 and R2), TROP-2, Tumor-associated glycoprotein 72 (TAG-72), uPAR, VEGFR, VEGFR-2, and xCT.

Embodiment 24. The method of any one of embodiments 1-23, wherein the first antibody does not bind Nectin-4.

Embodiment 25. The method of any one of embodiments 1-24, wherein the method does not comprise administering an antibody-drug conjugate comprising an antibody that binds Nectin-4.

Embodiment 26. The method of any one of embodiments 1-25, wherein the first antibody binds an antigen selected from CD71, Ax1, AMHRII, and LGR5, Ax1, CA9, CD142, CD20, CD22, CD228, CD248, CD30, CD33, CD37, CD48, CD7, CD71, CD79b, CLDN18.2, CLDN6, c-MET, EGFR, EphA2, ETBR, FCRH5, GCC, Globo H, gpNMB, HER-2, IL7R, Integrin beta-6, KAAG-1, LGR5, LIV-1, LRRC15, Ly6E, Mesothelin (MSLN), MET, MRC2, MUC16, NaPi2b, Nectin-4, OT-MUC1 (onco-tethered-MUC1), PSMA, ROR1, SLAMF7, SLC44A4, SLITRK6, STEAP-1, STn, TIM-1, TRA-1-60, and Tumor-associated glycoprotein 72 (TAG-72).

Embodiment 27. The method of any one of embodiments 1-25, wherein the first antibody binds an antigen selected from BCMA, GPC1, CD30, cMET, SAIL, HER3, CD70, CD46, CD48, HER2, 5T4, ENPP3, CD19, EGFR, and EphA2.

Embodiment 28. The method of any one of embodiments 1-25, wherein the first antibody binds an antigen selected from Her2, TROP2, BCMA, cMet, integrin alphVbeta6 (integrin aVP6), CD22, CD79b, CD30, CD19, CD70, CD228, CD47, and CD48.

Embodiment 29. The method of any one of embodiments 1-25, wherein the first antibody binds an antigen selected from CD142, Integrin beta-6, integrin alphaVbeta6, ENPP3, CD19, Ly6E, cMET, C4.4a, CD37, MUC16, STEAP-1, LRRC15, SLITRK6, ETBR, FCRH5, Ax1, EGFR, CD79b, BCMA, CD70, PSMA, CD79b, CD228, CD48, LIV-1, EphA2, SLC44A4, CD30, and sTn.

Embodiment 30. The method of any one of embodiments 1-26, wherein the tubulin disrupter is an auristatins, a tubulysin, a colchicine, a vinca alkaloid, a taxane, a cryptophycin, a maytansinoid, or a hemiasterlin.

Embodiment 31. The method of embodiment 30, wherein the tubulin disrupter is an auristatin.

Embodiment 32. The method of any one of embodiments 1-31, wherein tubulin disrupter is dolostatin-10, MMAE (N-methylvaline-valine-dolaisoleuine-dolaproine-norephedrine), MMAF (N-methylvaline-valine-dolaisoleuine-dolaproine-phenylalanine), auristatin F, AEB, AEVB, or AFP (auristatin phenylalanine phenylenediamine).

Embodiment 33. The method of any one of embodiments 1-32, wherein the tubulin disrupter is MMAE.

Embodiment 33-1. The method of embodiment 33, wherein the antibody-drug conjugate comprises MMAE and is selected from: DP303c, also known as SYSA1501, targeting HER-2 (CSPC Pharmaceutical; Dophen Biomed), SIA01-ADC, also known as ST1, targeting STn (Siamab Therapeutics), Ladiratuzumab vedotin, also known as SGN-LIV1A, targeting LIV-1 (Merck & Co., Inc.; Seagen (Seattle Genetics) Inc.), ABBV-085, also known as Samrotamab vedotin, targeting LRRC15 (Abbvie; Seagen (Seattle Genetics) Inc.), DMOT4039A, also known as RG7600; αMSLN-MMAE, targeting Mesothelin (MSLN) (Roche-Genentech), RC68, also known as Remegen EGFR ADC, targeting EGFR (RemeGen (Rongchang Biopharmaceutical (Yantai) Co., Ltd.)), RC108, also known as RC108-ADC, targeting c-MET (RemeGen (Rongchang Biopharmaceutical (Yantai) Co., Ltd.)), CMG901, also known as MRG005, targeting CLDN18.2 (Keymed Biosciences; Lepu biotech; Shanghai Miracogen Inc. (Shanghai Meiya Biotechnology Co., Ltd)), YBL-001, also known as LCB67, targeting DLK-1 (Lego Chem Biosciences; Pyxis Oncology; Y-Biologics), DCDS0780A, also known as Iladatuzumab vedotin; RG7986, targeting CD79b (Roche-Genentech; Seagen (Seattle Genetics) Inc.), Tisotumab vedotin, also known as Humax-TF-ADC; tf-011-mmae; TIVDAK™ targeting CD142 (GenMab; Seagen (Seattle Genetics) Inc.), GO-3D1-ADC, also known as humAb-3D1-MMAE ADC, targeting MUC1-C(Genus Oncology LLC), ALT-P7, also known as HM2-MMAE, targeting HER-2 (Alteogen, Inc.; Levena Biopharma; 3SBio, Inc.), Vandortuzumab vedotin, also known as DSTP3086S; RG7450, targeting STEAP-1 (Roche-Genentech; Seagen (Seattle Genetics) Inc.), Lifastuzumab Vedotin, also known as DNIB0600A; NaPi2b ADC; RG7599, targeting NaPi2b (Roche-Genentech), Sofituzumab vedotin, also known as DMUC5754A; RG7458, targeting MUC16 (Seagen (Seattle Genetics) Inc.; Roche-Genentech), RG7841, also known as DLYE5953A, targeting Ly6E (Roche-Genentech; Seagen (Seattle Genetics) Inc.), RG7598, also known as DFRF4539A, targeting FCRH5 (Roche-Genentech; Seagen (Seattle Genetics) Inc.), RG7636, also known as DEDN6526A, targeting ETBR (Seagen (Seattle Genetics) Inc.; Roche-Genentech), Pinatuzumab vedotin, also known as DCDT2980S; RG7593, targeting CD22 (Roche-Genentech), Polatuzumab vedotin, also known as DCDS4501A; POLIVY™; RG7596; RO-5541077, targeting CD79b (Chugai Pharmaceutical; Roche-Genentech; Seagen (Seattle Genetics) Inc.), DMUC4064A, also known as D-4064a; RG7882, targeting MUC16 (Roche-Genentech; Seagen (Seattle Genetics) Inc.), SYSA1801, also known as CPO102, targeting CLDN18.2 (Conjupro Biotherapeutics Inc.; CSPC ZhongQi Pharmaceutical Technology Co.), RC 118, also known as Claudin18.2- ADC; YH005, targeting CLDN18.2 (RemeGen (Rongchang Biopharmaceutical (Yantai) Co., Ltd.); Biocytogen), VLS-101, also known as Cirmtuzumab vedotin; MK-2140; UC-961ADC3; Zilovertamab Vedotin, targeting ROR1 (VelosBio. Inc), Glembatumumab vedotin, also known as CDX-011; CR011-vcMMAE, targeting gpNMB (Celldex Therapeutics), BA3021, also known as CAB-ROR2-ADC; Ozuriftamab Vedotin, targeting ROR2 (Bioatla; Himalaya Therapeutics), BA3011, also known as CAB-AXL-ADC; Mecbotamab Vedotin, targeting Ax1 (Bioatla; Himalaya Therapeutics), CM-09, also known as Bstrongximab-ADC, targeting TRA-1-60 (CureMeta), ABBV-838, also known as Azintuxizumab vedotin, targeting SLAMF7 (Abbvie), Enapotamab vedotin, also known as AXL-107-MMAE; HuMax-AXL-ADC, targeting Axl (GenMab; Seagen (Seattle Genetics) Inc.), ARC-01, also known as anti-CD79b ADC, targeting CD79b (Araris Biotech AG), Disitamab vedotin, also known as Aidexi®; RC48, targeting HER-2 (RemeGen (Rongchang Biopharmaceutical (Yantai) Co., Ltd.); Seagen (Seattle Genetics) Inc.), ASG-5ME, also known as AGS-5; AGS-5ME, targeting SLC44A4 (Agensys, Inc.; Astellas Pharma Inc.; Seagen (Seattle Genetics) Inc.), Enfortumab vedotin, also known as AGS-22M6E; ASG-22CE; ASG-22ME; PADCEV™, targeting Nectin-4 (Astellas Pharma Inc.; Seagen (Seattle Genetics) Inc.), ASG-15ME, also known as AGS-15E; Sirtratumab vedotin, targeting SLITRK6 (Seagen (Seattle Genetics) Inc.; Astellas Pharma Inc.), Brentuximab vedotin, also known as Adcetris; cAC10-vcMMAE; SGN-35, targeting CD30 (Seagen (Seattle Genetics) Inc.; Takeda), Telisotuzumab vedotin, also known as ABBV-399, targeting c-MET (Abbvie), Losatuxizumab vedotin, also known as ABBV-221, targeting EGFR (Abbvie), CX-2029, also known as ABBV-2029, targeting CD71 (Abbvie; CytomX Therapeutics), AB-3A4-ADC, also known as AB-3A4-vcMMAE, targeting KAAG-1 (Alethia Biotherapeutics), Indusatumab vedotin, also known as 5F9-vcMMAE; MLN0264; TAK-264, targeting GCC (Takeda; Millennium Pharmaceuticals, Inc), FOR46 targeting CD46 (Fortis Therapeutics, Inc.), LR004-VC-MMAE targeting EGFR (Chinese Academy of Medical Sciences Peking Union Medical College Hospital), CD30-ADCs targeting CD30 (NBE Therapeutics; Boehringer Ingelheim), Anti-endosialin-MC—VC-PABC-MMAE targeting CD248 (Genzyme), OBI-998 targeting SSEA-4 (OBI Pharma), MRG002 targeting HER-2 (Lepu biotech; Shanghai Miracogen Inc. (Shanghai Meiya Biotechnology Co., Ltd)), TRS005 targeting CD20 (Teruisi Pharmaceuticals), Oba01 targeting DR5 (Death receptor 5) (Obio Technology (Shanghai) Corp., Ltd.; Yantai Obioadc Biomedical Technology Ltd.), PSMA ADC targeting PSMA (Progenics Pharmaceuticals, Inc; Seagen (Seattle Genetics) Inc.), SGN-CD48A targeting CD48 (Seagen (Seattle Genetics) Inc.), IMAB362-vcMMAE targeting CLDN18.2 (Astellas Pharma Inc.; Ganymed), GB251 targeting HER-2 (Genor Biopharma Co., Ltd.), Innate Pharma BTG-ADCs targeting CD30 (Innate Pharma; Sanofi), ADCendo uPARAP ADC targeting MRC2 (ADCendo), XCN-010 targeting actM (Xiconic Pharmaceuticals, LLC), ANT-043 targeting HER-2 (Antikor Biopharma), OBI-999 targeting Globo H (Abzena; OBI Pharma), LY3343544 targeting MET (Eli Lilly and Company), Tagworks anti-TAG72 ADC targeting TAG-72 (Tagworks Pharmaceuticals), IMAB027-vcMMAE targeting CLDN6 (Ganymed; Astellas Pharma Inc.), LGR5-ADC targeting LGR5 (Genentech, Inc.), Philochem B12-MMAE ADC targeting IL-7R (Instituto de Medicina Molecular João Lobo Antunes; Philochem AG), TE-1522 targeting CD19 (Immunwork), SGN-STNV targeting STn (Seagen (Seattle Genetics) Inc.), HTI-1511 targeting EGFR (Abzena; Halozyme Therapeutics), Peptron PAb001-ADC targeting OT-MUC1 (onco-tethered-MUC1) (Peptron; Qilu Pharmaceutical co. Ltd.), LM-102 targeting CLDN18.2 (LaNova Medicines Limited), Anwita Biosciences MSLN-MMAE targeting Mesothelin (MSLN) (Anwita biosciences), SGN-CD228A targeting CD228 (Seagen (Seattle Genetics) Inc.), NBT828 targeting HER-2 (NewBio Therapeutics; Genor Biopharma Co., Ltd.), Gamamabs GM103 targeting AMHR2 (GamaMabs Pharma; Exelixis), LCB14-0302 targeting HER-2 (Lego Chem Biosciences), BAY79-4620 targeting carbonic anhydrase IX (CAIX) (Bayer; MorphoSys), NBT508 targeting CD79b (NewBio Therapeutics), PAT-DX3-MMAE targeting Undisclosed (Patrys; Yale University), AGS67E targeting CD37 (Astellas Pharma Inc.; Seagen (Seattle Genetics) Inc.), CDX-014 targeting TIM-1 (Celldex Therapeutics), BVX001 targeting CD33; CD7 (Bivictrix therapeutics), SGN-B6A targeting Integrin beta-6 (Seagen (Seattle Genetics) Inc.), MRG003 targeting EGFR (Lepu biotech; Shanghai Miracogen Inc. (Shanghai Meiya Biotechnology Co., Ltd)), and PYX-202 targeting DLK-1 (Pyxis Oncology; Lego Chem Biosciences).

Embodiment 34. The method of embodiment 33, wherein the MMAE is conjugated to the first antibody through a linker that comprises valine and citrulline.

Embodiment 35. The method of embodiment 34, wherein the linker-MMAE is vcMMAE.

Embodiment 36. The method of embodiment 33, wherein the MMAE is conjugated to the first antibody through a linker that comprises leucine, alanine, and glutamic acid.

Embodiment 37. The method of embodiment 36, wherein the linker-MMAE is dLAE-MMAE.

Embodiment 38. The method of any one of embodiments 1-32, wherein the tubulin disrupter is MMAF.

Embodiment 38-1. The method of embodiment 38, wherein the antibody-drug conjugate comprises MMAF and is selected from: CD70-ADC targeting CD70 (Kochi University; Osaka University), IGN786 targeting SAIL (AstraZeneca; Igenica Biotherapeutics), PF-06263507 targeting 5T4 (Pfizer), GPC1-ADC targeting GPC-1 (Kochi University), ADC-AVP10 targeting CD30 (Avipep), M290-MC-MMAF targeting CD103 (The Second Affiliated Hospital of Harbin Medical University), BVX001 targeting CD33; CD7 (Bivictrix therapeutics), Tanabe P3D12-vc-MMAF targeting c-MET (Tanabe Research Laboratories), LILRB4-Targeting ADC targeting LILRB4 (The University of Texas Health Science Center, Houston), TSD101, also known as ABL201, targeting BCMA (TSD Life Science; ABL Bio; Lego Chem Biosciences), Depatuxizumab mafodotin, also known as ABT-414, targeting EGFR (Abbvie; Seagen (Seattle Genetics) Inc.), AGS16F, also known as AGS-16C3F; AGS-16M8F, targeting ENPP3 (Astellas Pharma Inc.; Seagen (Seattle Genetics) Inc.), AVG-A11 BCMA ADC, also known as AVG-A11-mcMMAF, targeting BCMA (Avantgen), Belantamab mafodotin, also known as BLENREP; GSK2857916; J6MO-mcMMAF, targeting BCMA (GlaxoSmithKline; Seagen (Seattle Genetics) Inc.), MP-HER3-ADC, also known as HER3-ADC, targeting HER-3 (MediaPharma), FS-1502, also known as LCB14-0110, targeting HER-2 (Lego Chem Biosciences; Shanghai Fosun Pharmaceutical Development Co, Ltd.), MEDI-547, also known as MI-CP177, targeting EphA2 (AstraZeneca; Seagen (Seattle Genetics) Inc.), Vorsetuzumab mafodotin, also known as SGN-75, targeting CD70 (Seagen (Seattle Genetics) Inc.), Denintuzumab mafodotin, also known as SGN-CD19A, targeting CD19 (Seagen (Seattle Genetics) Inc.), and HTI-1066, also known as SHR-A1403, targeting c-MET (Jiangsu HengRui Medicine Co., Ltd).

Embodiment 39. The method of embodiment any one of embodiments 1-30, wherein the tubulin disrupter is a tubulysin.

Embodiment 40. The method of embodiment 39, wherein the tubulysin is selected from tubulysin D, tubulysin M, tubuphenylalanine, and tubutyrosine.

Embodiment 41. The method of any one of embodiments 1-32, wherein the antibody-drug conjugate is selected from AbGn-107 (Ab1-18Hr1), AGS62P1 (ASP1235), ALT-P7 (HM2-MMAE), BA3011 (CAB-AXL-ADC), belantamab mafodotin, brentuximab vedotin, cirmtuzumab vedotin (VLS-101, UC-961ADC3), cofetuzumab pelidotin (PF-06647020, PTK7-ADC, PF-7020, ABBV-647), CX-2029 (ABBV-2029), disitamab vedotin (RC48), enapotamab vedotin (HuMax-AXL-ADC, AXL-107-MMAE), enfortumab vedotin (EV), FS-1502 (LCB14-0110), gemtuzumab ozogamicin, HTI-1066 (SHR-A1403), inotuzumab ozogamicin, PF-06804103 (NG-HER2 ADC), polatuzumab vedotin, sacituzumab govitecan, SGN-B6A, SGN-CD228A, SGN-STNV, STI-6129 (CD38 ADC, LNDS1001, CD38-077 ADC), telisotuzumab vedotin (ABBV-399), tisotumab vedotin (Humax-TF-ADC, tf-011-mmae, TV), trastuzumab deruxtecan, trastuzumab emtansine, and vorsetuzumab mafodotin.

Embodiment 42. The method of any one of embodiments 1-41, 33-1, and 38-1, wherein the first antibody is an anti-claudin-18.2 antibody that comprises a heavy chain CDR1, CDR2, and CDR3, and a light chain CDR1, CDR2, and CDR3 respectively comprising the amino acid sequences of SEQ ID NOs:61-66.

Embodiment 43. The method of embodiment 42, wherein the anti-claudin-18.2 antibody comprises a heavy chain variable region (VH) comprising the amino acid sequence of SEQ ID NO:59 and a light chain variable region (VL) comprising the amino acid sequence of SEQ ID NO:60.

Embodiment 44. The method of embodiment 43, wherein the anti-claudin-18.2 antibody is zolbetuximab (175D10).

Embodiment 45. The method of any one of embodiments 1-41, 33-1, and 38-1, wherein the first antibody is an anti-claudin-18.2 antibody that comprises a heavy chain CDR1, CDR2, and CDR3, and a light chain CDR1, CDR2, and CDR3 respectively comprising the amino acid sequences of SEQ ID NOs: 69-74.

Embodiment 46. The method of embodiment 45, wherein the anti-claudin-18.2 antibody comprises a heavy chain variable region (VH) comprising the amino acid sequence of SEQ ID NO:67 and a light chain variable region (VL) comprising the amino acid sequence of SEQ ID NO:68.

Embodiment 47. The method of any one of embodiments 1-41, 33-1, and 38-1, wherein the first antibody is an anti-PD-L1 antibody that comprises a heavy chain CDR1, CDR2, and CDR3, and a light chain CDR1, CDR2, and CDR3 respectively comprising the amino acid sequences of SEQ ID NOs:77-82.

Embodiment 48. The method of embodiment 47, wherein the anti-PD-L1 antibody comprises a heavy chain variable region (VH) comprising the amino acid sequence of SEQ ID NO:75 and a light chain variable region (VL) comprising the amino acid sequence of SEQ ID NO:76.

Embodiment 49. The method of any one of embodiments 1-41, 33-1, and 38-1, wherein the first antibody is an anti-ALP antibody that comprises a heavy chain CDR1, CDR2, and CDR3, and a light chain CDR1, CDR2, and CDR3 respectively comprising the amino acid sequences of SEQ ID NOs:85-90.

Embodiment 50. The method of embodiment 49, wherein the anti-ALP antibody comprises a heavy chain variable region (VH) comprising the amino acid sequence of SEQ ID NO:83 and a light chain variable region (VL) comprising the amino acid sequence of SEQ ID NO:84.

Embodiment 51. The method of any one of embodiments 1-41, 33-1, and 38-1, wherein the first antibody comprises an anti-B7H4 antibody that comprises a heavy chain CDR1, CDR2, and CDR3, and a light chain CDR1, CDR2, and CDR3 respectively comprising the amino acid sequences of SEQ ID NOs:93-98.

Embodiment 52. The method of embodiment 51, wherein the anti-B7H4 antibody comprises a heavy chain variable region (VH) comprising the amino acid sequence of SEQ ID NO:91 and a light chain variable region (VL) comprising the amino acid sequence of SEQ ID NO:92.

Embodiment 53. The method of any one of embodiments 1-41, 33-1, and 38-1, wherein the first antibody is an anti-HER2 antibody that comprises a heavy chain comprising the amino acid sequence of SEQ ID NO:99 and a light chain comprising the amino acid sequence of SEQ ID NO:100.

Embodiment 54. The method of embodiment 53, wherein the antibody-drug conjugate is disitamab vedotin.

Embodiment 55. The method of any one of embodiments 1-41, 33-1, and 38-1, wherein the first antibody is an anti-NaPi2B antibody that comprises a heavy chain comprising the amino acid sequence of SEQ ID NO:101 and a light chain comprising the amino acid sequence of SEQ ID NO:102.

Embodiment 56. The method of embodiment 55, wherein the antibody-drug conjugate is lifastuzumab vedotin.

Embodiment 57. The method of any one of embodiments 1-41, 33-1, and 38-1, wherein the first antibody is an anti-nectin-4 antibody that comprises a heavy chain CDR1, CDR2, and CDR3, and a light chain CDR1, CDR2, and CDR3 respectively comprising the amino acid sequences of SEQ ID NOs:105-110.

Embodiment 58. The method of embodiment 57, wherein the anti-nectin-4 antibody is an antibody that comprises a heavy chain variable region (VH) comprising the amino acid sequence of SEQ ID NO:103 and a light chain variable region (VL) comprising the amino acid sequence of SEQ ID NO:104.

Embodiment 59. The method of embodiment 58, wherein the antibody-drug conjugate is enfortumab vedotin.

Embodiment 60. The method of any one of embodiments 1-41, 33-1, and 38-1, wherein the first antibody is an anti-AVB6 antibody that comprises a heavy chain CDR1, CDR2, and CDR3, and a light chain CDR1, CDR2, and CDR3 respectively comprising the amino acid sequences of SEQ ID NOs:113-118.

Embodiment 61. The method of embodiment 60, wherein the anti-AVB6 antibody comprises a heavy chain variable region (VH) comprising the amino acid sequence of SEQ ID NO:111 and a light chain variable region (VL) comprising the amino acid sequence of SEQ ID NO:112.

Embodiment 62. The method of any one of embodiments 1-41, 33-1, and 38-1, wherein the first antibody is an anti-AVB6 antibody that comprises a heavy chain CDR1, CDR2, and CDR3, and a light chain CDR1, CDR2, and CDR3 respectively comprising the amino acid sequences of SEQ ID NOs:121-126.

Embodiment 63. The method of embodiment 62, wherein the anti-AVB6 antibody comprises a heavy chain variable region (VH) comprising the amino acid sequence of SEQ ID NO:119 and a light chain variable region (VL) comprising the amino acid sequence of SEQ ID NO:120.

Embodiment 64. The method of any one of embodiments 1-41, 33-1, and 38-1, wherein the first antibody is an anti-CD228 antibody that comprises a heavy chain CDR1, CDR2, and CDR3, and a light chain CDR1, CDR2, and CDR3 respectively comprising the amino acid sequences of SEQ ID NOs:129-134.

Embodiment 65. The method of embodiment 64, wherein the anti-CD228 antibody comprises a heavy chain variable region (VH) comprising the amino acid sequence of SEQ ID NO:127 and a light chain variable region (VL) comprising the amino acid sequence of SEQ ID NO:128.

Embodiment 66. The method of any one of embodiments 1-41, 33-1, and 38-1, wherein the first antibody is an anti-LIV-1 antibody that comprises a heavy chain CDR1, CDR2, and CDR3, and a light chain CDR1, CDR2, and CDR3 respectively comprising the amino acid sequences of SEQ ID NOs:137-142.

Embodiment 67. The method of embodiment 66, wherein the anti-LIV-1 antibody comprises a heavy chain variable region (VH) comprising the amino acid sequence of SEQ ID NO:135 and a light chain variable region (VL) comprising the amino acid sequence of SEQ ID NO:136.

Embodiment 68. The method of any one of embodiments 1-41, 33-1, and 38-1, wherein the first antibody is an anti-tissue factor antibody that comprises heavy chain CDR1, CDR2, and CDR3, and a light chain CDR1, CDR2, and CDR3 respectively comprising the amino acid sequences of SEQ ID NOs:145-150.

Embodiment 69. The method of embodiment 68, wherein the anti-tissue factor antibody comprises a heavy chain variable region (VH) comprising the amino acid sequence of SEQ ID NO:143 and a light chain variable region (VL) comprising the amino acid sequence of SEQ ID NO:144.

Embodiment 70. The method of embodiment 69, wherein the antibody-drug conjugate is tisotumab vedotin.

Embodiment 71. The method of any one of embodiments 1-70, 33-1, and 38-1, wherein the second antibody binds an immune cell engager selected from anti-Mullerian Hormone Receptor II (A_MHR2), B7, B7H1, B7H2, B7H3, B7H4, BAFF-R, BCMA (B-cell maturation antigen), Bst1/CD157, C5 complement, CC chemokine receptor 4 (CCR4), CD123, CD137, CD19, CD20, CD25 (IL2RA), CD276, CD278, CD3, CD32, CD33, CD37, CD38, CD4 and HIV-1 gp120-binding sites, CD40, CD70, CD70 (a member of the TNF receptor ligand family), CD80, CD86, Claudin 18.2, c-MET, CSF1R, CTLA-4, EGFR, EGFR MET proto-oncogene, EPHA3, ERBB2, ERBB3, FGFR2b, FLT3, GITR, glucocorticoid-induced TNF receptor (GITR), HER2, HER3, HLA, ICOS, IDO1, IFNAR1, IFNAR2, IGF-1R, IL-3Ralpha (CD123), IL-5R, IL-5Ralpha, LAG-3, MET proto-oncogene, OX40 (CD134), PD-1, PD-L1, PD-L2, PVRIG, respiratory syncytial virus (RSV) heavily glycosylated mucin-like domain of EBOV glycoprotein (GP), Rhesus (Rh) D, sialic acid immunoglobulin-like lectins 8 (Siglec-8), signaling lymphocyte activation molecule (SLAMF7/CS1), T-cell receptor cytotoxic T-lymphocyte-associated antigen 4 (CTLA4), TIGIT, TIM3 (HAVCR2), tumor specific glycoepitope of Muc1 (TA-Muc1), VSIR (VISTA), and VTCN1.

Embodiment 72. The method of any one of embodiments 1-71, 33-1, and 38-1, wherein the second antibody binds TIGIT.

Embodiment 73. The method of embodiment 72, wherein the second antibody comprises: (a) a heavy chain CDR1 comprising an amino acid sequence selected from SEQ ID NOs: 7-9; (b) a heavy chain CDR2 comprising an amino acid sequence selected from SEQ ID NOs: 10-13; (c) a heavy chain CDR3 comprising an amino acid sequence selected from SEQ ID NOs: 14-16; (d) a light chain CDR1 comprising the amino acid sequence of SEQ ID NO: 17; (e) a light chain CDR2 comprising the amino acid sequence of SEQ ID NO: 18; and (f) a light chain CDR3 comprising the amino acid sequence of SEQ ID NO: 19.

Embodiment 74. The method of embodiment 72, wherein the second antibody comprises a heavy chain CDR1, CDR2, and CDR3 and a light chain CDR1, CDR, and CDR3 comprising the sequences of: (a) SEQ ID NOs: 7, 10, 14, 17, 18, and 19, respectively; or (b) SEQ ID NOs: 8, 11, 14, 17, 18, and 19, respectively; or (c) SEQ ID NOs: 9, 12, 15, 17, 18, and 19, respectively; or (d) SEQ ID NOs: 8, 13, 16, 17, 18, and 19, respectively; or (e) SEQ ID NOs: 8, 12, 16, 17, 18, and 19, respectively.

Embodiment 75. The method of embodiment 72, wherein the second antibody comprises a heavy chain variable region comprising an amino acid sequence selected from SEQ ID NOs: 1-5 and a light chain variable region comprising the amino acid sequence of SEQ ID NO: 6.

Embodiment 76. The method of embodiment 72, wherein the second antibody comprises a heavy chain comprising an amino acid sequence selected from SEQ ID NOs: 20-24 and a light chain comprising the amino acid sequence of SEQ ID NO: 25.

Embodiment 77. The method of any one of embodiments 1-71, wherein the second antibody binds CD40.

Embodiment 78. The method of embodiment 77, wherein the second antibody comprises a heavy chain CDR1, CDR2, and CDR3 and a light chain CDR1, CDR, and CDR3 comprising the sequences of: (a) SEQ ID NOs: 30, 31, 32, 33, 34, and 35, respectively; or (b) SEQ ID NOs: 30, 36, 32, 33, 34, and 35, respectively.

Embodiment 79. The method of embodiment 77, wherein the second antibody comprises a heavy chain variable region comprising the amino acid sequence of SEQ ID NO: 28 and a light chain variable region comprising the amino acid sequence of SEQ ID NO: 29.

Embodiment 80. The method of embodiment 77, wherein the second antibody comprises a heavy chain comprising the amino acid sequence of SEQ ID NO: 26 and a light chain comprising the amino acid sequence of SEQ ID NO: 27.

Embodiment 81. The method of any one of embodiments 1-71, 33-1, and 38-1, wherein the second antibody binds CD70.

Embodiment 82. The method of embodiment 81, wherein the second antibody comprises a heavy chain CDR1, CDR2, and CDR3 and a light chain CDR1, CDR, and CDR3 comprising the sequences of SEQ ID NOs: 53-58, respectively.

Embodiment 83. The method of embodiment 81, wherein the second antibody comprises a heavy chain variable region comprising the amino acid sequence of SEQ ID NO: 41 and a light chain variable region comprising the amino acid sequence of SEQ ID NO: 42.

Embodiment 84. The method of any one of embodiments 1-71, 33-1, and 38-1, wherein the second antibody binds BCMA.

Embodiment 85. The method of embodiment 84, wherein the second antibody comprises a heavy chain CDR1, CDR2, and CDR3 and a light chain CDR1, CDR, and CDR3 comprising the sequences of SEQ ID NOs: 47-52, respectively.

Embodiment 86. The method of embodiment 84, wherein the second antibody comprises a heavy chain variable region comprising the amino acid sequence of SEQ ID NO: 45 and a light chain variable region comprising the amino acid sequence of SEQ ID NO: 46.

Embodiment 87. The method of any one of embodiments 1-86, 33-1, and 38-1, wherein the second antibody is an IgG1 or IgG3 antibody.

Embodiment 88. The method of any one of embodiments 1-87, 33-1, and 38-1, wherein the second antibody is comprised in a composition of antibodies, wherein at least 90%, at least 91%, at least 92%, at least 93%, at least 94%, at least 95%, at least 96%, at least 97%, at least 98%, or at least 99% of the antibodies in the composition are nonfucosylated.

Embodiment 89. The method of embodiment 88, wherein each antibody in the composition comprises the same heavy chain and light chain amino acid sequences as the second antibody.

Embodiment 90. The method of any one of embodiments 1-89, 33-1, and 38-1, wherein the Fc of the second antibody has enhanced binding to one or more activating FcγRs as compared to a corresponding wild-type Fc of the same isotype, wherein the activating FcγRs include one or more of FcγRIIIa, FcγRIIa, and/or FcγRI.

Embodiment 91. The method of embodiment 90, wherein the Fe of the second antibody has enhanced binding to FcγRIIIa.

Embodiment 92. The method of any one of embodiments 1-91, 33-1, and 38-1, wherein the Fc of the second antibody has reduced binding to one or more inhibitory FcγRs as compared to a corresponding wild-type Fc of the same isotype.

Embodiment 93. The method of embodiment 92, wherein the Fc of the second antibody has reduced binding to FcγRIIb.

Embodiment 94. The method of any one of embodiments 1-93, 33-1, and 38-1, wherein the Fc of the second antibody has enhanced binding to FcγRIIIa and reduced binding to FcγRIIb.

Embodiment 95. The method of any one of embodiments 1-94, 33-1, and 38-1, wherein the second antibody is a monoclonal antibody.

Embodiment 96. The method of any one of embodiments 1-95, 33-1, and 38-1, wherein the second antibody is a humanized antibody or a human antibody.

Embodiment 97. The method of any one of embodiments 1-96, 33-1, and 38-1, wherein the cancer is bladder cancer, breast cancer, uterine cancer, cervical cancer, ovarian cancer, prostate cancer, testicular cancer, esophageal cancer, gastrointestinal cancer, gastric cancer, pancreatic cancer, colorectal cancer, colon cancer, kidney cancer, clear cell renal carcinoma, head and neck cancer, lung cancer, lung adenocarcinoma, stomach cancer, germ cell cancer, bone cancer, liver cancer, thyroid cancer, skin cancer, melanoma, neoplasm of the central nervous system, mesothelioma, lymphoma, leukemia, chronic lymphocytic leukemia, diffuse large B cell lymphoma, follicular lymphoma, Hodgkin lymphoma, myeloma, or sarcoma.

Embodiment 98. The method of any one of embodiments 1-97, 33-1, and 38-1, wherein the cancer is lymphoma, leukemia, chronic lymphocytic leukemia, diffuse large B cell lymphoma, follicular lymphoma, or Hodgkin lymphoma.

Embodiment 99. The method of any one of embodiments 1-98, 33-1, and 38-1, wherein the antibody-drug conjugate and the second antibody are administered concurrently.

Embodiment 100. The method of embodiment 99, 33-1, and 38-1, wherein the antibody-drug conjugate and the second antibody are administered in a single pharmaceutical composition.

Embodiment 101. The method of any one of embodiments 1-98, 33-1, and 38-1, wherein the antibody-drug conjugate and the second antibody are administered sequentially.

Embodiment 102. The method of embodiment 101, wherein at least a first dose of the antibody-drug conjugate is administered prior to a first dose of the second antibody; or wherein at least a first dose of the second antibody is administered prior to a first dose of the antibody-drug conjugate.

Embodiment 103. The method of any one of embodiments 1-102, 33-1, and 38-1, wherein the second antibody depletes T regulatory cells (Tregs).

Embodiment 104. The method of any one of embodiments 1-103, 33-1, and 38-1, wherein the antibody-drug conjugate induces immune memory against cells expressing the antigen bound by the antibody-drug conjugate.

Embodiment 105. The method of embodiment 104, wherein the induction of immune memory comprises induction of memory T cells.

Embodiment 106. The method of any one of embodiments 1-105, 33-1, and 38-1, wherein the second antibody activates antigen presenting cells (APCs).

Embodiment 107. The method of any one of embodiments 1-106, 33-1, and 38-1, wherein the second antibody enhances CD8 T cell responses.

Embodiment 108. The method of any one of embodiments 1-107, 33-1, and 38-1, wherein the second antibody upregulates co-stimulatory receptors.

Embodiment 109. The method of any one of embodiments 1-108, 33-1, and 38-1, wherein administration of the ADC and the second antibody promotes release of an immune activating cytokine.

Embodiment 110. The method of embodiment 109, wherein the immune activating cytokine is CXCL10 or IFNγ.

Embodiment 111. The method of any one of embodiments 1-110, 33-1, and 38-1, wherein the ADC and the second antibody act synergistically.

Embodiment 112. The method of any one of embodiments 1-111, 33-1, and 38-1, wherein administration of the ADC and the second antibody in combination has a toxicity profile comparable to that of the ADC or the second antibody when either is administered as monotherapy.

Embodiment 113. The method of any one of embodiments 1-112, 33-1, and 38-1, wherein the effective dose of the ADC and/or the second antibody when dosed in combination is less than when administered as monotherapy.

Embodiment 114. The method of any one of embodiments 1-113, 33-1, and 38-1, wherein the cancer has high tumor mutation burden.

Embodiment 115. The method of any one of embodiments 1-114, 33-1, and 38-1, wherein the cancer has microsatellite instability.

### BRIEF DESCRIPTION OF THE DRAWINGS

FIG. 1 shows that non-directed chemotherapeutic agents impair T cell responses.

FIG. 2 shows brentuximab vedotin (BV) treatment of CD30+CD8 T cells. Vedotin ADCs that have directed delivery to T cells do not inhibit proliferation.

FIG. 3A-E show that endoplasmic reticulum (ER) stress induction is superior for auristatin antibody-drug conjugates (ADCs), such as vedotin-based ADCs, as compared to ADCs with different payloads. FIG. 3A shows a table of various clinically approved ADC payloads. FIG. 3B shows a graphic of ER stress signaling response. FIG. 3C shows a Western blot analysis of MIA-PaCa-2 cells treated with ADCs with differing payloads or paclitaxel at IC50 concentrations for 36 or 48 hours. FIG. 3D-E show MIA-PaCa-2 cells expressing CHOP-driven luciferase reporter (Signosis, Inc.) that were treated with ADCs with different payloads over a dose range (FIG. 3D) or at IC50 dose (cytotoxicity) (FIG. 3E). CHOP induction is expressed by fold induction compared to untreated cells.

FIG. 4A-B show immunogenic cell death (ICD) potential of clinical ADC payloads. Supernatants were collected from MIA-PaCa-2 pancreatic tumor cells that were treated with 1 μg/mL ADCs with different payloads for 72 hours. FIG. 4A shows TP released as determined by Cell Titer Glo. FIG. 4B shows HMGB1 secretion as determined by ELISA.

FIG. 5A-C show immune activation assessment of ADC payloads. Upregulation of MHC-Class II (HLA-DR) on myeloid cells within peripheral blood mononuclear cells (PBMC) was assessed by flow cytometry following a 48-hour co-incubation of PBMC with L540cy cells dosed with ADCs with different payloads (24 hours at IC50 concentration). 24-hour supernatants were assessed by Luminex multiplex assay for cytokine levels. FIG. 5A shows immune activation by ADC. FIG. 5B shows MHCII expression on monocytes in response to ADC exposure. FIG. 5C shows innate cytokine CXCL-10/IP10 expression in response to ADC exposure as a measure of immune cell activity.

FIG. 6A-E show payload evaluation on trastuzumab backbone. FIG. 6A shows a table of trastuzumab ADCs that were evaluated. FIG. 6B shows a graphic of ER stress signaling response. FIG. 6C shows a Western blot of BT474 cells treated with ADCs or drug for 72 hours.

FIG. 6D-E show that Vedotin ADC demonstrate strong activation of multiple ICD hallmarks. Trastuzumab ADCs dosed at 1 μg/mL or free MMAE (100 nM) demonstrate different ICD responses in SKBR3 HER2 expressing breast cancer cells. After 48 or 72 hours of treatment, media was collected and used to measure ATP release (FIG. 6D) and HMGB1 levels (FIG. 6E).

FIG. 7A further illustrates the immunogenic cell death (ICD) pathway.

FIG. 7B provides information regarding the payloads of certain ADCs used in FIG. 7B-E.

FIG. 7C-F show INK signaling activation generated in response to treatment with MMAE-ADCs compared to maytansine-ADCs (FIG. 7C), to camptothecin-ADCs (FIG. 7D), to anthracycline-ADCs (FIG. 7E), and calicheamicin-ADCs (FIG. 7F).

FIG. 8A-D show CHOP induction generated in response to treatment with MMAE-ADCs compared to maytansine-ADCs (FIG. 8A), to camptothecin-ADCs (FIG. 8B), to anthracycline-ADCs (FIG. 8C), and to other types of ADCs, including ozogamycin-ADCs, teserine-ADCs, and AT-ADCs (FIG. 8D).

FIG. 9A-D show release of ATP and HMGB1 generated in response to treatment with MMAE-ADCs compared to maytansine-ADCs (FIG. 9A), to camptothecin-ADCs (FIG. 9B), to anthracycline-ADCs (FIG. 9C), and to other types of ADCs, including ozogamycin-ADCs, teserine-ADCs, and AT-ADCs (FIG. 9D).

FIG. 10A-D show MHCII expression and CXCL-10/IP10 release generated in response to treatment with MMAE-ADCs compared to maytansine-ADCs (FIG. 10A), to camptothecin-ADCs (FIG. 10B), to anthracycline-ADCs (FIG. 10C), and to other types of ADCs, including ozogamycin-ADCs and teserine-ADCs (FIG. 10D).

FIG. 10E summarizes the results in FIG. 7B-E, FIG. 8A-D, FIG. 9A-D, and FIG. 10A-D.

FIG. 11A-D show antibody binding to FcγRIIa, FcγRIIb, and FcγRIIIa in Chinese hamster ovary (CHO cells). FIG. 11A shows antibodies assessed. FIG. 11B shows binding to FcγRIIa. FIG. 11C shows binding to FcγRIIIa. FIG. 11D shows binding to FcγRIIb.

FIG. 12A-D show levels of CXCL10 (FIG. 12A), IFNγ (FIG. 12B), IL10 (FIG. 12C), and macrophage derived cytokine (MDC; CCL22) (FIG. 12D) in MIA-PaCa 2 pancreatic cancer cells treated with CD40 agonists and chemotherapy agents.

FIG. 13A-D show levels of CXCL10 (FIG. 13A-B) and IL10 (FIG. 13C-D) in melanoma cell lines treated with CD40 agonists and chemotherapy.

FIG. 14A-C show levels of CXCL14 (FIG. 14A), IL14 (FIG. 14B), and IFNγ (FIG. 14C) in tumor cells from melanoma, lung, breast, and pancreas.

FIG. 15 shows in vivo data for SEA-CD40 Vedotin in combination with ADC chemotherapy combination as assessed by a human CD40 transgenic model where the tumor target antigen was Thy1.1.

FIG. 16 shows CXCL10 levels in tumor cell lines that were treated with an ADC-MMAE directed to a tumor-associated antigen in combination with TIGIT targeted antibodies with various effector function backbones.

FIG. 17 shows IFNγ levels in tumor cell lines that were treated with an ADC-MMAE directed to a tumor-associated antigen in combination with TIGIT targeted antibodies with various effector function backbones.

FIG. 18A-C show in vitro and in vivo data demonstrating the enhanced activity of a nonfucosylated TIGIT antibody and a vc-MMAE ADC (“Vedotin ADC”). FIG. 18A shows that a nonfucosylated TIGIT antibody having an enhanced (nonfucosylated) IgG1 Fc backbone (SEA-TGT) was significantly better at driving immune activation via cytokine IP10 induction as compared to either a corresponding TIGIT antibody with an effector null backbone (LALA) or a standard wildtype IgG1 Fc backbone when these antibodies were co-cultured with tumor cells killed by a targeting vc-MMAE ADC. The combination with SEA-TGT exhibited synergistic immune cell activation. FIG. 18B and FIG. 18C show the anti-tumor response when mice implanted with either CT26 syngeneic tumor cells (FIG. 18B) or Renca syngeneic tumor cells (FIG. 18C) were treated with: 1) a sub-optimal dose of an ADC (Thy1.1 vc-MMAE ADC in FIG. 18B and EphA2 vc-MMAE ADC in FIG. 18C); 2) a series of sub-optimal doses of mIgG2a SEA-TGT (the SEA-TGT antibody reformatted as a nonfucosylated mouse IgG2a that corresponds to a nonfucosylated human IgG1 backbone); or 3) a combination of both agents. As can be seen from these figures, co-administration of these two agents significantly increased treatment efficacy, including generating a high percentage of curative responses in these distinct tumor models.

FIG. 19 shows in vivo data demonstrating the enhanced activity of a nonfucosylated TIGIT antibody (SEA-TGT) and SGN-B7H4 vedotin ADC (B7H4V). FIG. 19 shows the anti-tumor response when mice implanted with Renca syngeneic tumor cells were treated with a subtherapeutic dose of SEA-TGT and a subtherapeutic dose of B7H4V, or a subtherapeutic dose of SEA-TGT and a therapeutic dose of oxaliplatin. As shown in FIG. 19, co-administration of SEA-TGT and B7H4V significantly increased treatment efficacy, even at subtherapeutic doses of each, including generating a high percentage of curative responses.

FIG. 20A-B show combinatorial effects of SEA-CD70, a nonfucosylated anti-CD70 antibody, and SGN-35, an anti-CD30 ADC containing MMAE. FIG. 20A shows in vivo tumor growth evaluation of a non-Hodgkin lymphoma (NHL) xenograft model. FIG. 20B shows Kaplan-Meyer survival evaluation of an NHL xenograft model at a 500 mm³ tumor size endpoint. Tx (treatment) and arrow indicate the treatment starting day (19 days post implantation).

FIG. 21A-B show synergistic effects of SEA-BCMA, a nonfucosylated anti-BCMA antibody, and SGN-CD48A, an anti-CD48 ADC containing MMAE. FIG. 21A shows in vivo survival evaluation of a xenograft model, and FIG. 21B shows in vivo luciferase evaluation of a xenograft model.

FIG. 22A-C show vedotin ADC induces immune cell recruitment and activation in vivo. FIG. 22A: Tumor xenografts isolated from animals treated with a vc-MMAE ADC or non-binding vc-MMAE isotype ADC for 8 days, and subject to flow cytometry or cytokine profiling. FIG. 22B: CD45 positive immune cells were stained for CD11c and activation observed by staining for the expression of MHC-Class II on the cell surface. FIG. 22C: Intratumoral cytokines were measured by Luminex.

FIG. 23A-B show induction of T cell memory by vc-MMAE ADC. In a Renca syngeneic model, mice were cured with a vc-MMAE ADC treatment (FIG. 23A). The mice cured with the treatment were rechallenged with Renca tumor cells, and those mice rejected the subsequently implanted tumor cells (FIG. 23B).

FIG. 24 shows protective anti-tumor immunity conferred by MMAE or vc-MMAE ADC-treated cells. A20 cancer cells were treated with brentuximab vedotin (BV) or MMAE, and the dying and dead cells were administered to mice. FIG. 24 shows that immunized mice displayed stronger immune responses rejecting subsequently implanted A20 cells.

FIG. 25 shows an exemplary model of receptor clustering and agonism by a nonfucosylated anti-CD40 antibody, SEA-CD40; and an exemplary model of receptor agonist and synapse formation by a nonfucosylated anti-TIGIT antibody, SEA-TGT. As shown, the SEA-CD40 antibody can bind CD-40 expressed on antigen presenting cells (APCs) with the Fc portion of the antibody binding to FcγRIIIa expressed on natural killer (NK) cells or on monocytes, which promotes receptor clustering. The SEA-TGT antibody, in contrast, binds to TIGIT expressed on T-cells and the Fc region of the antibody binds to FcγRIIIa expressed on APCs.

### DETAILED DESCRIPTION

I. Introduction

Cell death through apoptosis is a silent, tolerogenic process. However, certain cytotoxic agents, including specific antitumor agents such as anthracyclines, oxaliplatin, or radiation, induce a characteristic form of cell death termed Immunogenic Cell Death (ICD). ICD is a mode of regulated cell death/that generates immune responses of PBMCs and T-cells against the apoptotic cancerous cells. As demonstrated herein, treatment with certain tubulin disrupting agents such as auristatins (e.g., MMAE and MMAF) cause proteins normally found within the ER to become exposed on the cell surface. Increased phagocytic uptake and presentation of tumor antigens to T cells subsequently prime the adaptive immune system. As further shown herein, auristatins such as MMAE and MMAF are distinctly capable of driving ICD induction, thereby enabling the immune system to recognize and mount cytotoxic activity against tumors. In essence, cells dying from ICD serve as a vaccine to stimulate tumor-specific immune responses against any residual disease, or in the event of relapse/recurrence.

As demonstrated by the experimental results described herein, tumor cells undergoing ICD in response to auristatins such as MMAE and MMAF display a unique set of characteristics that potentiate their immunogenicity and apoptosis, including: translocation of calreticulin to the cell surface, secretion of ATP during apoptosis, and release of the nuclear protein HMGB1. ICD induces release of specific MAMPS and danger-associated molecular patterns (DAMPS) which have the unique capability to establish a pro-inflammatory environment that promotes T cell recognition of tumor antigens. As shown herein, while other chemotherapeutic agents can induce apoptosis, not all can induce as robust of an ICD response as auristatins such as MMAE and MMAF.

Key steps of ICD generate a series of signals that activate the innate immune system to recognize tumor cells and clear them. First, a drug induces ER stress. and this in turn results in surface exposure of DAMPs including calreticulin, heat shock proteins (HSP70 and HSP90), secretion of ATP, and release of high mobility group protein B1 (HMGB1). Exposure of these DAMPs and secretion of the immune modulatory agents during the process of ICD can act in concert to initiate immune responses including activation of dendritic cells and other antigen presenting cells, leading to phagocytosis and destruction of the ER-stressed cell.

As noted above, initiation of ICD is linked to ER stress. Overloading the ER's capacity for unfolded polypeptides or disruption of the protein-folding environment initiates ER stress responses. The ER is intimately connected to the microtubule network which provides structure and elasticity through dynamic assembly and contraction. Disruption of microtubule network impinges on the ER network and results in severe ER stress, which triggers expression of the characteristics required for ICD induction and results in a stress response, referred to as the unfolded protein response (UPR). The UPR response has multiple arms which include increased pIRE1, downstream phosphorylation of INK, and ATF4 cleavage.

The present invention is based in part on the finding that certain tubulin disrupting agents such as auristatins (e.g., MMAE and MMAF) are capable of generating an unique ICD response compared with other cytotoxic agents and, in particular, as compared to other payloads that are used on antibody-drug conjugates. The invention is further based on the discovery that pairing the unique ability of such agents to drive ICD with agents that enhance an immune response can amplify anti-tumor activity. This was particularly found to be the case when such immune agonism was achieved using antibodies having the ability to bind certain targets involved in immune signaling and having enhanced Fc binding characteristics and effector function. The desired Fc binding characteristics included activities such as enhanced binding to activating FcγRs, decreased binding to inhibitory FcγRs, enhanced ADCC activity, and/or enhanced ADCP activity. Certain such antibodies with the desired activities were nonfucosylated.

Based upon these collective findings, the present inventors have demonstrated as described in greater detail herein that inducing ICD using particular tubulin disrupters such as MMAE and MMAF in combination with antibodies directed to immune cell engagers that also have enhanced Fc activity results in synergistically improved anti-tumor responses. The use of ADCs rather than standard chemotherapy agents was also shown to mitigate the impairment of T cell responses seen with traditional chemotherapy, thus providing a further advantage to this particular combination approach.

Accordingly, some embodiments provided herein are combination therapies which comprise administering to a subject with cancer: (1) an antibody-drug conjugate comprising a tubulin disrupter conjugated to a first antibody that binds a tumor-associated antigen; and (2) antibody that binds to an immune cell engager, wherein the second antibody comprises an Fc with enhanced binding to one or more activating FcγRs. In some embodiments, the second antibody is nonfucosylated. In certain embodiments, the second antibody has enhanced ADCC and/or ADCP activity.

II. DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention.

As used herein, the singular forms “a”, “an” and “the” include plural referents unless the content clearly dictates otherwise. Thus, for example, reference to “an antibody” optionally includes a combination of two or more such molecules, and the like.

The term “about,” as used herein, refers to the usual error range for the respective value readily known to the skilled person in this technical field.

The term “antibody” includes intact antibodies and antigen-binding fragments thereof, wherein the antigen-binding fragments comprise the antigen-binding region and at least a portion of the heavy chain constant region comprising asparagine (N) 297, located in CH2. Typically, the “variable region” contains the antigen-binding region of the antibody and is involved in specificity and affinity of binding. See, Fundamental Immunology 7^{th} Edition, Paul, ed., Wolters Kluwer Health/Lippincott Williams & Wilkins (2013). Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively.

The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

The term “antibody” includes an antibody by itself (naked antibody) or an antibody conjugated to a cytotoxic or cytostatic drug.

A “monoclonal antibody” refers to an antibody obtained from a population of substantially homogeneous antibodies, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, for example, U.S. Pat. No. 4,816,567). The “monoclonal antibodies” may also be isolated from phage antibody libraries using the techniques described in Clackson et al. (1991) Nature, 352:624-628 and Marks et al. (1991) J. Mol. Biol., 222:581-597, for example or may be made by other methods. The antibodies described herein are monoclonal antibodies.

Specific binding of a monoclonal antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region, means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. (See generally, Fundamental Immunology (Paul, W., ed., 2nd ed. Raven Press, N.Y., 1989, Ch. 7, incorporated by reference in its entirety for all purposes).

The mature variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989), or a composite of Kabat and Chothia, or IMGT (ImMunoGeneTics information system), AbM or Contact or other conventional definition of CDRs. Kabat also provides a widely used numbering convention (Kabat numbering) in which corresponding residues between different heavy chains or between different light chains are assigned the same number. Unless otherwise apparent from the context, Kabat numbering is used to designate the position of amino acids in the variable regions. Unless otherwise apparent from the context EU numbering is used to designated positions in constant regions.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

As used herein, the term “chimeric antibody” refers to an antibody molecule in which (a) the constant region, or a portion thereof, is replaced so that the antigen binding site (variable region, CDR, or portion thereof) is linked to a constant region of a different species.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The phrase “specifically binds” refers to a molecule (e.g., antibody or antibody fragment) that binds to a target with greater affinity, avidity, more readily, and/or with greater duration to that target in a sample than it binds to a non-target compound. In some embodiments, an antibody that specifically binds a target is an antibody that binds to the target with at least 2-fold greater affinity than non-target compounds, such as, for example, at least 4-fold, 5-fold, 6-fold, 7-fold, 8-fold, 9-fold, 10-fold, 20-fold, 25-fold, 50-fold, or 100-fold greater affinity. For example, an antibody that specifically binds TIGIT will typically bind to TIGIT with at least a 2-fold greater affinity than to a non-TIGIT target. It will be understood by a person of ordinary skill in the art reading this definition, for example, that an antibody (or moiety or epitope) that specifically or preferentially binds to a first target may or may not specifically or preferentially bind to a second target. As such, “specific binding” does not necessarily require (although it can include) exclusive binding.

The term “binding affinity” is herein used as a measure of the strength of a non-covalent interaction between two molecules, e.g., an antibody, or fragment thereof, and an antigen. The term “binding affinity” is used to describe monovalent interactions (intrinsic activity).

Binding affinity between two molecules, e.g. an antibody, or fragment thereof, and an antigen, through a monovalent interaction may be quantified by determination of the dissociation constant (K_{D}). In turn, K_{D} can be determined by measurement of the kinetics of complex formation and dissociation using, as a nonlimiting example, the surface plasmon resonance (SPR) method (Biacore™). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those, for example, set forth in Caceci et al. (1984, Byte 9: 340-362). For example, the K_{D} may be established using a double-filter nitrocellulose filter binding assay such as that disclosed by Wong & Lohman (1993, Proc. Nat. Acad. Sci. USA 90: 5428-5432). Other standard assays to evaluate the binding ability of ligands such as antibodies towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art or as described in the Examples section below, such as Surface Plasmon Resonance (SPR), e.g. by using a Biacore™ system; kinetic exclusion assays such as KinExA®; and BioLayer interferometry (e.g., using the ForteBio® Octet platform). In some embodiments, binding affinity is determined using a BioLayer interferometry assay. See, e.g., Wilson et al., Biochemistry and Molecular Biology Education, 38:400-407 (2010); Dysinger et al., J. Immunol. Methods, 379:30-41 (2012); and Estep et al., Mabs, 2013, 5:270-278.

The term “cross-reacts,” as used herein, refers to the ability of an antibody to bind to an antigen other than the antigen against which the antibody was raised. In some embodiments, cross-reactivity refers to the ability of an antibody to bind to an antigen from another species than the antigen against which the antibody was raised. As a non-limiting example, an anti-TIGIT antibody as described herein that is raised against a human TIGIT antigen can exhibit cross-reactivity with TIGIT from a different species (e.g., mouse or monkey).

An “isolated” antibody refers to an antibody that has been identified and separated and/or recovered from components of its natural environment and/or an antibody that is recombinantly produced. A “purified antibody” is an antibody that is typically at least 50% w/w pure of interfering proteins and other contaminants arising from its production or purification but does not exclude the possibility that the monoclonal antibody is combined with an excess of pharmaceutical acceptable carrier(s) or other vehicle intended to facilitate its use. Interfering proteins and other contaminants can include, for example, cellular components of the cells from which an antibody is isolated or recombinantly produced. Sometimes monoclonal antibodies are at least 60%, 70%, 80%, 90%, 95 or 99% w/w pure of interfering proteins and contaminants from production or purification. The antibodies described herein, including rat, chimeric, veneered and humanized antibodies can be provided in isolated and/or purified form.

The term “LAE” refers to the tripeptide linker leucine-alanine-glutamic acid. The term “dLAE” refers to the tripeptide linker D-leucine-alanine-glutamic acid, wherein the leucine in the tripeptide linker is in the D-configuration.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision.

The terms “therapy,” “treatment,” and “amelioration” refer to any reduction in the severity of symptoms. In the case of treating cancer, treatment can refer to reducing, e.g., tumor size, number of cancer cells, growth rate, metastatic activity, cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent or severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

As used herein, a “therapeutic amount” or “therapeutically effective amount” of an agent (e.g., an antibody as described herein) is an amount of the agent that prevents, alleviates, abates, ameliorates, or reduces the severity of symptoms of a disease (e.g., a cancer) in a subject.

The terms “administer,” “administered,” or “administering” refer to methods of delivering agents, compounds, or compositions to the desired site of biological action. These methods include, but are not limited to, topical delivery, parenteral delivery, intravenous delivery, intradermal delivery, intramuscular delivery, colonic delivery, rectal delivery, or intraperitoneal delivery. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

III. Exemplary Antibodies that Bind an Immune Cell Engager

As noted above, the present inventors have found that inducing ICD by administering an antibody-drug conjugate comprising certain tubulin disrupters (e.g., auristatins, including for instance MMAE and MMAF) in combination with triggering an immune response using an antibody that binds a protein directly or indirectly involved in immune regulation and that has enhanced Fc activity can result in improved anti-tumor responses, including synergistic responses. As such, in some embodiments, the methods provided herein comprise administering to a subject with cancer an antibody that binds a target involved in regulating an immune response, wherein such binding induces, promotes, or enhances an immune response. The target of such an antibody can be referred to as an “immune cell engager.” An “immune cell engager” as used herein refers to a molecule (e.g., a transmembrane protein) that is involved in modulating an immune cell response, either positively or negatively. In some embodiments, the antibody binds to a receptor on immune cells or tumors and results in direct immune cell engagement or releases a negative inhibitory signal. In some embodiments, the immune cell engager is a molecule involved in T-cell signaling. In some embodiments, the immune cell engager modulates (e.g. activates) antigen-present cells (APCs). The immune cell engager in certain embodiments is an immune checkpoint protein. Other examples of potential immune cell engagers are listed below. In some embodiments, the antibody binds to a receptor on immune cells or tumors. Any of the antibodies that bind an immune cell engager described herein may be combined with any of the antibody-drug conjugates described herein.

The antibody that binds the target involved in immune regulation (e.g, the immune cell engager) also comprises an Fc that has one or more or all of the following features in any combination: 1) enhanced binding to one or more activating FcγRs, 2) reduced binding to inhibitory FcγRs, 3) is nonfucosylated, 4) has enhanced ADCC activity, 5) has enhanced ADCP activity, 6) activates antigen presenting cells (APCs), 7) enhances CD8 T cell responses, 8) upregulates co-stimulatory receptors, 9) activates an innate cell immune response, and/or 10) engages NK cells.

Thus, in some embodiments, the antibody comprises an Fc with enhanced binding to one or more activating FcγRs and/or reduced binding to one or more inhibitory FcγRs to obtain the desired enhanced FcγR binding profile. Activating FcγRs include one or more of FcγRIIIa, FcγRIIa, and/or FcγRI. Inhibitory FcγRs include, for example, FcγRIIb.

In certain embodiments, the antibody comprises an Fc with enhanced binding to at least FcγRIIIa. In other embodiments, the antibody comprises an Fc with enhanced binding to at least FcγRIIIa and FcγRIIa. In some embodiments, the antibody comprises an Fc with enhanced binding to at least FcγRIIIa and FcγRI. In certain embodiments, the antibody comprises an Fc with enhanced binding to FcγRIIIa, FcγRIIa, and FcγRI.

In some embodiments, the antibody, in addition to or separately from enhanced binding to an activating FcγR, has reduced binding to one or more inhibitory FcγRs. Thus, in some embodiments, the antibody has reduced binding to FcγRIIa and/or FcγRIIb.

In some embodiments, the antibody is nonfucosylated. In some embodiments, the antibody further has one of the FcγR binding profiles described above.

In certain embodiments, the Fc of the antibody comprises amino acid changes relative to a wild-type Fc to enhance binding to an activating FcγR, and/or reduce binding to one or more inhibitory FcγRs to obtain an FcγR binding profile such as described above. For example, in some embodiments the Fc of the antibody comprises the substitutions S293D, A330L, and I332E in the heavy chain constant region.

Additional details on methods for obtaining antibodies with the desired FcγR profile are provided below, as are methods for obtaining nonfucosylated antibodies.

A. Exemplary Immune Cell Engagers

Nonlimiting exemplary targets or immune cell engagers to which an antibody can be targeted include: Mullerian Hormone Receptor II (AMHR2), B7, B7H1, B7H2, B7H3, B7H4, BAFF-R, BCMA (B-cell maturation antigen), Bst1/CD157, C5 complement, CC chemokine receptor 4 (CCR4), CD123, CD137, CD19, CD20, CD25 (IL2RA), CD276, CD278, CD3, CD32, CD33, CD37, CD38, CD4 and HIV-1 gp120-binding sites, CD40, CD70, CD70 (a member of the TNF receptor ligand family), CD80, CD86, Claudin 18.2, c-MET, CSF1R, CTLA-4, EGFR, EGFR MET proto-oncogene, EPHA3, ERBB2, ERBB3, FGFR2b, FLT3, GITR, glucocorticoid-induced TNF receptor (GITR), HER2, HER3, HLA, ICOS, IDO1, IFNAR1, IFNAR2, IGF-1R, IL-3Ralpha (CD123), IL-5R, IL-5Ralpha, LAG-3, MET proto-oncogene, OX40 (CD134), PD-1, PD-L1, PD-L2, PVRIG, respiratory syncytial virus (RSV) heavily glycosylated mucin-like domain of EBOV glycoprotein (GP), Rhesus (Rh) D, sialic acid immunoglobulin-like lectins 8 (Siglec-8), signaling lymphocyte activation molecule (SLAMF7/CS1), T-cell receptor cytotoxic T-lymphocyte-associated antigen 4 (CTLA4), TIGIT, TIM3 (HAVCR2), tumor specific glycoepitope of Muc1 (TA-Muc1), VSIR (VISTA), and VTCN1.

In some embodiments, an antibody is an agonist of an immune cell engager. In some such embodiments, an antibody is an agonist of an immune cell engager selected from CD80, CD86, OX40 (CD134), GITR, CD137, CD40, VTCN1, CD276, IFNAR2, IFNAR1, CSF1R, VSIR (VISTA), and HLA.

In some embodiments, an antibody is an antagonist of an immune cell engager. In some such embodiments, an antibody is an antagonist of an immune cell engager selected from CTLA-4, PD-1, PD-L1, PD-L2, LAG-3, B7, TIM3 (HAVCR2), PVRIG, TIGIT, CD25 (IL2RA), and IDO1.

In certain embodiments, the antibody that binds an immune cell engager for the methods provided herein can be an inhibitor against a checkpoint protein. In some embodiments, the antibody that binds an immune cell engager for the methods provided herein can be a PD-1 inhibitor, a PD-L1 inhibitor, a PD-L2 inhibitor, a CTLA-4 inhibitor, a LAG-3 inhibitor, a B7 inhibitor, a TIM3 (HAVCR2) inhibitor, an OX40 (CD134) inhibitor, a GITR agonist, a CD137 agonist, or a CD40 agonist, a VTCN1 inhibitor, an IDO1 inhibitor, a CD276 inhibitor, a PVRIG inhibitor, a TIGIT inhibitor, a CD25 (IL2RA) inhibitor, an IFNAR2 inhibitor, an IFNAR1 inhibitor, a CSF1R inhibitor, a VSIR (VISTA) inhibitor, or a therapeutic agent targeting HLA. Such inhibitors, activators, or therapeutic agents are further provided below. In any of the embodiments herein, the antibody that binds an immune cell engager may be an antibody comprising an Fc with enhanced binding to one or more activating FcγRs. In some embodiments, the antibody that binds an immune cell engager is a nonfucosylated antibody.

In some embodiments, the antibody that binds an immune cell engager is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206) or a nonfucosylated version thereof. In another embodiment, the anti-CTLA-4 antibody is ipilimumab (also known as MDX-010 or MDX-101) or a nonfucosylated version thereof. Ipilimumab is a fully human monoclonal IgG antibody that binds to CTLA-4. Ipilimumab is marketed under the trade name Yervoy™.

In certain embodiments, the antibody that binds an immune cell engager is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, W0201008941.1, WO2010036959, WO2011066342, WO2011159877, WO2011082400, and WO2011161699, all of which are incorporated herein in their entireties.

In some embodiments, the antibody that binds an immune cell engager is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106), pembrolizumab (also known as MK-3475, SCH 900475, or lambrolizumab), or a nonfucosylated version thereof. In one embodiment, the anti-PD-1 antibody is nivolumab or a nonfucosylated version thereof. Nivolumab is a human IgG4 anti-PD-1 monoclonal antibody, and is marketed under the trade name Opdivo™. In another embodiment, the anti-PD-1 antibody is pembrolizumab or a nonfucosylated version thereof. Pembrolizumab is a humanized monoclonal IgG4 antibody and is marketed under the trade name Keytruda™. In yet another embodiment, the anti-PD-1 antibody is CT-011, a humanized antibody, or a nonfucosylated version thereof. CT-011 administered alone has failed to show response in treating acute myeloid leukemia (AML) at relapse. In yet another embodiment, the anti-PD-1 antibody is AMP-224, a fusion protein, or a nonfucosylated version thereof. In another embodiment, the PD-1 antibody is BGB-A317, or a nonfucosylated version thereof. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab or a nonfucosylated version thereof. In another embodiment, the PD-1 antibody is camrelizumab or a nonfucosylated version thereof. In a further embodiment, the PD-1 antibody is sintilimab or a nonfucosylated version thereof. In some embodiments, the PD-1 antibody is tislelizumab or a nonfucosylated version thereof. In certain embodiments, the PD-1 antibody is TSR-042 or a nonfucosylated version thereof. In yet another embodiment, the PD-1 antibody is PDR001 or a nonfucosylated version thereof. In yet another embodiment, the PD-1 antibody is toripalimab or a nonfucosylated version thereof.

In certain embodiments, the antibody that binds an immune cell engager is a PD-L1 inhibitor. In one embodiment, the PD-L1 inhibitor is an anti-PD-L1 antibody. In one embodiment, the anti-PD-L1 antibody is MEDI4736 (durvalumab) or a nonfucosylated version thereof. In another embodiment, the anti-PD-L1 antibody is BMS-936559 (also known as MDX-1105-01) or a nonfucosylated version thereof. In yet another embodiment, the PD-L1 inhibitor is atezolizumab (also known as MPDL3280A, and Tecentriq®) or a nonfucosylated version thereof. In a further embodiment, the PD-L1 inhibitor is avelumab or a nonfucosylated version thereof.

In one embodiment, the antibody that binds an immune cell engager is a PD-L2 inhibitor. In one embodiment, the PD-L2 inhibitor is an anti-PD-L2 antibody. In one embodiment, the anti-PD-L2 antibody is rHIgM12B7A or a nonfucosylated version thereof.

In one embodiment, the antibody that binds an immune cell engager is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211), or a nonfucosylated version thereof. In another embodiment, the LAG-3 inhibitor is BMS-986016 or a nonfucosylated version thereof.

In one embodiment, the antibody that binds an immune cell engager is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834), or a nonfucosylated version thereof. In some embodiments, the B7 inhibitor is a B7-H4 inhibitor. A nonlimiting exemplary B7-H4 inhibitor is FPA150, a nonfucosylated antibody against B7-H4. See PCT/US2018/047805.

In one embodiment, the antibody that binds an immune cell engager is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the antibody that binds an immune cell engager is an OX40 (CD134) agonist antibody. In certain embodiments, the anti-OX40 antibody is MEDI6469 or a nonfucosylated version thereof.

In one embodiment, the antibody that binds an immune cell engager is a GITR agonist. In one embodiment, the immune cell engager is an anti-GITR antibody or a nonfucosylated version thereof. In one embodiment, the anti-GITR antibody is TRX518 or a nonfucosylated version thereof.

In one embodiment, the antibody that binds an immune cell engager is a CD137 agonist. In one embodiment, the immune cell engager is an anti-CD137 antibody. In one embodiment, the anti-CD137 antibody is urelumab or a nonfucosylated version thereof. In another embodiment, the anti-CD137 antibody is PF-05082566 or a nonfucosylated version thereof.

In one embodiment, the antibody that binds an immune cell engager is a CD40 agonist. In one embodiment, the antibody that binds an immune cell engager is an anti-CD40 antibody. In one embodiment, the anti-CD40 antibody is CF-870,893 or a nonfucosylated version thereof. In one embodiment, the anti-CD40 antibody is MP0317 (Molecular Partners) or a nonfucosylated version thereof. In one embodiment, the anti-CD40 antibody is YH003 (Eucure Biopharma) or a nonfucosylated version thereof. In one embodiment, the anti-CD40 antibody is CDX-1140 (Celldex Therapeutics) or a nonfucosylated version thereof. In one embodiment, the anti-CD40 antibody is YH003 (Eucure Biopharma) or a nonfucosylated version thereof. In one embodiment, the anti-CD40 antibody is mitazalimab (Alligator Bioscience) or a nonfucosylated version thereof. In one embodiment, the anti-CD40 antibody is ABBV-927 (AbbVie) or a nonfucosylated version thereof. In one embodiment, the anti-CD40 antibody is sotigalimab (Apexigen) or a nonfucosylated version thereof. In one embodiment, the anti-CD40 antibody is GEN1042 (Genmab) or a nonfucosylated version thereof. In one embodiment, the anti-CD40 antibody is 2141 V-11 (Rockefeller University) or a nonfucosylated version thereof. In one embodiment, the anti-CD40 antibody is selicrelumab (Roche) or a nonfucosylated version thereof. In one embodiment, the anti-CD40 antibody is SEA-CD40 (Seagen), which is a nonfucosylated, humanized version of murine S2C6 and which comprises heavy chain CDR1, CDR2, and CDR3, and light chain CDR1, CDR2, and CDR3 comprising the amino acid sequences of SEQ ID NOs: 30-35, respectively. The corresponding VH and VL comprise the amino acid sequences of SEQ ID NOs: 28 and 29, respectively. SEA-CD40 is described in US Patent Publication Nos. 2017/0333556 and 2017/0137528, both of which are herein incorporated by reference.

In some embodiments, the antibody that binds an immune cell engager is an antibody that binds CD70. In some embodiments, the antibody is SEA-CD70. See, e.g., U.S. Pat. No. 8,067,546; Table of Sequences herein.

In some embodiments, the antibody that binds an immune cell engager is an antibody that binds BCMA. In some embodiments, the antibody is SEA-BCMA. See, e.g., US Publication No. 2017/0233484 and WO 2017/143069 (VH and VL of SEQ ID NOs: 13 and 19, respectively; CDRs of SEQ ID NOs: 60, 61, 62, 90, 91, 92, see US Publication No. 2017/0233484); see also Table of Sequences herein (VH and VL of SEQ ID NOs: 45 and 46, respectively; CDRs of SEQ ID NOs: 47-52).

In one embodiment, the antibody that binds an immune cell engager is an anti-interleukin-15 antibody.

In one embodiment, the antibody that binds an immune cell engager is a VTCN inhibitor. In one embodiment, the VTCN inhibitor is FPA150 or a nonfucosylated version thereof.

In one embodiment, the antibody that binds an immune cell engager is an anti-IDO antagonist antibody. In some embodiments, the antibody that binds an immune cell engager is a TIGIT inhibitor. In certain embodiments, the TIGIT inhibitor is an anti-TIGIT antibody. In one embodiment, the TIGIT inhibitor is MTIG7192A or a nonfucosylated version thereof. In another embodiment, the TIGIT inhibitor is BMS-986207 (BMS) or a nonfucosylated version thereof. In yet another embodiment, the TIGIT inhibitor is OMP-313M32 or a nonfucosylated version thereof. In one embodiment, the TIGIT inhibitor is MK-7684. In another embodiment, the TIGIT inhibitor is AB154 or a nonfucosylated version thereof. In yet another embodiment, the TIGIT inhibitor is CGEN-15137 or a nonfucosylated version thereof. In one embodiment, the TIGIT inhibitor is SEA-TGT. In another embodiment, the TIGIT inhibitor is ASP8374 (Astellas) or a nonfucosylated version thereof. In yet another embodiment, the TIGIT inhibitor is AJUD008 or a nonfucosylated version thereof. In one embodiment, the TIGIT inhibitor is AB308 (Arcus Biosciences) or a nonfucosylated version thereof. In another embodiment, the TIGIT inhibitor is AGEN1327 (Agenus) or a nonfucosylated version thereof. In yet another embodiment, the TIGIT inhibitor is AK127 (Akeso Biopharma) or a nonfucosylated version thereof. In another embodiment, the TIGIT inhibitor is BAT6005 (Bio-Thera Solutions) or a nonfucosylated version thereof. In another embodiment, the TIGIT inhibitor is BAT6021 (Bio-Thera Solutions) or a nonfucosylated version thereof. In one embodiment, the TIGIT inhibitor is CASC-674 (Seagen) or a nonfucosylated version thereof. In another embodiment, the TIGIT inhibitor is COM902 (Compugen) or a nonfucosylated version thereof. In yet another embodiment, the TIGIT inhibitor is domvanalimab (Arcus Biosciences) or a nonfucosylated version thereof. In one embodiment, the TIGIT inhibitor is etigilimab (Mereo BioPharma) or a nonfucosylated version thereof. In another embodiment, the TIGIT inhibitor is GSK4428859 (GSK) or a nonfucosylated version thereof. In yet another embodiment, the TIGIT inhibitor is HL186 (HanAll Biopharma) or a nonfucosylated version thereof. In one embodiment, the TIGIT inhibitor is MIL-100 (Beijing Mabworks Biotech) or a nonfucosylated version thereof. In another embodiment, the TIGIT inhibitor is YH-29143 (Yu Han) or a nonfucosylated version thereof. In one embodiment, the TIGIT inhibitor is HLX53 (Shanghai Henlius Biotech) or a nonfucosylated version thereof. In another embodiment, the TIGIT inhibitor is IBI939 (Innovent Biologics) or a nonfucosylated version thereof. In yet another embodiment, the TIGIT inhibitor is JS006 (Junshi Biosciences) or a nonfucosylated version thereof. In one embodiment, the TIGIT inhibitor is M6223 (Merck KGaA) or a nonfucosylated version thereof. In another embodiment, the TIGIT inhibitor is MG1131 (Mogam Institute) or a nonfucosylated version thereof. In yet another embodiment, the TIGIT inhibitor is ociperlimab (BeiGene) or a nonfucosylated version thereof. In one embodiment, the TIGIT inhibitor is tiragolumab (Roche; described in U.S. Pat. No. 10,047,158) or a nonfucosylated version thereof. In another embodiment, the TIGIT inhibitor is TJT6 (I-Mab Biopharma) or a nonfucosylated version thereof. In yet another embodiment, the TIGIT inhibitor is vibostolimab (MSD; described in U.S. Pat. No. 10,618,958) or a nonfucosylated version thereof. In one embodiment, the TIGIT inhibitor is YBL-012 (Y Biologics) or a nonfucosylated version thereof. In another embodiment, the TIGIT inhibitor is IBI-939 (Innovent) or a nonfucosylated version thereof. In yet another embodiment, the TIGIT inhibitor is AZD2936 (AstraZeneca) or a nonfucosylated version thereof. In one embodiment, the TIGIT inhibitor is EOS-448 (iTeos/GSK) or a nonfucosylated version thereof. In another embodiment, the TIGIT inhibitor is BAT6005 (Bio Thera) or a nonfucosylated version thereof. In yet another embodiment, the TIGIT inhibitor is AGEN1777 (BMS/Agenus) or a nonfucosylated version thereof.

In some embodiments, the antibody that binds an immune cell engager is a VSIR inhibitor. In certain embodiments, the VSIR inhibitor is an anti-VSIR antibody. In one embodiment, the VSIR inhibitor is MTIG7192A or a nonfucosylated version thereof. In another embodiment, the VSIR inhibitor is CA-170 or a nonfucosylated version thereof. In yet another embodiment, the VSIR inhibitor is JNJ 61610588 or a nonfucosylated version thereof. In one embodiment, the VSIR inhibitor is HIMIBD-002 or a nonfucosylated version thereof.

In some embodiments, the antibody that binds an immune cell engager is a TIM3 inhibitor. In certain embodiments, the TIM3 inhibitor is an anti-TIM3 antibody. In one embodiment, the TIM3 inhibitor is AJUD009 or a nonfucosylated version thereof.

In some embodiments, the antibody that binds an immune cell engager is a CD25 (TL2RA) inhibitor. In certain embodiments, the CD25 (IL2RA) inhibitor is an anti-CD25 (TL2RA) antibody. In one embodiment, the CD25 (IL2RA) inhibitor is daclizumab or a nonfucosylated version thereof. In another embodiment, the CD25 (IL2RA) inhibitor is basiliximab or a nonfucosylated version thereof.

In some embodiments, the antibody that binds an immune cell engager is an IFNAR1 inhibitor. In certain embodiments, the IFNAR1 inhibitor is an anti-IFNAR1 antibody. In one embodiment, the IFNAR1 inhibitor is anifrolumab or a nonfucosylated version thereof. In another embodiment, the IFNAR1 inhibitor is sifalimumab or a nonfucosylated version thereof.

In some embodiments, the antibody that binds an immune cell engager is a CSF1R inhibitor. In certain embodiments, the CSF1R inhibitor is an anti-CSF1R antibody. In one embodiment, the CSF1R inhibitor is pexidartinib or a nonfucosylated version thereof. In another embodiment, the CSF1R inhibitor is emactuzumab or a nonfucosylated version thereof. In yet another embodiment, the CSF1R inhibitor is cabiralizumab or a nonfucosylated version thereof. In one embodiment, the CSF1R inhibitor is ARRY-382 or a nonfucosylated version thereof. In another embodiment, the CSF1R inhibitor is BLZ945 or a nonfucosylated version thereof. In yet another embodiment, the CSF1R inhibitor is AJUD010 or a nonfucosylated version thereof. In one embodiment, the CSF1R inhibitor is AMG820 or a nonfucosylated version thereof. In another embodiment, the CSF1R inhibitor is IMC-CS4 or a nonfucosylated version thereof. In yet another embodiment, the CSF1R inhibitor is JNJ-40346527 or a nonfucosylated version thereof. In one embodiment, the CSF1R inhibitor is PLX5622 or a nonfucosylated version thereof. In another embodiment, the CSF1R inhibitor is FPA008 or a nonfucosylated version thereof.

In various embodiments, an antibody that binds an immune cell engager has one or more or all of the following activities in any combination: 1) depletes T regulatory (Treg) cells, 2) activates antigen presenting cells (APCs), 3) enhances CD8 T cell responses, 4) upregulates co-stimulatory receptors, and/or 5) promotes release of immune activating cytokines (such as CXCL10 and/or IFNγ). In some embodiments, the antibody that binds an immune cell engager promotes release of immune-activating cytokines (e.g., CXCL10 and IFNγ) to a greater extent than immune suppressive cytokines (such as IL10 and/or MDC).

B. Exemplary Anti-TIGIT Antibodies

In one aspect, antibodies that bind to human TIGIT (T-cell immunoreceptor with Ig and ITIM domains) are provided as the antibodies against the immune cell engager. As described herein, in some embodiments, the anti-TIGIT antibody inhibits interaction between TIGIT and one or both of the ligands CD155 and CD112. In some embodiments, the anti-TIGIT antibody inhibits the interaction between TIGIT and CD155 in a functional bioassay, allowing CD155-CD226 signaling to occur. In some embodiments, the anti-TIGIT antibody exhibits synergy with an anti-PD-1 agent (e.g., an anti-PD-1 antibody) or an anti-PD-L1 agent (e.g., an anti-PD-L1 antibody). In some embodiments, an anti-TIGIT antibody for use in the present methods is SEA-TGT, which is a nonfucosylated IgG1 antibody comprising heavy chain CDR1, CDR2, and CDR3, and light chain CDR1, CDR2, and CDR3 comprising the amino acid sequences of SEQ ID NOs: 7, 10, 14, 17, 18, and 19, respectively. The corresponding VH and VL comprise the amino acid sequences of SEQ ID NOs: 1 and 6, respectively.

The present inventors found that, surprisingly, anti-TIGIT antibodies with enhanced effector function, such as may be achieved with nonfucosylated IgG1 antibodies, deplete Treg cells and show improved efficacy in vivo. Accordingly, in various embodiments, nonfucosylated anti-TIGIT antibodies are provided.

In some embodiments, an anti-TIGIT antibody, such as a nonfucosylated anti-TIGIT antibody, binds to human TIGIT protein (SEQ ID NO:218) or a portion thereof with high affinity. In some embodiments, the antibody has a binding affinity (K_{D}) for human TIGIT of less than 5 nM, less than 1 nM, less than 500 pM, less than 250 pM, less than 150 pM, less than 100 pM, less than 50 pM, less than 40 pM, less than 30 pM, less than 20 pM, or less than about 10 pM. In some embodiments, the antibody has a binding affinity (K_{D}) for human TIGIT of less than 50 pM. In some embodiments, the antibody has a K_{D} for human TIGIT in the range of about 1 pM to about 5 nM, e.g., about 1 pM to about 1 nM, about 1 pM to about 500 pM, about 5 pM to about 250 pM, or about 10 pM to about 100 pM.

In some embodiments, in addition to binding to human TIGIT with high affinity, a nonfucosylated anti-TIGIT antibody exhibits cross-reactivity with cynomolgus monkey (“cyno”) TIGIT and/or mouse TIGIT. In some embodiments, the anti-TIGIT antibody binds to mouse TIGIT with a binding affinity (K_{D}) of 100 nM or less. In some embodiments, the anti-TIGIT antibody binds to human TIGIT with a K_{D} of 5 nM or less, and cross-reacts with mouse TIGIT with a K_{D} of 100 nM or less. In some embodiments, an anti-TIGIT antibody that binds to a human TIGIT also exhibits cross-reactivity with both cynomolgus monkey TIGIT and mouse TIGIT.

In some embodiments, antibody cross-reactivity is determined by detecting specific binding of the anti-TIGIT antibody to TIGIT that is expressed on a cell (e.g., a cell line that expresses human TIGIT, cynomolgus monkey TIGIT, or mouse TIGIT, or a primary cell that endogenously expresses TIGIT, e.g., primary T cells that endogenously express human TIGIT, cyno TIGIT, or mouse TIGIT). In some embodiments, antibody binding and antibody cross-reactivity is determined by detecting specific binding of the anti-TIGIT antibody to purified or recombinant TIGIT (e.g., purified or recombinant human TIGIT, purified or recombinant cyno TIGIT, or purified or recombinant mouse TIGIT) or a chimeric protein comprising TIGIT (e.g., an Fc-fusion protein comprising human TIGIT, cynomolgus monkey TIGIT, or mouse TIGIT, or a His-tagged protein comprising human TIGIT, cyno TIGIT, or mouse TIGIT).

In some embodiments, the anti-TIGIT antibodies provided herein inhibit interaction between TIGIT and the ligand CD155. In some embodiments, the anti-TIGIT antibodies provided herein inhibit interaction between TIGIT and the ligand CD 112. In some embodiments, the anti-TIGIT antibodies provided herein inhibit interaction between TIGIT and both of the ligands CD155 and CD112.

In some embodiments, an anti-TIGIT antibody that binds to human TIGIT comprises a light chain variable region sequence, or a portion thereof, and/or a heavy chain variable region sequence, or a portion thereof, derived from any of the following antibodies described herein: Clone 13, Clone 13A, Clone 13B, Clone 13C, or Clone 13D. The amino acid sequences of the CDR, light chain variable domain (VL), and heavy chain variable domain (VH) of the anti-TIGIT antibodies Clone 13, Clone 13A, Clone 13B, Clone 13C, and Clone 13D are set forth in the Table of Sequences below.

In some embodiments, an anti-TIGIT antibody comprises one or more (e.g., one, two, three, four, five, or six) of:
a heavy chain CDR1 sequence comprising an amino acid sequence selected from SEQ ID NO:7, SEQ ID NO:8, and SEQ ID NO:9;
a heavy chain CDR2 sequence comprising an amino acid sequence selected from SEQ ID NO:10, SEQ ID NO:11, SEQ ID NO:12, and SEQ ID NO:13;
a heavy chain CDR3 sequence comprising an amino acid sequence selected from SEQ ID NO:14, SEQ ID NO:15 and 16;
a light chain CDR1 sequence comprising an amino acid sequence of SEQ ID NO:17;
a light chain CDR2 sequence comprising an amino acid sequence of SEQ ID NO:18; and/or
a light chain CDR3 sequence comprising the amino acid sequence of SEQ ID NO:19.

In some embodiments, an anti-TIGIT antibody comprises a heavy chain CDR1 sequence comprising the amino acid sequence of SEQ ID NO: 7, SEQ ID NO:8, or SEQ ID NO:9; a heavy chain CDR2 sequence comprising the amino acid sequence of SEQ ID NO:10, SEQ ID NO: 11, SEQ ID NO:12, or SEQ ID NO:13; and a heavy chain CDR3 sequence comprising the amino acid sequence of SEQ ID NO: 14, SEQ ID NO:15, or 16.

In some embodiments, an anti-TIGIT antibody comprises a light chain CDR1 sequence comprising the amino acid sequence of SEQ ID NO:17; a light chain CDR2 sequence comprising the amino acid sequence of SEQ ID NO:18; and a light chain CDR3 sequence comprising the amino acid sequence of SEQ ID NO:19.

In some embodiments, an anti-TIGIT antibody comprises a heavy chain CDR1 sequence comprising the amino acid sequence of SEQ ID NO: 7, SEQ ID NO:8, or SEQ ID NO: 9; a heavy chain CDR2 sequence comprising the amino acid sequence of SEQ ID NO:10, SEQ ID NO: 11, SEQ ID NO:12, or SEQ ID NO:13; a heavy chain CDR3 sequence comprising the amino acid sequence of SEQ ID NO:14, SEQ ID NO:15, or SEQ ID NO: 16; a light chain CDR1 sequence comprising the amino acid sequence of SEQ ID NO:17; a light chain CDR2 sequence comprising the amino acid sequence of SEQ ID NO: 18; and a light chain CDR3 sequence comprising the amino acid sequence of SEQ ID NO: 19.

In some embodiments, an anti-TIGIT antibody comprises a heavy chain CDR1, CDR2, and CDR3, and a light chain CDR1, CDR2, and CDR3 comprising the amino acid sequences of:
(a) SEQ ID NOs: 7, 10, 14, 17, 18, and 19, respectively; or
(b) SEQ ID NOs: 8, 11, 14, 17, 18, and 19, respectively; or
(c) SEQ ID NOs: 9, 12, 15, 17, 18, and 19, respectively; or
(d) SEQ ID NOs: 8, 13, 16, 17, 18, and 19, respectively; or
(e) SEQ ID NOs: 8, 12, 16, 17, 18, and 19, respectively.

In some embodiments, an anti-TIGIT antibody comprises a heavy chain variable region (VH) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:1, SEQ ID NO:2, SEQ ID NO:3, SEQ ID NO:4, or SEQ ID NO:5. In some embodiments, an anti-TIGIT antibody comprises a VH comprising the amino acid sequence of SEQ ID NO:1, SEQ ID NO:2, SEQ ID NO:3, SEQ ID NO:4, or SEQ ID NO:5. In some embodiments, a VH sequence having at least 90% sequence identity to a reference sequence (e.g., SEQ ID NO:1, SEQ ID NO:2, SEQ ID NO:3, SEQ ID NO:4, or SEQ ID NO:5) contains one, two, three, four, five, six, seven, eight, nine, ten or more substitutions (e.g., conservative substitutions), insertions, or deletions relative to the reference sequence but retains the ability to bind to human TIGIT and optionally, retains the ability to block binding of CD155 and/or CD 112 to TIGIT.

In some embodiments, an anti-TIGIT antibody comprises a light chain variable region (VL) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:6. In some embodiments, an anti-TIGIT antibody comprises a VL comprising the amino acid sequence of SEQ ID NO:6. In some embodiments, a VL sequence having at least 90% sequence identity to a reference sequence (e.g., SEQ ID NO:6) contains one, two, three, four, five, six, seven, eight, nine, ten or more substitutions (e.g., conservative substitutions), insertions, or deletions relative to the reference sequence but retains the ability to bind to human TIGIT and optionally, retains the ability to block binding of CD155 and/or CD 112 to TIGIT.

In some embodiments, an anti-TIGIT antibody comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:1, SEQ ID NO:2, SEQ ID NO:3, SEQ ID NO:4, or SEQ ID NO:5, and comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:6. In some embodiments, an anti-TIGIT antibody comprises a heavy chain variable region comprising the amino acid sequence of SEQ ID NO:1, SEQ ID NO:2, SEQ ID NO:3, SEQ ID NO:4, or SEQ ID NO:5, and comprises a light chain variable region comprising the amino acid sequence of SEQ ID NO:6.

In some embodiments, an anti-TIGIT antibody comprises:
(a) a VH comprising the amino acid sequence of SEQ ID NO:1 and a VL comprising the amino acid sequence of SEQ ID NO:6;
(b) a VH comprising the amino acid sequence of SEQ ID NO:2 and a VL comprising the amino acid sequence of SEQ ID NO:6; or
(c) a VH comprising the amino acid sequence of SEQ ID NO:3 and a VL comprising the amino acid sequence of SEQ ID NO:6; or
(d) a VH comprising the amino acid sequence of SEQ ID NO:4 and a VL comprising the amino acid sequence of SEQ ID NO:6; or
(f) a VH comprising the amino acid sequence of SEQ ID NO:5 and a VL comprising the amino acid sequence of SEQ ID NO:6.

In some embodiments, an anti-TIGIT antibody comprises a heavy chain comprising an amino acid sequence selected from SEQ ID NOs: 20, 21, 22, 23, and 24; and a light chain comprising the amino acid sequence of SEQ ID NO: 25.

In some embodiments, an anti-TIGIT antibody for use in the present methods is a nonfucosylated version of an anti-TIGIT antibody disclosed in US 2009/0258013, US 2016/0176963, US 2016/0376365, or WO 2016/028656.

C. Exemplary Anti-CD40 Antibodies

As noted above, in some embodiments, the antibody that binds an immune cell engager is an agonist anti-CD40 antibody. Agonistic CD40 monoclonal antibodies including dacetuzumab have shown encouraging clinical activity in single-agent and combination chemotherapy settings. Dacetuzumab demonstrated some clinical activity in a phase 1 study in NHL and a phase 2 study in diffuse large B-cell lymphoma (DLBCL). See, e.g., Advani et al., J. Clin. Oncol. 27:4371-4377 (2009) and De Vos et al., J. Hematol. Oncol. 7:1-9 (2014). Additionally, CP-870,893, a humanized IgG2 agonist antibody to CD40, showed encouraging activity in solid tumor indications when combined with paclitaxel or carboplatin or gemcitabine. In these studies, activation of antigen presenting cells, cytokine production, and generation of antigen- specific T cells were seen. See, e.g., Beatty et al., Clin. Cancer Res. 19:6286-6295 (2013) and Vonderheide et al., Oncoimmunology 2:e23033 (2013).

In some embodiments, a nonfucosylated anti-CD40 antibody is provided for use in the present methods. In some embodiments, the nonfucosylated anti-CD40 antibody is SEA-CD40, which is a nonfucosylated, humanized version of murine S2C6 and which comprises heavy chain CDR1, CDR2, and CDR3, and light chain CDR1, CDR2, and CDR3 comprising the amino acid sequences of SEQ ID NOs: 30-35, respectively. The corresponding VH and VL comprise the amino acid sequences of SEQ ID NOs: 28 and 29, respectively. SEA-CD40 is described in US Patent Publication Nos. 2017/0333556 and 2017/0137528, both of which are herein incorporated by reference. S2C6 was originally isolated as a murine monoclonal antibody raised against a human bladder carcinoma, referred to herein as mS2C6. See, e.g., Paulie et al., Cancer Immunol. Immunother. 17:165-179 (1984). The S2C6 antibody is a partial agonist of the CD40 signaling pathway and, in some embodiments, has the following activities: binding to human CD40 protein, binding to cynomolgus CD40 protein, activation of the CD40 signaling pathway, potentiation of the interaction of CD40 with its ligand, CD40L. See, e.g., U.S. Pat. No. 6,946,129.

S2C6 was humanized and this humanized antibody is referred to as humanized S2C6, herein, and alternatively as dacetuzumab, which is fucosylated humanized S2C6 (fhS2C6, or SGN-40). See, e.g., WO 2006/128103, which is incorporated herein by reference for any purpose. SEA-CD40 is a nonfucosylated humanized S2C6 antibody. Other versions of humanized S2C6 are disclosed at WO2008/091954; these can be nonfucosylated and used in the methods disclosed herein.

In some embodiments, an anti-CD40 antibody comprises one or more (e.g., one, two, three, four, five, or six) of:
a heavy chain CDR1 sequence comprising the amino acid sequence of SEQ ID NO:30;
a heavy chain CDR2 sequence comprising the amino acid sequence of SEQ ID NO:31 or SEQ ID NO: 36;
a heavy chain CDR3 sequence comprising the amino acid sequence of SEQ ID NO:32;
a light chain CDR1 sequence comprising the amino acid sequence is SEQ ID NO:33;
a light chain CDR2 sequence comprising the amino acid is SEQ ID NO:34;
and/or a light chain CDR3 sequence comprising the amino acid sequence of SEQ ID NO:35.

In some embodiments, an anti-CD40 antibody comprises a heavy chain CDR1 sequence comprising the amino acid sequence of SEQ ID NO:30; a heavy chain CDR2 sequence comprising the amino acid sequence of any of SEQ ID NO:31 or SEQ ID NO:36; and a heavy chain CDR3 sequence comprising the amino acid sequence of SEQ ID NO:32.

In some embodiments, an anti-CD40 antibody comprises a light chain CDR1 sequence comprising the amino acid sequence of SEQ ID NO:33; a light chain CDR2 sequence comprising the amino acid of SEQ ID NO:34; and a light chain CDR3 sequence comprising the amino acid sequence of SEQ ID NO:35.

In some embodiments, an anti-CD40 antibody comprises a heavy chain CDR1 sequence comprising the amino acid sequence of SEQ ID NO:30; a heavy chain CDR2 sequence comprising the amino acid sequence of SEQ ID NO:31 or SEQ ID NO:36; a heavy chain CDR3 sequence comprising the amino acid sequence of SEQ ID NO:32; a light chain CDR1 sequence comprising the amino acid sequence of SEQ ID NO:33; a light chain CDR2 sequence comprising the amino acid sequence of SEQ ID NO:34; and a light chain CDR3 sequence comprising the amino acid sequence of SEQ ID NO:35.

In some embodiments, an anti-CD40 antibody comprises a heavy chain CDR1, CDR2, and CDR3, and a light chain CDR1, CDR2, and CDR3 comprising the amino acid sequences of:
(a) SEQ ID NOs: 30, 31, 33, 34, and 35, respectively; or
(b) SEQ ID NOs: 30, 36, 33, 34, and 35, respectively.

In some embodiments, an anti-CD40 antibody comprises a heavy chain variable region (VH) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:28. In some embodiments, an anti-CD40 antibody comprises a VH comprising the amino acid sequence of NO:28. In some embodiments, a VH sequence having at least 90% sequence identity to a reference sequence (e.g., SEQ ID NO:28) contains one, two, three, four, five, six, seven, eight, nine, ten or more substitutions (e.g., conservative substitutions), insertions, or deletions relative to the reference sequence but retains the ability to bind to human CD40.

In some embodiments, an anti-CD40 antibody comprises a light chain variable region (VL) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:29. In some embodiments, an anti-CD40 antibody comprises a VL comprising the amino acid sequence of SEQ ID NO:29. In some embodiments, a VL sequence having at least 90% sequence identity to a reference sequence (e.g., SEQ ID NO:29) contains one, two, three, four, five, six, seven, eight, nine, ten or more substitutions (e.g., conservative substitutions), insertions, or deletions relative to the reference sequence but retains the ability to bind to human CD40.

In some embodiments, an anti-CD40 antibody comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:28, and comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:29. In some embodiments, an anti-CD40 antibody comprises a heavy chain variable region comprising the amino acid sequence of SEQ ID NO:28, and comprises a light chain variable region comprising the amino acid sequence of SEQ ID NO:29.

In some embodiments, the anti-CD40 antibody comprises the heavy chain variable region and light chain variable region disclosed as SEQ ID NO:28 and 29, respectively. In some embodiments, the anti-CD40 antibody comprises the heavy chain and light chain disclosed as SEQ ID NO:26 and 27, respectively.

D. Exemplary Anti-CD70 Antibodies

In some embodiments, a nonfucosylated anti-CD70 antibody is provided for use in the present methods as the antibody that binds an immune cell engager. In some embodiments, the nonfucosylated anti-CD70 antibody is SEA-CD70, as described in U.S. Pat. No. 8,067,546 and which comprises heavy chain CDR1, CDR2, and CDR3, and light chain CDR1, CDR2, and CDR3 comprising the amino acid sequences of SEQ ID NOs: 53-58, respectively. The corresponding VH and VL comprise the amino acid sequences of SEQ ID NOs: 41 and 42, respectively. The CD70 molecule is a member of the tumor necrosis factor (TNF) ligand superfamily (TNFSF) and it binds to the related receptor, CD27 (TNFRSF7). The interaction between the two molecules activates intracellular signals from both receptors. In normal conditions, CD70 expression is transient and limited to activated T and B cells, mature dendritic, and natural killer (NK) cells. Similarly, CD27 is expressed on both naïve and activated effector T cells, as well as NK and activated B cells. However, CD70 is also aberrantly expressed in various hematologic cancers, including acute myeloid leukemia (AML), myelodysplastic syndrome (MDS), and non-Hodgkin lymphoma (NHL), as well as carcinomas, and plays a role in both tumor cell survival and/or tumor immune evasion. SEA-CD70 acts through blocking CD70/CD27 axis signaling, eliciting antibody dependent cellular phagocytosis (ADCP) and complement dependent cytotoxicity (CDC), and enhancing antibody dependent cellular cytotoxicity (ADCC).

In some embodiments, an anti-CD70 antibody comprises one or more (e.g., one, two, three, four, five, or six) of:
a heavy chain CDR1 sequence comprising the amino acid sequence of SEQ ID NO:53;
a heavy chain CDR2 sequence comprising the amino acid sequence of SEQ ID NO:54;
a heavy chain CDR3 sequence comprising the amino acid sequence of SEQ ID NO:55;
a light chain CDR1 sequence comprising the amino acid sequence is SEQ ID NO:56;
a light chain CDR2 sequence comprising the amino acid is SEQ ID NO:57;
and/or a light chain CDR3 sequence comprising the amino acid sequence of SEQ ID NO:58.

In some embodiments, an anti-CD70 antibody comprises a heavy chain CDR1 sequence comprising the amino acid sequence of SEQ ID NO:53; a heavy chain CDR2 sequence comprising the amino acid sequence of any of SEQ ID NO:54; and a heavy chain CDR3 sequence comprising the amino acid sequence of SEQ ID NO:55.

In some embodiments, an anti-CD70 antibody comprises a light chain CDR1 sequence comprising the amino acid sequence of SEQ ID NO:56; a light chain CDR2 sequence comprising the amino acid of SEQ ID NO:57; and a light chain CDR3 sequence comprising the amino acid sequence of SEQ ID NO:58.

In some embodiments, an anti-CD70 antibody comprises a heavy chain CDR1 sequence comprising the amino acid sequence of SEQ ID NO:53; a heavy chain CDR2 sequence comprising the amino acid sequence of SEQ ID NO:54; a heavy chain CDR3 sequence comprising the amino acid sequence of SEQ ID NO:55; a light chain CDR1 sequence comprising the amino acid sequence of SEQ ID NO:56; a light chain CDR2 sequence comprising the amino acid sequence of SEQ ID NO:57; and a light chain CDR3 sequence comprising the amino acid sequence of SEQ ID NO:58.

In some embodiments, an anti-CD70 antibody comprises a heavy chain variable region (VH) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:41. In some embodiments, an anti-CD70 antibody comprises a VH comprising the amino acid sequence of NO:41. In some embodiments, a VH sequence having at least 90% sequence identity to a reference sequence (e.g., SEQ ID NO:41) contains one, two, three, four, five, six, seven, eight, nine, ten or more substitutions (e.g., conservative substitutions), insertions, or deletions relative to the reference sequence but retains the ability to bind to human CD70.

In some embodiments, an anti-CD70 antibody comprises a light chain variable region (VL) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:42. In some embodiments, an anti-CD70 antibody comprises a VL comprising the amino acid sequence of SEQ ID NO:42. In some embodiments, a VL sequence having at least 90% sequence identity to a reference sequence (e.g., SEQ ID NO:42) contains one, two, three, four, five, six, seven, eight, nine, ten or more substitutions (e.g., conservative substitutions), insertions, or deletions relative to the reference sequence but retains the ability to bind to human CD70.

In some embodiments, an anti-CD70 antibody comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:41, and comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:42. In some embodiments, an anti-CD70 antibody comprises a heavy chain variable region comprising the amino acid sequence of SEQ ID NO:41, and comprises a light chain variable region comprising the amino acid sequence of SEQ ID NO:42.

In some embodiments, the anti-CD70 antibody comprises the heavy chain variable region and light chain variable region disclosed as SEQ ID NO:41 and 42, respectively.

E. Exemplary Anti-BCMA Antibodies

In some embodiments, a nonfucosylated anti-BCMA antibody is provided for use in the present methods as the antibody that binds an immune cell engager. In some embodiments, the nonfucosylated anti-BCMA antibody is SEA-BCMA, which is an antibody targeting B-cell maturation antigen (BCMA) and which comprises heavy chain CDR1, CDR2, and CDR3, and light chain CDR1, CDR2, and CDR3 comprising the amino acid sequences of SEQ ID NOs: 47-52, respectively. The corresponding VH and VL comprise the amino acid sequences of SEQ ID NOs: 45 and 46, respectively. BCMA is expressed on multiple myeloma (MM). The antibody acts through blocking ligand mediated BCMA cell signaling, antibody dependent cellular phagocytosis (ADCP), and enhanced antibody dependent cellular cytotoxicity (ADCC).

In some embodiments, an anti-BCMA antibody comprises one or more (e.g., one, two, three, four, five, or six) of:
a heavy chain CDR1 sequence comprising the amino acid sequence of SEQ ID NO:47;
a heavy chain CDR2 sequence comprising the amino acid sequence of SEQ ID NO:48;
a heavy chain CDR3 sequence comprising the amino acid sequence of SEQ ID NO:49;
a light chain CDR1 sequence comprising the amino acid sequence is SEQ ID NO:50;
a light chain CDR2 sequence comprising the amino acid is SEQ ID NO:51; and/or

a light chain CDR3 sequence comprising the amino acid sequence of SEQ ID NO:52.

In some embodiments, an anti-BCMA antibody comprises a heavy chain CDR1 sequence comprising the amino acid sequence of SEQ ID NO:47; a heavy chain CDR2 sequence comprising the amino acid sequence of any of SEQ ID NO:48; and a heavy chain CDR3 sequence comprising the amino acid sequence of SEQ ID NO:49.

In some embodiments, an anti-BCMA antibody comprises a light chain CDR1 sequence comprising the amino acid sequence of SEQ ID NO:50; a light chain CDR2 sequence comprising the amino acid of SEQ ID NO:51; and a light chain CDR3 sequence comprising the amino acid sequence of SEQ ID NO:52.

In some embodiments, an anti-BCMA antibody comprises a heavy chain CDR1 sequence comprising the amino acid sequence of SEQ ID NO:47; a heavy chain CDR2 sequence comprising the amino acid sequence of SEQ ID NO:48; a heavy chain CDR3 sequence comprising the amino acid sequence of SEQ ID NO:49; a light chain CDR1 sequence comprising the amino acid sequence of SEQ ID NO:50; a light chain CDR2 sequence comprising the amino acid sequence of SEQ ID NO:51; and a light chain CDR3 sequence comprising the amino acid sequence of SEQ ID NO:52.

In some embodiments, an anti-BCMA antibody comprises a heavy chain variable region (VH) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:45. In some embodiments, an anti-BCMA antibody comprises a VH comprising the amino acid sequence of NO:45. In some embodiments, a VH sequence having at least 90% sequence identity to a reference sequence (e.g., SEQ ID NO:45) contains one, two, three, four, five, six, seven, eight, nine, ten or more substitutions (e.g., conservative substitutions), insertions, or deletions relative to the reference sequence but retains the ability to bind to human BCMA.

In some embodiments, an anti-BCMA antibody comprises a light chain variable region (VL) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:46. In some embodiments, an anti-BCMA antibody comprises a VL comprising the amino acid sequence of SEQ ID NO:46. In some embodiments, a VL sequence having at least 90% sequence identity to a reference sequence (e.g., SEQ ID NO:46) contains one, two, three, four, five, six, seven, eight, nine, ten or more substitutions (e.g., conservative substitutions), insertions, or deletions relative to the reference sequence but retains the ability to bind to human BCMA.

In some embodiments, an anti-BCMA antibody comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:45, and comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:46. In some embodiments, an anti-BCMA antibody comprises a heavy chain variable region comprising the amino acid sequence of SEQ ID NO:45, and comprises a light chain variable region comprising the amino acid sequence of SEQ ID NO:46.

In some embodiments, the anti-BCMA antibody comprises the heavy chain variable region and light chain variable region disclosed as SEQ ID NO:45 and 46, respectively.

F. Enhanced Fc Backbone

As noted above, the antibodies that bind the immune cell engager comprise an Fc that has one or more of the following activities: enhanced binding to one or more activating FcγRs; reduced binding to inhibitory FcγRs; enhanced ADCC activity; and/or enhanced ADCP activity. Antibodies having Fc with such activities and the desired activity profile can be generated in a variety of ways, including producing a nonfucosylated protein and/or by engineering the Fc to contain certain mutations that yield the desired activity. This section provides additional details on methods for generating nonfucosylated antibodies and exemplary engineering approaches. Additional guidance on selection of constant regions and manufacturing of antibodies is provided in other sections below.

Antibodies may be glycosylated at conserved positions in their constant regions (Jefferis and Lund, (1997) Chem. Immunol. 65:111-128; Wright and Morrison, (1997) TibTECH 15:26-32). The oligosaccharide side chains of the immunoglobulins affect the protein's function (Boyd et al., (1996) Mol. Immunol. 32:1311-1318; Wittwe and Howard, (1990) Biochem. 29:4175-4180), and the intramolecular interaction between portions of the glycoprotein which can affect the conformation and presented three-dimensional surface of the glycoprotein (Jefferis and Lund, supra; Wyss and Wagner, (1996) Current Opin. Biotech. 7:409-416). Oligosaccharides may also serve to target a given glycoprotein to certain molecules based upon specific recognition structures. For example, it has been reported that in agalactosylated IgG, the oligosaccharide moiety ‘f1ips’ out of the inter-CH2 space and terminal N-acetylglucosamine residues become available to bind mannose binding protein (Malhotra et al., (1995) Nature Med. 1:237-243). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., (1996) Mol. Immunol. 32:1311-1318), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al. (1999) Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc.

Addition of glycosylation sites to the antibody can be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of the antibody.

The amino acid sequence is usually altered by altering the underlying nucleic acid sequence. These methods include isolation from a natural source (in the case of naturally-occurring amino acid sequence variants) or preparation by oligonucleotide-mediated (or site-directed) mutagenesis, PCR mutagenesis, and cassette mutagenesis of an earlier prepared variant or a non-variant version of the antibody.

The glycosylation (including glycosylation pattern) of antibodies may also be altered without altering the amino acid sequence or the underlying nucleotide sequence. See, e.g., Pereira et al., 2018, MAbs, 10(5): 693-711. Glycosylation largely depends on the host cell used to express the antibody. Since the cell type used for expression of recombinant glycoproteins, e.g., antibodies, as potential therapeutics is rarely the native cell, significant variations in the glycosylation pattern of the antibodies can be expected. See, e.g., Hse et al., (1997) J. Biol. Chem. 272:9062-9070. In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261; 5,278,299). Glycosylation, or certain types of glycosylation, can be enzymatically removed from the glycoprotein, for example using endoglycosidase H (Endo H). In addition, the recombinant host cell can be genetically engineered, e.g., make defective in processing certain types of polysaccharides. These and similar techniques are known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-P-galactosidase), elimination using harsh alkaline environment to release mainly O-linked structures, and chemical methods using anhydrous hydrazine to release both N- and O-linked oligosaccharides

A preferred form of modification of glycosylation of antibodies is reduced core fucosylation. “Core fucosylation” refers to addition of fucose (“fucosylation”) to N-acetylglucosamine (“GlcNAc”) at the reducing terminal of an N-linked glycan.

A “complex N-glycoside-linked sugar chain” is typically bound to asparagine 297 (according to the number of Kabat). As used herein, the complex N-glycoside-linked sugar chain has a biantennary composite sugar chain, mainly having the following structure:

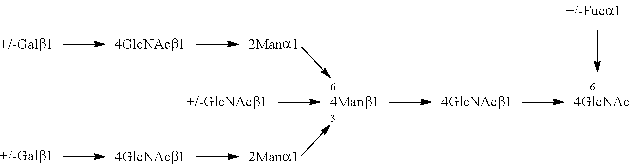

where± indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1, 6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has zero, one or more branches of galactose-N-acetylglucosamine (also referred to as “gal-GlcNAc”) and the non-reducing terminal side of gal-GlcNAc optionally has a sialic acid, bisecting N-acetylglucosamine or the like; and 2) a hybrid type, in which the non-reducing terminal side of the core structure has both branches of a high mannose N-glycoside-linked sugar chain and complex N-glycoside-linked sugar chain.

In some methods as provided herein, only a minor amount of fucose is incorporated into the complex N-glycoside-linked sugar chain(s) of the antibodies. For example, in various embodiments, less than about 60%, less than about 50%, less than about 40%, less than about 30%, less than about 20%, less than about 15%, less than about 10%, less than about 5%, or less than about 3% of the antibodies in a composition have core fucosylation by fucose. In some embodiments, about 2% of the antibodies in the composition have core fucosylation by fucose. In various embodiments, when less that 60% of the antibodies in a composition have core fucosylation by fucose, the antibodies of the composition may be referred to as “nonfucosylated” or “afucosylated.” In some embodiments, at least 90%, at least 91%, at least 92%, at least 93%, at least 94%, at least 95%, at least 96%, at least 97%, at least 98%, or at least 99% of the antibodies in the composition are nonfucosylated.

In certain embodiments, only a minor amount of a fucose analog (or a metabolite or product of the fucose analog) is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 3% of the antibodies have core fucosylation by a fucose analog or a metabolite or product of the fucose analog. In some embodiments, about 2% of the antibodies have core fucosylation by a fucose analog or a metabolite or product of the fucose analog.

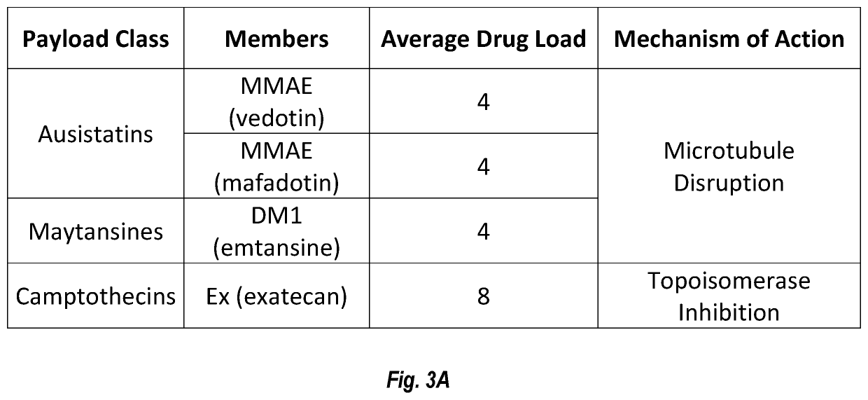

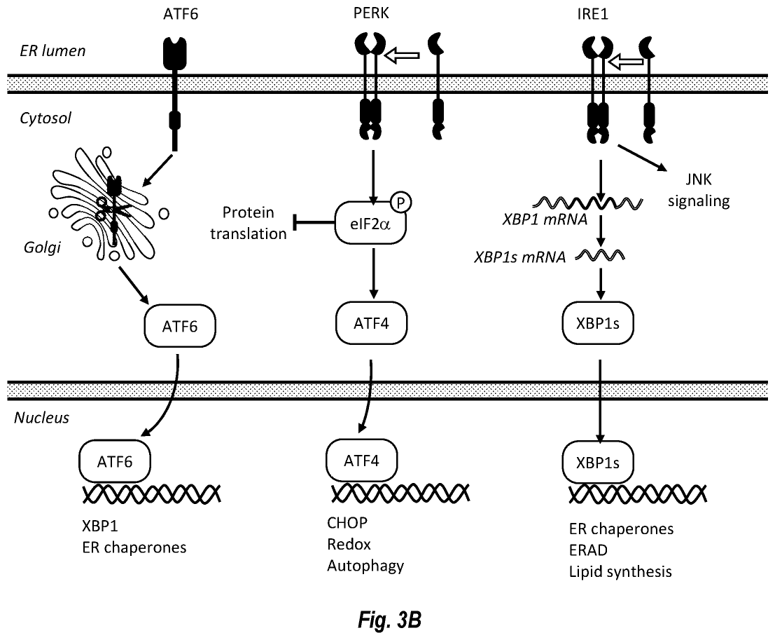

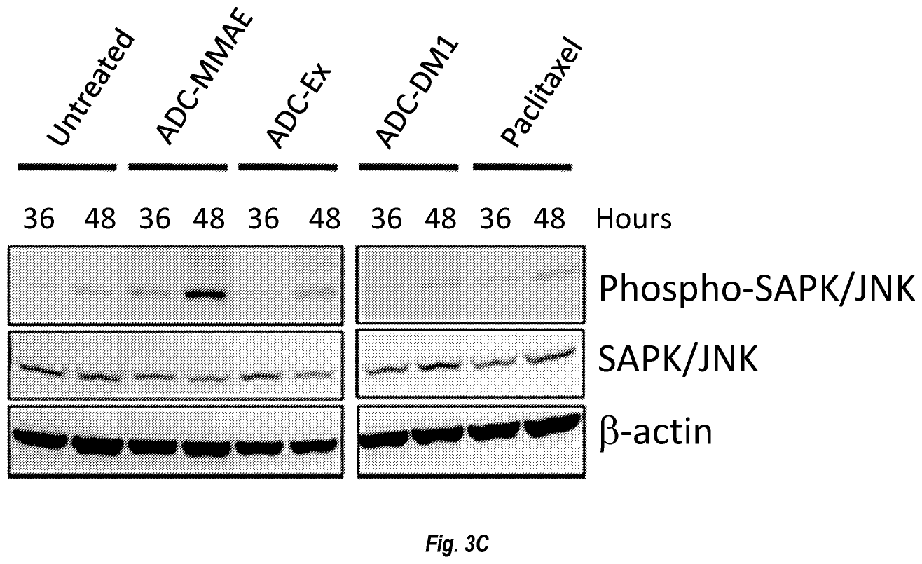

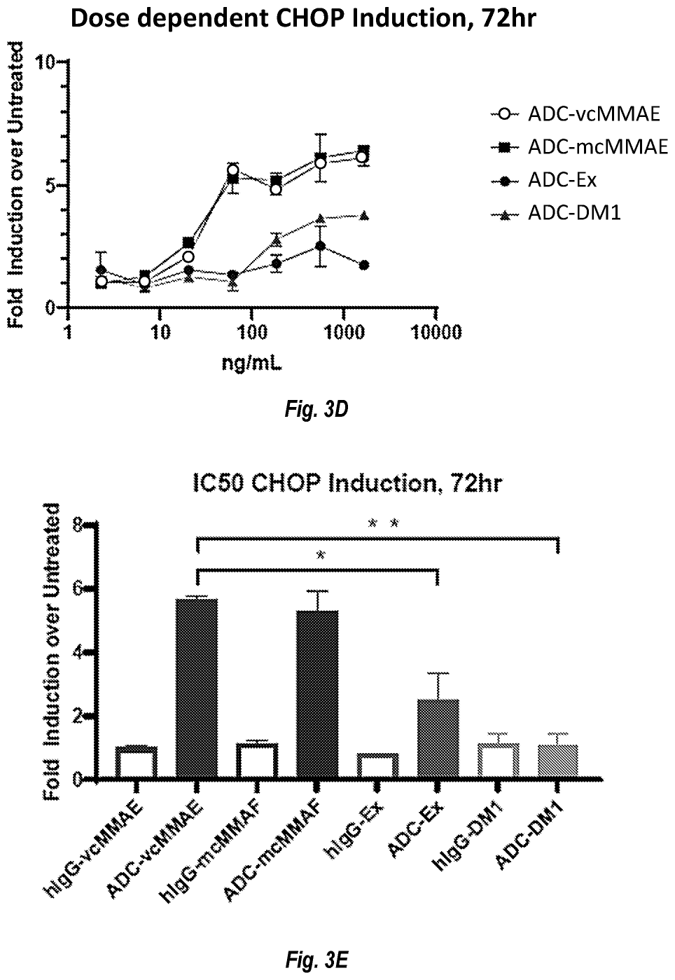

In some embodiments, less that about 60%, less than about 50%, less than about 40%, less than about 30%, less than about 20%, less than about 15%, less than about 10%, less than about 5%, or less than about 3% of the antibodies in a composition have a fucose residue on a GO, G1, or G2 glycan structure. (See, e.g., Raju et al., 2012, MAbs 4: 385-391, FIG. 3.) In some embodiments, about 2% of the antibodies in the composition have a fucose residue on a GO, G1, or G2 glycan structure. In various embodiments, when less than 60% of the antibodies in a composition have a fucose residue on a GO, G1, or G2 glycan structure, the antibodies of the composition may be referred to as “nonfucosylated.” In some embodiments, at least 90%, at least 91%, at least 92%, at least 93%, at least 94%, at least 95%, at least 96%, at least 97%, at least 98%, or at least 99% of the antibodies in the composition lack fucose on a G0, G1, or G2 glycan structure. It should be noted that G0 glycans include G0-GN glycans. G0-GN glycans are monoantenary glycans with one terminal GlcNAc residue. G1 glycans include G1-GN glycans. G1-GN glycans are monoantenary glycans with one terminal galactose residue. G0-GN and G1-GN glycans can be fucosylated or nonfucosylated.

A variety of methods for generating nonfucosylated antibodies can be utilized. Exemplary strategies include the use of cell lines lacking certain biosynthetic enzymes involved in fucosylation pathways or the inhibition or the knockout of certain genes involved in the fucosylation pathway. A review of such approaches is provided by Pereira, et al. (2018) MABS 10:693-711, which is incorporated herein by reference in its entirety.

For example, methods of making nonfucosylated antibodies by incubating antibody-producing cells with a fucose analogue are described, e.g., in WO2009/135181. Briefly, cells that have been engineered to express the antibodies are incubated in the presence of a fucose analogue or an intracellular metabolite or product of the fucose analog. An intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In one embodiment, the fucose analogue is 2-f1urofucose. Methods of using fucose analogues in growth medium and other fucose analogues are disclosed, e.g., in WO 2009/135181, which is herein incorporated by reference.

Other methods for engineering cell lines to reduce core fucosylation included gene knock-outs, gene knock-ins and RNA interference (RNAi). See, e.g., Pereira et al., 2018, MAbs, 10(5): 693-711. In gene knock-outs, the gene encoding FUT8 (alpha 1,6- fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely.

Other strategies that may be used include GlycoMAb® (U.S. Pat. No. 6,602,684) and Potelligent® (BioWa).

Any of these methods can be used to generate a cell line that would be able to produce a nonfucosylated antibody.

Various engineering approaches can also be utilized to obtain Fc regions with the desired FcγR activity and effector function. In some embodiments, the Fc is engineered to have the following combination of mutations: S239D, A330L and 1332E, which increases the affinity of the Fc domain for FcγRIIIA and consequently increases ADCC. Additional substitutions that enhance affinity for FcγRIIIa include, for example, T256A, K290A, S298A, E333A, and K334A. Substitutions that enhance binding to activating FcγRIIIa and reduced binding to inhibitory FcγRIIIb include, for example, F243L/R292P/Y300L/V305I/P396L and F243L/R292P/Y300L/L235V/P396L. In some embodiments, the substitutions are in an IgG1 Fc background.

Oligosaccharides covalently attached to the conserved Asn297 are involved in the ability of the Fc region of an IgG to bind FcγR (Lund et al., 1996, J. Immunol. 157:4963-69; Wright and Morrison, 1997, Trends Biotechnol. 15:26-31). Engineering of this glycoform on IgG can significantly improve IgG-mediated ADCC. Addition of bisecting N-acetylglucosamine modifications (Umana et al., 1999, Nat. Biotechnol. 17:176-180; Davies et al., 2001, Biotech. Bioeng. 74:288-94) to this glycoform or removal of fucose (Shields et al., 2002, J. Biol. Chem. 277:26733-40; Shinkawa et al., 2003, J. Biol. Chem. 278:6591-604; Niwa et al., 2004, Cancer Res. 64:2127-33) from this glycoform are two examples of IgG Fc engineering that improves the binding between IgG Fc and FcγR, thereby enhancing Ig-mediated ADCC activity.

A systemic substitution of solvent-exposed amino acids of human IgG1 Fc region has generated IgG variants with altered FcγR binding affinities (Shields et al., 2001, J. Biol. Chem. 276:6591-604). When compared to parental IgG1, a subset of these variants involving substitutions at Thr256/Ser298, Ser298/Glu333, Ser298/Lys334, or Ser298/Glu333/Lys334 to Ala demonstrate increased in both binding affinity toward FcγR and ADCC activity (Shields et al., 2001, J. Biol. Chem. 276:6591-604; Okazaki et al., 2004, J. Mol. Biol. 336:1239-49).

Many methods are available to determine the amount of fucosylation on an antibody. Methods include, e.g., LC-MS via PLRP-S chromatography, electrospray ionization quadrupole TOF MS, Capillary Electrophoresis with Laser-Induced Fluorescence (CE-LIF), and Hydrophilic Interaction Chromatography with Fluorescence Detection (HILIC).

IV. Exemplary Antibody-Drug Conjugates (ADCs)

The preceding section described the relevant aspects of the antibody that binds to a target that is involved in immune regulation (an immune cell engager). As noted above, some of the methods that are provided herein also comprise administering an antibody-drug conjugate (ADC) comprising a tubulin disrupter (e.g., auristatins, including for instance MMAE and MMAF) in combination with the antibody that binds to the immune cell engager. In various embodiments, the antibody-drug conjugate comprises an antibody conjugated to a cytotoxic agent. In some embodiments, the cytotoxic agent is a tubulin disrupter. In some embodiments, the antibody binds an antigen expressed on a tumor cell. Further details regarding the ADC that is utilized in the methods provided herein are set forth in this section and in the Examples below. Any of the ADCs described herein may be combined with any of the antibodies that bind an immune cell engager described herein.

A. Exemplary Target Antigens

In some embodiments, the ADC binds an antigen expressed on a tumor cell.

In some embodiments, an ADC used in the methods provided herein comprises an antibody conjugated to a cytotoxic agent, wherein the antibody specifically binds an antigen selected from 5T4 (TPBG), ADAM-9, AG-7, ALK, ALP, AMHRII, APLP2, ASCT2, AVB6, AXL (UFO), B7-H3 (CD276), B7-H4, BCMA, C3a, C3b, C4.4a (LYPD3), C5, C5a, CA6, CA9, CanAg, carbonic anhydrase IX (CAIX), Cathepsin D, CCR7, CD1, CD10, CD100, CD101, CD102, CD103, CD104, CD105, CD106, CD107a, CD107b, CD108, CD109, CD111, CD112, CD113, CD116, CD117, CD118, CD119, CD11A, CD11b, CD11c, CD120a, CD121a, CD121b, CD122, CD123, CD124, CD125, CD126, CD127, CD13, CD130, CD131, CD132, CD133, CD135, CD136, CD137, CD138, CD14, CD140a, CD140b, CD141, CD142, CD143, CD144, CD146, CD147, CD148, CD15, CD150, CD151, CD154, CD155, CD156a, CD156b, CD156c, CD157, CD158b2, CD158e, CD158f1, CD158h, CD158i, CD159a, CD16, CD160, CD161, CD162, CD163, CD164, CD166, CD167b, CD169, CD16a, CD16b, CD170, CD171, CD172a, CD172b, CD172g, CD18, CD180, CD181, CD183, CD184, CD185, CD19, CD194, CD197, CD1a, CD1b, CD1c, CD 1d, CD2, CD20, CD200, CD201, CD202b, CD203c, CD204, CD205, CD206, CD208, CD21, CD213al, CD213a2, CD217, CD218a, CD22, CD220, CD221, CD222, CD224, CD226, CD228, CD229, CD23, CD230, CD232, CD239, CD243, CD244, CD248, CD249, CD25, CD26, CD265, CD267, CD269, CD27, CD272, CD273, CD274, CD275, CD279, CD28, CD280, CD281, CD282, CD283, CD284, CD289, CD29, CD294, CD295, CD298, CD3, CD3 epsilon, CD30, CD300f, CD302, CD304, CD305, CD307, CD31, CD312, CD315, CD316, CD317, CD318, CD319, CD32, CD321, CD322, CD324, CD325, CD326, CD327, CD328, CD32b, CD33, CD331, CD332, CD333, CD334, CD337, CD339, CD34, CD340, CD344, CD35, CD352, CD36, CD37, CD38, CD39, CD3d, CD3g, CD4, CD41, CD42d, CD44, CD44v6, CD45, CD46, CD47, CD48, CD49a, CD49b, CD49c, CD49d, CD49e, CD49f, CD5, CD50, CD51, CD51 (integrin alpha-V), CD52, CD53, CD54, CD55, CD56, CD58, CD59, CD6, CD61, CD62L, CD62P, CD63, CD64, CD66a-e, CD67, CD68, CD69, CD7, CD70, CD70L, CD71, CD71 (TfR), CD72, CD73, CD74, CD79a, CD79b, CD8, CD80, CD82, CD83, CD84, CD85f, CD85i, CD85j, CD86, CD87, CD89, CD90, CD91, CD92, CD95, CD96, CD97, CD98, CDH6, CDH6 (cadherin 6), CDw210a, CDw210b, CEA, CEACAM5, CEACAM6, CFC1B, cKIT, CLDN18.2 (claudin 18.2), CLDN6, CLDN9, CLL-1, c-MET, complement factors C3, Cripto, CSP-1, CXCR5, DCLK1, DLK-1, DLL3, DPEP3, DR5 (Death receptor 5), Dysadherin, EFNA4, EGFR, EGFR wild type, EGFRviii, EGP-1 (TROP-2), EGP-2, EMP2, ENPP3, EpCAM, EphA2, EphA3, Ephrin-A4 (EFNA4), ETBR, FAP, FcRH5, FGFR2, FGFR3, FLT3, FOLR, FOLR1, FOLR-alpha, FSH, GCC, GD2, GD3, globo H, GPC1, GPC-1, GPC3, GPNMB, GPR20, HER2, HER-2, HER3, HER-3, HGFR (c-Met), HLA-DR, HM1.24, HSP90, Ia, IGF-1R, IL-13R, IL-15, IL1RAP, IL-2, IL-3, IL-4, IL7R, integrin alphaVbeta3 (integrin aVP3), integrin beta-6, Interleukin-4 Receptor (IL4R), KAAG-1, KLK2, LAMP-1, Le(y), Lewis Y antigen, LGALS3BP, LGR5, LH/hCG, LHRH, Lipid raft, LIV-1 (SLC39A6 or ZIP6), LRP-1, LRRC15, LY6E, macrophage mannose receptor 1, MAGE, Mesothelin (MSLN), MET, MHC class I chain-related protein A and B (MICA and MICB), MN/CA IX, MRC2, MT1-MMP, MTX3, MTX5, MUC1, MUC16, MUC2, MUC3, MUC4, MUC5, MUC5ac, NaPi2b, NCA-90, NCA-95, Nectin-4, Notch3, Nucleolin, OAcGD2, OT-MUC1 (onco-tethered-MUC1), OX001L, P1GF, PAM4 antigen, p-cadherin (cadherin 3), PD-L1, Phosphatidyl Serine(PS), PRLR, Prolactin Receptor (PRLR), Pseudomonas, PSMA, PTK4, PTK7, Receptor tyrosine kinase (RTK), RNF43, ROR1, ROR2, SAIL, SEZ6, SLAMF7, SLC44A4, SLITRK6, SLMAMF7 (CS1), SLTRK6, Sortilin (SORT1), SSEA-4, SSTR2, Staphylococcus aureus (antibiotic agent), STEAP-1, STING, STn, T101, TAA, TAC, TDGF1, tenascin, TENB2, TGF-B, Thomson-Friedenreich antigens, Thy1.l, TIM-1, tissue factor (TF; CD142), TM4SF1, Tn antigen, TNF-alpha (TNFα), TRA-1-60, TRAIL receptor (R1 and R2), TROP-2, Tumor-associated glycoprotein 72 (TAG-72), uPAR, VEGFR, VEGFR-2, and xCT

In some embodiments, the ADC binds an antigen selected from EGFR, KAAG1, MET, CD30, HER2, CD30, IL7R, CD248, Tumor-associated glycoprotein 72 (TAG-72), MRC2, EGFR, CD71, TRA-1-60, STn, CLDN18.2, CLDN6, HER-2, CD33, CD7, OT-MUC1 (onco-tethered-MUC1), TRA-1-60, TIM-1, GCC, Mesothelin (MSLN), EGFR, gpNMB, CD20, AMHRII, NaPi2b, CD142, ROR1, Integrin beta-6, Ly6E, cMET, CD37, MUC16, STEAP-1, LRRC15, SLITRK6, MUC16, ETBR, FCRH5, Ax1, CD79b, Globo H, SLAMF7, PSMA, CD22, CD228, CD48, LIV-1, EphA2, SLC44A4, CA9, Ax1, and LGR5.

In some embodiments, the ADC binds an antigen selected from BCMA, GPC1, CD30, cMET, SAIL, HER3, CD70, c-MET, CD46, HER2, 5T4, ENPP3, CD19, EGFR, BCMA, CD70, BCMA, and EphA2.

In some embodiments, the ADC binds an antigen selected from Her2, TROP2, BCMA, cMet, integrin alphVbeta6 (integrin aVP6), CD22, CD79b, CD30, CD19, CD70, CD228, and CD47.

In some embodiments, the ADC binds an antigen selected from CD142, Integrin beta-6, ENPP3, CD19, Ly6E, cMET, C4.4a, CD37, MUC16, STEAP-1, LRRC15, SLITRK6, MUC16, ETBR, FCRH5, Ax1, EGFR, CD79b, BCMA, CD70, PSMA, CD79b, CD228, CD48, LIV-1, EphA2, SLC44A4, CD30, and sTn.

In some embodiments, the ADC binds an antigen selected from 5T4, ADAM-9, AG-7, ALK, AMHRII, APLP2, ASCT2, Ax1, B7-H3, B7-H4, BCMA, C4.4a, CA6, CA9, CanAg, carbonic anhydrase IX (CAIX), Cathepsin D, CCR7, CD103, CD123, CD133, CD138, CD142, CD147, CD16, CD166, CD184, CD19, CD20, CD205, CD206, CD22, CD228, CD248, CD25, CD3, CD3 epsilon, CD30, CD300f, CD317, CD33, CD352, CD37, CD38, CD44v6, CD45, CD46, CD47, CD48, CD51, CD56, CD7, CD70, CD71, CD74, CD79b, CDH6, CEA, CEACAM5, CEACAM6, cKIT, CLDN18.2, CLDN6, CLDN9, CLL-1, c-MET, Cripto, CSP-1, CXCR5, DCLK1, DLK-1, DLL3, DPEP3, DR5 (Death receptor 5), Dysadherin, EFNA4, EGFR, EGFR wild type, EGFRviii, EMP2, ENPP3, EpCAM, EphA2, EphA3, ETBR, FAP, FCRH5, FGFR2, FGFR3, FLT3, FOLR, FOLR-alpha, FSH, GCC, GD2, GD3, Globo H, GPC-1, GPC3, gpNMB, GPR20, HER-2, HER-3, HLA-DR, HSP90, IGF-1R, IL-13R, IL-15, IL1RAP, IL-2, IL-3, IL-4, IL7R, Integrin beta-6, Interleukin-4 Receptor (IL4R), KAAG-1, KLK2, LAMP-1, Lewis Y antigen, LGALS3BP, LGR5, LH/hCG, LHRH, Lipid raft, LIV-1, LRP-1, LRRC15, Ly6E, Macrophage mannose receptor 1, MAGE, Mesothelin (MSLN), MET, MHC class I chain-related protein A and B (MICA and MICB), MRC2, MT1-MMP, MTX3, MTX5, MUC-1, MUC16, NaPi2b, Nectin-4, NOTCH3, Nucleolin, OAcGD2, OT-MUC1 (onco-tethered-MUC1), OX001L, P-Cadherin, PD-L1, Phosphatidyl Serine, Phosphatidylserine (PS), Prolactin Receptor (PRLR), Pseudomonas, PSMA, PTK7, Receptor tyrosine kinase (RTK), RNF43, ROR1, ROR2, SAIL, SEZ6, SLAMF7, SLC44A4, SLITRK6, Sortilin (SORT1), SSEA-4, SSTR2, Staphylococcus aureus (antibiotic agent), STEAP-1, STING, STING (payload target), STn, TAA, TGF-B, TIM-1, TM4SF1, TNF-alpha, TRA-1-60, TROP-2, Tumor-associated glycoprotein 72 (TAG-72), VEGFR-2, xCT.

In some embodiments, the ADC binds an antigen selected from ANMRII, Ax1, CA9, CD142, CD20, CD22, CD228, CD248, CD30, CD33, CD7, CD48, CD71, CD79b, CLDN18.2, CLDN6, c-MET, EGFR, EphA2, ETBR, FCRH5, GCC, Globo H, gpNMB, HER-2, IL7R, Integrin beta-6, KAAG-1, LGR5, LIV-1, LRRC15, Ly6E, Mesothelin (MSLN), MET, MRC2, MUC16, NaPi2b, Nectin-4, OT-MUC1 (onco-tethered-MUC1), PSMA, ROR1, SLAMF7, SLC44A4, SLITRK6, STEAP-1, STn, TIM-1, TRA-1-60, Tumor-associated glycoprotein 72 (TAG-72).

In some embodiments, the ADC binds an antigen selected from BCMA, GPC-1, CD30, c-MET, SAIL, HER-3, CD70, CD46, HER-2, 5T4, ENPP3, CD19, EGFR, EphA2.

In some embodiments, the antibody of the ADC does not bind Nectin-4.

Typically, the antibody of the ADC and the antibody that binds the immune cell engager are two separate antibodies. In certain embodiments, however, the antibodies may form a bispecific antibody.

B. Exemplary Cytotoxic Agents

In various embodiments, the methods provided herein comprise administering an antibody-drug conjugate, wherein the antibody-drug conjugate comprises an antibody conjugated to a tubulin disrupting agent.

Various categories of tubulin disrupting agent are known in the field, including, but not limited to, dolastatins, auristatins, tubulysins, colchicine, Vinca alkaloids, taxanes, T67 (Tularik), cryptophycins, maytansinoids, hemiasterlins, and other tubulin disrupting agents.

Auristatins are derivatives of the natural product dolastatin. Exemplary auristatins include dolostatin-10, auristatin E, auristatin T, MMAE (N-methylvaline-valine-dolaisoleuine-dolaproine-norephedrine or monomethyl auristatin E) and MMAF (N-methylvaline-valine-dolaisoleuine-dolaproine-phenylalanine or dovaline-valine-dolaisoleunine-dolaproine-phenylalanine), AEB (ester produced by reacting auristatin E with paraacetyl benzoic acid), AEVB (ester produced by reacting auristatin E with benzoylvaleric acid), and AFP (dimethylvaline-valine-dolaisoleuine- dolaproine-phenylalanine-p-phenylenediamine or auristatin phenylalanine phenylenediamine). WO 2015/057699 describes PEGylated auristatins including MMAE. Additional dolostatin derivatives contemplated for use are disclosed in U.S. Pat. No. 9,345,785, incorporated herein by reference for any purpose. Exemplary auristatin embodiments include the N-terminus linked monomethylauristatin drug units DE and DF, disclosed in “Senter et al, Proceedings of the American Association for Cancer Research, Volume 45, Abstract Number 623, presented Mar. 28, 2004 and described in U.S. Patent Publication No. 2005/0238649, the disclosure of which is expressly incorporated by reference in its entirety.

In certain embodiments, the ADC cytotoxic agent is MMAE.

In other embodiments, the cytotoxic agent conjugated to the ADC is MMAF.

Tubulysins include, but are not limited to, tubulysin D, tubulysin M, tubuphenylalanine and tubutyrosine. WO 2017-096311 and WO 2016-040684 describe nonlimiting tubulysin analogs including tubulysin M.

Colchicines include, but are not limited to, colchicine and CA-4.

Vinca alkaloids include, but are not limited to, Vinblastine (VBL), vinorelbine (VRL), vincristine (VCR) and vindfesine (VDS).

Taxanes include, but are not limited to, Taxol© (paclitaxel) and Taxotere© (docetaxel).

Cryptophycins include but are not limited to cryptophycin-1 and cryptophycin-52. Maytansinoids include, but are not limited to, maytansine, maytansinol, maytansine analogs, DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/− dechloro (U.S. Pat. No. 4,294,757) (prepared by acylation using acyl chlorides), and those having modifications at other positions. Maytansinoid drug moieties also include those having modifications such as: C-9-SH (U.S. Pat. No. 4,424,219) (prepared by the reaction of maytansinol with H.sub.25 or P.sub.2S.sub.5); C-14-alkoxymethyl(demethoxy/CH.sub.20R) (U.S. Pat. No. 4,331,598); C-14-hydroxymethyl or acyloxymethyl (CH.sub.20H or CH.sub.20Ac) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol). The cytotoxicity of the TA.1-maytansonoid conjugate that binds HER-2 (Chari et al., Cancer Research 52:127-131 (1992) was tested in vitro on the human breast cancer cell line SK-BR-3. The drug conjugate achieved a degree of cytotoxicity similar to the free maytansinoid drug, which could be increased by increasing the number of maytansinoid molecules per antibody molecule.

Hemiasterlins include but are not limited to, hemiasterlin and HTI-286.

Other tubulin disrupting agents include taccalonolide A, taccalonolide B, taccalonolide AF, taccalonolide AJ, taccalonolide AI-epoxide, discodermolide, baccatin derivatives, taxane analogs (e.g., epothilone A and epothilone B), nocodazole, colchicine, colcimid, estramustine, cemadotin, combretastatins, discodermolide, eleutherobin, eribulin, prolabolin, phomopsin, and laulimalide.

The ADC for use in the methods herein may, in some embodiments, comprise linker units. For example, the ADC may comprise a linker region between the cytotoxic agent and the antibody. In some embodiments, the linker is a protease cleavable linker, an acid-cleavable linker, a disulfide linker, or a self-stabilizing linker. In various embodiments, the linker is cleavable under intracellular conditions, such that cleavage of the linker releases the therapeutic agent from the antibody in the intracellular environment.

The ADC for use in the methods herein may comprise a linker wherein the therapeutic agent (e.g., tubulin disrupter) can be conjugated to the antibody in a manner that reduces its activity unless it is detached from the antibody (e.g., by hydrolysis, by antibody degradation, or by a cleaving agent). Such therapeutic agent can be attached to the antibody via a linker. A therapeutic agent conjugated to a linker is also referred to herein as a drug linker. The nature of the linker can vary widely. The components that make up the linker are chosen on the basis of their characteristics, which may be dictated in part, by the conditions at the site to which the conjugate is delivered.

The therapeutic agent can be attached to the antibody with a cleavable linker that is sensitive to cleavage in the intracellular environment of a target cell but is not substantially sensitive to the extracellular environment, such that the conjugate is cleaved from the antibody when it is internalized by the cancer cell (e.g., in the endosomal or, for example by virtue of pH sensitivity or protease sensitivity, in the lysosomal environment or in the caveolear environment). The therapeutic agent can also be attached to the antibody with a non-cleavable linker.

As indicated, the linker may comprise a cleavable unit. In some such embodiments, the structure and/or sequence of the cleavable unit is selected such that it is cleaved by the action of enzymes present at the target site (e.g., the target cell). In other embodiments, cleavable units that are cleavable by changes in pH (e.g. acid or base labile), temperature or upon irradiation (e.g. photolabile) may also be used.

In some embodiments, the cleavable unit may comprise one amino acid or a contiguous sequence of amino acids. The amino acid sequence may be the target substrate for an enzyme.

In some aspects, the cleavable unit is a peptidyl unit and is at least two amino acids long. Cleaving agents can include cathepsins B and D and plasmin (see, e.g., Dubowchik and Walker, 1999, Pharm. Therapeutics 83:67-123). Most typical are cleavable unit that are cleavable by enzymes that are present in the target cells, i.e., an enzyme cleavable linker. Accordingly, the linker can be cleaved by an intracellular peptidase or protease enzyme, including a lysosomal or endosomal protease. For example, a linker that is cleavable by the thiol-dependent protease cathepsin-B, which is highly expressed in cancerous tissue, can be used (e.g., a linker comprising a Phe-Leu or a Val-Cit peptide or a Val-Ala peptide).

In some embodiments, the linker will comprise a cleavable unit (e.g., a peptidyl unit) and the cleavable unit will be directly conjugated to the therapeutic agent. In other embodiments, the cleavable unit will be conjugated to the therapeutic agent via an additional functional unit, e.g., a self-immolative spacer unit or a non-self-immolative spacer unit. A non-self-immolative spacer unit is one in which part or all of the spacer unit remains bound to the drug unit after cleavage of a cleavable unit (e.g., amino acid) from the antibody-drug conjugate. To liberate the drug, an independent hydrolysis reaction takes place within the target cell to cleave the spacer unit from the drug.

With a self-immolative spacer unit, the drug is released without the need for drug for a separate hydrolysis step. In one embodiment, wherein the linker comprises a cleavable unit and a self immolative group, the cleavable unit is cleavable by the action of an enzyme and after cleavage of the cleavable unit, the self-immolative group(s) release the therapeutic agent. In some embodiments, the cleavable unit of the linker will be directly or indirectly conjugated to the therapeutic agent on one end and on the other end will be directly or indirectly conjugated to the antibody. In some such embodiments, the cleavable unit will be directly or indirectly (e.g., via a self-immolative or non-self-immolative spacer unit) conjugated to the therapeutic agent on one end and on the other end will be conjugated to the antibody via a stretcher unit. A stretcher unit links the antibody to the rest of the drug and/or drug linker. In one embodiment, the connection between the antibody and the rest of the drug or drug linker is via a maleimide group, e.g., via a maleimidocaproyl linker. In some embodiments, the antibody will be linked to the drug via a disulfide, for example the disulfide linked maytansinoid conjugates SPDB-DM4 and SPP-DM1.

The connection between the antibody and the linker can be via a number of different routes, e.g., through a thioether bond, through a disulfide bond, through an amide bond, or through an ester bond. In one embodiment, the connection between the antibody and the linker is formed between a thiol group of a cysteine residue of the antibody and a maleimide group of the linker. In some embodiments, the interchain bonds of the antibody are converted to free thiol groups prior to reaction with the functional group of the linker. In some embodiments, a cysteine residue is an introduced into the heavy or light chain of an antibody and reacted with the linker. Positions for cysteine insertion by substitution in antibody heavy or light chains include those described in Published U.S. Application No. 2007-0092940 and International Patent Publication WO2008070593, each of which are incorporated by reference herein in its entirety and for all purposes.

In some embodiments, the antibody-drug conjugates have the following formula I.

L-(LU-D)p (I)
wherein L is an antibody, LU is a Linker unit and D is a Drug unit (i.e., the therapeutic agent). The subscript p ranges from 1 to 20. Such conjugates comprise an antibody covalently linked to at least one drug via a linker. The Linker Unit is connected at one end to the antibody and at the other end to the drug.

The drug loading is represented by p, the number of drug molecules per antibody. Drug loading may range from 1 to 20 Drug units (D) per antibody. In some aspects, the subscript p will range from 1 to 20 (i.e., both integer and non-integer values from 1 to 20). In some aspects, the subscript p will be an integer from 1 to 20, and will represent the number of drug-linkers on a singular antibody. In other aspects, p represents the average number of drug-linker molecules per antibody, e.g., the average number of drug-linkers per antibody in a reaction mixture or composition (e.g., pharmaceutical composition), and can be an integer or non-integer value. Accordingly, in some aspects, for compositions (e.g., pharmaceutical compositions), p represents the average drug loading of the antibody-drug conjugates in the composition, and p ranges from 1 to 20.

In some embodiments, p is from about 1 to about 8 drugs per antibody. In some embodiments, p is 1. In some embodiments, p is 2. In some embodiments, p is from about 2 to about 8 drugs per antibody. In some embodiments, p is from about 2 to about 6, 2 to about 5, or 2 to about 4 drugs per antibody. In some embodiments, p is about 2, about 4, about 6 or about 8 drugs per antibody.

The average number of drugs per antibody unit in a preparation from a conjugation reaction may be characterized by conventional means such as mass spectroscopy, ELISA assay, HIC, and HPLC. The quantitative distribution of conjugates in terms of p may also be determined.

Exemplary antibody-drug conjugates include auristatin based antibody-drug conjugates, i.e., conjugates wherein the drug component is an auristatin drug. Auristatins bind tubulin, have been shown to interfere with microtubule dynamics and nuclear and cellular division, and have anticancer activity. Typically, the auristatin based antibody-drug conjugate comprises a linker between the auristatin drug and the antibody. The auristatins can be linked to the antibody at any position suitable for conjugation to a linker. The linker can be, for example, a cleavable linker (e.g., a peptidyl linker) or a non-cleavable linker (e.g., linker released by degradation of the antibody). The auristatin can be auristatin E or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include MMAF (monomethyl auristatin F), and MMAE (monomethyl auristatin E). The synthesis and structure of exemplary auristatins are described in U.S. Pat. or Publication Nos. 7,659,241, 7,498,298, 2009-0111756, 2009-0018086, and 7,968, 687 each of which is incorporated herein by reference in its entirety and for all purposes.

Exemplary auristatin based antibody-drug conjugates include vcMMAE, vcMMAF and mcMMAF antibody-drug conjugates as shown below wherein Ab is an antibody as described herein and val-cit represents the valine-citrulline dipeptide:

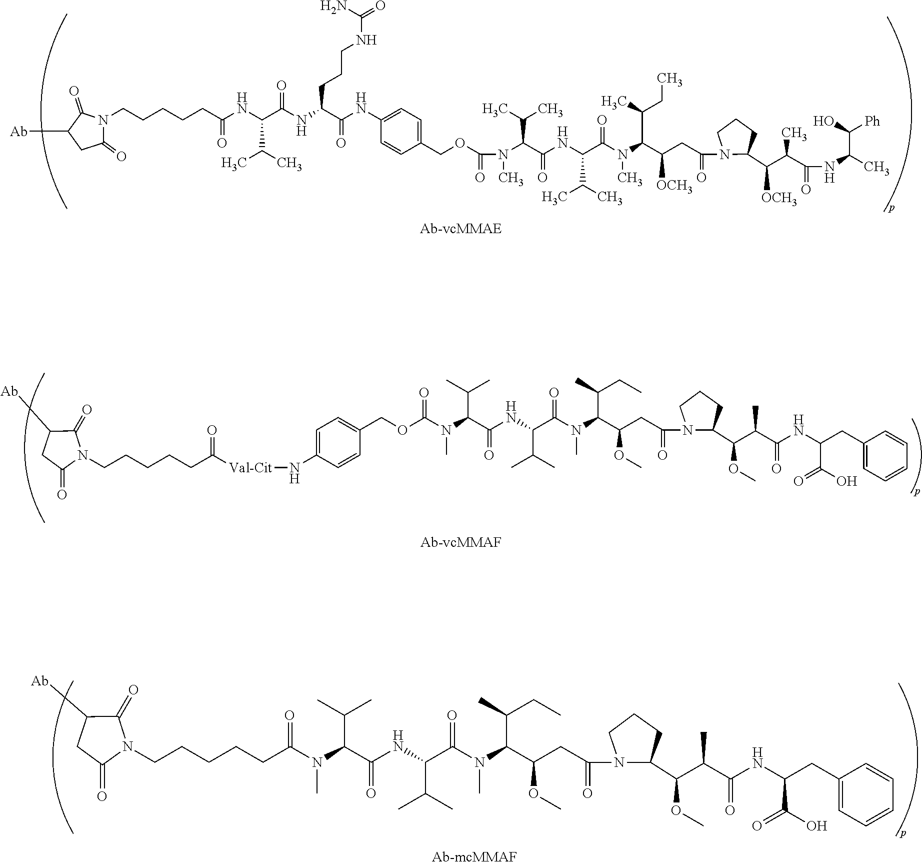

or a pharmaceutically acceptable salt thereof. The drug loading is represented by p, the number of drug-linker molecules per antibody. Depending on the context, p can represent the average number of drug-linker molecules per antibody, also referred to the average drug loading. The variable p ranges from 1 to 20 and is preferably from 1 to 8. In some preferred embodiments, when p represents the average drug loading, p ranges from about 2 to about 5. In some embodiments, p is about 2, about 3, about 4, or about 5. In some aspects, the antibody is conjugated to the linker via a sulfur atom of a cysteine residue. In some aspects, the cysteine residue is one that is engineered into the antibody. In other aspects, the cysteine residue is an interchain disulfide cysteine residue.

In some other embodiments, the antibody-drug conjugates have the linker units disclosed in the application US20160310612A1 (PCT/US2014/060477) herein incorporated in its entirety by reference. In some other embodiments, the antibody- drug conjugates have following formula (II).

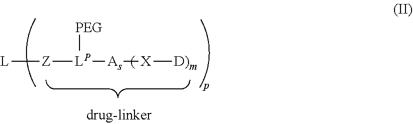

wherein D is a drug unit, PEG is the polyethylene glycol unit that masks the hydrophobicity of the drug-linker, LP is the parallel connector unit that allows for a PEG Unit to be in a parallel orientation with respect to X-D, A is a branching unit when m is greater than 1, optionally comprised of subunits, or A is absent when m is 1, X is a Releasable Assembly unit that provides for release of each D from the LDC and Z is an optional spacer unit through which LP is bonded to L, which is the antibody.

In some embodiments, the antibody-drug conjugates have the following formula III:

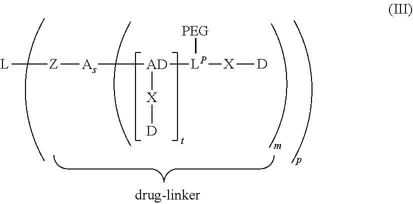

wherein AD is a drug attachment unit that allows for additional attachment of X-D moieties indicated by t in parallel orientation to the PEG Unit and L, L^{P}, Z, A, X, D, m, p and s are as defined for Formula II

In yet other principle embodiments an LDC of the present invention is represented by the structure of Formula IV below:

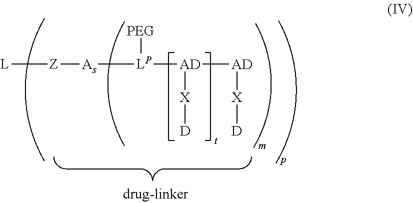

wherein AD, L, L^{P}, PEG, Z, A, X, D, m, p, s and t are as defined for Formula III.

In some embodiments, the antibody-drug conjugates have the following formula 1:

L-[LU-D′]ₚ (1)
or a salt thereof, in particular a pharmaceutically acceptable salt, wherein
L is an antibody;
LU is a Linker Unit; and
D′ represents from 1 to Drug Units (D) in each drug linker moiety of formula -LU-D′; and
subscript p is a number from 1 to 12, from 1 to 10 or from 1 to 8 or is about 4 or about 8,
wherein the antibody is capable of selective binding to an antigen of tumor tissue for subsequent release of the Drug Unit as free cytotoxic agent,
wherein the drug linker moiety of formula -LU-D′ in each of the antibody-drug conjugate of the composition has the structure of Formula 1A:

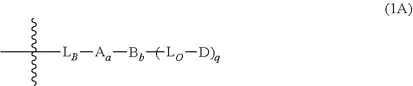

or a salt thereof, in particular a pharmaceutically acceptable salt,
wherein the wavy line indicates covalent attachment to L;
D is the Drug Unit of the cytotoxic agent;
L_{B} is an antibody covalent binding moiety;
A is a first optional Stretcher Unit;
subscript a is 0 or 1 indicating the absence of presence of A, respectively;
B is an optional Branching Unit;
subscript b is 0 or 1, indicating the absence of presence of B, respectively;
L₀ is a secondary linker moiety, wherein the secondary linker has the formula of;

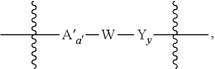

wherein the wavy line adjacent to Y indicates the site of covalent attachment of Lo to the Drug Unit and the wavy line adjacent A′ to indicates the site of covalent attachment to the remainder of the drug linker moiety;
A′ is a second optional Stretcher Unit, which in the absence of B becomes a subunit of A,
subscript a′ is 0 or 1, indicating the absence or presence of A′, respectively,
   W is a Peptide Cleavable Unit, wherein the Peptide Cleavable Unit is a contiguous sequence of up to 12 (e.g., 3-12 or 3-10) amino acids, wherein the sequence is comprised of a selectivity conferring tripeptide that provides improved selectivity for exposure of tumor tissue over normal tissue to free cytotoxic agent released from the antibody-drug conjugates of the composition in comparison to the cytotoxic agent released from antibody-drug conjugate composition of a comparator antibody-drug conjugate composition in which the peptide sequence of its Peptide Cleavable Unit is the dipeptide -valine-citrulline-or -valine-alanine-;
wherein the tumor and normal tissues are of rodent species and wherein the Formula 1 composition provides said improved exposure selectivity demonstrated by:
retaining efficacy in a tumor xenograft model of the comparator antibody-drug conjugate composition when administered at the same effective amount and dose schedule previously determined for the comparator antibody-drug conjugate composition, and
showing a reduction in plasma concentration of the free cytotoxic agent released from the antibody-drug conjugates of the composition, and/or preservation of normal cells in tissue when administered at the same effective amount and dose schedule as in the tumor xenograft model to a non-tumor bearing rodent in comparison to the equivalent (e.g., same) administration of the comparator antibody-drug conjugate composition in which the antibody of both conjugate compositions are replaced by a non-binding antibody,
wherein cytotoxicity to cells in human tissue of the same type as the normal cells in the tissue of the non-tumor bearing rodent is responsible at least in part to an adverse event in a human subject to whom is administered a therapeutically effective amount of the comparator conjugate composition;
   Y is a self-immolative Spacer Unit; and
   subscript y is 0, 1 or 2 indicating the absence or presence of 1 or 2 of Y, respectively;
subscript q is an integer ranging from 1 to 4,
provided that subscript q is 1 when subscript b is 0 and subscript q is 2, 3 or 4 when subscript b is 1; and
wherein the antibody-drug conjugates of the composition have the structure of Formula 1 in which subscript p is replaced by subscript p′, wherein subscript p′ is an integer from 1 to 12, 1to 10 or 1 to 8 or is 4 or 8.

A related embodiment provides for a Drug Linker of Formula V:

LU′−(D′) (V)
or a salt thereof, in particular a pharmaceutically acceptable salt thereof, wherein LU′ is capable of providing a covalent bond between L and LU of Formula 1, and therefore is sometimes referred to as a Linker Unit precursor; and D′ represents from 1 to 4 Drug Units, wherein the Drug Linker is further defined by the structure of Formula VI:

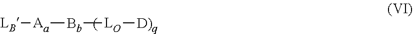

wherein LB′ is capable of transformation to L_{B} of Formula VI thereby forming a covalent bond to L of Formula 1, and therefore is sometimes referred to an antibody covalent binding precursor moiety, and the remaining variable groups of Formula VI are as defined for Formula VI.

In some embodiments, the ADC comprises an antibody (e.g., any antibody as described herein) conjugated to mc-vc-PABC-MMAE (also referred to herein as vcMMAE or 1006), mc-vc-PABC-MMAF, me-MMAF, or mp-dLAE-PABC-MMAE (also referred to herein as dLAE-MMAE, mp-dLAE-MMAE, or 7092), or a pharmaceutically acceptable salt thereof mp-dLAE-PABC-MMAE is described in PCT Publication No. WO 2021/055865 A1. Such ADCs are shown below, wherein Ab comprises an antigen-binding protein (e.g., any antibody as described herein), me represents a maleimidocaproyl group, mp refers to maleimidopropionyl:

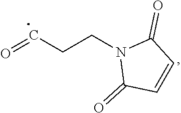

val-cit (vc) represents a valine-citrulline dipeptide, PABC represents ap-aminobenzyloxycarbonyl group, and dLAE represents a D-leucine-alanine-glutamic acid tripeptide:

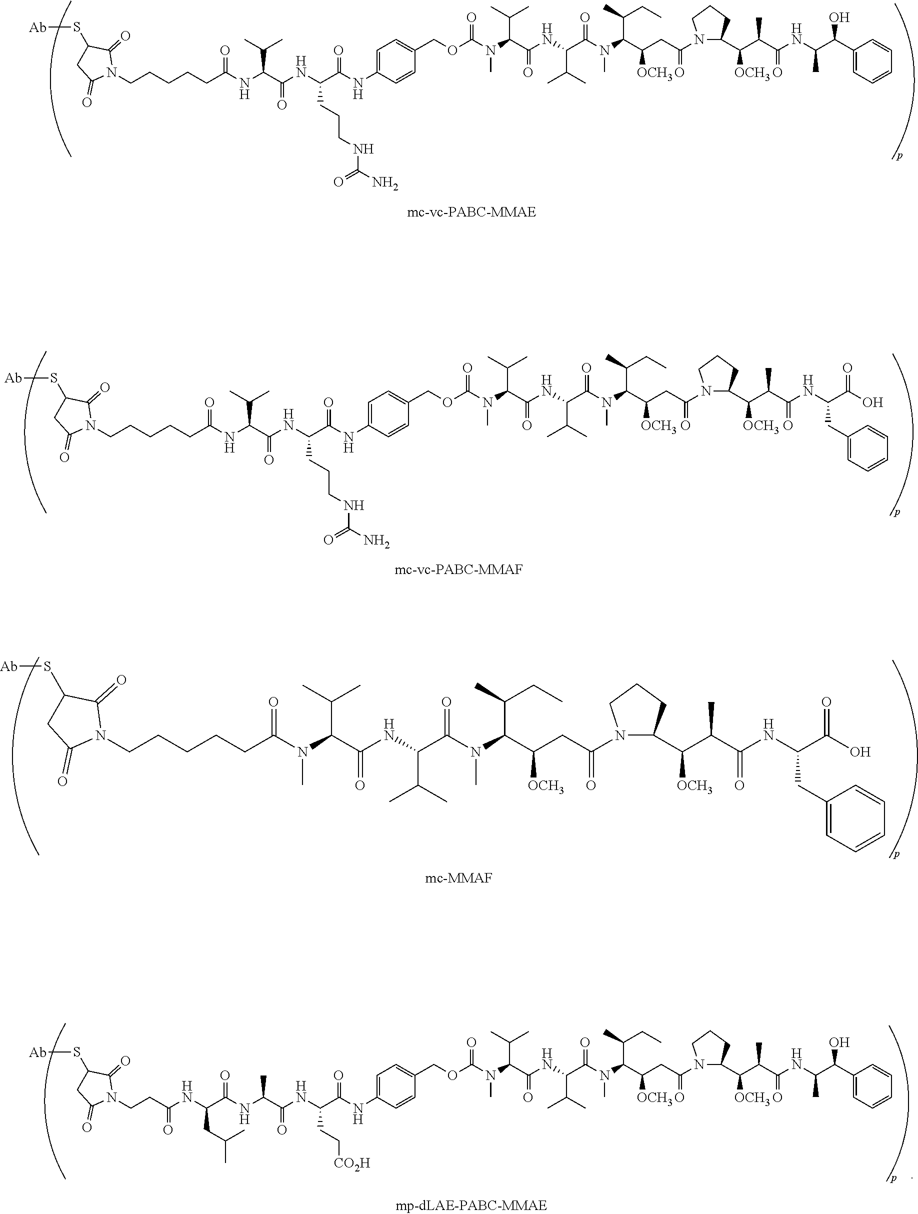

In some embodiments, the drug loading is represented by p, the number of drug-linker molecules per antibody. In some embodiments, p can represent the average number of drug-linker molecules per antibody in a composition of antibodies, also referred to the average drug loading. In some embodiments, p ranges from 1 to 20. In some embodiments, p ranges from 1 to 8. In some embodiments, when p represents the average drug loading, p ranges from about 2 to about 5. In some embodiments, p is about 2, about 3, about 4, or about 5. In some embodiments, the average number of drugs per antibody in a preparation may be characterized by conventional means such as mass spectroscopy, HIC, ELISA assay, and HPLC. In some embodiments, the antigen-binding protein (e.g,. an antibody) is attached to the drug-linker through a cysteine residue of the antibody. In some embodiments, the cysteine residue is one that is engineered into the antibody. In some embodiments, the cysteine residue is an interchain disulfide cysteine residue.

C. Exemplary ADCs

Nonlimiting exemplary ADCs for use in the present methods include ADCs comprising an antibody that binds any of the exemplary targets discussed herein conjugated to any of the tubulin disrupters described herein.

In some embodiments, the ADC is an anti-sialyl Tn antigen antibody-ADC, which comprises an antibody that binds to sialyl Tn antigen (sTn) and MMAE. See, e.g., US Patent Publication No. 2018/0327509A1; WO2017083582A1; Table of Sequences herein.

In some embodiments, the ADC is belantamab mafodotin, which comprises an antibody that binds to B-cell maturation antigen (BCMA) and MMAF. See, e.g., U.S. Pat. No. 9,273,141.

In some embodiments, the ADC is an anti-claudin-18.2 ADC, comprising an auristatin and an antibody as follows:
zolbetuximab (175D10), disclosed in U.S. Pat. No. 8,168,427, and comprising a heavy chain variable region (VH) comprising the amino acid sequence of SEQ ID NO:59 and a light chain variable region (VL) comprising the amino acid sequence of SEQ ID NO:60 or comprising a heavy chain CDR1, CDR2, and CDR3, and a light chain CDR1, CDR2, and CDR3 respectively comprising the amino acid sequences of SEQ ID NOs:61-66; 163E12, disclosed in U.S. Pat. No. 8,168,427, and comprising a heavy chain variable region (VH) comprising the amino acid sequence of SEQ ID NO:67 and a light chain variable region (VL) comprising the amino acid sequence of SEQ ID NO:68 or comprising a heavy chain CDR1, CDR2, and CDR3, and a light chain CDR1, CDR2, and CDR3 respectively comprising
the amino acid sequences of SEQ ID NOs:69-74;
any anti-claudin-18.2 antibody disclosed in PCT Publication No. WO 2020/135674 A1; or any anti-claudin-18.2 antibody disclosed in PCT Publication No. WO 2021/032157 A1.

In some embodiments, the ADC is SGN-PDL1V, comprising an anti-PD-L1 antibody and MMAE, the antibody comprising a heavy chain variable region (VH) comprising the amino acid sequence of SEQ ID NO:75 and a light chain variable region (VL) comprising the amino acid sequence of SEQ ID NO:76 or comprising a heavy chain CDR1, CDR2, and CDR3, and a light chain CDR1, CDR2, and CDR3 respectively comprising the amino acid sequences of SEQ ID NOs:77-82.

In some embodiments, the ADC is SGN-ALPV, comprising an anti-ALP antibody and MMAE, the antibody comprising a heavy chain variable region (VH) comprising the amino acid sequence of SEQ ID NO:83 and a light chain variable region (VL) comprising the amino acid sequence of SEQ ID NO:84 or comprising a heavy chain CDR1, CDR2, and CDR3, and a light chain CDR1, CDR2, and CDR3 respectively comprising the amino acid sequences of SEQ ID NOs:85-90.

In some embodiments, the ADC is SGN-B7H4V, comprising an anti-B7H4 antibody and MMAE, the antibody comprising a heavy chain variable region (VH) comprising the amino acid sequence of SEQ ID NO:91 and a light chain variable region (VL) comprising the amino acid sequence of SEQ ID NO:92 or comprising a heavy chain CDR1, CDR2, and CDR3, and a light chain CDR1, CDR2, and CDR3 respectively comprising the amino acid sequences of SEQ ID NOs:93-98.

In some embodiments, the ADC is disitamab vedotin, comprising an anti-HER2 antibody and MMAE, the antibody comprising a heavy chain comprising the amino acid sequence of SEQ ID NO:99 and a light chain comprising the amino acid sequence of SEQ ID NO:100.

In some embodiments, the ADC is lifastuzumab vedotin, comprising an anti-NaPi2B antibody and MMAE, the antibody comprising a heavy chain comprising the amino acid sequence of SEQ ID NO:101 and a light chain comprising the amino acid sequence of SEQ ID NO:102.

In some embodiments, the ADC is enfortumab vedotin, which comprises an antibody that binds nectin-4 and MMAE. See, e.g., U.S. Pat. No. 8,637,642; WO 2012/047724. In some embodiments, the antibody of enfortumab vedotin comprises a heavy chain variable region (VH) comprising the amino acid sequence of SEQ ID NO:103 and a light chain variable region (VL) comprising the amino acid sequence of SEQ ID NO:104 or comprising a heavy chain CDR1, CDR2, and CDR3, and a light chain CDR1, CDR2, and CDR3 respectively comprising the amino acid sequences of SEQ ID NOs:105-110.

In some embodiments, the ADC is SGN-B6A, which comprises an antibody that binds to AVB6 and MMAE. In some embodiments, SGN-B6A comprises a heavy chain variable region (VH) comprising the amino acid sequence of SEQ ID NO: 37 and a light chain variable region (VL) comprising the amino acid sequence of SEQ ID NO: 38. In some embodiments, the ADC comprises an anti-AVB6 antibody comprising a heavy chain variable region (VH) comprising the amino acid sequence of SEQ ID NO:111 and a light chain variable region (VL) comprising the amino acid sequence of SEQ ID NO:112 or comprising a heavy chain CDR1, CDR2, and CDR3, and a light chain CDR1, CDR2, and CDR3 respectively comprising the amino acid sequences of SEQ ID NOs:113-118. In some embodiments, the ADC comprises an anti-AVB6 antibody comprising a heavy chain variable region (VH) comprising the amino acid sequence of SEQ ID NO:119 and a light chain variable region (VL) comprising the amino acid sequence of SEQ ID NO:120 or comprising a heavy chain CDR1, CDR2, and CDR3, and a light chain CDR1, CDR2, and CDR3 respectively comprising the amino acid sequences of SEQ ID NOs:121-126.

In some embodiments, the ADC is an anti-CD228 antibody-ADC, which comprises an antibody that binds CD228 and MMAE. See, e.g., US Patent Publication No. 2020/0246479A1; WO2020/163225A1. In some embodiments, the ADC is SGN-CD228A, comprising an anti-CD228 antibody and MMAE, the antibody comprising a heavy chain variable region (VH) comprising the amino acid sequence of SEQ ID NO:127 and a light chain variable region (VL) comprising the amino acid sequence of SEQ ID NO:128 or comprising a heavy chain CDR1, CDR2, and CDR3, and a light chain CDR1, CDR2, and CDR3 respectively comprising the amino acid sequences of SEQ ID NOs:129-134.

In some embodiments, the ADC is SGN-LIV1A (ladiratuzumab vedotin; LV), comprising an anti-LIV-1 antibody and MMAE, the antibody comprising a heavy chain variable region (VH) comprising the amino acid sequence of SEQ ID NO:135 and a light chain variable region (VL) comprising the amino acid sequence of SEQ ID NO:136 or comprising a heavy chain CDR1, CDR2, and CDR3, and a light chain CDR1, CDR2, and CDR3 respectively comprising the amino acid sequences of SEQ ID NOs:137-142;
wherein SGN-LIV1A comprises the anti-LIV-1 antibody conjugated to mc-vc-PABC-MMAE, mc-vc-PABC-MMAF, me-MMAF, or mp-dLAE-PABC-MMAE.

In some embodiments, the ADC is tisotumab vedotin (TV), which comprises an antibody that binds tissue factor (TF) and MMAE. See, e.g., U.S. Pat. Nos. 9,168,314 and 9,150,658; WO 2011/157741; WO 2010/066803. In some embodiments, the antibody of TV comprises a heavy chain variable region (VH) comprising the amino acid sequence of SEQ ID NO:143 and a light chain variable region (VL) comprising the amino acid sequence of SEQ ID NO:144 or comprising a heavy chain CDR1, CDR2, and CDR3, and a light chain CDR1, CDR2, and CDR3 respectively comprising the amino acid sequences of SEQ ID NOs:145-150.

In some embodiments, an ADC comprises MMAE and binds a target selected from AMHRII, Ax1, CA9, CD142, CD20, CD22, CD228, CD248, CD30, CD33, CD7, CD48, CD71, CD79b, CLDN18.2, CLDN6, c-MET, EGFR, EphA2, ETBR, FCRH5, GCC, Globo H, gpNMB, HER-2, IL7R, Integrin beta-6, KAAG-1, LGR5, LIV-1, LRRC15, Ly6E, Mesothelin (MSLN), MET, MRC2, MUC16, NaPi2b, Nectin-4, OT-MUC1 (onco-tethered-MUC1), PSMA, ROR1, SLAMF7, SLC44A4, SLITRK6, STEAP-1, STn, TIM-1, TRA-1-60, Tumor-associated glycoprotein 72 (TAG-72).

In some embodiments, an ADC comprises MMAE and is one of: DP303c, also known as SYSA1501, targeting HER-2 (CSPC Pharmaceutical; Dophen Biomed), SIA01-ADC, also known as ST1, targeting STn (Siamab Therapeutics), Ladiratuzumab vedotin, also known as SGN-LIV1A, targeting LIV-1 (Merck & Co., Inc.; Seagen (Seattle Genetics) Inc.), ABBV-085, also known as Samrotamab vedotin, targeting LRRC15 (Abbvie; Seagen (Seattle Genetics) Inc.), DMOT4039A, also known as RG7600; αMSLN-MMAE, targeting Mesothelin (MSLN) (Roche-Genentech), RC68, also known as Remegen EGFR ADC, targeting EGFR (RemeGen (Rongchang Biopharmaceutical (Yantai) Co., Ltd.)), RC108, also known as RC108-ADC, targeting c-MET (RemeGen (Rongchang Biopharmaceutical (Yantai) Co., Ltd.)), CMG901, also known as MRG005, targeting CLDN18.2 (Keymed Biosciences; Lepu biotech; Shanghai Miracogen Inc. (Shanghai Meiya Biotechnology Co., Ltd)), YBL-001, also known as LCB67, targeting DLK-1 (Lego Chem Biosciences; Pyxis Oncology; Y-Biologics), DCDS0780A, also known as Iladatuzumab vedotin; RG7986, targeting CD79b (Roche-Genentech; Seagen (Seattle Genetics) Inc.), Tisotumab vedotin, also known as Humax-TF-ADC; tf-011-mmae; TIVDAK™ targeting CD142 (GenMab; Seagen (Seattle Genetics) Inc.), GO-3D1-ADC, also known as humAb-3D1-MMAE ADC, targeting MUC1-C(Genus Oncology LLC), ALT-P7, also known as HM2-MMAE, targeting HER-2 (Alteogen, Inc.; Levena Biopharma; 3SBio, Inc.), Vandortuzumab vedotin, also known as DSTP3086S; RG7450, targeting STEAP-1 (Roche-Genentech; Seagen (Seattle Genetics) Inc.), Lifastuzumab Vedotin, also known as DNIB0600A; NaPi2b ADC; RG7599, targeting NaPi2b (Roche-Genentech), Sofituzumab vedotin, also known as DMUC5754A; RG7458, targeting MUC16 (Seagen (Seattle Genetics) Inc.; Roche-Genentech), RG7841, also known as DLYE5953A, targeting Ly6E (Roche-Genentech; Seagen (Seattle Genetics) Inc.), RG7598, also known as DFRF4539A, targeting FCRH5 (Roche-Genentech; Seagen (Seattle Genetics) Inc.), RG7636, also known as DEDN6526A, targeting ETBR (Seagen (Seattle Genetics) Inc.; Roche-Genentech), Pinatuzumab vedotin, also known as DCDT2980S; RG7593, targeting CD22 (Roche-Genentech), Polatuzumab vedotin, also known as DCDS4501A; POLIVY™; RG7596; RO-5541077, targeting CD79b (Chugai Pharmaceutical; Roche-Genentech; Seagen (Seattle Genetics) Inc.), DMUC4064A, also known as D-4064a; RG7882, targeting MUC16 (Roche-Genentech; Seagen (Seattle Genetics) Inc.), SYSA1801, also known as CPO102, targeting CLDN18.2 (Conjupro Biotherapeutics Inc.; CSPC ZhongQi Pharmaceutical Technology Co.), RC 118, also known as Claudin18.2- ADC; YH005, targeting CLDN18.2 (RemeGen (Rongchang Biopharmaceutical (Yantai) Co., Ltd.); Biocytogen), VLS-101, also known as Cirmtuzumab vedotin; MK-2140; UC-961ADC3; Zilovertamab Vedotin, targeting ROR1 (VelosBio. Inc), Glembatumumab vedotin, also known as CDX-011; CR011-vcMMAE, targeting gpNMB (Celldex Therapeutics), BA3021, also known as CAB-ROR2-ADC; Ozuriftamab Vedotin, targeting ROR2 (Bioatla; Himalaya Therapeutics), BA3011, also known as CAB-AXL-ADC; Mecbotamab Vedotin, targeting Axl (Bioatla; Himalaya Therapeutics), CM-09, also known as Bstrongximab-ADC, targeting TRA-1-60 (CureMeta), ABBV-838, also known as Azintuxizumab vedotin, targeting SLAMF7 (Abbvie), Enapotamab vedotin, also known as AXL-107-MMAE; HuMax-AXL-ADC, targeting Axl (GenMab; Seagen (Seattle Genetics) Inc.), ARC-01, also known as anti-CD79b ADC, targeting CD79b (Araris Biotech AG), Disitamab vedotin, also known as Aidexi®; RC48, targeting HER-2 (RemeGen (Rongchang Biopharmaceutical (Yantai) Co., Ltd.); Seagen (Seattle Genetics) Inc.), ASG-5ME, also known as AGS-5; AGS-5ME, targeting SLC44A4 (Agensys, Inc.; Astellas Pharma Inc.; Seagen (Seattle Genetics) Inc.), Enfortumab vedotin, also known as AGS-22M6E; ASG-22CE; ASG-22ME; PADCEV™, targeting Nectin-4 (Astellas Pharma Inc.; Seagen (Seattle Genetics) Inc.), ASG-15ME, also known as AGS-15E; Sirtratumab vedotin, targeting SLITRK6 (Seagen (Seattle Genetics) Inc.; Astellas Pharma Inc.), Brentuximab vedotin, also known as Adcetris; cAC10-vcMMAE; SGN-35, targeting CD30 (Seagen (Seattle Genetics) Inc.; Takeda), Telisotuzumab vedotin, also known as ABBV-399, targeting c-MET (Abbvie), Losatuxizumab vedotin, also known as ABBV-221, targeting EGFR (Abbvie), CX-2029, also known as ABBV-2029, targeting CD71 (Abbvie; CytomX Therapeutics), AB-3A4-ADC, also known as AB-3A4-vcMMAE, targeting KAAG-1 (Alethia Biotherapeutics), Indusatumab vedotin, also known as 5F9-vcMMAE; MLN0264; TAK-264, targeting GCC (Takeda; Millennium Pharmaceuticals, Inc), FOR46 targeting CD46 (Fortis Therapeutics, Inc.), LR004-VC-MMAE targeting EGFR (Chinese Academy of Medical Sciences Peking Union Medical College Hospital), CD30-ADCs targeting CD30 (NBE Therapeutics; Boehringer Ingelheim), Anti-endosialin-MC—VC-PABC-MMAE targeting CD248 (Genzyme), OBI-998 targeting SSEA-4 (OBI Pharma), MRG002 targeting HER-2 (Lepu biotech; Shanghai Miracogen Inc. (Shanghai Meiya Biotechnology Co., Ltd)), TRS005 targeting CD20 (Teruisi Pharmaceuticals), Oba01 targeting DR5 (Death receptor 5) (Obio Technology (Shanghai) Corp., Ltd.; Yantai Obioadc Biomedical Technology Ltd.), PSMA ADC targeting PSMA (Progenics Pharmaceuticals, Inc; Seagen (Seattle Genetics) Inc.), SGN-CD48A targeting CD48 (Seagen (Seattle Genetics) Inc.), IMAB362-vcMMAE targeting CLDN18.2 (Astellas Pharma Inc.; Ganymed), GB251 targeting HER-2 (Genor Biopharma Co., Ltd.), Innate Pharma BTG-ADCs targeting CD30 (Innate Pharma; Sanofi), ADCendo uPARAP ADC targeting MRC2 (ADCendo), XCN-010 targeting actM (Xiconic Pharmaceuticals, LLC), ANT-043 targeting HER-2 (Antikor Biopharma), OBI-999 targeting Globo H (Abzena; OBI Pharma), LY3343544 targeting MET (Eli Lilly and Company), Tagworks anti-TAG72 ADC targeting TAG-72 (Tagworks Pharmaceuticals), IMAB027-vcMMAE targeting CLDN6 (Ganymed; Astellas Pharma Inc.), LGR5-ADC targeting LGR5 (Genentech, Inc.), Philochem B12-MMAE ADC targeting IL-7R (Instituto de Medicina Molecular João Lobo Antunes; Philochem AG), TE-1522 targeting CD19 (Immunwork), SGN-STNV targeting STn (Seagen (Seattle Genetics) Inc.), HTI-1511 targeting EGFR (Abzena; Halozyme Therapeutics), Peptron PAb001-ADC targeting OT-MUC1 (onco-tethered-MUC1) (Peptron; Qilu Pharmaceutical co. Ltd.), LM-102 targeting CLDN18.2 (LaNova Medicines Limited), Anwita Biosciences MSLN-MMAE targeting Mesothelin (MSLN) (Anwita biosciences), SGN-CD228A targeting CD228 (Seagen (Seattle Genetics) Inc.), NBT828 targeting HER-2 (NewBio Therapeutics; Genor Biopharma Co., Ltd.), Gamamabs GM103 targeting AMHR2 (GamaMabs Pharma; Exelixis), LCB14-0302 targeting HER-2 (Lego Chem Biosciences), BAY79-4620 targeting carbonic anhydrase IX (CAIX) (Bayer; MorphoSys), NBT508 targeting CD79b (NewBio Therapeutics), PAT-DX3-MMAE targeting Undisclosed (Patrys; Yale University), AGS67E targeting CD37 (Astellas Pharma Inc.; Seagen (Seattle Genetics) Inc.), CDX-014 targeting TIM-1 (Celldex Therapeutics), BVX001 targeting CD33; CD7 (Bivictrix therapeutics), SGN-B6A targeting Integrin beta-6 (Seagen (Seattle Genetics) Inc.), MRG003 targeting EGFR (Lepu biotech; Shanghai Miracogen Inc. (Shanghai Meiya Biotechnology Co., Ltd)), and PYX-202 targeting DLK-1 (Pyxis Oncology; Lego Chem Biosciences).

In some embodiments an ADC comprises MMAF and binds a target selected from BCMA, GPC-1, CD30, c-MET, SAIL, HER-3, CD70, CD46, HER-2, 5T4, ENPP3, CD19, EGFR, EphA2.

In some embodiments, an ADC comprises MMAF and is one of: CD70-ADC targeting CD70 (Kochi University; Osaka University), IGN786 targeting SAIL (AstraZeneca; Igenica Biotherapeutics), PF-06263507 targeting 5T4 (Pfizer), GPC1-ADC targeting GPC-1 (Kochi University), ADC-AVP10 targeting CD30 (Avipep), M290-MC-MMAF targeting CD103 (The Second Affiliated Hospital of Harbin Medical University), BVX001 targeting CD33; CD7 (Bivictrix therapeutics), Tanabe P3D12-vc-MMAF targeting c-MET (Tanabe Research Laboratories), LILRB4-Targeting ADC targeting LILRB4 (The University of Texas Health Science Center, Houston), TSD101, also known as ABL201, targeting BCMA (TSD Life Science; ABL Bio; Lego Chem Biosciences), Depatuxizumab mafodotin, also known as ABT-414, targeting EGFR (Abbvie; Seagen (Seattle Genetics) Inc.), AGS16F, also known as AGS-16C3F; AGS-16M8F, targeting ENPP3 (Astellas Pharma Inc.; Seagen (Seattle Genetics) Inc.), AVG-A11 BCMA ADC, also known as AVG-A11-mcMMAF, targeting BCMA (Avantgen), Belantamab mafodotin, also known as BLENREP; GSK2857916; J6MO-mcMMAF, targeting BCMA (GlaxoSmithKline; Seagen (Seattle Genetics) Inc.), MP-HER3-ADC, also known as HER3-ADC, targeting HER-3 (MediaPharma), FS-1502, also known as LCB14-0110, targeting HER-2 (Lego Chem Biosciences; Shanghai Fosun Pharmaceutical Development Co, Ltd.), MEDI-547, also known as MI-CP177, targeting EphA2 (AstraZeneca; Seagen (Seattle Genetics) Inc.), Vorsetuzumab mafodotin, also known as SGN-75, targeting CD70 (Seagen (Seattle Genetics) Inc.), Denintuzumab mafodotin, also known as SGN-CD19A, targeting CD19 (Seagen (Seattle Genetics) Inc.), and HTI-1066, also known as SHR-A1403, targeting c-MET (Jiangsu HengRui Medicine Co., Ltd).

In some embodiments, an ADC is selected from the ADCs in Table A, Table B, or Table C. In Table A, Table B, and Table C, the ADCs with sequences provided in the Table of Sequences are marked with an asterisk (*). In some embodiments the ADC is not enfortumab vedotin. In certain embodiments, the ADC is not brentuximab vedotin. In some embodiments, the ADC is not tisotumab vedotin. In some embodiments, the ADC is not ladiratuzumab vedotin. In some embodiments, the ADC is not SGN-CD228A.

TABLE A

| Patent | Drug Names | Target | Moiety | Linker | Payload |
|---|---|---|---|---|---|
| U.S. Pat. No. 8,545,850 | Polatuzumab | CD79b | Polatuzumab | Valine-Citrulline | MMAE |
| | vedotin | | Isotype: IgG1 | | |
| | | | Origin: Humanized | | |
| | Gemtuzumab | CD33 | Gemtuzumab | AcBut acyl | Calicheamicin |
| | ozogamicin | | Isotype: IgG4 | hydrazone-disulfide | |
| | | | Origin: Humanized | | |
| U.S. Pat. No. 9,273,141 | Belantamab | BCMA | Belantamab (J6M0) | mc | MMAF |
| | mafodotin | | Isotype: IgG1 | | |
| | | | Origin: Humanized | | |
| | | | Format: mAB | | |
| U.S. Pat. No. 9,808,537 | Trastuzumab | HER-2 | Trastuzumab | GGFG | DXd/DX8951 |
| | deruxtecan | | Isotype: IgG1 | (Glycine-Glycine- | (MAAA-1181a) |
| | | | Origin: Humanized | Phenylalanine- | |
| | | | | Glycine) | |
| U.S. Pat. No. 8,637,642 | Enfortumab | Nectin-4 | Enfortumab | Valine-Citrulline | MMAE |
| (WO 2012/047724) | vedotin* | | Isotype: IgG1k | | |
| | | | Origin: Human | | |
| U.S. Pat. No. 8,153,768 | Inotuzumab | CD22 | Inotuzumab | AcBut acyl | Calicheamicin |
| | ozogamicin | | Isotype: IgG4 | hydrazone-disulfide | |
| | | | Origin: Humanized | | |
| WO 2004/010957 | Brentuximab | CD30 | Brentuximab | Valine-Citrulline | MMAE |
| | vedotin | | Isotype: IgG1 | | |
| | (SGN-35)* | | Origin: Chimeric | | |
| U.S. Pat. No. 7,517,964; | Sacituzumab | TROP-2 | Sacituzumab (hRS7) | CL2A | SN-38 |
| U.S. Pat. No. 8,877,901 | govitecan | | Isotype: IgG1 | | |
| | | | Origin: Humanized | | |
| | | | Format: mAB | | |
| U.S. Pat. No. 8,337,856 | Trastuzumab | HER-2 | Trastuzumab | SMCC | DM1 |
| | emtansine | | Isotype: IgG1 | | |
| | | | Origin: Humanized | | |

TABLE B

| | Other Drug | | | | |
|---|---|---|---|---|---|
| Drug Names | Names | Target | Moiety | Linker | Payload |
| CX-2029 | ABBV-2029 | CD71 | Probody | Valine-Citrulline | MMAE |
| DP303c | | HER-2 | | Unknown | MMAE |
| HTI-1066 | SHR-A1403 | c-MET | anti-C-MET Isotype: IgG2 Origin: | 3-proplythio-mc | MMAF |
| | | | Humanized Format: mAB | | |
| FOR46 | | CD46 | 23AG2 Isotype: IgG1 Origin: | Valine-Citrulline | MMAF |
| | | | Human Format: mAB Other: | | |
| | | | anti-CD46 humanized | | |
| AGS62P1 | ASP1235 | FLT3 | Anti-FLT3 monoclonal antibody | Oxime | AGD-0182 |
| | | | Isotype: IgG1 Origin: Human | | |
| BT8009 | | Nectin-4 | | Valine-Citrulline | MMAE |
| ARX788 | | HER-2 | | Oxime | Amberstatin269 |
| XMT-1536 | | NaPi2b | XMT-1535 Isotype: IgG1 Origin: | Fleximer Polymer | Auristatin |
| | | | Humanized Format: mAB | | F-HPA |
| ALT-P7 | HM2-MMAE | HER-2 | Trastuzumab Isotype: IgG1 | Valine-Citrulline | MMAE |
| | | | Origin: Humanized Format: | | |
| | | | mAB Other: NexMab variant | | |
| | | | of trastuzumab | | |
| FS-1502 | LCB14-0110 | HER-2 | Trastuzumab | β-glucuronidase | MMAF |
| | | | | (BG) linker | |
| Cofetuzumab | PF-06647020, | PTK7 | h6M24 Isotype: IgG1 Origin: | Valine-Citrulline | PF-06380101 |
| pelidotin | PTK7-ADC, | | Humanized Format: mAB | | |
| | PF-7020, | | | | |
| | ABBV-647 | | | | |
| TRS005 | | CD20 | Rituximab Isotype: IgG1 | Valine-Citrulline | MMAE |
| | | | Origin: Chimeric Format: mAb | | |
| STI-6129 | CD38 ADC, | CD38 | STI-5171 Isotype: IgG1 | Unknown | Duostatin 5.2 |
| | LNDS1001, | | Origin: Human Format: mAB | | |
| | CD38-077 ADC | | | | |
| Tisotumab | Humax-TF-ADC, | CD142 | HuMab (HuMax antibody) | Valine-Citrulline | MMAE |
| vedotin* | tf-011-mmae | | Isotype: IgG1 Origin: Human | | |
| Cirmtuzumab | VLS-101, | ROR1 | Cirmtuzumab (UC-961) | Protease | MMAE |
| vedotin | UC-961ADC3 | | Isotype: IgG1 Origin: | Cleavable | |
| | | | Humanized Format: mAB | | |
| AbGn-107 | Ab1-18Hr1 | AG-7 | AbGn-7 Isotype: IgG1 Origin: | Valine-Citrulline | MMAD |
| | | | Humanized Format: mAB | | |
| ASN-004 | | 5T4 | Undisclosed Format: | Fleximer Polymer | Dolastatin |
| | | | scFvFc antibody | | |
| SGN-B6A* | | Integrin | h2A2 Origin: Humanized | Valine-Citrulline | MMAE |
| | | beta-6 | | | |
| Disitamab | RC48 | HER-2 | Hertuzumab Isotype: IgG1 | Valine-Citrulline | MMAE |
| vedotin* | | | Origin: Humanized | | |
| | | | Format: mAB | | |
| Telisotuzumab | ABBV-399 | c-MET | ABT-700 | Valine-Citrulline | MMAE |
| vedotin | | | | | |
| Enapotamab | HuMax-AXL-ADC, | Axl | HuMax Antibody Origin: | Valine-Citrulline | MMAE |
| vedotin | AXL-107-MMAE | | Human Format: Full length | | |
| OBI-999 | | Globo H | OBI-888 Isotype: IgG1 Origin: | Disulphide | MMAE |
| | | | Humanized Format: mAb | | |
| XMT-1592 | | NaPi2b | Unknown | Unknown | Auristatin |
| | | | | | F-HPA |
| SGN-CD228A | | CD228 | hL49 (anti-CD228A monoclonal | β-glucuronidase | MMAE |
| | | | antibody) Origin: Humanized | (BG) linker | |
| | | | Format: mAB | | |
| Ladiratuzumab | SGN-LIV1A | LIV-1 | Ladiratuzumab (hLIV-22) | Valine-Citrulline | MMAE |
| vedotin* | | | Isotype: IgG1 Origin: | | |
| | | | Humanized Format: mAB | | |
| BT5528 | | EphA2 | Unknown | Valine-Citrulline | MMAE |
| PF-06804103 | NG-HER2 ADC | HER-2 | Unknown Isotype: IgG1 | Valine-Citrulline | PF-06380101 |
| | | | Origin: Human | | (Aur 101) |
| W0101 | | IGF-1R | hz208F2-4 (anti-IGF1R antibody) | mc | Auristatin |
| | | | Origin: Humanized Format: mAB | | |
| BA3011 | CAB-AXL-ADC | Axl | CAB-Axl Other: Conditionally | Unknown | MMAE |
| | | | Active Biologics (CAB) | | |
| | | | anti-Axl antibody | | |
| MRG002 | | HER-2 | anti-HER2 Isotype: IgG1 Origin: | Unknown | MMAE |
| | | | Humanized Format: mAB | | |
| ZW49 | | HER-2, | ZW25 Isotype: IgG1 Format: | Valine-Citrulline | Auristatin |
| | | HER-2 | Bispecific, ZW25 Isotype: IgG1 | | |
| | | | Format: Bispecific | | |

TABLE C

| Patent | ADC names | targets: |
|---|---|---|
| U.S. Pat. No. 9,168,314 | Tisotumab | CD142 |
| (WO 2011/157741) | vedotin (TV)* | (tissue |
| | | factor) |
| U.S. Pat. No. 9,273,141 | Belantamab | BCMA |
| | mafodotin | |
| U.S. Pat. No. 8,637,642 | Enfortumab | Nectin-4 |
| (WO 2012/047724) | vedotin (EV)* | |
| U.S. Pat. No. 11,028,181 | SGN-STNV | STN |
| (WO2017083582A1); see | | |
| Table of Sequences herein | | |
| | Enapotamab | Axl |
| | vedotin | |
| US 2020/0246479 (WO | SGN-CD228A* | CD228 |
| 2020/163225; VH/VL of SEQ | | |
| ID NO: 7 and 8, respectively | | |
| (CDRs SEQ ID NOs: 1-6)); see | | |
| Table of Sequences herein | | |
| US 2021/0198367 (claiming | SGN-B6A* | Integrin |
| priority to USSN 62/943,959 | | beta-6 |
| and USSN 62/012,584); See | | |
| Table of Sequences herein | | |
| WO 2012/078688 | Ladiratuzumab | LIV-1 |
| | vedotin (LV)* | |
| WO 2004/010957 | Brentuximab | CD30 |
| | vedotin (SGN-35)* | |
| U.S. Pat. No. 8,329,173 | Telisotuzumab | c-MET |
| | vedotin | |
| U.S. Pat. No. 8,545,850 | Polatuzumab | CD79b |
| | vedotin | |
| U.S. Pat. No. 7,662,387 | Vorsetuzumab | CD70 |
| | mafodotin | |

D. Preparation of Antibodies

For preparing an antibody, many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)).

The genes encoding the heavy and light chains of an antibody of interest can be cloned from a cell, e.g., the genes encoding a monoclonal antibody can be cloned from a hybridoma that expresses the antibody and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. (2010) PEDS 23:311; and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778, 4,816,567) can also be adapted to produce antibodies. Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or antibodies covalently bound to immunotoxins (see, e.g., U.S. Pat. No. 4,676,980, WO 91/00360; and WO 92/200373).

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a heavy chain and light chain, the heavy chain and heavy chain and light chain may be expressed using a single vector, e.g., in a di-cistronic expression unit, or be under the control of different promoters. In other embodiments, the heavy chain and light chain region may be expressed using separate vectors. Heavy chains and light chains as described herein may optionally comprise a methionine at the N-terminus.

In some embodiments, antibody fragments (such as a Fab, a Fab′, a F(ab′)₂, a scFv, or a diabody) are generated. Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)₂ fragments (see, e.g., Carter et al., BioTechnology, 10:163-167 (1992)). According to another approach, F(ab′)₂ fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870.

In some embodiments, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007).

In some embodiments, multispecific antibodies are provided, e.g., a bispecific antibody. Multispecific antibodies are antibodies that have binding specificities for at least two different antigens or for at least two different epitopes of the same antigen. Methods for making multispecific antibodies include, but are not limited to, recombinant co-expression of two pairs of heavy chain and light chain in a host cell (see, e.g., Zuo et al., Protein Eng Des Sel, 2000, 13:361-367); “knobs-into-holes” engineering (see, e.g., Ridgway et al., Protein Eng Des Sel, 1996, 9:617-721); “diabody” technology (see, e.g., Hollinger et al., PNAS (USA), 1993, 90:6444-6448); and intramolecular trimerization (see, e.g., Alvarez-Cienfuegos et al., Scientific Reports, 2016, doi:/10.1038/srep28643); See also, Spiess et al., Molecular Immunology, 2015, 67(2), Part A:95-106.

Selection of Constant Region

Heavy and light chain variable regions of the antibodies described herein can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 weak complement-dependent cytotoxicity and human IgG4 lacks complement-dependent cytotoxicity. Human IgG1 and IgG3 also induce stronger cell mediated effector functions than human IgG2 and IgG4. Light chain constant regions can be lambda or kappa. Antibodies can be expressed as tetramers containing two light and two heavy chains, as separate heavy chains, light chains, as Fab, Fab′, F(ab′)₂, and Fv, or as single chain antibodies in which heavy and light chain variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of one or more other isotypes.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

For constructing desired antibody-drug conjugates, in some embodiments, exemplary substitution include the amino acid substitution of the native amino acid to a cysteine residue is introduced at amino acid position 234, 235, 237, 239, 267, 298, 299, 326, 330, or 332, preferably an S239C mutation in a human IgG1 isotype (numbering is according to the EU index (Kabat, Sequences of Proteins of Immunological Interest (National Institutes of Health, Bethesda, Md., 1987 and 1991); see US 20100158909, which is herein incorporated reference). The presence of an additional cysteine residue may allow interchain disulfide bond formation. Such interchain disulfide bond formation can cause steric hindrance, thereby reducing the affinity of the Fc region-FcγR binding interaction. The cysteine residue(s) introduced in or in proximity to the Fc region of an IgG constant region can also serve as sites for conjugation to therapeutic agents (i.e., coupling cytotoxic drugs using thiol specific reagents such as maleimide derivatives of drugs. The presence of a therapeutic agent causes steric hindrance, thereby further reducing the affinity of the Fc region-FcγR binding interaction. Other substitutions at any of positions 234, 235, 236 and/or 237 reduce affinity for Fcγ receptors, particularly FcγRI receptor (see, e.g., U.S. Pat. Nos. 6,624,821, 5,624,821.)

The in vivo half-life of an antibody can also impact its effector functions. The half-life of an antibody can be increased or decreased to modify its therapeutic activities. FcRn is a receptor that is structurally similar to MHC Class I antigen that non-covalently associates with 02-microglobulin. FcRn regulates the catabolism of IgGs and their transcytosis across tissues (Ghetie and Ward, 2000, Annu. Rev. Immunol. 18:739-766; Ghetie and Ward, 2002, Immunol. Res. 25:97-113). The IgG-FcRn interaction takes place at pH 6.0 (pH of intracellular vesicles) but not at pH 7.4 (pH of blood); this interaction enables IgGs to be recycled back to the circulation (Ghetie and Ward, 2000, Ann. Rev. Immunol. 18:739-766; Ghetie and Ward, 2002, Immunol. Res. 25:97-113). The region on human IgG1 involved in FcRn binding has been mapped (Shields et al., 2001, J. Biol. Chem. 276:6591-604). Alanine substitutions at positions Pro238, Thr256, Thr307, Gln311, Asp312, Glu380, Glu382, or Asn434 of human IgG1 enhance FcRn binding (Shields et al., 2001, J. Biol. Chem. 276:6591-604). IgG1 molecules harboring these substitutions have longer serum half-lives. Consequently, these modified IgG1 molecules may be able to carry out their effector functions, and hence exert their therapeutic efficacies, over a longer period of time compared to unmodified IgG1. Other exemplary substitutions for increasing binding to FcRn include a Gln at position 250 and/or a Leu at position 428. EU numbering is used for all positions in the constant region.

Complement fixation activity of antibodies (both C1q binding and CDC activity) can be improved by substitutions at Lys326 and Glu333 (Idusogie et al., 2001, J. Immunol. 166:2571-2575). The same substitutions on a human IgG2 backbone can convert an antibody isotype that binds poorly to C1q and is severely deficient in complement activation activity to one that can both bind C1q and mediate CDC (Idusogie et al., 2001, J. Immunol. 166:2571-75). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al., 1995, J. Immunol. 154:2226-36). Also, substituting Ser444 located close to the carboxy-terminal of IgG1 heavy chain with Cys induced tail-to-tail dimerization of IgG1 with a 200-fold increase of CDC activity over monomeric IgG1 (Shopes et al., 1992, J. Immunol. 148:2918-22). In addition, a bispecific diabody construct with specificity for C1q also confers CDC activity (Kontermann et al., 1997, Nat. Biotech. 15:629-31).

Complement activity can be reduced by mutating at least one of the amino acid residues 318, 320, and 322 of the heavy chain to a residue having a different side chain, such as Ala. Other alkyl-substituted non-ionic residues, such as Gly, Ile, Leu, or Val, or such aromatic non-polar residues as Phe, Tyr, Trp and Pro in place of any one of the three residues also reduce or abolish C1q binding. Ser, Thr, Cys, and Met can be used at residues 320 and 322, but not 318, to reduce or abolish C1q binding activity. Replacement of the 318 (Glu) residue by a polar residue may modify but not abolish C1q binding activity. Replacing residue 297 (Asn) with Ala results in removal of lytic activity but only slightly reduces (about three fold weaker) affinity for C1q. This alteration destroys the glycosylation site and the presence of carbohydrate that is required for complement activation. Any other substitution at this site also destroys the glycosylation site. The following mutations and any combination thereof also reduce C1q binding: D270A, K322A, P329A, and P311S (see WO 06/036291).

Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcγ receptor binding or increase binding to FcRN.

Nucleic Acids, Vectors, and Host Cells

In some embodiments, the antibodies described herein are prepared using recombinant methods. Accordingly, in some aspects, the invention provides isolated nucleic acids comprising a nucleic acid sequence encoding any of the antibodies described herein (e.g., any one or more of the CDRs described herein); vectors comprising such nucleic acids; and host cells into which the nucleic acids are introduced that are used to replicate the antibody-encoding nucleic acids and/or to express the antibodies. In some embodiments, the host cell is eukaryotic, e.g., a Chinese Hamster Ovary (CHO) cell; or a human cell.

In some embodiments, a polynucleotide (e.g., an isolated polynucleotide) comprises a nucleotide sequence encoding an antibody described herein. In some embodiments, the polynucleotide comprises a nucleotide sequence encoding one or more amino acid sequences (e.g., CDR, heavy chain, light chain, and/or framework regions) disclosed herein. In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to a sequence (e.g., a CDR, heavy chain, light chain, or framework region sequence) disclosed herein.

In a further aspect, methods of making an antibody described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. Cloning vectors are available from commercial vendors such as BioRad, Stratagene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Expression of Recombinant Antibodies

Antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

Mammalian cells are a preferred host for expressing nucleotide segments encoding immunoglobulins or fragments thereof. See Winnacker, From Genes to Clones, (VCH Publishers, N Y, 1987). A number of suitable host cell lines capable of secreting intact heterologous proteins have been developed in the art, and include CHO cell lines (e.g., DG44), various COS cell lines, HeLa cells, HEK293 cells, L cells, and non-antibody-producing myelomas including Sp2/0 and NSO. Preferably, the cells are nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., J. Immunol. 148:1149 (1992).

Once expressed, antibodies can be purified according to standard procedures of the art, including HPLC purification, column chromatography, gel electrophoresis and the like (see generally, Scopes, Protein Purification (Springer-Verlag, NY, 1982)).

Antibody Characterization

Methods for analyzing binding affinity, binding kinetics, and cross-reactivity are known in the art. See, e.g., Ernst et al., Determination of Equilibrium Dissociation Constants, Therapeutic Monoclonal Antibodies (Wiley & Sons ed. 2009). These methods include, but are not limited to, solid-phase binding assays (e.g., ELISA assay), immunoprecipitation, surface plasmon resonance (SPR, e.g., Biacore™ (GE Healthcare, Piscataway, N.J.)), kinetic exclusion assays (e.g. KinExA®), flow cytometry, fluorescence-activated cell sorting (FACS), BioLayer interferometry (e.g., Octet™ (ForteBio, Inc., Menlo Park, Calif.)), and Western blot analysis. SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given wavelength is shined on the surface, the changes to the optical reflectivity of the gold indicate binding, and the kinetics of binding. In some embodiments, kinetic exclusion assays are used to determine affinity. This technique is described, e.g., in Darling et al., Assay and Drug Development Technologies Vol. 2, number 6 647-657 (2004). In some embodiments, BioLayer interferometry assays are used to determine affinity. This technique is described, e.g., in Wilson et al., Biochemistry and Molecular Biology Education, 38:400-407 (2010); Dysinger et al., J. Immunol. Methods, 379:30-41 (2012).

IV. Therapeutic Methods

In some embodiments, methods for treating cancer in a subject are provided. In some embodiments, the method comprises administering to the subject (1) an antibody-drug conjugate (ADC) that comprises a first antibody that binds a tumor-associated antigen and a cytotoxic agent, wherein the cytotoxic agent is a tubulin disrupter; and (2) a second antibody that binds to an immune cell engager, wherein the second antibody comprises an Fc with enhanced binding to one or more activating FcγRs. In some embodiments, the Fc of the second antibody has enhanced binding to one or more of FcγRIIIa, FcγRIIa, and/or FcγRI. In some embodiments, the Fc of the second antibody has reduced binding to one or more inhibitory FcγRs. In some embodiments, the Fc of the second antibody has reduced binding to FcγRIIb.

In some embodiments, a method of treating cancer comprises administering to a subject with cancer (1) an antibody-drug conjugate (ADC), wherein the ADC comprises a first antibody that binds a tumor-associated antigen and a cytotoxic agent, wherein the cytotoxic agent is a tubulin disrupter, and (2) a second antibody that binds an immune cell engager, wherein the second antibody comprises an Fc with enhanced ADCC activity relative to a corresponding wild-type Fc of the same isotype. In some embodiments, the second antibody comprises an Fc with enhanced ADCC and ADCP activity relative to a corresponding wild-type Fc of the same isotype. In some embodiments, the Fc of the second antibody has enhanced binding to one or more of FcγRIIIa, FcγRIIa, and/or FcγRI. In some embodiments, the Fc of the second antibody has reduced binding to one or more inhibitory FcγRs. In some embodiments, the Fc of the second antibody has reduced binding to FcγRIIb.

In various embodiments, the second antibody is a nonfucosylated antibody. In various such embodiments, the second antibody is comprised in a composition of antibodies, wherein at least 90%, at least 91%, at least 92%, at least 93%, at least 94%, at least 95%, at least 96%, at least 97%, at least 98%, or at least 99% of the antibodies in the composition are nonfucosylated.

In some embodiments, the second antibody binds TIGIT. In some embodiments, the second antibody binds CD40. In some embodiments, the second antibody binds an immune cell engager provided herein.

In various embodiments, the tubulin disrupter conjugated to the first antibody in the ADC is an auristatins, a tubulysin, a colchicine, a vinca alkaloid, a taxane, a cryptophycin, a maytansinoid, or a hemiasterlin. In some embodiments, the ADC comprises MMAE or MMAF. In various embodiments, the first antibody binds a tumor-associated antigen, such as a tumor-associated antigen provided herein.

Any of the ADCs described herein may be combined with any of the antibodies that bind an immune cell engager described herein. For example, in some embodiments, the ADC is SGN-PDL1V, and the second antibody is SEA-BCMA. In some embodiments, the ADC is SGN-ALPV, and the second antibody is SEA-BCMA. In some embodiments, the ADC is SGN-B7H4V, and the second antibody is SEA-BCMA. In some embodiments, the ADC is lifastuzumab vedotin, and the second antibody is SEA-BCMA. In some embodiments, the ADC is SEA-CD40, and the second antibody is SEA-BCMA. In some embodiments, the ADC is SEA-CD70, and the second antibody is SEA-BCMA. In some embodiments, the ADC is SGN-B6A, and the second antibody is SEA-BCMA. In some embodiments, the ADC is SGN-CD228A, and the second antibody is SEA-BCMA. In some embodiments, the ADC is SGN-LIV1A, and the second antibody is SEA-BCMA. In some embodiments, the ADC is SGN-STNV, and the second antibody is SEA-BCMA. In some embodiments, the ADC is brentuximab vedotin (SGN-35), and the second antibody is SEA-BCMA. In some embodiments, the ADC is enfortumab vedotin, and the second antibody is SEA-BCMA. In some embodiments, the ADC is disitamab vedotin, and the second antibody is SEA-BCMA. In some embodiments, the ADC is tisotumab vedotin, and the second antibody is SEA-BCMA.

In some embodiments, the ADC is SGN-PDL1V, and the second antibody is SEA-CD40. In some embodiments, the ADC is SGN-ALPV, and the second antibody is SEA-CD40. In some embodiments, the ADC is SGN-B7H4V, and the second antibody is SEA-CD40. In some embodiments, the ADC is lifastuzumab vedotin, and the second antibody is SEA-CD40. In some embodiments, the ADC is SEA-CD40, and the second antibody is SEA-CD40. In some embodiments, the ADC is SEA-CD70, and the second antibody is SEA-CD40. In some embodiments, the ADC is SGN-B6A, and the second antibody is SEA-CD40. In some embodiments, the ADC is SGN-CD228A, and the second antibody is SEA-CD40. In some embodiments, the ADC is SGN-LIV1A, and the second antibody is SEA-CD40. In some embodiments, the ADC is SGN-STNV, and the second antibody is SEA-CD40. In some embodiments, the ADC is brentuximab vedotin (SGN-35), and the second antibody is SEA-CD40. In some embodiments, the ADC is enfortumab vedotin, and the second antibody is SEA-CD40. In some embodiments, the ADC is disitamab vedotin, and the second antibody is SEA-CD40. In some embodiments, the ADC is tisotumab vedotin, and the second antibody is SEA-CD40.

In some embodiments, the ADC is SGN-PDL1V, and the second antibody is SEA-CD70. In some embodiments, the ADC is SGN-ALPV, and the second antibody is SEA-CD70. In some embodiments, the ADC is SGN-B7H4V, and the second antibody is SEA-CD70. In some embodiments, the ADC is lifastuzumab vedotin, and the second antibody is SEA-CD70. In some embodiments, the ADC is SEA-CD40, and the second antibody is SEA-CD70. In some embodiments, the ADC is SEA-CD70, and the second antibody is SEA-CD70. In some embodiments, the ADC is SGN-B6A, and the second antibody is SEA-CD70. In some embodiments, the ADC is SGN-CD228A, and the second antibody is SEA-CD70. In some embodiments, the ADC is SGN-LIV1A, and the second antibody is SEA-CD70. In some embodiments, the ADC is SGN-STNV, and the second antibody is SEA-CD70. In some embodiments, the ADC is brentuximab vedotin (SGN-35), and the second antibody is SEA-CD70. In some embodiments, the ADC is enfortumab vedotin, and the second antibody is SEA-CD70. In some embodiments, the ADC is disitamab vedotin, and the second antibody is SEA-CD70. In some embodiments, the ADC is tisotumab vedotin, and the second antibody is SEA-CD70.

In some embodiments, the ADC is SGN-PDL1V, and the second antibody is SEA-TGT. In some embodiments, the ADC is SGN-ALPV, and the second antibody is SEA-TGT. In some embodiments, the ADC is SGN-B7H4V, and the second antibody is SEA-TGT. In some embodiments, the ADC is lifastuzumab vedotin, and the second antibody is SEA-TGT. In some embodiments, the ADC is SEA-CD40, and the second antibody is SEA-TGT. In some embodiments, the ADC is SEA-CD70, and the second antibody is SEA-TGT. In some embodiments, the ADC is SGN-B6A, and the second antibody is SEA-TGT. In some embodiments, the ADC is SGN-CD228A, and the second antibody is SEA-TGT. In some embodiments, the ADC is SGN-LIV1A, and the second antibody is SEA-TGT. In some embodiments, the ADC is SGN-STNV, and the second antibody is SEA-TGT. In some embodiments, the ADC is brentuximab vedotin (SGN-35), and the second antibody is SEA-TGT. In some embodiments, the ADC is enfortumab vedotin, and the second antibody is SEA-TGT. In some embodiments, the ADC is disitamab vedotin, and the second antibody is SEA-TGT. In some embodiments, the ADC is tisotumab vedotin, and the second antibody is SEA-TGT.

In some embodiments, the subject is a human.

In some embodiments, the cancer is bladder cancer, breast cancer, uterine cancer, cervical cancer, ovarian cancer, prostate cancer, testicular cancer, esophageal cancer, gastrointestinal cancer, gastric cancer, pancreatic cancer, colorectal cancer, colon cancer, kidney cancer, clear cell renal carcinoma, head and neck cancer, lung cancer, lung adenocarcinoma, stomach cancer, germ cell cancer, bone cancer, liver cancer, thyroid cancer, skin cancer, melanoma, neoplasm of the central nervous system, mesothelioma, lymphoma, leukemia, chronic lymphocytic leukemia, diffuse large B cell lymphoma, follicular lymphoma, Hodgkin lymphoma, myeloma, or sarcoma. In some embodiments, the cancer is selected from gastric cancer, testicular cancer, pancreatic cancer, lung adenocarcinoma, bladder cancer, head and neck cancer, prostate cancer, breast cancer, mesothelioma, and clear cell renal carcinoma. In some embodiments, the cancer is a lymphoma or a leukemia, including but not limited to acute myeloid, chronic myeloid, acute lymphocytic or chronic lymphocytic leukemia, diffuse large B-cell lymphoma, follicular lymphoma, mantle cell lymphoma, small lymphocytic lymphoma, primary mediastinal large B-cell lymphoma, splenic marginal zone B-cell lymphoma, or extranodal marginal zone B-cell lymphoma. In some embodiments, the cancer is selected from chronic lymphocytic leukemia, diffuse large B cell lymphoma, follicular lymphoma, and Hodgkin lymphoma. In some embodiments, the cancer is a metastatic cancer.

In some embodiments, the cancer is one with high tumor mutation burden as such cancers have more antigen to drive T cell responses. Thus, in some embodiments, the cancer is a high mutational burden cancer such as lung, melanoma, bladder, or gastric cancer. In some embodiments, the cancer has microsatellite instability.

In various embodiments, the second antibody depletes T regulatory (Treg) cells, activates antigen presenting cells (APCs), enhances CD8 T cell responses, upregulates co-stimulatory receptors, and/or promotes release of immune activating cytokines (such as CXCL10 and/or IFNγ). In some embodiments, the second antibody promotes release of immune activating cytokines to a greater extent than immune suppressive cytokines (such as IL10 and/or MDC).

The ADC and second antibody may be administered concurrently or sequentially. For sequential administration, a first dose of the ADC may be administered before the first dose of the second antibody, or a first dose of the second antibody may be administered before the ADC. For concurrent administration, in some embodiments, the ADC and second antibody may be administered as separate pharmaceutical composition or in the same pharmaceutical composition.

In some embodiments, a therapeutic agent is administered at a therapeutically effective amount or dose. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

In some embodiments, the enhanced activity observed with the particular combination therapies described herein have certain benefits as compared to corresponding monotherapy treatment. For example, in some embodiments, administration of the ADC and the second antibody in combination has a toxicity profile comparable to that of the ADC or the second antibody when either is administered as monotherapy. In some embodiments, the effective dose of the ADC and/or the second antibody when dosed in combination is less than when administered as monotherapy. In some embodiments, administration of the ADC and the second antibody in combination provide a longer duration of response as compared to corresponding monotherapy treatment. In some embodiments, administration of the ADC and the second antibody in combination results in longer progression-free survival as compared to corresponding monotherapy. In some embodiments, the administration of the ADC and the second antibody can be used to treat recurrent cancer that recurs following monotherapy treatment with either agent individually.

The route of administration of a pharmaceutical composition can be oral, intraperitoneal, transdermal, subcutaneous, intravenous, intramuscular, inhalational, topical, intralesional, rectal, intrabronchial, nasal, transmucosal, intestinal, ocular or otic delivery, or any other methods known in the art. In some embodiments, one or more therapeutic agents are administered orally, intravenously, or intraperitoneally.

Co-administered therapeutic agents can be administered together or separately, simultaneously or at different times. When administered, the therapeutic agents independently can be administered once, twice, three, four times daily or more or less often, as needed. In some embodiments, the administered therapeutic agents are administered once daily. In some embodiments, the administered therapeutic agents are administered at the same time or times, for instance as an admixture. In some embodiments, one or more of the therapeutic agents is administered in a sustained-release formulation.

In some embodiments, therapeutic agents are administered concurrently. In some embodiments, the therapeutic agents are administered sequentially. For example, in some embodiments, a first therapeutic agent is administered, for example for about 1, 2, 3, 4, 5, 6, 7, 8, 9, 10, 15, 20, 25, 30, 40, 50, 60, 70, 80, 90, 100 days or more prior to administering a second therapeutic agent.

In some embodiments, the treatment provided herein is administered to the subject over an extended period of time, e.g., for at least 30, 40, 50, 60, 70, 80, 90, 100, 150, 200, 250, 300, 350 days or longer.

V. Compositions and Kits

In another aspect, compositions and kits for use in treating or preventing a cancer in a subject are provided.

Pharmaceutical Compositions

In some embodiments, pharmaceutical compositions for use in the present methods are provided. In some embodiments, the ADC is administered in a first pharmaceutical composition and the antibody that binds an immune cell engager is administered in a second pharmaceutical composition. In some embodiments, the ADC and the antibody that binds an immune cell engager are administered in a single pharmaceutical composition.

Guidance for preparing formulations for use in the present invention is found in, for example, Remington: The Science and Practice of Pharmacy, 21^{st} Ed., 2006, supra; Martindale: The Complete Drug Reference, Sweetman, 2005, London: Pharmaceutical Press; Niazi, Handbook of Pharmaceutical Manufacturing Formulations, 2004, CRC Press; and Gibson, Pharmaceutical Preformulation and Formulation: A Practical Guide from Candidate Drug Selection to Commercial Dosage Form, 2001, Interpharm Press, which are hereby incorporated herein by reference. The pharmaceutical compositions described herein can be manufactured in a manner that is known to those of skill in the art, i.e., by means of conventional mixing, dissolving, granulating, dragee-making, emulsifying, encapsulating, entrapping or lyophilizing processes. The following methods and excipients are merely exemplary and are in no way limiting.

In some embodiments, one or more therapeutic agents are prepared for delivery in a sustained-release, controlled release, extended-release, timed-release or delayed-release formulation, for example, in semi-permeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

For oral administration, a therapeutic agent can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

A therapeutic agent can be formulated for parenteral administration by injection, e.g., by bolus injection or continuous infusion. For injection, the compound or compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, compounds can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

A therapeutic agent can be administered systemically by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplary transdermal delivery formulations include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. In some embodiments, the carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science andPractice of Pharmacy, 21st Edition, Philadelphia, Pa. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

Dosages and desired drug concentration of pharmaceutical compositions of the disclosure may vary depending on the particular use envisioned. The determination of the appropriate dosage or route of administration is well within the skill of one in the art. Suitable dosages are also described herein.

Kits

In some embodiments, kits for use in treating a subject having a cancer are provided. In some embodiments, the kit comprises:
an antibody-drug conjugate comprising a first antibody conjugated to a tubulin disrupter, as provided herein; and
a second antibody that binds an immune cell engager, as provided herein.

In some embodiments, the kits can further comprise instructional materials containing directions (i.e., protocols) for the practice of the methods of this invention (e.g., instructions for using the kit for treating a cancer). While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### VI. EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Non-directed Chemotherapeutic Agents Impair T Cell Responses

1.1 Materials and Methods

Human primary T cells were induced to undergo proliferation using CD3/CD28 coated beads. 20,000 carboxyfluorescein diacetate succinimidyl ester (CSFE) labeled, enriched CD3+T cells were incubated with anti-CD3 CD28 beads (1 bead per 4 T cells)+10 ng/mL IL-2 for 4 days. Cells were stained with LIVE/DEAD Fixable Dead Cell Stain (ThermoFisher) and live cells were counted via flow cytometry.

1.2 Results

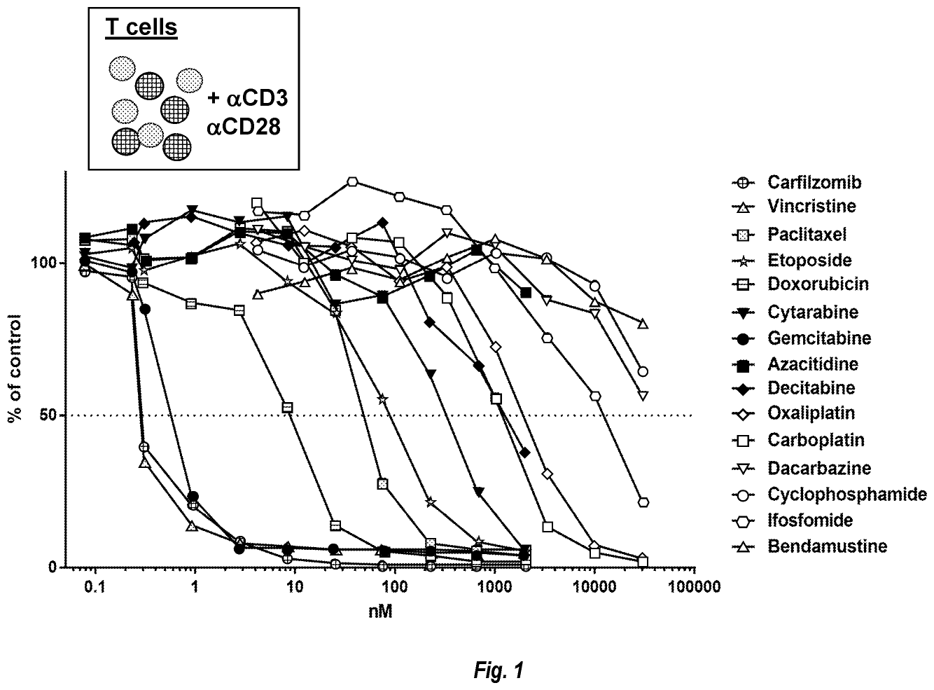

As shown in FIG. 1, proliferation of primary human T cells was significantly reduced by all the single free agent chemotherapeutics tested. These data suggest that systemic exposure to chemotherapeutic agents may limit T cell mediated activity in patients including responses from immune-oncology agents (e.g., antibodies that bind immune cell engagers).

### Example 2: Vedotin ADCs do not Inhibit T Cell Proliferation, Despite Directed Delivery to T Cells (BV (SGN-35) Treatment of CD30+CD8 T Cells)

2.1 Materials and Methods

Human primary CD8 T cells were labeled with CSFE and induced to undergo proliferation with anti-CD3-CD28 beads (1 bead per 4 T cells)+10 ng/mL IL-2 for 4 days. During activation, CD30 was upregulated on the surface of T cells. CD30+CD8 T cells were treated with either CD30 directed vc-MMAE (brentuximab vedotin; BV; SGN-35) or an isotype control. Cells were stained with LIVE/DEAD Fixable Dead Cell Stain (ThermoFisher) and live cells were counted via flow cytometry.

2.2 Results

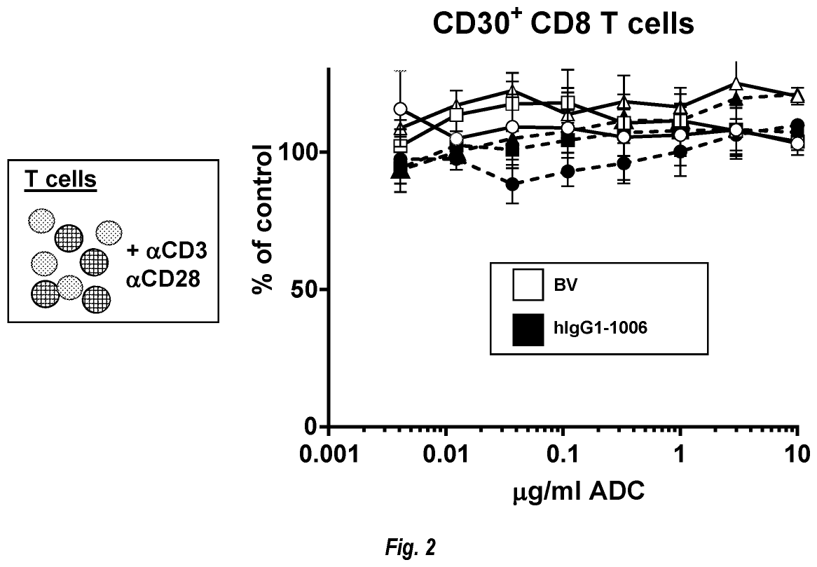

As shown in FIG. 2, cell proliferation of primary human CD30+CD8 T cells was not significantly altered by treatment with BV. These data suggest that systemic exposure of a vedotin ADC, even if targeted directly to CD8 T cells, does not impact CD8 mediated anti-tumor responses.

### Example 3: Endoplasmic Reticulum Stress Induction is Superior for Vedotin ADCs

3.1 Materials and Methods

Induction of endoplasmic reticulum (ER) stress is one of the first and required steps for the initiation of immunogenic cell responses (FIG. 3B). MIA-PaCa-2 pancreatic cancer cell lines were treated with ADCs conjugated to distinct payloads, including vedotin (MMAE), emtansine (DM1), the Exatecan DS-8201 (Ex), as well as the free microtubule stabilizing agent paclitaxel at IC50 concentrations that induce cell death in this system. After 36 or 48 hours of treatment, cells were harvested for western blot analysis and the upstream ER stress marker pJNK (FIG. 3B) was assessed by Western blot.

3.1.1 Western Blot

Treated cells were centrifuged at 14,000-16,000 rpm for 10 mins and stored at −20° C. Cell pellets were resuspended in 4X BOLT™ LDS sample buffer (Thermo Fisher Catalog #B0007) and lysed by heating at 95° C. for 5-10 minutes to produce cell lysates. Sample lysates were run on a Bis-Tris 4-12% gradient gel at 140V for 1 hr 40 mins in MOPS buffer. The Bis-Tris gel was then transferred onto a nitrocellulose membrane using an iBlot2. Membranes were washed once in 1× TBS and incubated overnight at 4° C. in Licor Blocking buffer. Membranes were washed four times in 1X TBS-T for 5-10 mins each. Images were developed on the Licor Odyssey System using the 84 μm resolution and auto intensities.

3.1.2 CHOP Luciferase Induction Assay

Assessment of downstream pathways to ER stress induction was performed using MIA-PaCa-2 cells transduced with the CHOP driven luciferase reporter cell line. CHOP is the last step in the ER stress response cascade and its expression levels are increased by ER stress. Several ADC payloads in clinical development (FIG. 3A) were used in the assessment. Induction of CHOP was measured using a reporter system for CHOP activity according to the manufacturer's instructions (Bright-Glo™ Luciferase Assay System, Promega). In brief, 100,000 cells/well were plated in a 96-well, f1at-bottom clear plate (aliquot 150 μL per well). 200 μL of media were aliquoted to outer wells of the plate to provide a media “blanket” around the wells of cells. At 24, 48, and 72 hours, plates were removed from the incubator and allowed to come to room temperature. 100 μL of media was removed from the wells. 100 μL of BrightGlo Reagent was added to each well. Plate was shaken for at least two minutes before reading. The Envision CTG 96-well Standard Protocol was used to read the plate.

3.2 Results

As shown in FIG. 3C-E, auristatin-based ADCs (MMAE-ADC or MMAF-ADC) treatment was the only condition found to induce the early ER stress response pJNK signal of the different ADC payloads tested. ER stress induction is tied to microtubule disruptions as the ER requires intact microtubules to expand and contract to accommodate the protein translational needs of the cell. The ability of MMAE as a microtubule disrupting agent to induce this ER stress is exemplified in the data shown.

FIG. 3D-E demonstrate that CHOP, a downstream signal in the ER stress pathway, is significantly induced by MMAE ADCs and that downstream pathways to ER stress are also driven differently by MMAE ADCs as compared to other payloads.

### Example 4: ICD Potential of Different Clinical ADC Payloads

4.1 Materials and Methods

Classical markers of ICD include surface exposure of calreticulin and release of ATP and HMGB1 which occur concomitantly with the induction of ER stress response. These molecules are considered danger signals and activate innate immune cells and increase tumor antigen specific T cell responses. MIA-PaCa-2 cancer cells were treated with IC50 concentrations of ADC-bearing payloads that are currently at the clinical stage, i.e., MMAE, DM1, and Exatecan (Ex). Treated cells were then analyzed for ICD marker induction. Cisplatin was used as a negative control because it is able to drive cell death but is not known to induce ICD.

100,000-150,000 cells were plated per well in a 96-well dish. Cells were allowed to reach 50-60% confluence. The media was removed and fresh culture media was added per well of cells. 1 μg/mL of 1 μM of drug was added to each well of cells. After 24 hours, 250 μL (for the ATP release assay) or 200 μL (for the HMGB1 assay) of media was collected and transferred into a labeled 1.5 mL Eppendorf tube. Each tube of sample was centrifuged at 10,000 rpm for 1 min. 50 μL of media was transferred to a well in a 96-well clear bottom plate. 50 μL of CTG was added to each well. The plate was shaken for 1-2 mins. The Envision Plate Reader was used to read the plate.

HMGB1 release levels were monitored by luminescent intensity per well using the Envision Plate Reader. HMGB1 and ATP release levels were reported as the fold change over the background values for untreated samples. Acquired values were converted to text file and exported and analyzed using Excel and GraphPad Prism.

4.2 Results

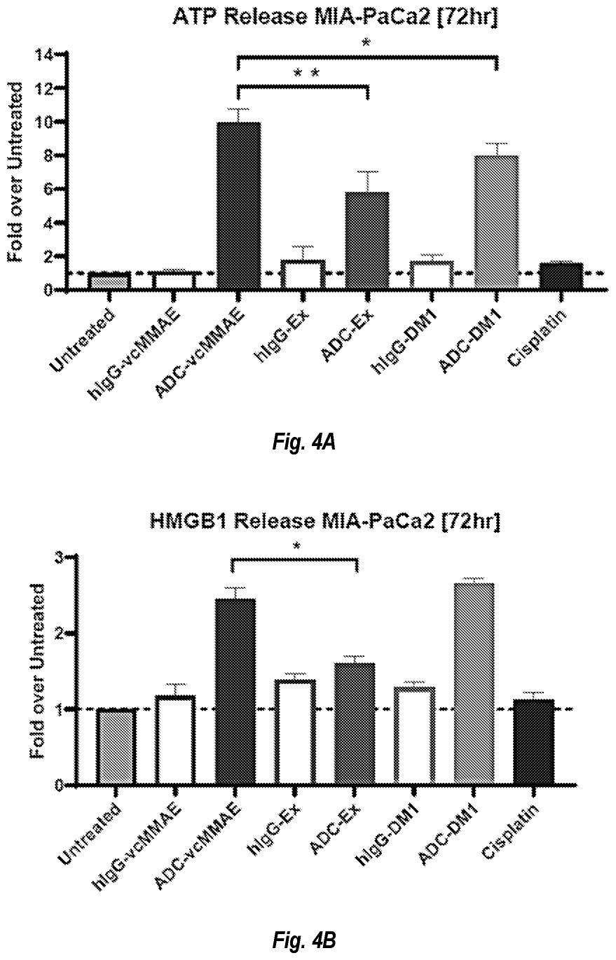

As shown in FIG. 4A, vc-MMAE was potent in driving ATP release compared to the other payloads tested. While HMGB1 release is associated with induction of ICD its release is also seen when cells begin to undergo necrosis and is not directly associated with robust immune cell engagement. Treatment of MIA-PaCa-2 cells with microtubilin disrupting agents vc-MMAE and DM1 resulted in robust HMGB1 release, which contrasts to the topoisomerase inhibitor Exatecan (Ex) (FIG. 4B).

### Example 5: Immune Activation Assessment of ADC Payloads

5.1 Materials and Methods

5.1.1 Cells

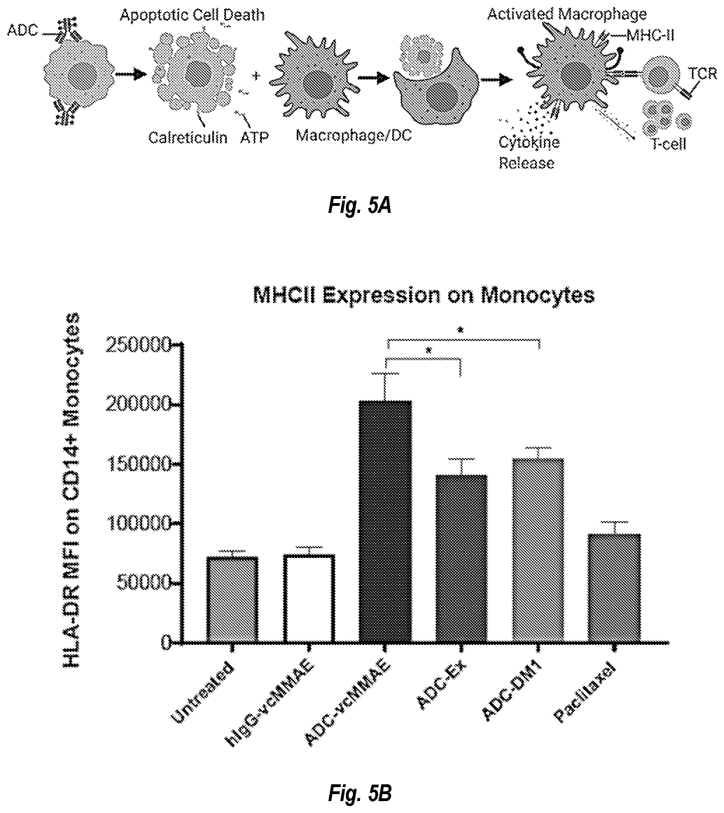

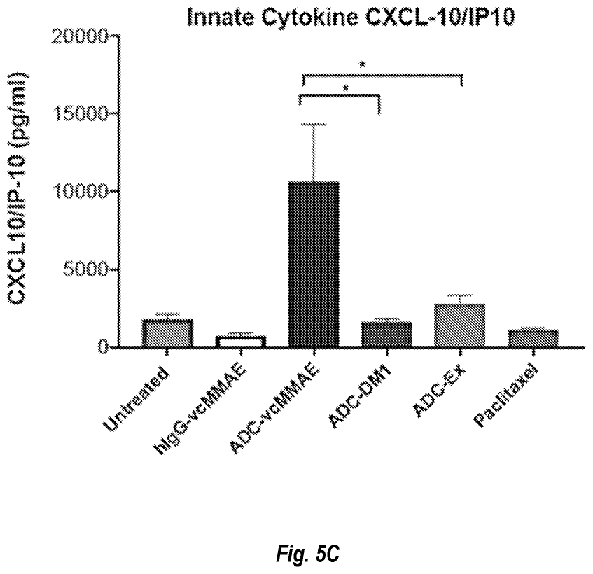

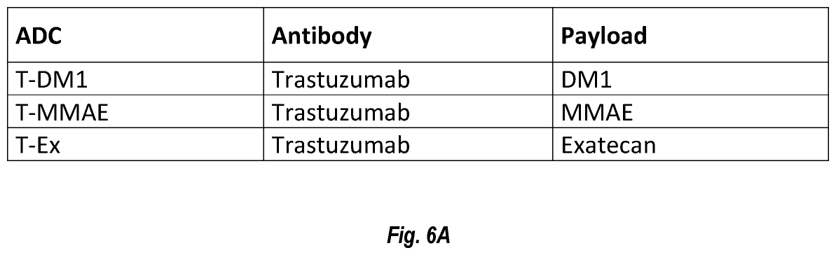

As shown in FIG. 5A, ADCs conjugated to MMAE disrupts microtubules, resulting in ER stress response, leading to immunogenic cell death (ICD). The dying cells in turn release immune-activating molecules- damage-associated molecular patterns (DAMPs)-such as HSP70, HSP90, ATP, HMGB1, and calreticulin (CRT). These DAMPs can bind receptors such as LPR1/CD91, P2RX7, P2RY2, AGER, TLR2, and TLR4, thereby activating the innate immune system. This activation results in, for example, upregulation of proteins such as CD80, CD86, HLA-DR, and CD40, an increase of MHCII expression on monocytes, and the release of cytokines such as CXCL-10/IP10 and IL-12, thereby initiating antitumor T cell responses. Such T cell responses can be further augmented by PD-1/L1 inhibitors. Here, the immunologic consequence of ICD was assessed in human peripheral blood mononuclear cell (PBMC) cultures. Cancer cells exposed to ADCs conjugated to distinct payloads were added to PBMCs.

L540cy cancer cells exposed to EC50 concentrations of ADC or free drug, for 18 hours (at 37° C. in 5% CO₂. were washed and 250 ul of PBMCs suspended at 10×10⁶ cell/mL were added to the cancer cell lines killed cells for 48 hrs. Tissue culture media was taken and cytokines measured by Luminex assessment.

Treatment was performed in triplicate for 2 independent PBMC donors.

5.1.2 Co-Stimulatory Molecule Surface Expression

After treatment, cell pellets were resuspended in 50 mL of BD FACs buffer and transferred to 96 well round-bottom microtiter plates. Fc receptors were blocked with human 100 pg/mL Fc-fragments for 30 minutes on ice. A master mix composed of PE-HLA-DR (MHCII) and APC-CD14 diluted at 1:100 was prepared in BD FACs buffer containing 100 mg/mL human purified Fc-fragments. 10 μl of the master mix was added to each well containing 90 μl of re-suspended cells and samples were incubated for 1 hour on ice. Cells were then centrifuged at 400×g in a pre-cooled Eppendorf 5810R centrifuge for 5 minutes. The supernatant was removed and cells were washed with 200 mL of BD FACs buffer. The wash was performed twice and cells were resuspended in 200 mL of FACs buffer and samples were analyzed on an Attune flow cytometer. HLA-DR mean f1uorescence was determined using FlowJo analysis software.

5.1.3 Cytokine Production

After treatment, PBMCs/cancer cell co-cultures were spun with a plate adapter in an Eppendorf 5810R at 800 rpm for 5 minutes. Serum or tissue culture supernatant were removed and transferred to a 96-strip tube rack and samples were frozen at −80° C. until processing. Frozen tissue culture supernatants and serum were thawed overnight at 4° C. and processed for cytokine production using a Luminex Multiplex Kit from Millipore

Tissue culture supernatant and serum samples were processed as per the manufacturer's instructions. Briefly, assay plates were washed with 200 μL of wash buffer per well, followed by addition of 25 μL standard or buffer, 25 μL matrix or sample, and 25 μL of multiplexed analyte beads to each well. Samples were incubated overnight with vigorous shaking at 4° C. Plates are washed the assay plates twice with wash buffer.

Detection antibodies (25 μL) were added to each well and incubated at room temperature for 1 hour. 25 μL of streptavidin-phycoerythrin (SA-PE) was added and samples incubated at room temperature for 30 minutes. The plate was washed twice with wash buffer, and beads were resuspended with 150 μL of sheath f1uid. The samples were analyzed using Luminex MagPix systems in combination with the Xponent software system. Cytokine levels were calculated from the standard curve.

5.2 Results

Innate cell activation was observed, as evidenced by increased surface activation markers (MHCII) and the release of inf1ammatory cytokines (CXCL-10/IP10) (FIG. 5B-C). Innate immune cells become activated when exposed to vc-MMAE treated tumor cells. Immune cell activation by vc-MMAE was more robust than activation by other ADC payloads (FIG. 5B-C).

Vc-MMAE-mediated ICD is regulated cell death that activates adaptive immune responses against antigens from dead and dying tumor cells and allows for the generation of robust innate immune cell activation and subsequent cytotoxic T-cell responses targeted towards specific tumor cell antigens. Here, it was demonstrated that vc-MMAE killed cancer cells elicited an increase of surface MHCII and release of the innate cytokine CXCL10 a strong chemotactic and inflammatory mediator, from monocyte/macrophages after uptake of dead cells.

### Example 6: Payload Evaluation on Trastuzumab Backbone

6.1 Materials and Methods

The ability of trastuzumab ADC conjugates bearing various clinical stage payloads to induce ER stress and downstream ICD markers ATP and HMGB1 was assessed. The payloads used were DM1, MMAE, and Exatecan (Ex).

6.2 Results

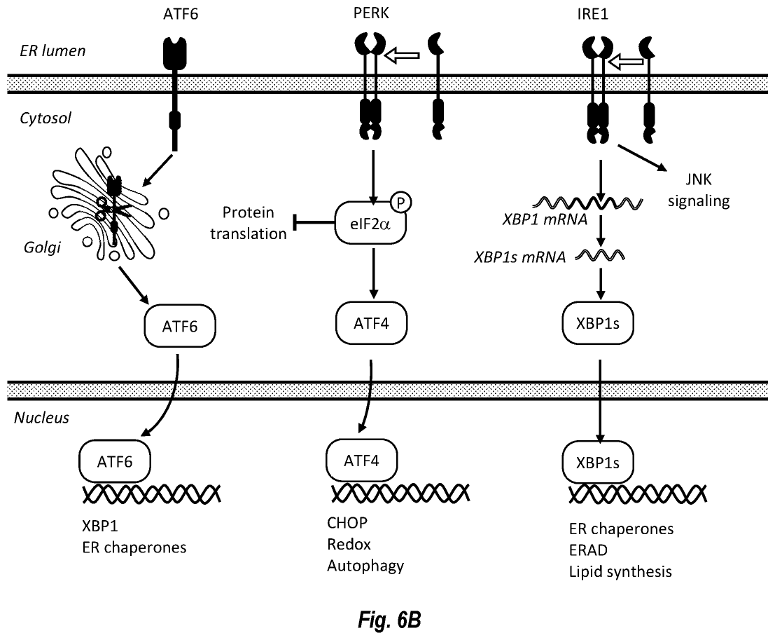

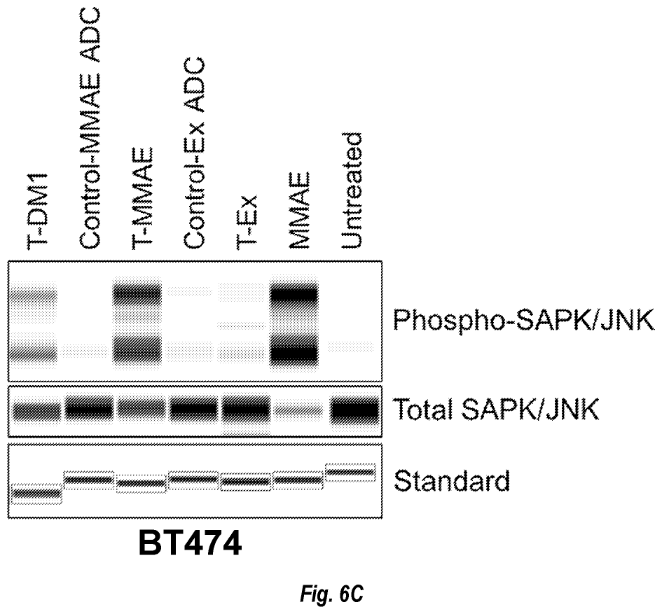

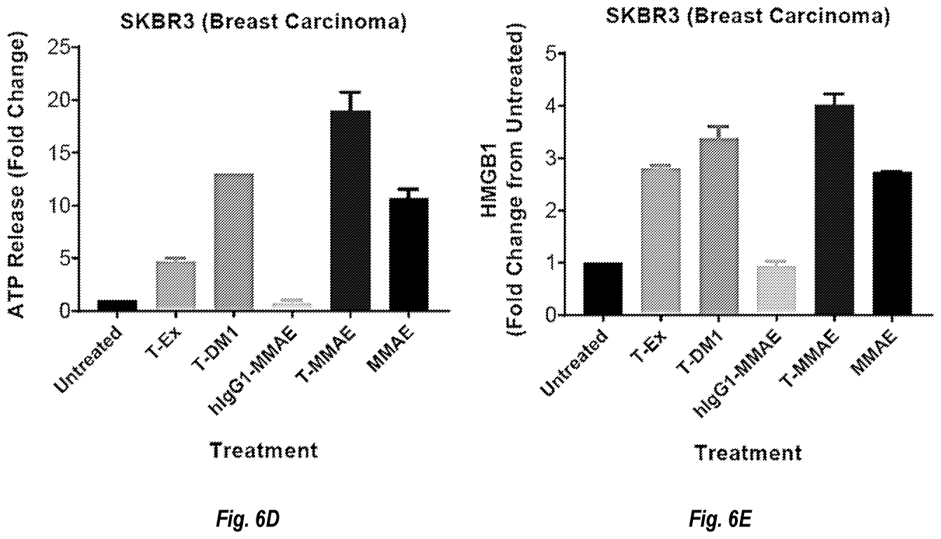

Two observations were made: (1) trastuzumab conjugated to vcMMAE drove the most robust ER stress response which was associated with induction of ATP and HMGB1; and (2) the late cell death marker HMGB1 seemed elevated for the other payload classes indicating that secondary necrosis maybe associated with these payload classes rather than frank ICD (FIG. 6C-E). The findings here are similar to findings described above in Example 4, which used Mia-PaCa-2 cells (FIG. 4A-B).

### Example 7: Induction of Early Stage ER Stress Markers (JNK Signaling Activation) is Generally Superior for MMAE ADCs

7.1 Materials and Methods

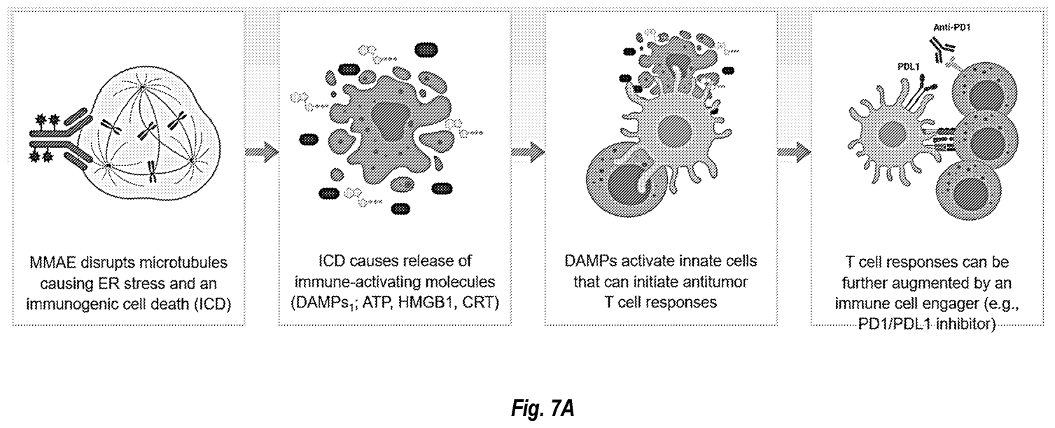

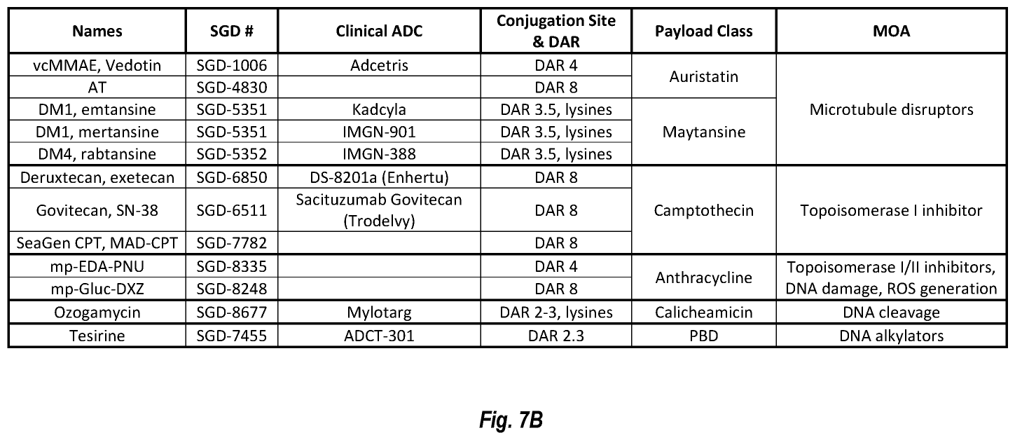

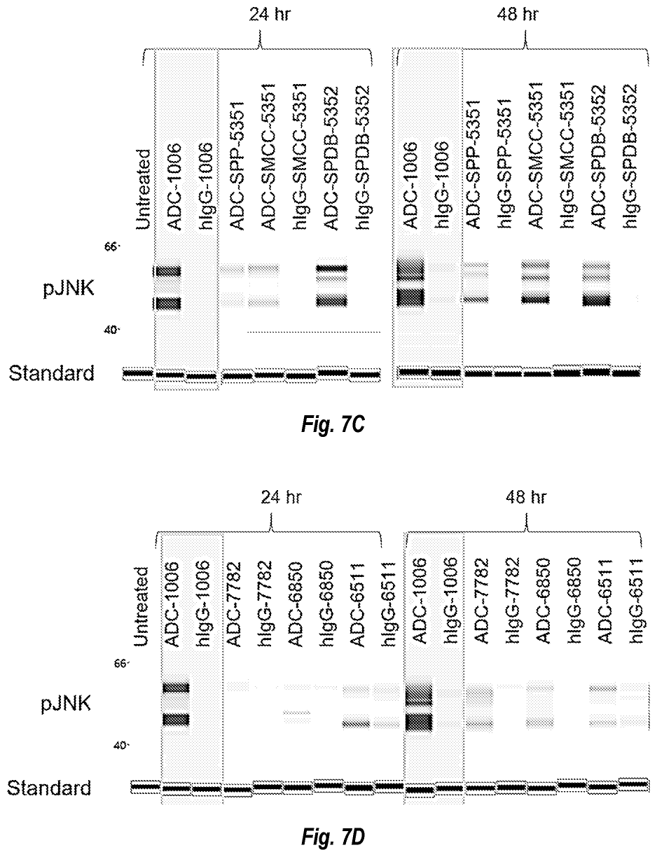

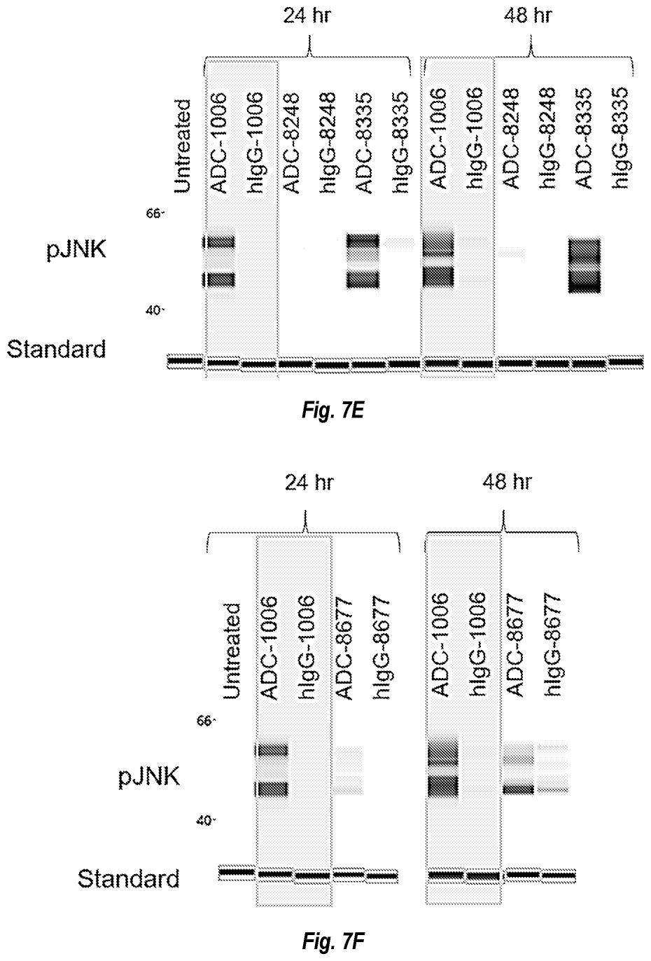

As described in Example 5 and shown in FIG. 5A, the ICD pathway involves various aspects. This pathway is illustrated further in FIG. 7A. As shown in FIG. 7A and noted above, a tubulin disrupter such MMAE in an initial stage disrupts microtubules, thereby causing ER stress and ICD. ICD in turn causes release of immune-activating molecules such as DAMPs, ATP, HMGB1, and CRT. These molecules can subsequently activate innate cells that are capable of initiating antitumor T cell responses and can induce T cell memory. Such T cell responses can be further augmented by combination with other immune modulators, such as the immune cell engagers described herein. Several of the following examples report the results of studies showing the effectiveness of MMAE compared to other ADC payloads to induce the foregoing different aspects of the ICD pathway.

Induction of endoplasmic reticulum (ER) stress is one of the first steps for the initiation of immunogenic cell responses, and JNK signaling activation is an indicator of ER stress (see FIG. 3B). To assess this indicator, MIA-PaCa-2 pancreatic cancer cells were treated with 1 pg/mL ADCs conjugated to distinct payloads, as shown in FIG. 7B. After 24 or 48 hours of treatment, cells were harvested for western blot analysis, and the upstream ER stress marker pJNK was assessed by Simple Western immunoassay (Wes™, Protein Simple).

Treated cells were dissociated from culture plates using cell scrapers. Suspended cells were centrifuged for 10 minutes at 1000 rpm, 4° C. Supernatant was removed and cell pellets were resuspended in lysis buffer (containing protease and phosphatase inhibitors). After a minimum of 10 minutes on ice, samples were centrifuged at 13,500 g for 10 min to pellet out cellular debris. Lysis solution was relocated to separate tubes and stored at −80° C. Lysate protein amount was quantified using Bio-Rad DC Protein Assay Kit (Cat. #5000112), to allow for equal lane loading. Sample lysates and reagents were loaded into an assay plate and placed in Wes™. Phospho-JNK was identified using a primary antibody (Cell Signaling Technologies Cat. #9251S) and immunoprobed using an HRP-conjugated secondary antibody (Protein Simple Cat. #042-206) and chemiluminescent substrate. The resulting chemiluminescent signal was detected, quantitated, and displayed by the integrated Compass software.

7.2 Results

As shown in FIG. 7C-F, of the different ADC payloads tested, MMAE-ADCs (SGD-1006) treatment was one of the strongest inducers of JNK phosphorylation, an early ER stress response. In general, MMAE-ADC treatment generated stronger pJNK signals compared to treatment with maytansine-ADCs (FIG. 7C), camptothecin-ADCs (FIG. 7D), anthracycline-ADCs (FIG. 7E), and calicheamicin-ADCs (FIG. 7F). (hIgGs in FIG. 7C-F are non-targeted conjugates with the same payload as the corresponding ADC.) The sole exception was treatment with an ADC containing the anthracycline mp-EDA-PNU (SGD-8335), which generated a pJNK signal comparable to that from treatment with MMAE-ADCs (FIG. 7E). ER stress induction is tied to microtubule disruptions as the ER requires intact microtubules to expand and contract to accommodate the protein translational needs of the cell. The ability of MMAE as a microtubule disrupting agent to induce this ER stress is exemplified in the data shown.

### Example 8: Induction of Late Stage ER Stress Markers (CHOP Induction) is Generally Superior for MMAE ADCs

8.1 Materials and Methods

CHOP is the last step in the ER stress response cascade and its expression levels are increased by ER stress (see FIG. 3B). Assessment of this downstream pathway to ER stress was performed using MIA-PaCa-2 cells transduced with a CHOP-driven luciferase reporter (Signosis, Inc.). Several ADCs comprising distinct payloads were used in the assessment. See FIG. 7A.

Induction of CHOP in the MIA-PaCa-2 cells was measured by detection of luciferase signal (Bright-Glo™ Luciferase Assay System, Promega). In brief, 10,000 cells/well were plated in 96-well, black-walled, f1at-bottom clear plates in 75 μL per well. ADCs were dosed in 25 μL per well to achieve a final IC50 concentration. At 36, 48, and 72 hours, plates were removed from the incubator and allowed to come to room temperature. 100 μL of Bright-Glo Reagent was added to each well. Plate was shaken for at least five minutes before reading. The Envision CTG 96-well Standard Protocol was used to read the plate.

8.2 Results

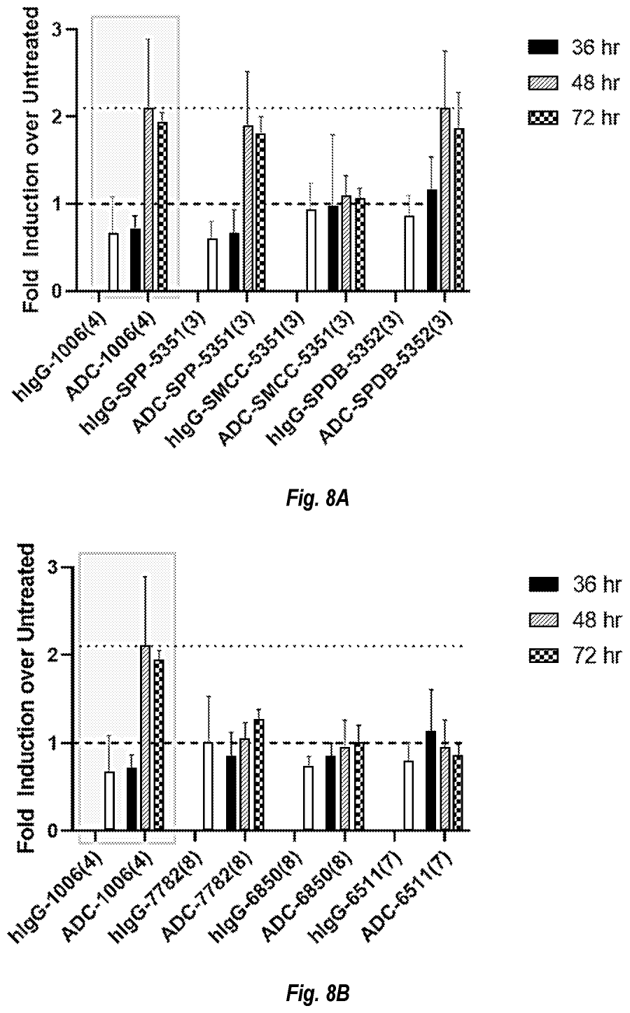

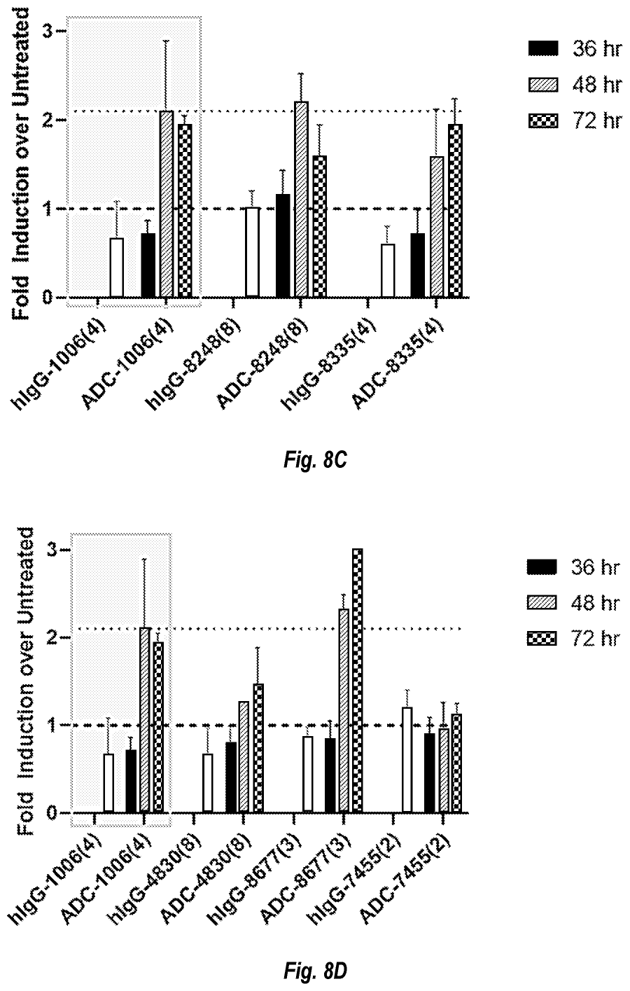

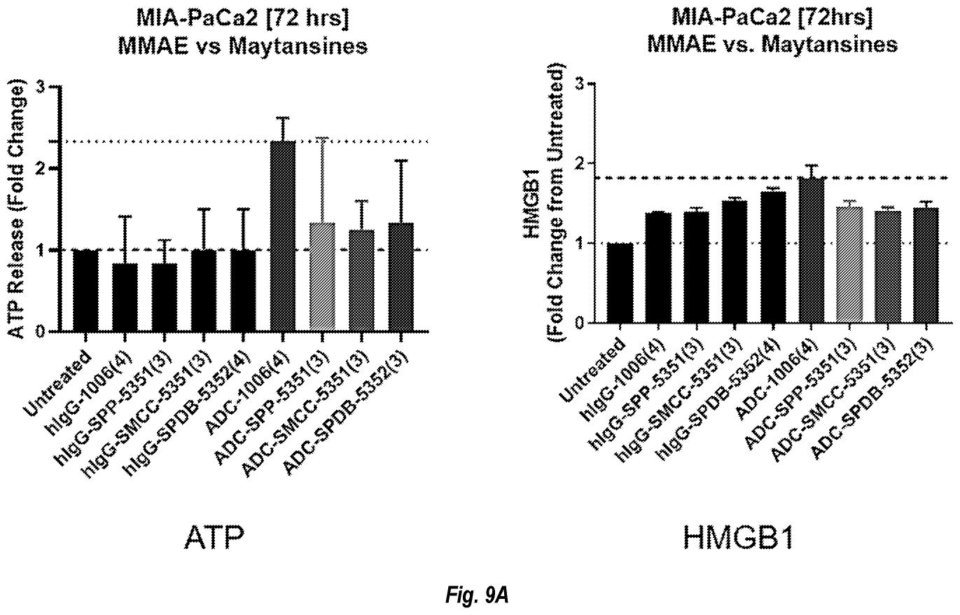

As shown in FIG. 8A-D, treatment with ADCs containing vc-MMAE (SGD-1006) resulted in CHOP induction that was comparable to CHOP induction from treatment with ADCs containing mertansine (SPP-5351) or ravtansine (SPDB-5352) (FIG. 8A) and from treatment with ADCs containing mp-EDA-PNU (SGD-8335) or mp-Gluc-DXZ (SGD-8248) (FIG. 8C). Also, treatment with ADCs containing MMAE (SGD-1006) resulted in CHOP induction that was stronger than CHOP induction from treatment with ADCs containing camptothecins (FIG. 8B), AT (SGD-4830) (FIG. 8D), or teserine (SGD-7455) (FIG. 8D). In contrast, treatment with ADCs containing ozogamycin (SGD-8677) resulted in CHOP induction that was slightly stronger than that from treatment with ADCs containing vc-MMAE (SGD-1006) FIG. 8D).

### Example 9: Induction of Immunostimulatory DAMPs is Generally Superior for MMAE ADCs

9.1 Materials and Methods

ICD causes release of immune-activating molecules-damage-associated molecular patterns (DAMPs)—such as ATP, HMGB1, and CRT. To measure ICD, ATP and HMGB1 release was assessed as follows. MIA-PaCa-2 cancer cells were treated with IC50 concentrations of ADCs with various payloads to assess in vitro ICD marker induction. See FIG. 7A.

200,000 cells were plated per well in 6-well TC plates and allowed to attach to plate ON. Cells reached 50-60% confluence. IC50 concentrations of ADCs were added to each treatment well. After 72 hours, 500 μL (for the ATP release assay) or 750 μL (for the HMGB1 assay) of culture supernatant was collected and transferred into a labeled 1.5 mL Eppendorf tube. Each tube of sample was centrifuged at 13,000 rpm for 1 minute. 50 μL of media was transferred to triplicate wells in a 96-well clear bottom plate. 50 μL of CellTiter-Glo® (Promega) was added to each well. The plate was shaken for 1-2 minutes. The CTG 96-well Standard Protocol on the Envision Plate Reader was used to read the plate. Each tube sample supernatant was then used to measure HMGB1 release levels, which were quantified by ELISA (IBL). HMGB1 and ATP release levels were reported as the fold change over the background values for untreated samples. Acquired values were converted to text file and exported and analyzed using Excel and GraphPad Prism.

9.2 Results

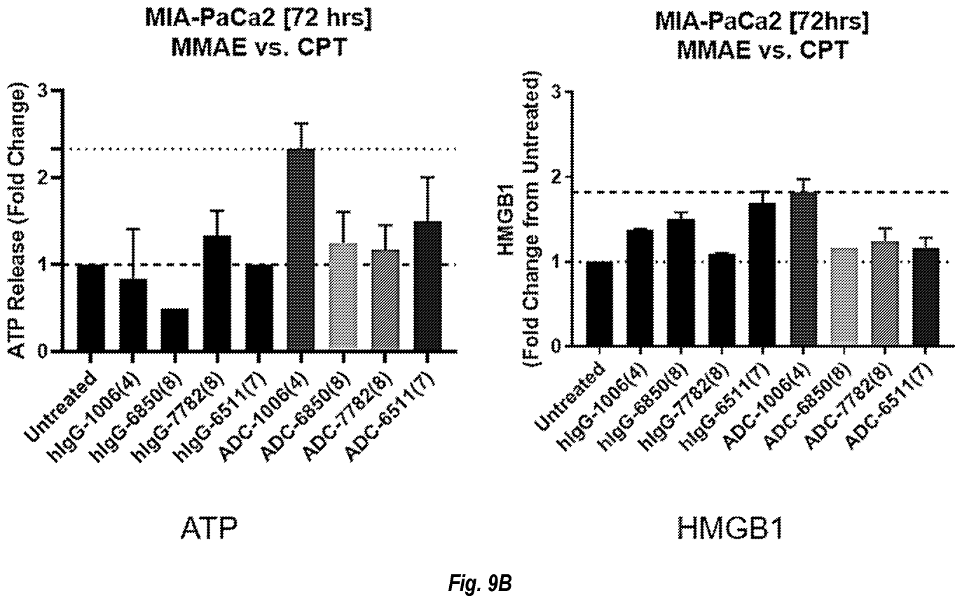

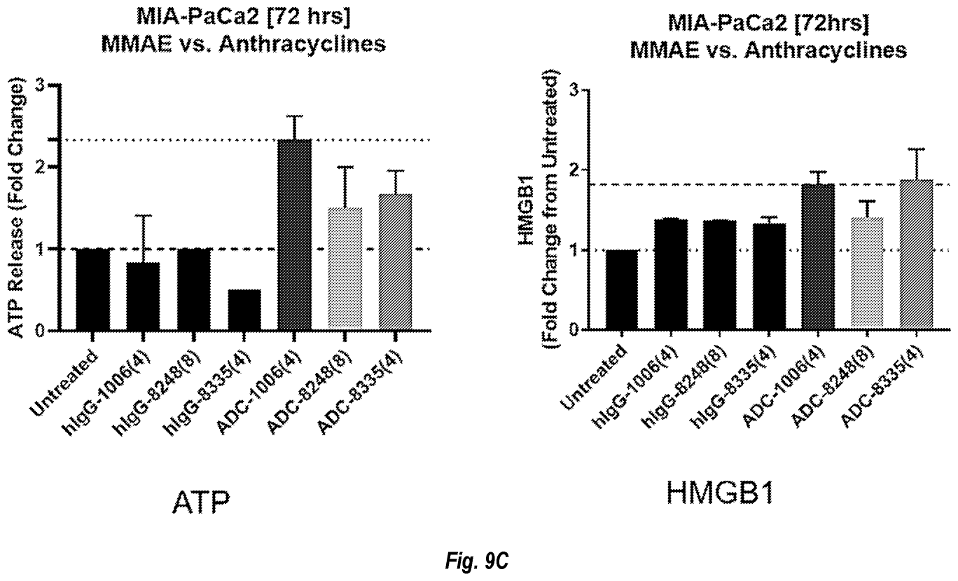

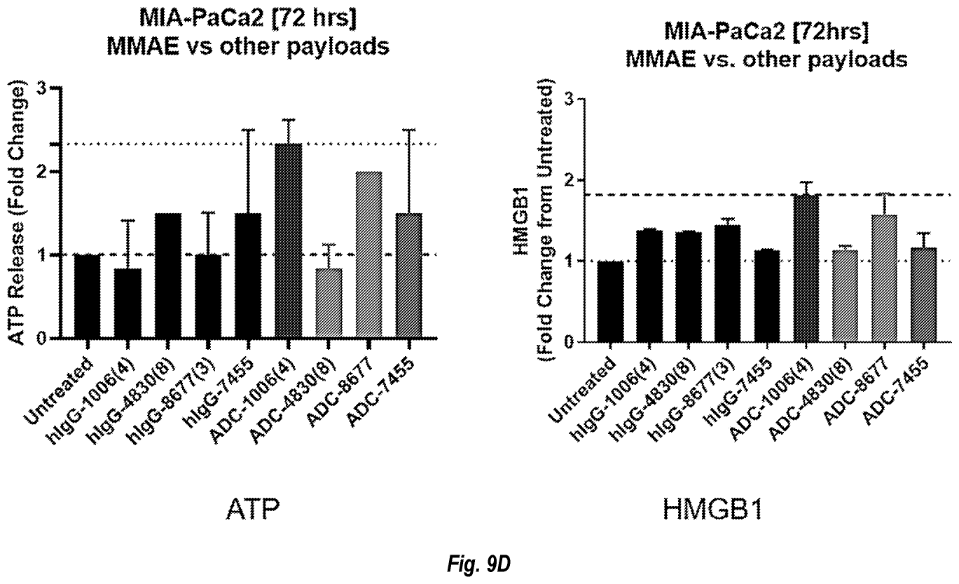

As shown in FIG. 9A-D, treatment with ADCs containing vc-MMAE (SGD-1006) resulted in ATP release and HMGB1 release that was stronger than that from treatment with ADCs containing maytansines (FIG. 9A) and from treatment with ADCs containing camptothecins (FIG. 9B). Also, treatment with ADCs containing vc-MMAE (SGD-1006) resulted in ATP release and HMGB1 release that is stronger than that from treatment with ADCs containing teserine (SGD-7455) or the auristatin AT (SGD-4830) (FIG. 9D).

Treatment with ADCs containing vc-MMAE (SGD-1006) resulted in ATP release and HMGB1 release that is comparable to that from treatment with ADCs containing anthracyclines (compared to HMGB1, ATP release is less robust) (FIG. 9C) and from treatment with ADCs containing ozogamycin (SGD-8677) (FIG. 9D).

### Example 10: Activation of Innate Cells (Cytokine Release) is Generally Superior for MMAE ADCs

10.1 Materials and Methods

DAMPs activate innate cells that can initiate antitumor T cell responses. For example, they can increase the expression of MHCII on monocytes and the release of innate cytokines such as CXCL-10/IP10. MHCII expression and CXCL-10/IP10 was assessed as follows.

L540cy cancer cells exposed to IC₅₀ concentrations of ADCs or paclitaxel for 24 hours (at 37° C. in 5% CO₂) were washed, and 0.2×10⁶ cells/well PBMCs were added to the killed cancer cells for a 1:10 L540cy:PBMC ratio. The payloads of the ADCs used in this experiment are described FIG. 7A. Co-cultures were incubated for 48 hours. Cell culture supernatants were collected at 24 hours, and cytokines were measured by Luminex assessment, including the innate cytokine CXCL-10/IP10.

Following the 48-hour co-culture incubation, cell pellets were resuspended in 50 μL of BD FACs buffer and transferred to 96-well round-bottom microtiter plates. Fc receptors were blocked with human Fc-fragments at 100 μg/mL for 30 minutes on ice. A master mix including PE-Cy7 anti-HLA-DR (MHCII), PE anti-CD14, PE-Dazzle 594 anti-CD11b, BV605 anti-CD3, and BV421 anti-CD19, diluted at 1:100, was prepared in BD FACs buffer containing 100 μg/mL human purified Fc-fragments. 10 μL of the master mix was added to each well containing 90 μL of re-suspended cells and samples were incubated for 1 hour on ice. Cells were then centrifuged at 400×g in a pre-cooled Eppendorf 5810R centrifuge for 5 minutes. The supernatant was removed, and the cells were washed with 200 mL of BD FACs buffer. The wash was performed twice, and the cells were resuspended in 200 mL of FACs buffer. Samples were analyzed on an Attune flow cytometer. Monocytes were defined as CD14+CD11b+CD3-CD19-. HLA-DR mean f1uorescence was determined using FlowJo analysis software.

10.2 Results

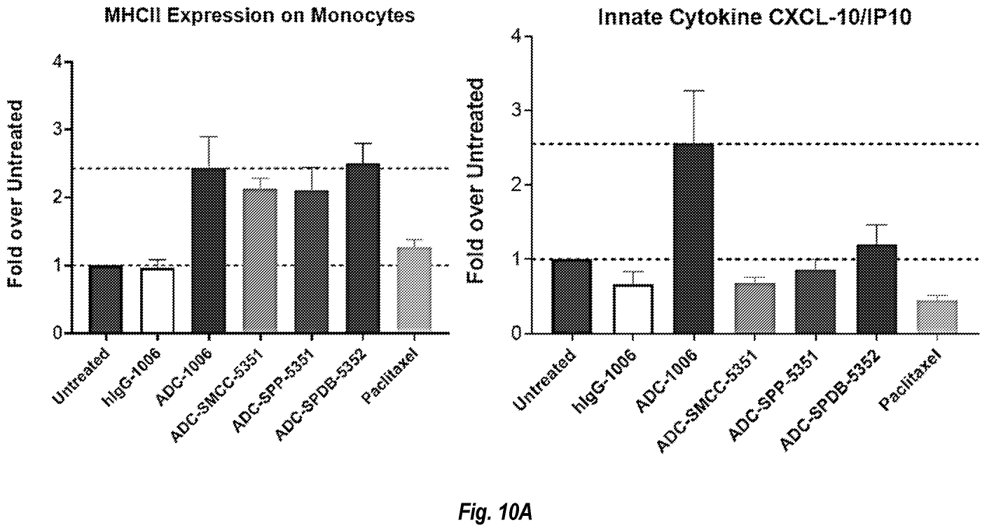

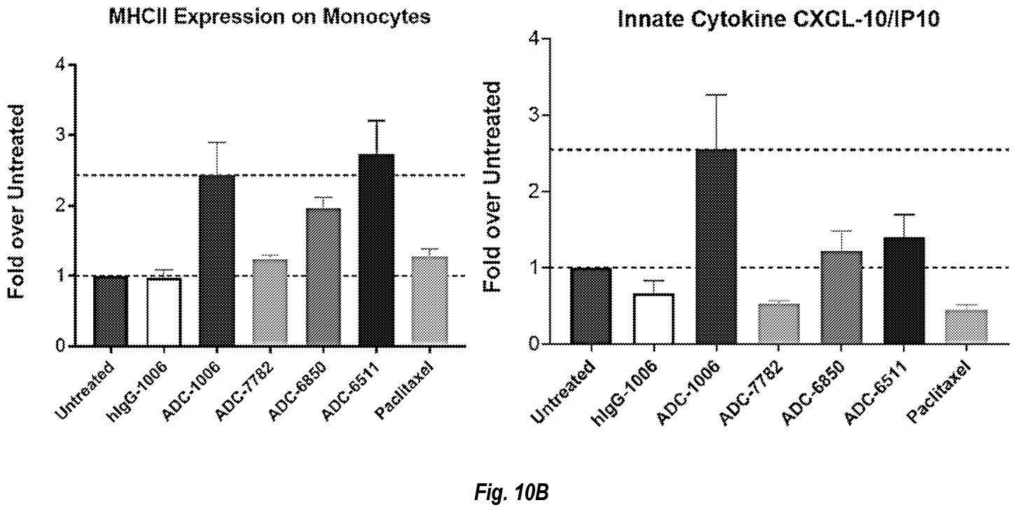

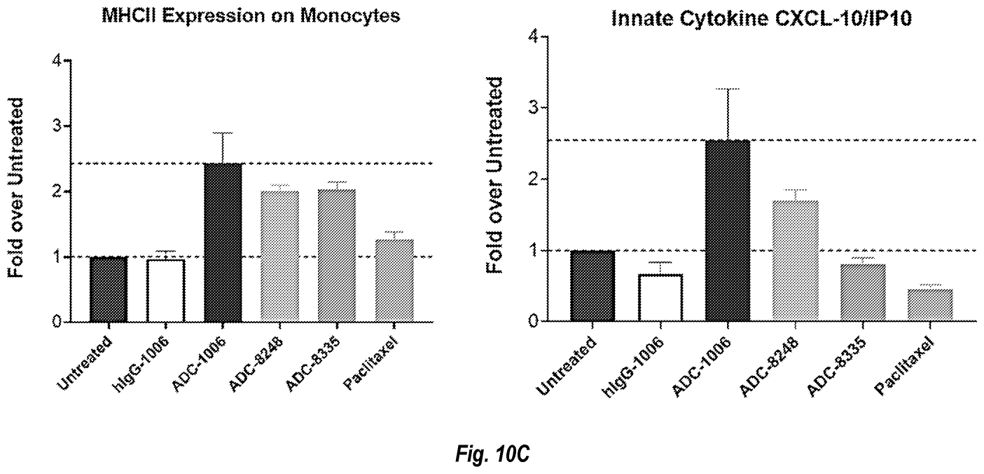

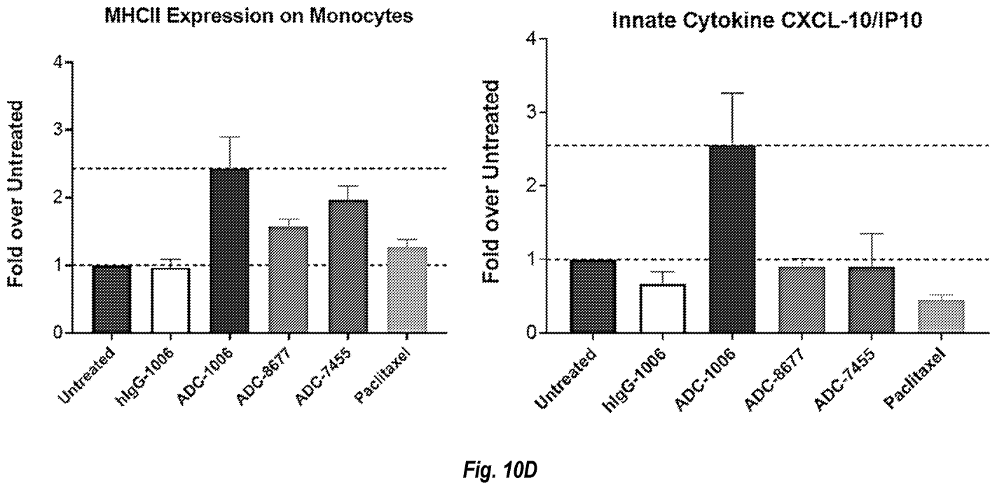

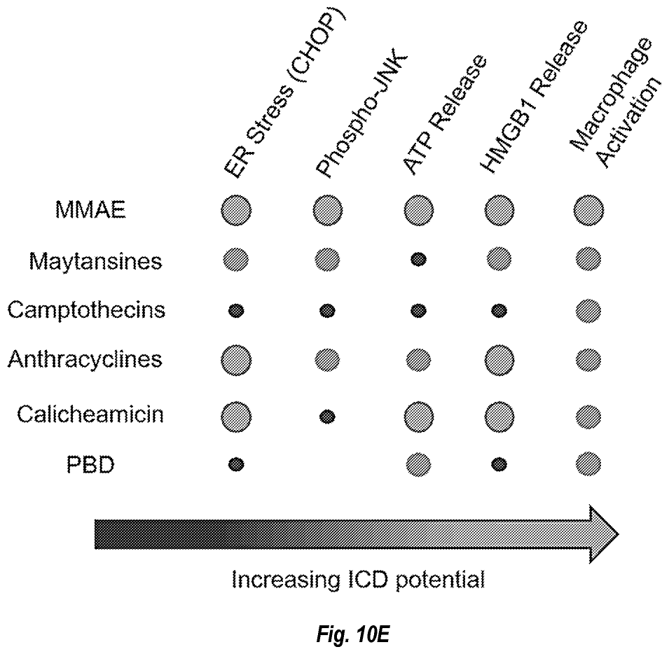

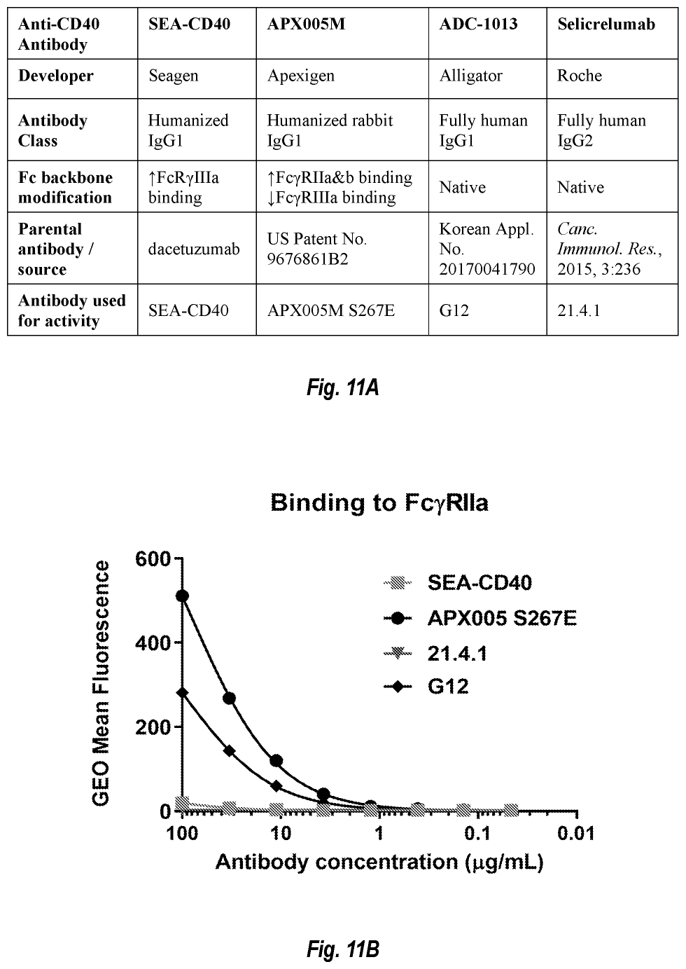

As shown in FIG. 10A-D, treatment with ADCs containing MMAE (SGD-1006) resulted in monocyte MHC II expression that was comparable to or higher than that from treatment with ADCs containing other payloads, including maytansines (FIG. 10A), camptothecins (FIG. 10B), anthracyclines (FIG. 10C), and the calicheamicin ozogamycin (SGD-8677) and the PBD teserine (SGD-7455) (FIG. 10D). Treatment with ADCs containing MMAE (SGD-1006) also resulted in release of innate cytokine CXCL-10/IP10 that was consistently higher than that from treatment with ADCs containing the same payloads (FIG. 10A-D).

10.3 Summary of Superior ICD Potential of MMAE ADCs

As shown in these experiments, MMAE-ADCs can induce various ICD hallmarks and various immunogenic cell responses, including induction of early stage ER stress (e.g., JNK activation), induction of late ER stress (e.g., CHOP induction), induction of immune-activating molecules (e.g., ATP and HMGB1 release), and activation of innate immune cells (e.g., macrophage activation). None of the other ADC payloads tested induced these ICD hallmarks consistently. FIG. 10E provides a summary of the ICD potential (as measured by the above hallmarks) of ADCs with different types of payloads, and illustrate the overall superiority of a tubulin disrupter, particularly MMAE.

### Example 11: Differential FcγR Binding to FcγRIIa, FcγRIIb or FcγRIIIa Based Upon Fc Backbone

11.1 Materials and Methods

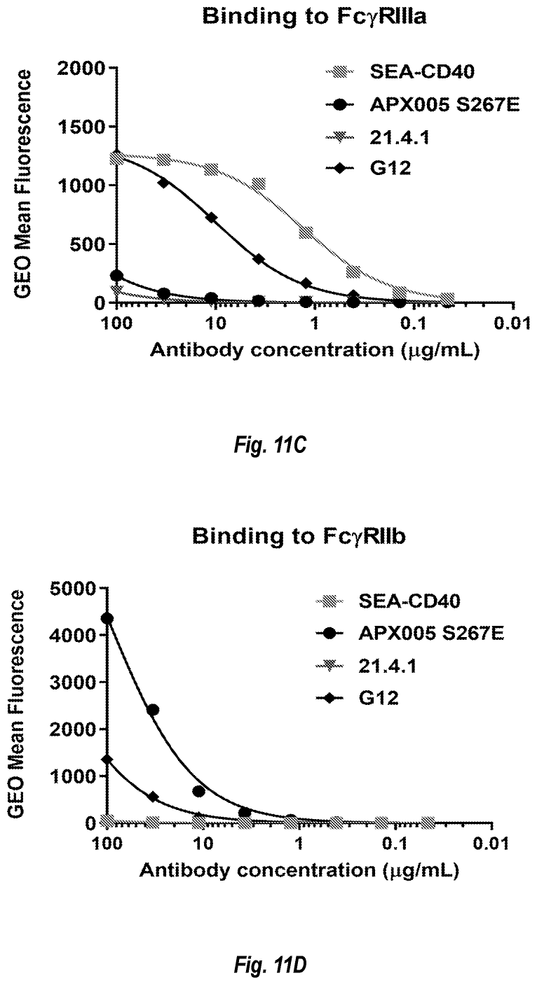

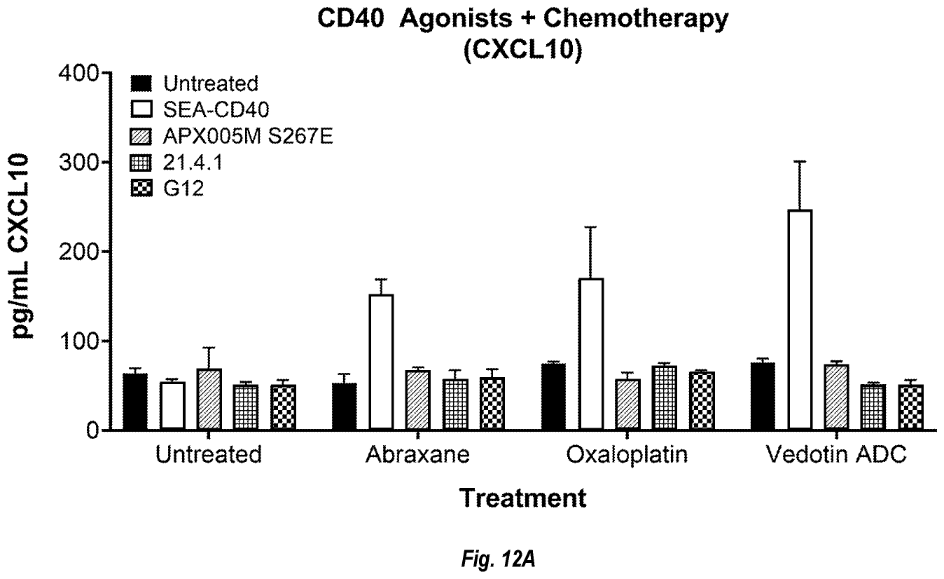

Antibodies SEA-CD40, APX005M, ADC-1013, and Selicrelumab (FIG. 11A) were assessed for FcγR binding using flow cytometry to CHO cells that were transfected with human FcγRIIa, FcγRIIb or FcγRIIIa. CHO cells were incubated with increasing concentrations of antibodies and a secondary antibody used to assess binding as monitored by flow cytometry.

For each cell line, 50 million cells were washed once in 50 mL of PBS. Cells were counted again and resuspended at 2.2 million cells/mL in BD stain buffer. Cells were plated in a 96-well round bottom plate at 0.1 mL of cells per well.

Antibody solutions were diluted to make the following final concentrations: 3 mg/mL, 1 mg/mL, 0.3 mg/mL, 0.1 mg/mL, 0.03 mg/mL, 0.01 mg/mL, 0.003 mg/mL, 0.001 mg/mL, 0.0003 mg/mL. Each antibody solution was diluted at 10×(i.e., 11 μL each antibody solution was added to 89 μL of media) to produce the following concentrations: 300, 100, 30, 10, 3, 1, 0.03. 0.01, 0.003, 0.001, and 0.0003 μg/ml. Media was removed from the cells and the cells were washed with media. 100 μL of antibody solution was added to each well. Antibody solutions were added with decreasing concentrations in the vertical direction. After a 1-hour incubation at 4° C., the plate was centrifuged, and each well of cells was washed twice with 200 μL BD stain buffer. Pelleted cells were resuspended by vortexing the plate.

Next, PE conjugated anti-human IgG Fc antibody was prepared (1/50 dilution of 1 mg/ml concentration=33 pg/mL saturating concentration) in BD stain buffer. The solution was incubated at 30 min in a dark fridge. After incubation, the plate was centrifuged, the supernatant was removed, and the cells were washed twice with 200 μL BD stain buffer per well. Cells were resuspended in PBS+1% paraformaldehyde and kept at 4° C. until they were analyzed by flow cytometry. Samples were analyzed using the Attune flow cytometer. Data points for the geometric mean of f1uorescence intensity (GEO mean f1uorescence) were graphed using Graphpad Prism.

11.2 Results

APX005 S267E exhibited the highest affinity for FcγRIIa and FcγIIb (FIG. 11B-D). SEA-CD40 had the highest affinity for FcγRIIIa with lowest affinity for FcγRIIb (FIG. 11B-D). The data demonstrate the potential of different Fc backbones to impact binding to different FcγRs. The SEA-CD40 nonfucosylated backbone shows differential binding as compared to the other CD40 antibodies in development in that it bound activating but not inhibitory FcγRs (FIG. 11B-D).

### Example 12: Induced Cell Death in MIA-PaCa-2 Cells

12.1 Materials and Methods

MIA-PaCa-2 pancreatic tumor cells were induced to undergo cell death with EC50 concentrations of the non-ICD inducing agent Abraxane (which acts similarly to paclitaxel in Example 3 above), or the 2 ICD inducing agents oxaliplatin or vc-MMAE. Cell were incubated with each agent for 18 hrs. Tumor cells were then added to human PBMCs plus various CD40-directed agonists (1 μg/ml) with differing Fc backbones (FIG. 11A) and immune activation assessed 48 hrs later.

12.2 Results

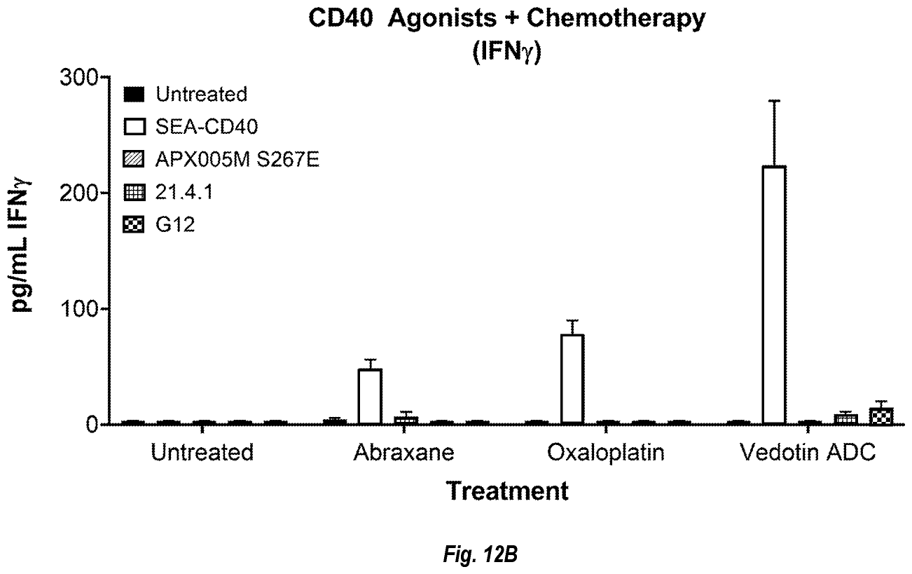

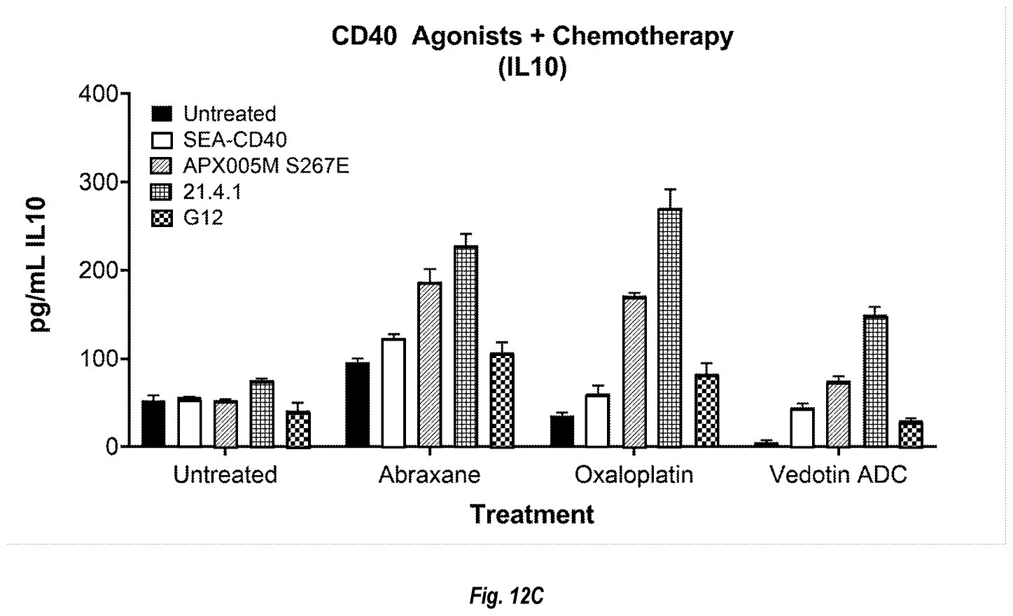

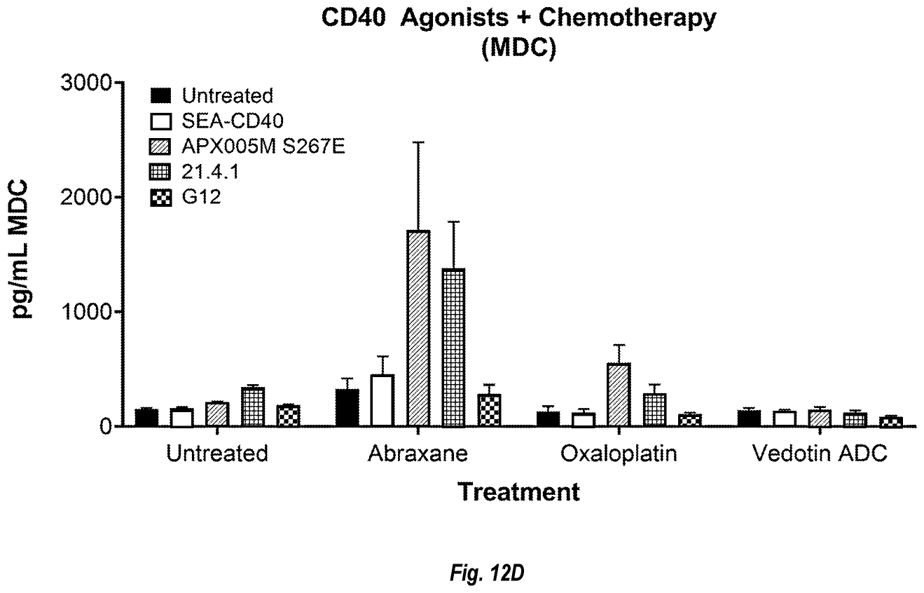

SEA-CD40 combined with vcMMAE ADC killed tumor cells, at least in part, by inducing superior release of immune activating cytokines (CXCL10 and IFNγ; FIG. 12A-B), while other CD40 agonists with differing Fc backbones amplified the immune dampening cytokines (IL-10 and MDC; FIG. 12C-D). This example illustrates the improved immune response observed with an Fc backbone that has enhanced binding to FcγRIIIa.

### Example 13: Differential Immune Activation to Apoptotic Melanoma Cells as a Function of Fc Backbone

13.1 Materials and Methods

Two melanoma cell lines, SK-MEL 12 and SK-MEL 28 were treated with Abraxane, oxaloplatin, or vc-MMAE at EC50 concentrations for 18 hrs. Human PBMCs plus 1 μg/ml of various CD40-directed agonists with differing Fc backbones (FIG. 11A) were added to the treated melanoma/tumor cell. Immune activation was assessed 48 hrs later.

13.2 Results

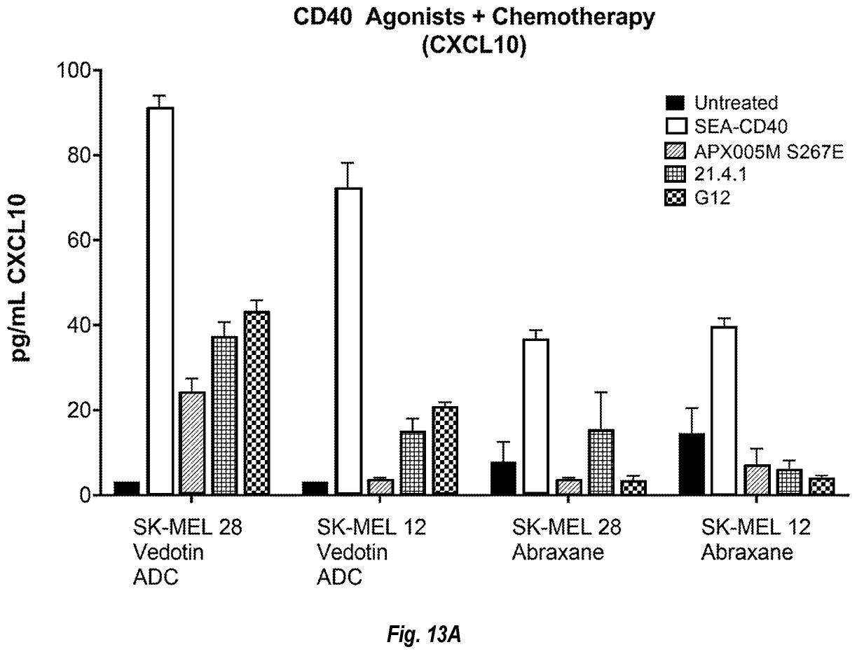

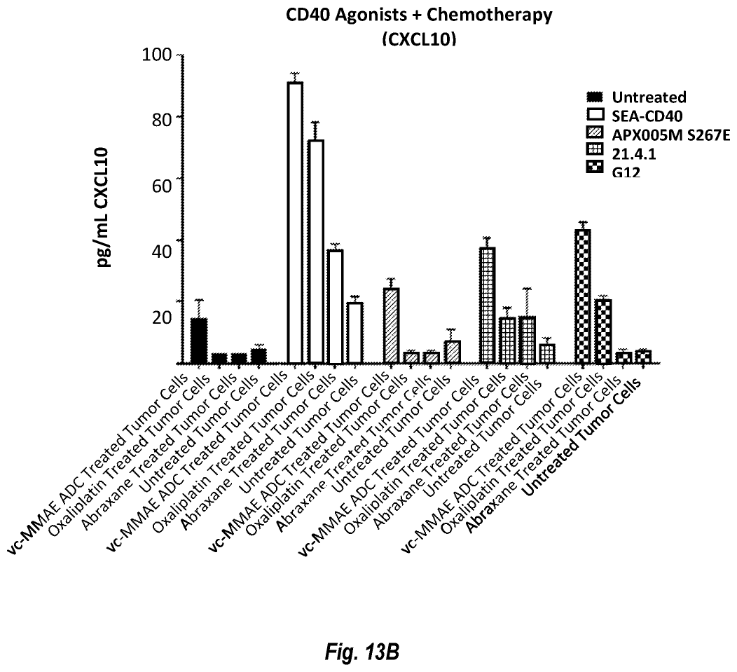

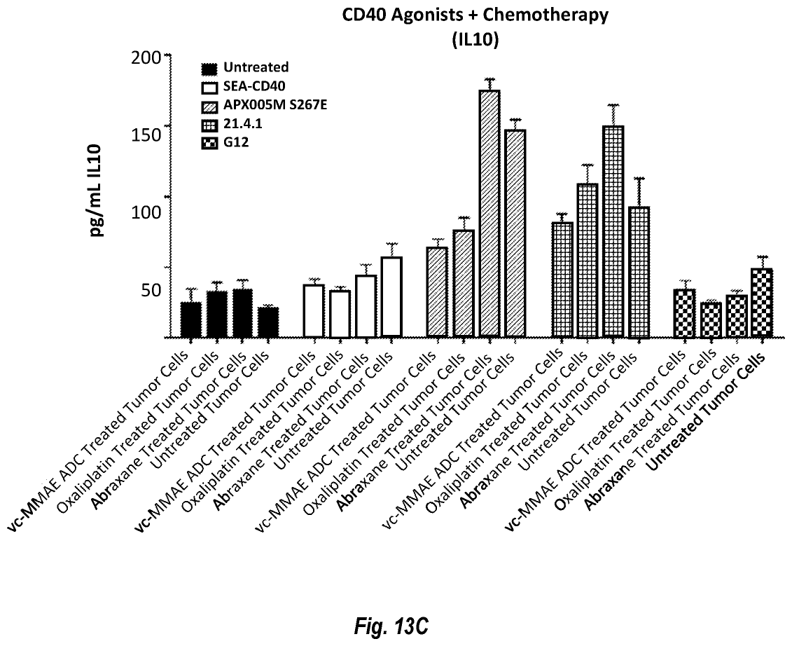

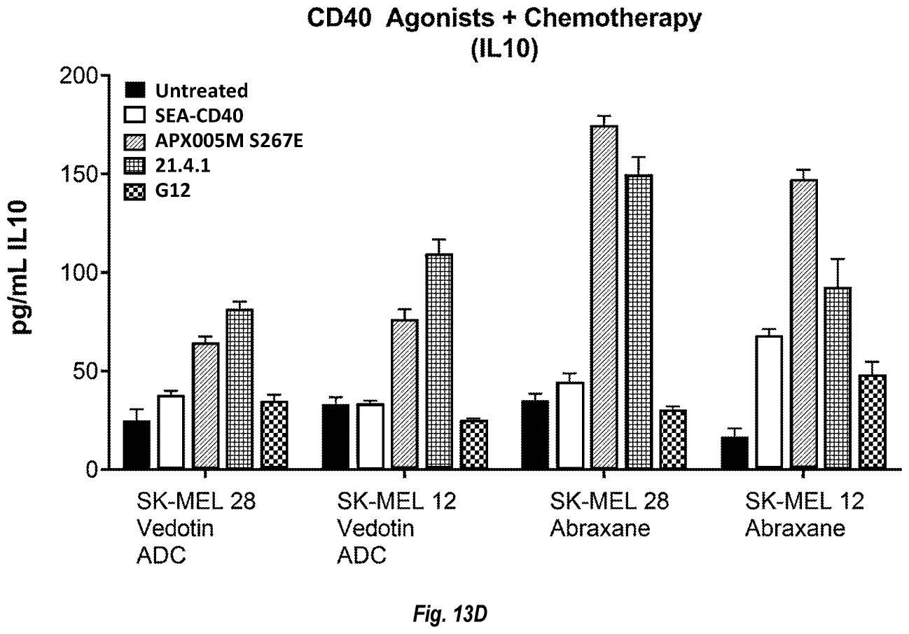

SEA-CD40 combined with vc-MMAE ADC induced release of immune activating cytokines (CXCL10; FIG. 13A-B) while other CD40 agonists amplified the immune dampening cytokines (IL-10; FIG. 13C-D).

### Example 14: Differential Immune Activation to a Variety of Apoptotic Tumor Cell Types as a Function of Fc Backbone

14.1 Materials and Methods

14.1.1 Cells

Cell death was induced in tumor cells from melanoma, lung, breast and pancreas using the ICD-inducing agent oxaliplatin or vc-MMAE at EC50 concentrations and 18 hours of incubation. Treated tumor cells were added to human PBMCs and various CD40-directed agonists with differing Fc backbones (FIG. 11A). Immune activation was assessed 48 hrs later. PBMCs and tumor cell lines were treated as described above in Example 5. Instead of MIA-PaCa-2 cancer cells (which was used in Example 5), the following cell lines were used: the melanoma cell lines SK-MEL 12 and SK-MEL 28, the lung cancer cell line A549, the breast cancer cell line MDA-MB-468, and the pancreatic cancer cell line MIA-PaCa-2. Cells were treated in triplicate.

14.1.2 Cytokine production

Cytokine production was assessed as described above in Example 5.

14.2 Results

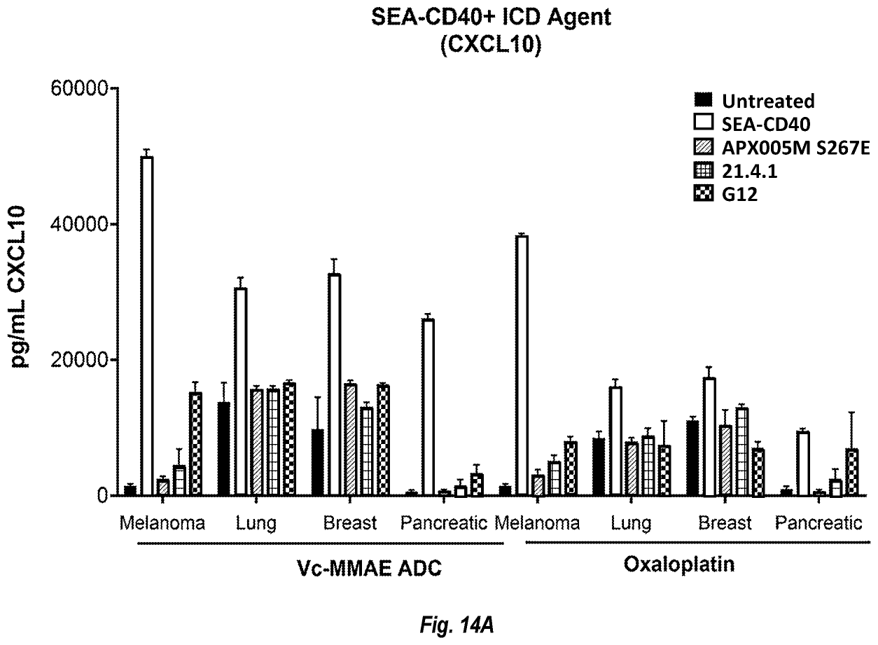

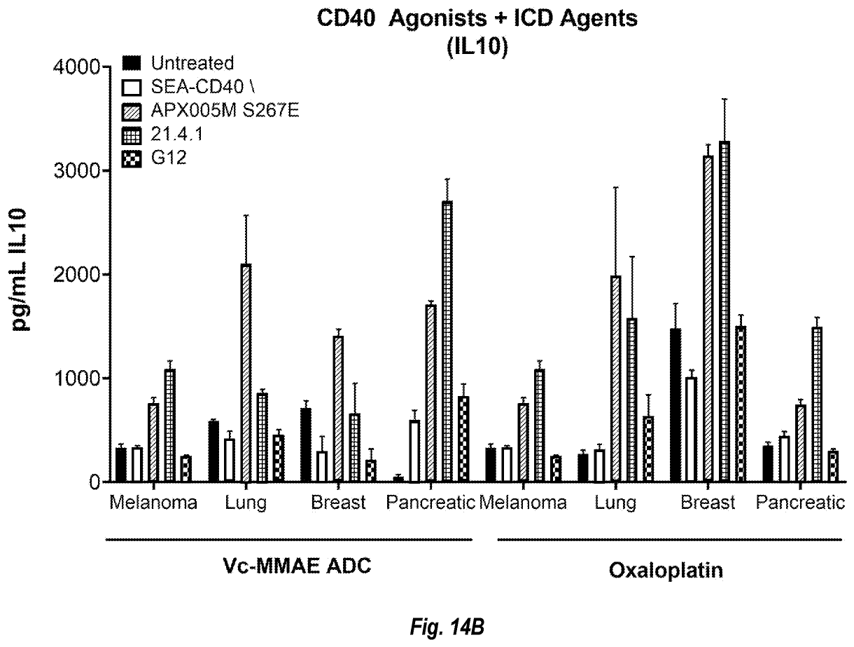

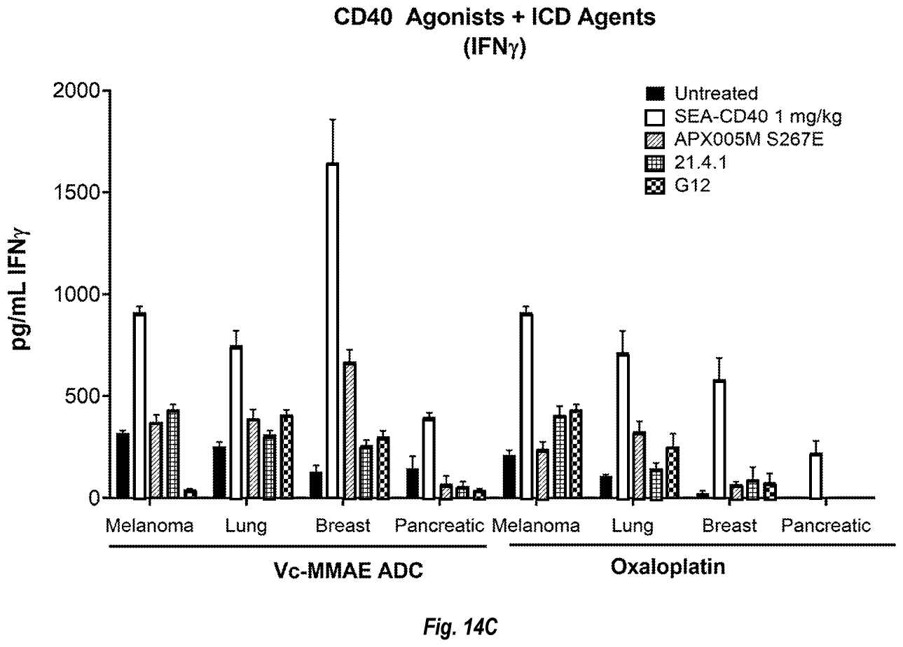

SEA-CD40 combined with vc-MMAE ADC drove superior release of immune activating cytokines (CXCL10 and IFNγ; FIGS. 14A and 14C) while other CD40 agonists amplified the immune dampening cytokines (IL-10; FIG. 14B). As with Examples 12 and 13, this example demonstrates the improved immune response observed with an Fc backbone that has enhanced binding to FcγRIIIa as compared to other Fc backbones.

### Example 15: Synergistic Effect with Combination of a Nonfucosylated SEA-CD40 Antibody and a Auristatin Based ADC

15.1 Materials and Methods

Human CD40 transgenic mice were implanted with A20 cells engineered to express the antigen Thy 1.1. Tumor cells were implanted subcutaneously in the f1ank on day 0. When mean tumor size (measured by using the formula Volume (mm³)=0.5 * Length * Width², where length is the longer dimension) of 100 mm³ was reached, mice were randomized into treatment groups of 5 mice per group. Animals were then treated with indicated treatments intraperitoneally; each treatment was given once every three days for a total of three treatments. Stock concentrations of antibody were diluted to the appropriate concentration and injected into animals in 100 μl volumes. Final dosages were 1 mg/kg for the SEA-CD40 and 1 mg/kg for the vc-MMAE ADC directed to Thy1.1. Tumor length, tumor width, and mouse weight were measured throughout the study and tumor volume was calculated using the formula above. Animals were followed until tumor volume was measured until they reached ˜1,000 mm³ when animals were then euthanized.

15.2 Results

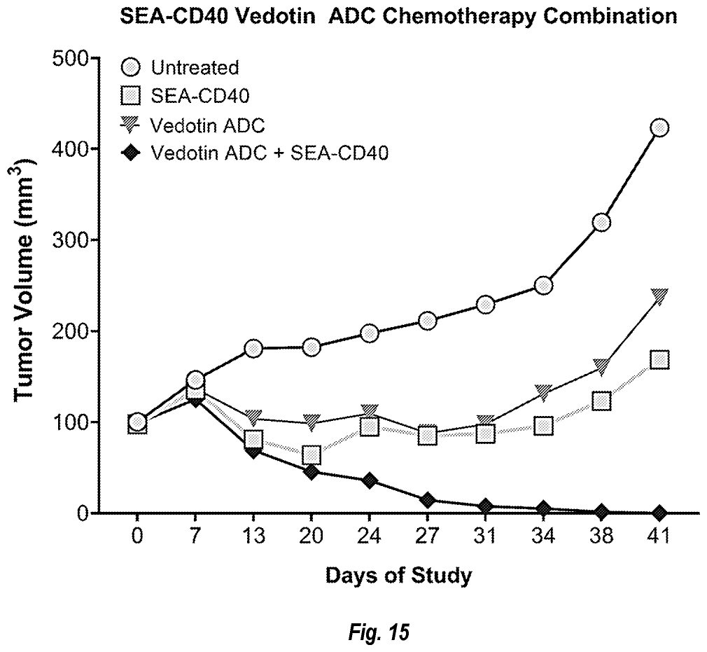

As shown in FIG. 15, treatment of the A20 tumor model with a subtherapeutic dose of SEA-CD40 resulted in reduced tumor growth and tumor growth delay. Similarly, the vc-MMAE containing antibody-drug conjugate showed mild tumor growth delay. However, when the two agents were administered in tandem to the animals, curative antitumor responses were observed. This data demonstrates the synergistic benefit in combining both the enhanced SEA-CD40 antibody with immunogenic cell death induction chemotherapy delivered via an ADC, including the possibility of achieving curative response.

### Example 16: Differential Activities in Various Tumor Cell Lines Treated with an Auristatin Based ADC Targeted to a Tumor-Associated Antigen and TIGIT Antibodies with Different Fc Backbones

16.1 Materials and Methods

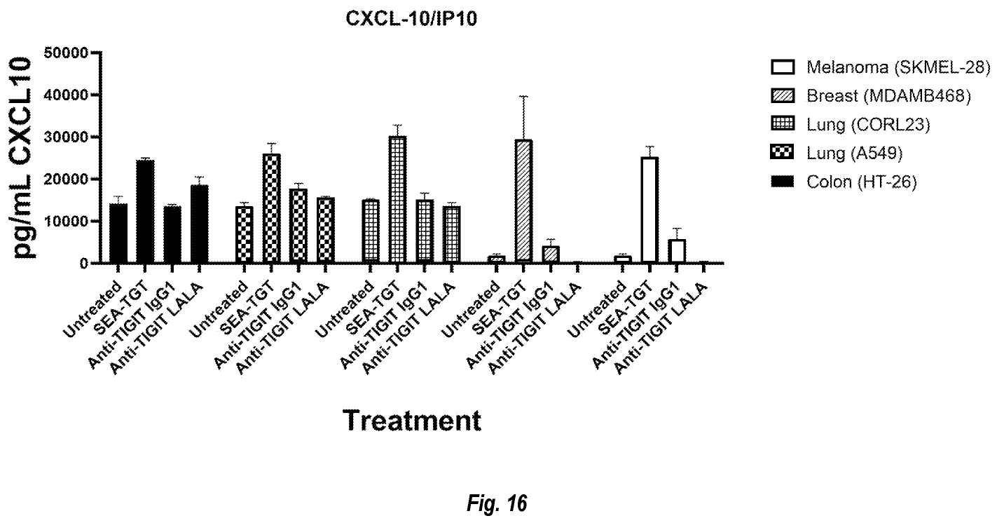

Five different human cancer cell lines, SK-MEL 28 (melanoma) MDA-MB-468 (breast), CORL23 (lung), A549 (lung), and HT-26 (colon), were used in this example. Each cell line was treated for 18 hrs with 1 μg/ml of a tumor-targeting antibody-vcMMAE ADC with a drug-to-antibody ratio (DAR) of 4. After incubation at 37° C., the tumor cells were washed and human PBMCs were added with 1 μg/ml of the anti-TIGIT antibody treatments as indicated in FIG. 16. Anti-TIGIT antibodies used had various levels of backbone effector function, with the LALA TIGIT antibody having no FcγR binding, and the SEA-TIGIT antibody having enhanced FcγR binding (increased binding to the activating FcγIIIaR, and decreased binding to the inhibitory FcγRIIbR. Table D below shows the relative activities of the different TGT antibodies. These cultures of immune cells, anti-TIGIT antibody treatment, and dead/dying tumor cells were incubated for an additional 48 hrs. The supernatant for each condition was harvested and evaluated using ELISA per the manufacturer's instructions for cytokine induction.

TABLE D
TGT antibodies

| | | T reg | CD8 T cell | APC |
|---|---|---|---|---|
| | CD155 | depletion | response | activation |
| Anti-TIGIT | ++ | − | − | − |
| LALA | | | | |
| Anti-TIGIT IgG1 | ++ | ++ | ++ | + |
| SEA-TGT | ++ | ++++ | +++ | +++ |

16.2 Results

As shown in FIG. 16, dead/dying tumor cells with PBMC incubation that didn't comprise any further immunomodulating treatments (“Untreated” in FIG. 16) had some stimulation of the immune cells as seen by the production of the innate type I interferon related cytokine IP10. The level of IP10 activation was dependent on tumor cell type as the SK-MEL 28, MDA-MB-468 and CORL23 induced much more activation of PBMCs than the A549 or HT-26 cells. Regardless of tumor cells though, subsequent addition of the enhanced effector function anti-TIGIT mAb SEA-TGT to the co-cultures resulted in further enhanced immune cell activation across the board. For the cell lines where co-incubation with dead cells alone wasn't enough to drive substantial activation, the inclusion of the nonfucosylated TIGIT mAb SEA-TGT showed the most substantial increases demonstrating its strong activation ability. In these cells the IgG1 backbone TIGIT antibody was able to drive immune cell activation that was muted when compared to the enhanced mAb. The LALA version of the anti-TIGIT, which has no FcγR engaging ability, was inactive at driving any immune activation.

### Example 17: Differential Activities in Various Tumor Cell Lines Treated with a Auristatin Based ADC Targeted to the Tumor-Associated Antigen and TIGIT Antibodies with Different Fc Backbones

17.1 Materials and Methods

Six different human cancer cell lines, HT-26 (colon), A549 (lung), CORL23 (lung), MDA-MB-468 (breast), SK-MEL 28 (melanoma), and Mia-PaCa-2 (pancreas), were used in this example. Each cell line was treated for 18 hrs with 1 μg/ml of a tumor-targeting antibody-vcMMAE ADC with a drug-to-antibody ratio (DAR) of 4. After incubation at 37° C., the tumor cells were washed and human PBMCs were added with 1 μg/ml of the anti-TIGIT antibody treatments as indicated in FIG. 16. These cultures of immune cells, anti-TIGIT antibody treatment, and dead/dying tumor cells were incubated for an additional 48 hrs. The supernatant for each condition was harvested and evaluated using ELISA per the manufacturer's instructions for cytokine induction.

17.2 Results

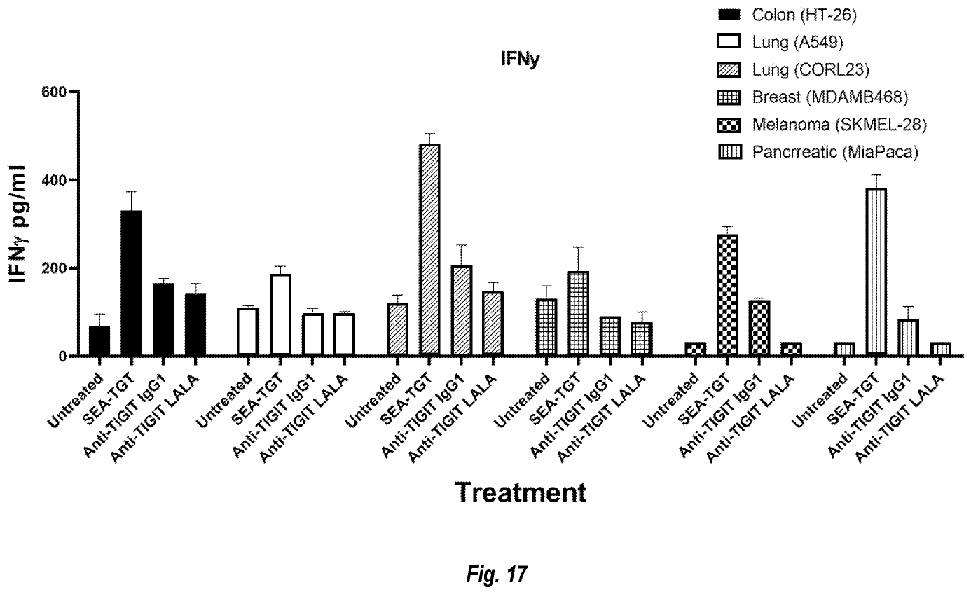

As shown in FIG. 17, dead/dying tumor cells with PBMC incubation that didn't comprise any further immunomodulating treatments (“Untreated” in FIG. 16) had some stimulation of the immune cells as seen by the production of the adaptive cytokine IFNγ. The level of IFNγ activation was dependent upon tumor cell type as SK-MEL 28, MDA-MB-468 and CORL23 induced more activation of PBMCs than A549 or HT-26 cells. Regardless of tumor cells though, subsequent addition of the nonfucosylated SEA-TGT mAb which has enhanced effector function to the co-cultures resulted in further enhanced immune cell activation across the board. For cell lines where co-incubation with dead cells alone wasn't enough to drive substantial activation, inclusion of the nonfucosylated TIGIT mAb SEA-TGT showed the most substantial increases demonstrating its strong activation ability. In these cells the IgG1 backbone TIGIT antibody was able to drive some immune cell activation but it was greatly muted vs. the enhanced mAb. The LALA version of the anti-TIGIT, which has no FcγR engaging ability, was inactive at driving any immune activation.

### Example 18: Synergistic Effect with Combination of a Nonfucosylated TIGIT Antibody and an Auristatin Based ADC

18.1 Materials and Methods

18.1.1 In vitro evaluation of a TIGIT antibody and an auristatin based ADC

A549 non-small cell lung cancer carcinoma cells were induced to undergo cell death with an EC₅₀ concentration of the ICD-inducing agent vc-MMAE conjugated to a tumor-cell-targeting antibody. The cells were incubated with the agent for 18 hours and then added to human PBMCs in concert with various concentrations (1, 0.1, 0.01 μg/ml) of anti-TIGIT antibodies with different Fc backbones, including anti-TIGIT LALA, SEA-TGT, and antibody 31C6 H4/L1, which is an IgG1 antibody (US 2018/0066055 A1). Then, immune activation was assessed by measuring cytokine (IP10) levels 48 hours after co-culture.

18.1.2 In vivo evaluation of a TIGIT antibody and an auristatin based ADC

Balb/c mice were implanted with the CT26 syngeneic tumor cell line that expresses the tumor antigen Thy1.1 subcutaneously in the f1ank on day 0. When mean tumor size (measured by using the formula Volume (mm³)=0.5 * Length * Width², where length is the longer dimension) of 100 mm³ was reached, mice were randomized into treatment groups of 5 mice per group. Animals were then treated with indicated treatments intraperitoneally; each treatment was given once every three days for a total of three treatments. Stock concentrations of antibody were diluted to the appropriate concentration and injected into animals in 100 μl volumes. Final dosages were 0.1 mg/kg for the SEA-TGT mIgG2a and 5 mg/kg for the tumor-targeting vc-MMAE Thy1.1 ADC. Both antibodies used were on mIgG2a backbones, and SEA-TGT mIgG2a is nonfucosylated. Tumor length, tumor width, and mouse weight were measured throughout the study, and tumor volume was calculated using the formula above. Animals were followed until tumor volume was measured until they reached ˜1,000 mm³ when animals were then euthanized.

Balb/c mice were implanted with Renca syngeneic tumor cell line that expresses the tumor antigen EphA2 subcutaneously in the f1ank on day 0. When mean tumor size (measured by using the formula Volume (mm³)=0.5 * Length * Width², where length is the longer dimension) of 100 mm³ was reached, mice were randomized into treatment groups of 5 mice per group. Animals were then treated with indicated treatments intraperitoneally; each treatment was given once every three days for a total of three treatments. Stock concentrations of antibody were diluted to the appropriate concentration and injected into animals in 100 μl volumes. Final dosages were 0.1 mg/kg for the SEA-TGT and 1 mg/kg for the tumor-targeting vc-MMAE EphA2 ADC. Both antibodies used were on mIgG2a backbones. Tumor length, tumor width, and mouse weight were measured throughout the study, and tumor volume was calculated using the formula above. Animals were followed until tumor volume was measured until they reached ˜1,000 mm³ when animals were then euthanized.

18.2 Results

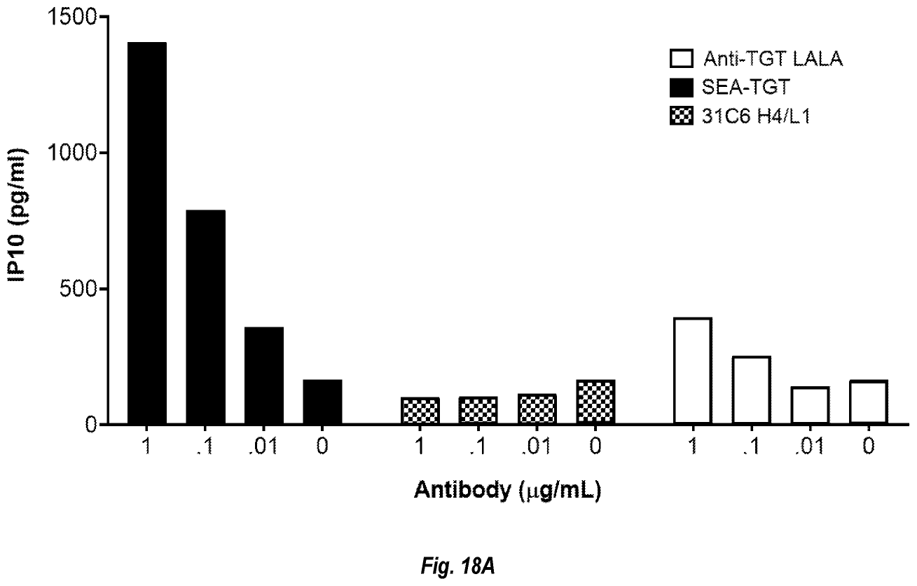

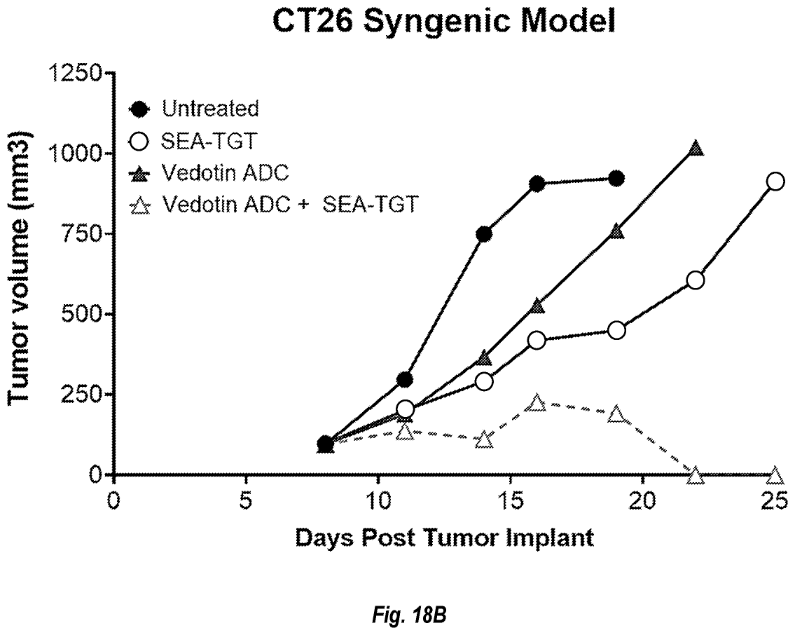

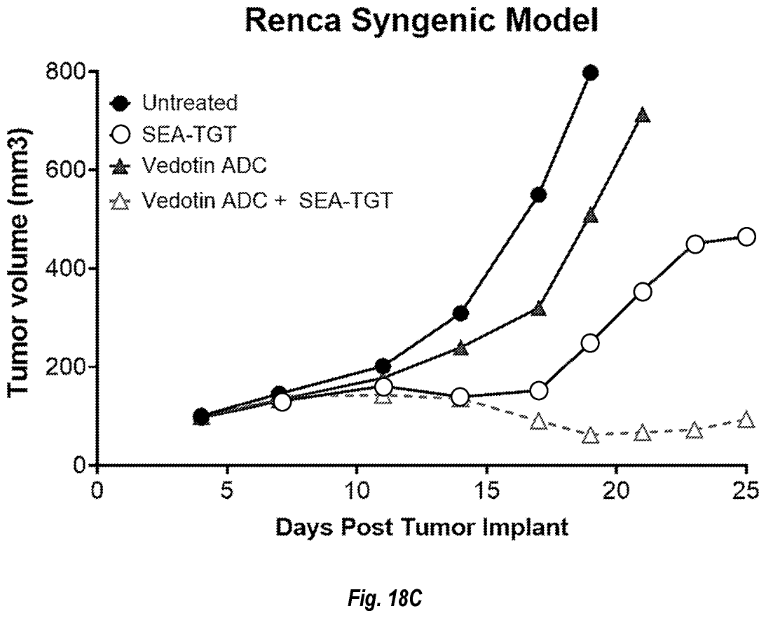

As shown in FIG. 18A, incubation of the ADC killed tumor cells with an immune population of cells, resulting in some activation of the immune cells because of the immunogenic cell death induced via the MMAE as measured by induction of the cytokine IP10 (see bars marked with “0” on X axis). Further, addition of increasing concentrations of SEA-TGT to the co-culture resulted in substantial increases in the induction of this cytokine. This was not seen with either the effector-null anti-TIGIT antibody (LALA) or the standard anti-TIGIT IgG1 antibody (31C6 H4/L1), showing that the nonfucosylated backbone of SEA-TGT drives synergy with immunogenic cell-death-inducing MMAE to provide superior immune cell activation.

As shown in FIG. 18B and FIG. 18C, treatment of the CT26 tumor model and the Renca tumor model, respectively, with a subtherapeutic dose of 0.1 mg/kg of SEA-TGT resulted in reduced tumor growth and tumor growth delay. The vc-MMAE (vedotin) ADC showed mild tumor growth delay on its own. When the two agents were administered in tandem to the animals, a substantial reduction in tumor growth was observed, as well as a curative response in 40% of the animals. These data demonstrate the synergistic benefit in combining the SEA-TGT mIgG2a antibody (the SEA-TGT antibody reformatted as a nonfucosylated mouse IgG2a that corresponds to a nonfucosylated human IgG1 backbone), with its enhanced effector function, with an ADC that induces immunogenic cell death.

The fact that such observations were made in two different tumor models with ADCs targeting two different tumor cell antigens suggests the anti-tumor activity of this combination may be broadly applicable to different tumor types.

### Example 19: Synergistic Effect with Combination of a Nonfucosylated TIGIT Antibody and Another Auristatin Based ADC

19.1 Materials and Methods

Renca cells engineered to express murine B7H4 were implanted subcutaneously in Balb/c mice. Tumors were allowed to grow to reach 100 mm³, at which time mice were treated with subtherapeutic doses of SEA-TGT and SGN-B7H4 MMAE ADC (B7H4V), or a subtherapeutic dose of SEA-TGT and a therapeutic dose of oxaliplatin. Compounds were given on the same day, and mice were treated for a total of 3 doses 7 days apart.

19.2 Results

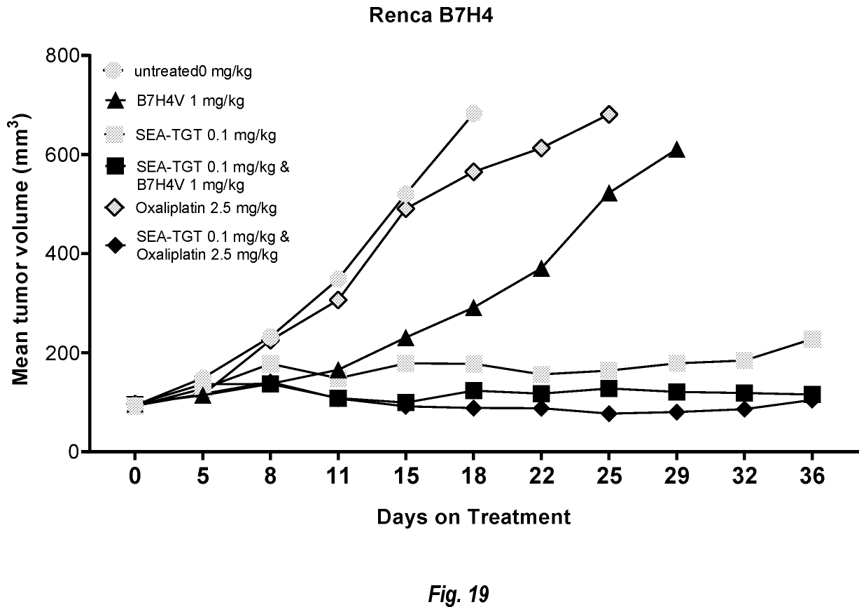

As shown in FIG. 19, SEA-TGT combinatorial activity extended to increased anti-tumor activity when a subtherapeutic dose of SEA-TGT was combined with a subtherapeutic dose of B7H4V. The combinatorial activity of SEA-TGT (subtherapeutic dose) and B7H4V (subtherapeutic dose) was similar to the combinatorial activity of SEA-TGT (subtherapeutic dose) and oxaliplatin (therapeutic dose), a known ICD inducer. Oxaliplatin, however, is associated with warnings of anaphylaxis and renal toxicity, and when given at the therapeutic dose, is not very active on its own and is often used in combination with a variety of other chemotherapies.

Also as shown in FIG. 19, the curative effect of SEA-TGT was significantly increased when it was combined with the B7H4V. Although not intending to be bound by theory, this effect is thought to be due to induction of ICD and the long-lived memory T cell response that is induced with such a combination (see also Example 23 below).

In summary, the results of this experiment are consistent with the other results described herein showing that a nonfucosylated antibody directed against an immune cell engager (in this experiment, an nonfucosylated anti-TIGIT antibody such as SEA-TGT) combines well with an agent that induces immune cell death, which in this particular example were both an MMAE ADC (i.e., B7H4V) and oxaliplatin. The combination with an MMAE ADC, however, is preferred because of the toxicity associated with oxaliplatin and because comparable therapeutic effects were observed at a subtherapeutic dose of the ADC as compared to a therapeutic dose of oxaliplatin.

### Example 20: Synergistic Effect with Combination of a Nonfucosylated SEA-CD70 Antibody and an Auristatin Based ADC

SEA-CD70 (SEA-h1F6) is a nonfucosylated antibody targeting the CD70 antigen. The CD70 molecule is a member of the tumor necrosis factor (TNF) ligand superfamily (TNFSF) and it binds to the related receptor, CD27 (TNFRSF7). The interaction between the two molecules activates intracellular signals from both receptors. In normal conditions, CD70 expression is transient and limited to activated T and B cells, mature dendritic, and natural killer (NK) cells. Similarly, CD27 is expressed on both naïve and activated effector T cells, as well as NK and activated B cells. However, CD70 is also aberrantly expressed in various hematologic cancers, including acute myeloid leukemia (AML), myelodysplastic syndrome (MDS), and non-Hodgkin's lymphoma (NHL), as well as carcinomas, and plays a role in both tumor cell survival and/or tumor immune evasion. SEA-CD70 (which comprises VH and VL of SEQ ID NOs: 41 and 42, respectively, and CDRs of SEQ ID NOs: 53-58), acts through blocking CD70/CD27 axis signaling, eliciting antibody dependent cellular phagocytosis (ADCP) and complement dependent cytotoxicity (CDC), and enhancing antibody dependent cellular cytotoxicity (ADCC). As described below, SEA-CD70 was tested in combination with brentuximab vedotin (BV, SGN-35, cAC10-MMAE) in a subcutaneous NHL model. Brentuximab vedotin, also referred to as SGN-35, is a CD30-targeting ADC containing MMAE conjugated to the monoclonal antibody cAC10. CD30 is expressed in Hodgkin's lymphoma as well as in a subset of NHL patients.

20.1 Materials and Methods

In Vivo Evaluation of Subcutaneous Tumor Growth in an NHL Xenograft Model

Farage cells (2.5×10⁶ cells/animal) were resuspended in 0.1 mL of 25% matrigel and injected subcutaneously into SCID mice, which contain active innate immune effector cells to mediate ADCP and ADCC. When mean tumor size of 100 mm³ was reached (measured by using the formula: volume (mm³)=0.5*length*width², where the length is the longer dimension), mice were randomized into treatment groups of 6 mice per group. Treatments were given intraperitoneally. Stock concentrations of antibody and chemotherapy were diluted to the appropriate concentration and injected into animals at 10 μL/g of body weight. Tumor length and width and animal weight were measured at least two times weekly throughout the study. Nineteen days post implant, dosing was initiated with 3 mg/kg SEA-CD70 and/or 1 mg/kg SGN-35. SEA-CD70 was dosed intraperitoneally (IP) every 4 days for 5 times and SGN-35 was dosed once IP on day 19. Animals were followed until tumor volume measured more than 500 mm³, at which time the animals were euthanized. A tumor size endpoint of 500 mm³ was chosen due to the tendency of tumors to become ulcerated at a larger size.

20.2 Results

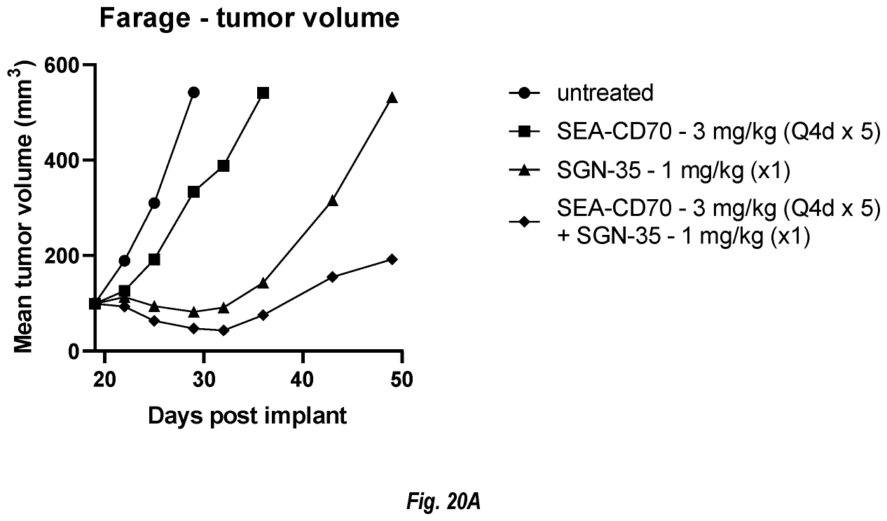

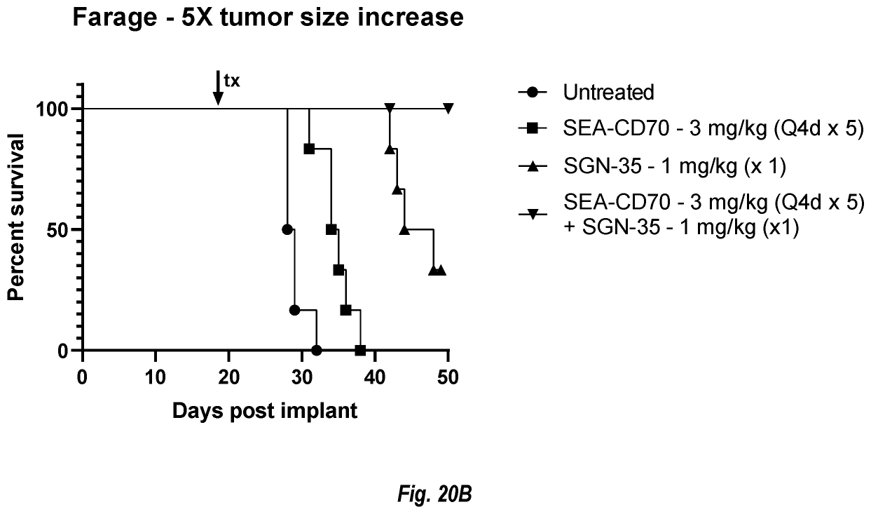

As shown in FIG. 20A, combining SGN-35 and SEA-CD70 delayed tumor growth compared to single agent SGN-35 or SEA-CD70 treatments. All untreated tumors increased volume more than 5 times their original size by day 28.5 (9.5 days post treatment), while tumors treated with single agent SEA-CD70 or single agent SGN-35 reached an average 5 fold increase at days 34.5 and 46 respectively (15.5 and 27 days post treatment). Notably, none of the tumors treated with the combination of SEA-CD70 and SGN-35 reached the established size endpoint by the time the experiment was concluded (day 50) (FIG. 20B). Compared to single SEA-CD70 or SGN-35 treatments, no overt toxicity or additional loss of weight was observed when combining SEA-CD70 and SGN-35. These data indicated that the combination of an ADC carrying an MMAE payload (SGN-35) and a nonfucosylated antibody (SEA-CD70) is efficacious and well-tolerated.

### Example 21: Synergistic Effect with Combination of a Nonfucosylated SEA-BCMA Antibody and a Auristatin ADC

SEA-BCMA is a nonfucosylated antibody targeting B-cell maturation antigen (BCMA), which is expressed on multiple myeloma (MM). SEA-BCMA (which has VH and VL of SEQ ID NOs: 45 and 46, respectively, and CDRs of SEQ ID NOs: 47-52), acts through blocking ligand mediated BCMA cell signaling, antibody dependent cellular phagocytosis (ADCP), and enhanced antibody dependent cellular cytotoxicity (ADCC). As described below, SEA-BCMA was tested in combination with SGN-CD48A in disseminated MM tumor xenograft models. SGN-CD48A is a CD48-targeting ADC containing a glucuronide linked MMAE. CD48 is broadly expressed in MM.

21.1 Materials and Methods

21.1.1 In Vivo Survival Evaluation of Xenograft Model

MM1S MM cells were injected IV into SCID animals, which contain active innate immune effector cells to mediate ADCP and ADCC. Seven days post implant, dosing was initiated with 0.1 mg/kg SEA-BCMA and/or 0.01 mg/kg SGN-CD48A, and animals were monitored for survival. SEA-BCMA was dosed weekly for 5 weeks IP and SGN-CD48A was dosed once IP. Animals were followed for 160 days for survival (N=8/group). By day 51, all untreated animals had been humanely euthanized according to IACUC protocols.

21.1.2 In Vivo Luciferase Evaluation of Xenograft Model

L363 luciferase MM cells were injected IV into SCID animals, and MM cells were allowed to home to the bone marrow. The luciferase signal was monitored over time. At thirty days post implant, dosing was initiated with 3 mg/kg SEA-BCMA and/or 0.3 mg/kg SGN-CD48A. SEA-BCMA was dosed weekly for 5 weeks IP and SGN-CD48A was dosed once IP. Animals were followed for 175 days (N=5/group). By day 58, all untreated animals had been humanely euthanized according to IACUC protocols.

21.2 Results

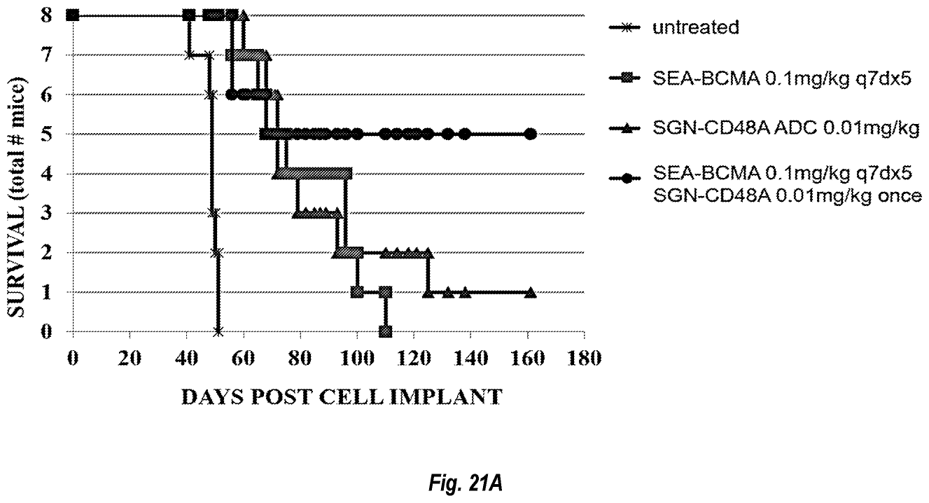

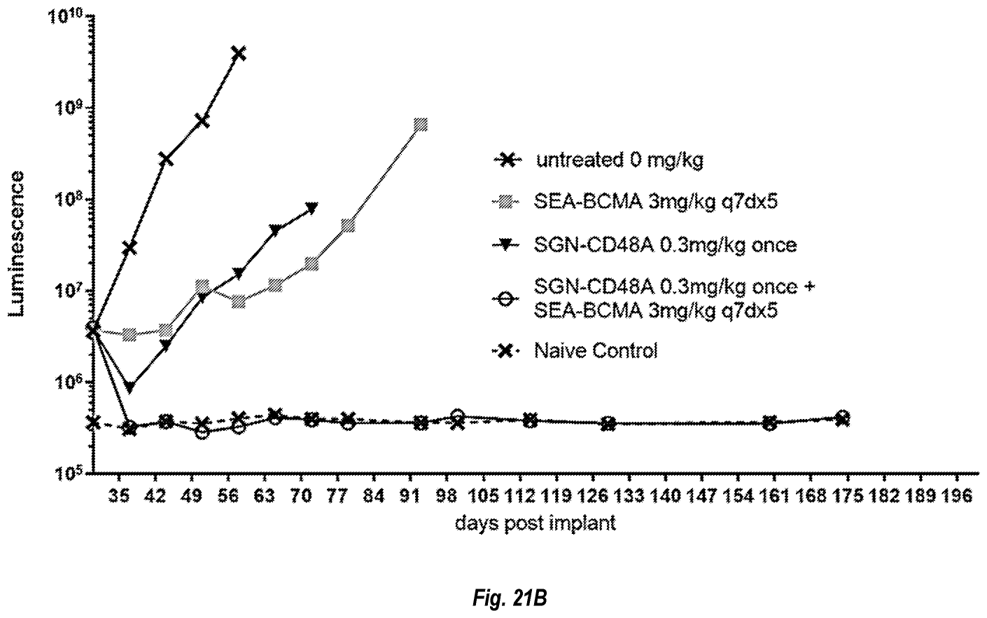

As shown in FIG. 21A-B, SGN-CD48A combined with SEA-BCMA induced complete remissions and prolonged survival in the mouse models tested. In FIG. 21A, by day 160, five of eight animals remained alive in the group that received the combination therapy, compared to zero animals treated with SEA-BCMA alone and one animal treated with SGN-CD8A alone. In FIG. 21B, by day 37, all animals treated with the combination therapy displayed no detectable luciferase signal, which remained absent until the end of study, at day 175. This striking synergy may be due to the unique combination of an ICD inducing auristatin ADC with the innate immune cell engaging SEA-BCMA.

### Example 22: Vedotin ADC Induces Immune Cell Recruitment and Activation in vivo

22.1 Materials and Methods

Tumor xenografts were isolated from animals treated with a vc-MMAE ADC or non-binding vc-MMAE isotype ADC for 8 days, and subject to flow cytometry or cytokine profiling. CD45 positive immune cells were stained for CD11c and activation observed by staining for the expression of MHC-Class II on the cell surface. Intratumoral cytokines were measured by Luminex.

22.2 Results

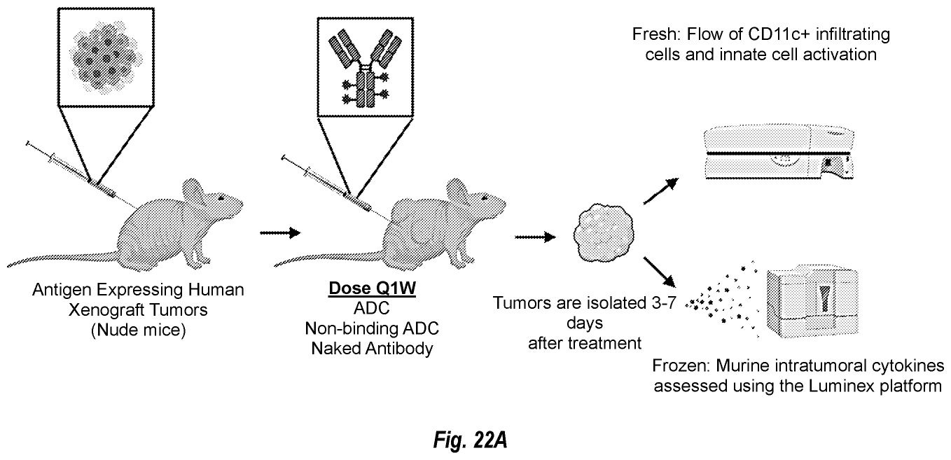

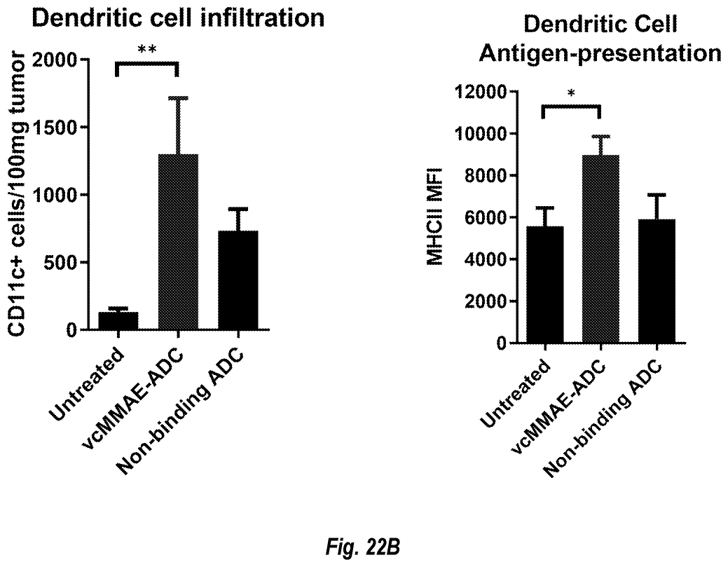

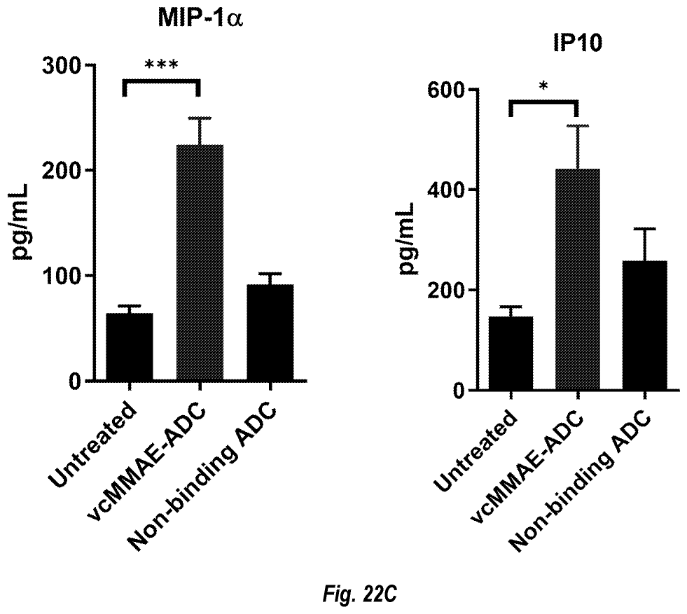

As shown in FIG. 22, Tumor-bearing mice treated with MMAE-based ADCs targeting a common tumor antigen (vc-MMAE ADC) resulted in the promotion of immune cell recruitment and activation in tumors. Dendritic cell infiltration and dendritic cell antigen-presenting were both significantly enhanced when treated with MMAE-based ADCs targeting the tumors (vcMMAE ADC) compared to the non-binding control (non-binding ADC) (FIG. 22B). Intratumoral cytokine levels were also significantly enhanced when treated with MMAE-based ADCs targeting the tumors (vc-MMAE ADC) (FIG. 22C). These data suggest that ADCs comprising tubulin disrupter induce ER stress and tumor cell death in a manner that results in promotion of immune cell recruitment and activation in tumors.

These results suggest that MMAE-based ADCs as preferred partners for immune checkpoint blockade agents.

### Example 23: Induction of T Cell Memory by Vedotin ADC

23.1 Materials and Methods

Balb/c mice were subcutaneously implanted with Renca syngeneic tumor cells, which express the tumor antigen Epha2, in the f1ank on day 0. When mean tumor size (measured by using the formula Volume (mm³)=0.5 * Length * Width², where length is the longer dimension) of 100 mm³ was reached, mice were randomized into treatment groups of 5 mice per group. Animals were then treated with indicated treatments intraperitoneally (FIG. 21A). Each treatment was given once. Stock concentrations of antibody were diluted to the appropriate concentration and injected into animals in 100 μL volumes. Final dosages were 5 mg/kg for the tumor-targeting ADCs (ADC-vcMMAE) and the non-binding ADCs (isotype-vcMMAE, also known as h00-vcMMAE). Tumor length, tumor width, and mouse weight were measured throughout the study, and tumor volume was calculated using the formula above. Animals were followed until tumor volume reached ˜ 1,000 mm³, when animals were then euthanized.

Mice who achieved a curative anti-tumor response were monitored. Then, 30 days after achieving a cure, mice were rechallenged with Renca tumor cells (FIG. 21), and outgrowth and rejection of the new tumors were assessed.

23.2 Results

As shown in FIG. 23A, in the Renca syngeneic model, treatment with a single dose of tumor-targeting MMAE ADCs (ADC-vcMMAE) resulted in strong anti-tumor activity and curative responses. As shown in FIG. 23B, when mice cured with the MMAE ADC treatment were rechallenged with Renca tumor cells to assess the induction of immune memory, such mice were able to reject the subsequently implanted tumor cells. Such results demonstrate an ability of MMAE ADCs to elicit a specific anti-tumor T cell response.

### Example 24: Brentuximab Vedotin (BV; SGN-35)-Treated Cells Confer Protective Anti-Tumor Immunity

24.1 Materials and Methods

A20 cells expressing human CD30 were treated with CD30-Auristatin ADC (BV; SGN-35) or MMAE for 18 hrs. Alternatively, one aliquot of cells was f1ash frozen. Ficoll centrifugation of treated samples was performed to remove live cells. All samples were analyzed for apoptosis and viability by flow cytometry using annexin V/7AAD. Dying and dead cells were washed, resuspended in PBS, and intraperitoneally injected into mice. The mice were immunized 2 times, 7 days apart. Immunized mice rested for 7 additional days, and then were challenged with A20 lymphoma cells, and tumor growth or rejection was monitored over time.

24.2 Results

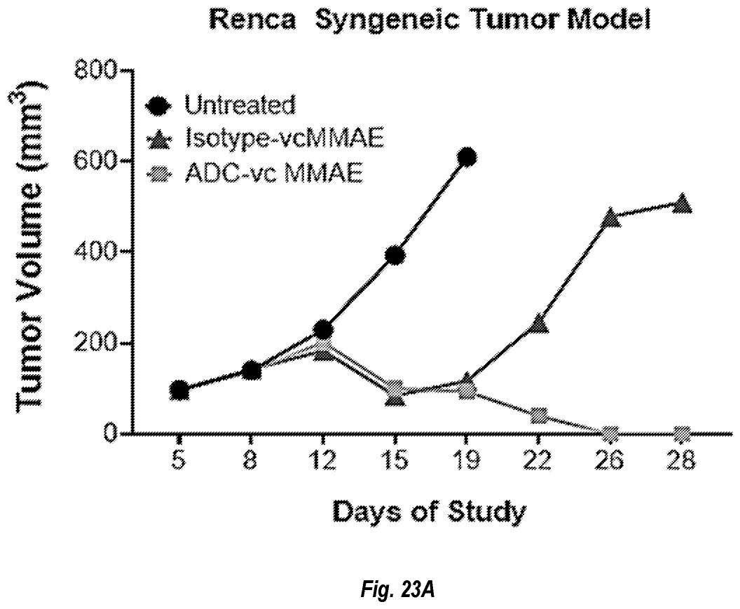

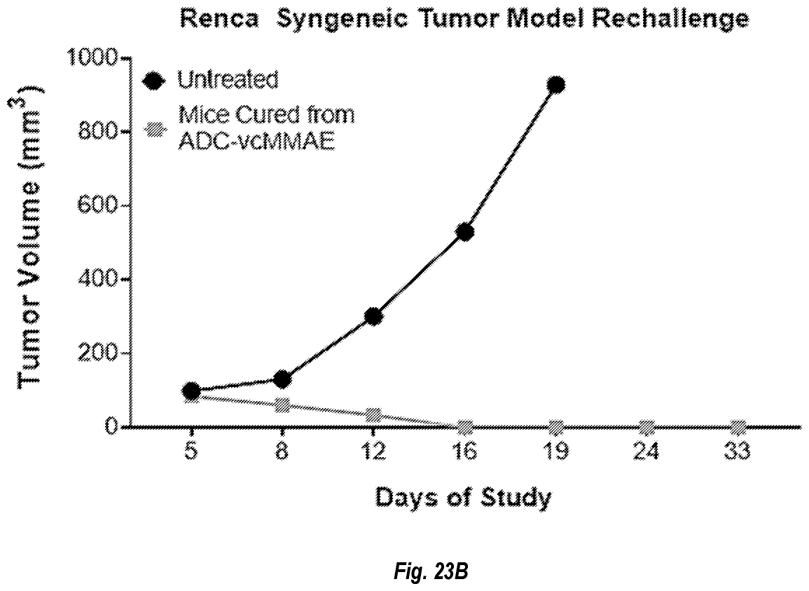

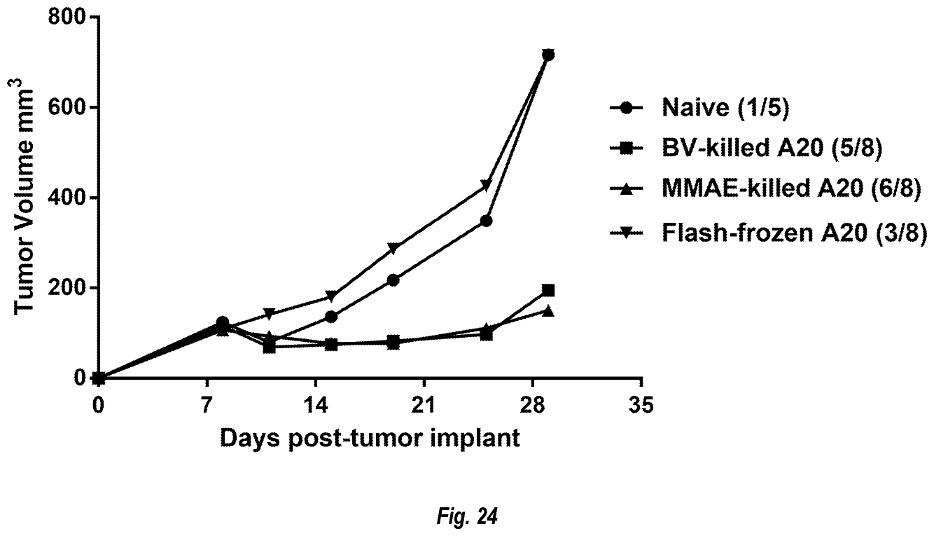

As shown in FIG. 24, mice immunized with CD30-expressing A20 cells that were killed using BV or MMAE displayed stronger immune responses rejecting implanted A20 cells, compared to mice immunized with CD30-expressing A20 cells that were killed with f1ash freezing, a non-ICD method of cell death. These results indicate an induction of a memory T cell response. Induction of immunologic memory is considered the gold standard for assessing the ICD activity of a molecule.

Collectively, the results presented in the forgoing Examples support the unique ability of auristatin based ADCs (e.g., MMAE and MMAF) to induce immunogenic cell death. As demonstrated by the foregoing examples, the mechanism of action of auristatins and their ability to disrupt microtubule networks appears associated with induction of ER stress responses that leads to the exposure and secretion of danger signals (DAMPs). Exposure of these DAMPs initiate an innate immune cell response that can lead to an antigen specific T cell response. Induction of new antigen specific T cells that can recognize tumor antigens can lead to curative anti-tumor activity preclinically that is associated with long term memory T cells responses that provide long term immune protection.

This population of memory T cells, which are induced by MMAE ADCs, may be further increased and/or enhanced by nonfucosylated antibodies. After establishing immunological memory (e.g., through the above memory T cell responses) resulting from MMAE ADC induced ICD, nonfucosylated antibodies such as SEA-TIGIT can further augment immune responses through tumor-blocking/inhibitory mechanisms, similar to the checkpoint inhibitory mechanisms of PD-1/PD-L1.

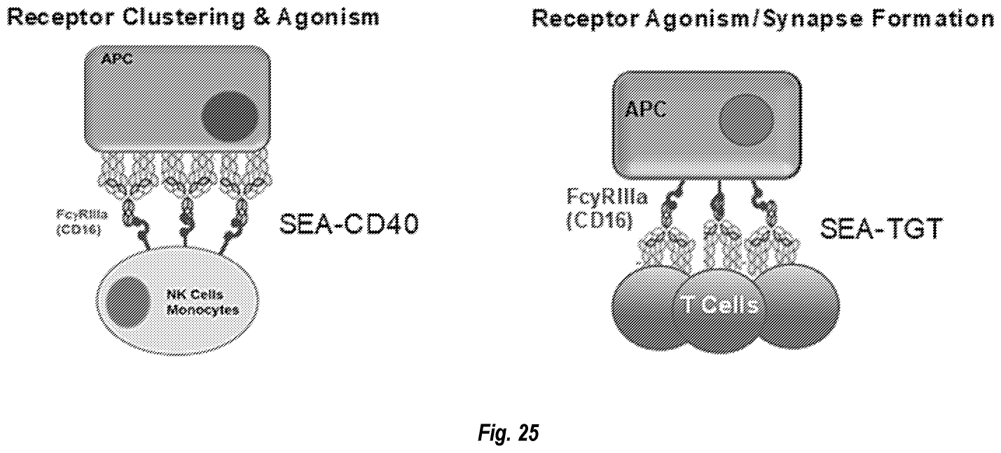

Further, pairing the ability of auristatin based ADCs (e.g., MMAE and MMAF), such as MMAE ADCs, to drive immunogenic cell death with immune cell agonism can amplify the anti-tumor activity. Immune agonism can be amplified by the use of nonfucosylated antibodies or antibodies that have been engineered to have enhanced binding to activating FcγRs and/or decreased binding to inhibitory FcγRs (e.g., as shown in the examples above with nonfucosylated CD40 and BCMA antibodies). Nonfucosylated antibodies can have increased binding to the activating FcγRIIIa receptor with and decreased or minimal binding to the inhibitory FcγRIIb receptor. This attribute is multimodal, depending on the nature of the antibody target. In the case of receptors like CD40 that are optimally active when clustered, nonfucosylated antibody binding to FcγRIIA+ cells increases receptor clustering and immune agonism and activation. See FIG. 25. As in the case of TIGIT, nonfucosylated antibodies increase the strength of an immune synapse between an antigen (+) T cells and an antigen presenting cells (FIG. 25). Engagement of the FcγRIIIa on the innate cell increases their activation and production of factors that can enhance an antigen specific T cell response. Lastly, the nonfucosylated backbone can, independently of the target antigen, bind to innate immune cells or other FcγRIIIa cells such as gamma delta T cells to induce an activated state that can help elicit a secondary antigen specific T cell response. All these mechanisms by which the nonfucosylated antibody work can lead to a T cell response that drives anti-tumor activity and long lived immune protection. The decreased or lack of binding to FcγRIIb means that there are no counter or inhibitory signals that reduce the immune activation driven by the nonfucosylated antibodies.

The mechanism of action of auristatin ADCs such as MMAE ADCs, coupled with the immune modulation by nonfucosylated mAbs, results in synergistic and complementary activity that was shown to result in enhanced immune activation and curative anti-tumor response as demonstrated herein.

All publications, patents, patent applications or other documents cited herein are hereby incorporated by reference in their entirety for all purposes to the same extent as if each individual publication, patent, patent application, or other document was individually indicated to be incorporated by reference for all purposes.

TABLE of Sequences

| | SEQ ID | |
|---|---|---|
| Name | NO | Sequence |
| Anti-TIGIT | 1 | QVQLVQSGAEVKKPGSSVKVSCKASGGTFSSYAISWVRQAPGQGLEWMG |
| antibody Clone 13 | | SIIPIFGTANYAQKFQGRVTITADESTSTAYMELSSLRSEDTAVYYCARGPSE |
| VH Protein | | VGAILGYVWFDPWGQGTLVTVSS |
| Anti-TIGIT | 2 | QVQLVQSGAEVKKPGSSVKVSCKASGGTFLSSAISWVRQAPGQGLEWMGS |
| antibody Clone 13A | | LIPYFGTANYAQKFQGRVTITADESTSTAYMELSSLRSEDTAVYYCARGPSE |
| VH | | VGAILGYVWFDPWGQGTLVTVSS |
| Anti-TIGIT | 3 | QVQLVQSGAEVKKPGSSVKVSCKASGGTFSAWAISWVRQAPGQGLEWMG |
| antibody Clone 13B | | SIIPYFGKANYAQKFQGRVTITADESTSTAYMELSSLRSEDTAVYYCARGPS |
| VH | | EVSGILGYVWFDPWGQGTLVTVSS |
| Anti-TIGIT | 4 | QVQLVQSGAEVKKPGSSVKVSCKASGGTFLSSAISWVRQAPGQGLEWMGS |
| antibody Clone 13C | | IIPLFGKANYAQKFQGRVTITADESTSTAYMELSSLRSEDTAVYYCARGPSE |
| VH | | VKGILGYVWFDPWGQGTLVTVSS |
| Anti-TIGIT | 5 | QVQLVQSGAEVKKPGSSVKVSCKASGGTFLSSAISWVRQAPGQGLEWMGS |
| antibody Clone 13D | | IIPYFGKANYAQKFQGRVTITADESTSTAYMELSSLRSEDTAVYYCARGPSE |
| VH | | VKGILGYVWFDPWGQGTLVTVSS |
| Clones 13, 13A, | 6 | DIVMTQSPLSLPVTPGEPASISCRSSQSLLHSNGYNYLDWYLQKPGQSPQLL |
| 13B, 13C, and 13D | | IYLGSNRASGVPDRFSGSGSGTDFTLKISRVEAEDVGVYYCMQARRIPITFG |
| VL Protein | | GGTKVEIK |
| Clone 13 VH CDR1 | 7 | GTFSSYAIS |
| Clones 13A, 13C, | 8 | GTFLSSAIS |
| and 13D VH CDR1 | | |
| Clone 13B VH | 9 | GTFSAWAIS |
| CDR1 | | |
| Clone 13 VH CDR2 | 10 | SIIPIFGTANYAQKFQG |
| Clone 13A VH | 11 | SLIPYFGTANYAQKFQG |
| CDR2 | | |
| Clones 13B and 13D | 12 | SIIPYFGKANYAQKFQG |
| VH CDR2 | | |
| Clone 13C VH | 13 | SIIPLFGKANYAQKFQG |
| CDR2 | | |
| Clones 13 and 13A | 14 | ARGPSEVGAILGYVWFDP |
| VH CDR3 | | |
| Clone 13B VH | 15 | ARGPSEVSGILGYVWFDP |
| CDR3 | | |
| Clones 13C and 13D | 16 | ARGPSEVKGILGYVWFDP |
| VH CDR3 | | |
| Clones 13, 13A, | 17 | RSSQSLLHSNGYNYLD |
| 13B, 13C, and 13D | | |
| VL CDR1 | | |
| Clones 13, 13A, | 18 | LGSNRAS |
| 13B, 13C, and 13D | | |
| VL CDR2 | | |
| Clones 13, 13A, | 19 | MQARRIPIT |
| 13B, 13C, and 13D | | |
| VL CDR3 | | |
| Clone 13 heavy | 20 | QVQLVQSGAEVKKPGSSVKVSCKASGGTFSSYAISWVRQAPGQGLEW |
| chain hIgG1 (and | | MGSIIPIFGTANYAQKFQGRVTITADESTSTAYMELSSLRSEDTAVYYCA |
| hIgG1 | | RGPSEVGAILGYVWFDPWGQGTLVTVSSASTKGPSVFPLAPSSKSTSGGT |
| nonfucosylated) | | AALGCLVKDYFPEPVTVSWNSGALTSGVHTFPAVLQSSGLYSLSSVVTVPS |
| amino acid | | SSLGTQTYICNVNHKPSNTKVDKKVEPKSCDKTHTCPPCPAPELLGGPSVFL |
| sequence; bold | | FPPKPKDTLMISRTPEVTCVVVDVSHEDPEVKFNWYVDGVEVHNAKTKPR |
| indicates VH; SEA- | | EEQYNSTYRVVSVLTVLHQDWLNGKEYKCKVSNKALPAPIEKTISKAKGQ |
| TGT | | PREPQVYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQKSLSL |
| | | SPGK |
| Clone 13A heavy | 21 | QVQLVQSGAEVKKPGSSVKVSCKASGGTFLSSAISWVRQAPGQGLEW |
| chain hIgG1 (and | | MGSLIPYFGTANYAQKFQGRVTITADESTSTAYMELSSLRSEDTAVYYC |
| hIgG1 | | ARGPSEVGAILGYVWFDPWGQGTLVTVSSASTKGPSVFPLAPSSKSTSGG |
| nonfucosylated) | | TAALGCLVKDYFPEPVTVSWNSGALTSGVHTFPAVLQSSGLYSLSSVVTVP |
| amino acid | | SSSLGTQTYICNVNHKPSNTKVDKKVEPKSCDKTHTCPPCPAPELLGGPSVF |
| sequence; bold | | LFPPKPKDTLMISRTPEVTCVVVDVSHEDPEVKFNWYVDGVEVHNAKTKP |
| indicates VH | | REEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSLS |
| | | LSPGK |
| Clone 13B heavy | 22 | QVQLVQSGAEVKKPGSSVKVSCKASGGTFSAWAISWVRQAPGQGLEW |
| chain hIgG1 (and | | MGSIIPYFGKANYAQKFQGRVTITADESTSTAYMELSSLRSEDTAVYYC |
| hIgG1 | | ARGPSEVSGILGYVWFDPWGQGTLVTVSSASTKGPSVFPLAPSSKSTSGG |
| nonfucosylated) | | TAALGCLVKDYFPEPVTVSWNSGALTSGVHTFPAVLQSSGLYSLSSVVTVP |
| amino acid | | SSSLGTQTYICNVNHKPSNTKVDKKVEPKSCDKTHTCPPCPAPELLGGPSVF |
| sequence; bold | | LFPPKPKDTLMISRTPEVTCVVVDVSHEDPEVKFNWYVDGVEVHNAKTKP |
| indicates VH | | REEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSLS |
| | | LSPGK |
| Clone 13C heavy | 23 | QVQLVQSGAEVKKPGSSVKVSCKASGGTFLSSAISWVRQAPGQGLEW |
| chain hIgG1 (and | | MGSIIPLFGKANYAQKFQGRVTITADESTSTAYMELSSLRSEDTAVYYC |
| hIgG1 | | ARGPSEVKGILGYVWFDPWGQGTLVTVSSASTKGPSVFPLAPSSKSTSGG |
| nonfucosylated) | | TAALGCLVKDYFPEPVTVSWNSGALTSGVHTFPAVLQSSGLYSLSSVVTVP |
| amino acid | | SSSLGTQTYICNVNHKPSNTKVDKKVEPKSCDKTHTCPPCPAPELLGGPSVF |
| sequence; bold | | LFPPKPKDTLMISRTPEVTCVVVDVSHEDPEVKFNWYVDGVEVHNAKTKP |
| indicates VH | | REEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSLS |
| | | LSPGK |
| Clone 13D heavy | 24 | QVQLVQSGAEVKKPGSSVKVSCKASGGTFLSSAISWVRQAPGQGLEW |
| chain hIgG1 (and | | MGSIIPYFGKANYAQKFQGRVTITADESTSTAYMELSSLRSEDTAVYYC |
| hIgG1 | | ARGPSEVKGILGYVWFDPWGQGTLVTVSSASTKGPSVFPLAPSSKSTSGG |
| nonfucosylated) | | TAALGCLVKDYFPEPVTVSWNSGALTSGVHTFPAVLQSSGLYSLSSVVTVP |
| amino acid | | SSSLGTQTYICNVNHKPSNTKVDKKVEPKSCDKTHTCPPCPAPELLGGPSVF |
| sequence; bold | | LFPPKPKDTLMISRTPEVTCVVVDVSHEDPEVKFNWYVDGVEVHNAKTKP |
| indicates VH | | REEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSLS |
| | | LSPGK |
| Clone 13, 13A, 13B, | 25 | DIVMTQSPLSLPVTPGEPASISCRSSQSLLHSNGYNYLDWYLQKPGQSP |
| 13C, and 13D light | | QLLIYLGSNRASGVPDRFSGSGSGTDFTLKISRVEAEDVGVYYCMQAR |
| chain hkappa (and | | RIPITFGGGTKVEIKRTVAAPSVFIFPPSDEQLKSGTASVVCLLNNFYPREA |
| nonfucosylated) | | KVQWKVDNALQSGNSQESVTEQDSKDSTYSLSSTLTLSKADYEKHKVYAC |
| amino acid | | EVTHQGLSSPVTKSFNRGEC |
| sequence; bold | | |
| indicates VL; SEA- | | |
| TGT | | |
| SEA-CD40 | 26 | EVQLVESGGGLVQPGGSLRLSCAASGYSFTGYYIHWVRQAPGKGLEWVAR |
| (nonfucosylated | | VIPNAGGTSYNQKFKGRFTLSVDNSKNTAYLQMNSLRAEDTAVYYCAREG |
| hS2C6) heavy chain | | IYWWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPP |
| | | SREEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| SEA-CD40 | 27 | DIQMTQSPSSLSASVGDRVTITCRSSQSLVHSNGNTFLHWYQQKPGKAPK |
| (nonfucosylated | | LLIYTVSNRFSGVPSRFSGSGSGTDFTLTISSLQPEDFATYFCSQTTHVP |
| hS2C6) light chain | | WTFGQGTKVEIKRTVAAPSVFIFPPSDEQLKSGTASVVCLLNNFYPREAK |
| | | VQWKVDNALQSGNSQESVTEQDSKDSTYSLSSTLTLSKADYEKHKVYACE |
| | | VTHQGLSSPVTKSFNRGEC |
| VH of SEA-CD40 | 28 | EVQLVESGGGLVQPGGSLRLSCAASGYSFTGYYIHWVRQAPGKGLEWVAR |
| | | VIPNAGGTSYNQKFKGRFTLSVDNSKNTAYLQMNSLRAEDTAVYYCAREG |
| | | TYWWGQGTLVTVSS |
| VL of SEA-CD40 | 29 | DIQMTQSPSSLSASVGDRVTITCRSSQSLVHSNGNTFLHWYQQKPGKAPK |
| | | LLIYTVSNRFSGVPSRFSGSGSGTDFTLTISSLQPEDFATYFCSQTTHVP |
| | | WTFGQGTKVEIK |
| SEA-CD40 VH | 30 | GYYIH |
| CDR1 | | |
| SEA-CD40 VH | 31 | RVIPNAGGTSYNQKFKG |
| CDR2 | | |
| SEA-CD40 VH | 32 | EGIYW |
| CDR3 | | |
| SEA-CD40 VL | 33 | RSSQSLVHSNGNTFLH |
| CDR1 | | |
| SEA-CD40 VL | 34 | TVSNRFS |
| CDR2 | | |
| SEA-CD40 VL | 35 | SQTTHVPWT |
| CDR3 | | |
| Alternative anti- | 36 | RVIPQAGGTSYNQKFKG |
| CD40 antibody | | |
| CDR2 | | |
| SGN-B6A heavy | 37 | QFQLVQSGAEVKKPGASVKVSCKASGYSFTDYNVNWVRQAPGQGLEWIGV |
| chain variable region | | INPKYGTTRYNQKFKGRATLTVDKSTSTAYMELSSLRSEDTAVYYCTRGL |
| | | NAWDYWGQGTLVTVSS |
| SGN-B6A light | 38 | DIQMTQSPSSLSASVGDRVTITCGASENIYGALNWYQQKPGKAPKLLIYG |
| chain variable region | | ATNLEDGVPSRFSGSGSGRDYTFTISSLQPEDIATYYCQNVLTTPYTFGQ |
| | | GTKLEIK |
| SGN-STNV heavy | 39 | EVQLVQSGAEVKKPGASVKVSCKASGYTFTDHAIHWVRQAPGQGLEWMGY |
| chain variable region | | FSPGNDDIKYNEKFRGRVTMTADKSSSTAYMELRSLRSDDTAVYFCKRSL |
| | | STPYWGQGTLVTVSS |
| SGN-STNV heavy | 40 | DIVMTQSPDSLAVSLGERATINCKSSQSLLNRGNHKNYLTWYQQKPGQPP |
| chain variable region | | KLLIYWASTRESGVPDRFSGSGSGTDFTLTISSLQAEDVAVYYCQNDYTY |
| | | PYTFGQGTKVEIK |
| SEA-CD70 heavy | 41 | QVQLVQSGAEVKKPGASVKVSCKASGYTFTNYGMNWVRQAPGQGLKWMGW |
| chain variable region | | INTYTGEPTYADAFKGRVTMTRDTSISTAYMELSRLRSDDTAVYYCARDY |
| | | GDYGMDYWGQGTTVTVSS |
| SEA-CD70 light | 42 | DIVMTQSPDSLAVSLGERATINCRASKSVSTSGYSFMHWYQQKPGQPPKL |
| chain variable region | | LIYLASNLESGVPDRFSGSGSGTDFTLTISSLQAEDVAVYYCQHSREVPW |
| | | TFGQGTKVEIK |
| SGN-CD228A | 43 | QVQLQESGPGLVKPSETLSLTCTVS [symbol] WIRQPPGKGLEYIG [symbol] |
| heavy chain variable | | SLKSRVTISRDTSKNQYSLKLSSVTAADTAVYYC [symbol] WGQGTLVTVSS |
| region | | |
| SGN-CD228A light | 44 | DFVMTQSPLSLPVTLGQPASISC [symbol] WYQQRPGQSPRLLIY [symbol] |
| chain variable region | | SGVPDRFSGSGSGTDFTLKISRVEAEDVGVYYC [symbol] FGQGTKLEIK (SEQ ID |
| SGN-BCMA heavy | 45 | QVQLVQSGAEVKKPGASVKLSCKASGYTFTDYYIHWVRQAPGQGL |
| chain variable region | | EWIGYINPNSGYTNYAQKFQGRATMTADKSINTAYVELSRLRSDDT |
| | | AVYFCTRYMWERVTGFFDFWGQGTMVTVSS |
| SGN-BCMA light | 46 | DIQMTQSPSSVSASVGDRVTITCLASEDISDDLAWYQQKPGKAPKV |
| chain variable region | | LVYTTSSLQSGVPSRFSGSGSGTDFTLTISSLQPEDFATYFCQQTYKF |
| | | PPTFGGGTKVEIK |
| SGN-BCMA VH | 47 | DYYIH |
| CDR1 | | |
| SGN-BCMA VH | 48 | YINPNSGYTNYAQKFQG |
| CDR2 | | |
| SGN-BCMA VH | 49 | YMWERVTGFFDF |
| CDR3 | | |
| SGN-BCMA VL | 50 | LASEDISDDLA |
| CDR1 | | |
| SGN-BCMA VL | 51 | TTSSLQS |
| CDR2 | | |
| SGN-BCMA VL | 52 | QQTYKFPPT |
| CDR3 | | |
| SEA-CD70 VH | 53 | NYGMN |
| CDR1 | | |
| SEA-CD70 VH | 54 | WINTYTGEPTYADAFKG |
| CDR2 | | |
| SEA-CD70 VH | 55 | DYGDYGMDY |
| CDR3 | | |
| SEA-CD70 VL | 56 | RASKSVSTSGYSFMH |
| CDR1 | | |
| SEA-CD70 VL | 57 | LASNLES |
| CDR2 | | |
| SEA-CD70 VL | 58 | QHSREVPWT |
| CDR3 | | |
| Zolbetuximab | 59 | QVQLQQPGAELVRPGASVKLSCKASGYTFTSYWINWVKQRPGQGL |
| (175D10) heavy | | EWIGNIYPSDSYTNYNQKFKDKATLTVDKSSSTAYMQLSSPTSEDSA |
| chain variable region | | VYYCTRSWRGNSFDYWGQGTTLTVSS |
| Zolbetuximab | 60 | DIVMTQSPSSLTVTAGEKVTMSCKSSQSLLNSGNQKNYLTWYQQKP |
| (175D10) light chain | | GQPPKLLIYWASTRESGVPDRFTGSGSGTDFTLTISSVQAEDLAVYY |
| variable region | | CQNDYSYPFTFGSGTK |
| Zolbetuximab | 61 | SYWIN |
| (175D10) VH CDR1 | | |
| Zolbetuximab | 62 | NIYPSDSYTNYNQKFKD |
| (175D10) VH CDR2 | | |
| Zolbetuximab | 63 | SWRGNSFDY |
| (175D10) VH CDR3 | | |
| Zolbetuximab | 64 | KSSQSLLNSGNQKNYLT |
| (175D10) VL CDR1 | | |
| Zolbetuximab | 65 | WASTRES |
| (175D10) VL CDR2 | | |
| Zolbetuximab | 66 | QNDYSYPFT |
| (175D10) VL CDR3 | | |
| 163E12 heavy chain | 67 | QIQLVQSGPELKKPGETVKISCKASGYTFTNYGMNWVKQAPGKGL |
| variable region | | KWMGWINTNTGEPTYAEEFKGRFAFSLETSASTAYLQINNLKNEDT |
| | | ATYFCARLGFGNAMDYWGQGTSVTVSS |
| 163E12 light chain | 68 | DIVMTQSPSSLTVTAGEKVTMSCKSSQSLLNSGNQKNYLTWYQQKP |
| variable region | | GQPPKLLIYWASTRESGVPDRFTGSGSGTDFTLTISSVQAEDLAVYY |
| | | CQNDYSYPLTFGAGTKLELK |
| 163E12 VH CDR1 | 69 | NYGMN |
| 163E12 VH CDR2 | 70 | WINTNTGEPTYAEEFKG |
| 163E12 VH CDR3 | 71 | LGFGNAMDY |
| 163E12 VL CDR1 | 72 | KSSQSLLNSGNQKNYLT |
| 163E12 VL CDR2 | 73 | WASTRES |
| 163E12 VL CDR3 | 74 | QNDYSYPLT |
| SGN-PDL1V heavy | 75 | QVQLVQSGAEVKKPGSSVKVSCKTSGDTFSTAAISWVRQAPGQGLE |
| chain variable region | | WMGGIIPIFGKAHYAQKFQGRVTITADESTSTAYMELSSLRSEDTAV |
| | | YFCARKFHFVSGSPFGMDVWGQGTTVTVSS |
| SGN-PDL1V light | 16 | EIVLTQSPATLSLSPGERATLSCRASQSVSSYLAWYQQKPGQAPRLLI |
| chain variable region | | YDASNRATGIPARFSGSGSGTDFTLTISSLEPEDFAVYYCQQRSNWP |
| | | TFGQGTKVEIK |
| SGN-PDL1V VH | 77 | TAAIS |
| CDR1 | | |
| SGN-PDLIV VH | 78 | GIIPIFGKAHYAQKFQG |
| CDR2 | | |
| SGN-PDL1V VH | 79 | KFHFVSGSPFGMDV |
| CDR3 | | |
| SGN-PDL1V VL | 80 | RASQSVSSYLA |
| CDR1 | | |
| SGN-PDL1V VL | 81 | DASNRAT |
| CDR2 | | |
| SGN-PDL1V VL | 82 | QQRSNWPT |
| CDR3 | | |
| SGN-ALPV heavy | 83 | EVQLVESGGGLVQPGRSLRLSCTASGFTFTDYYMSWVRQAPGKGL |
| chain variable region | | EWLALIRNKATGYTTEYTASVKGRFTISRDNSKSILYLQMNSLKTED |
| | | TAVYYCARASFYYDGKVLAYWGQGTLVTVSS |
| SGN-ALPV light | 84 | DTQMTQSPSSLSASVGDRVTITCQASQDINKYLAWYQYKPGKAPKL |
| chain variable region | | LIHYTSSLQSGVPSRFSGSGSGRDYTFTISSLQPEDIATYYCLQYDNL |
| | | YTFGQGTKLEIK |
| SGN-ALPV VH | 85 | DYYMS |
| CDR1 | | |
| SGN-ALPV VH | 86 | LIRNKATGYTTEYTASVKG |
| CDR2 | | |
| SGN-ALPV VH | 87 | ASFYYDGKVLAY |
| CDR3 | | |
| SGN-ALPV VL | 88 | QASQDINKYLA |
| CDR1 | | |
| SGN-ALPV VL | 89 | YTSSLQS |
| CDR2 | | |
| SGN-ALPV VL | 90 | LQYDNLYT |
| CDR3 | | |
| SGN-B7H4V heavy | 91 | QLQLQESGPGLVKPSETLSLTCTVSGGSIKSGSYYWGWIRQPPGKGL |
| chain variable region | | EWIGNIYYSGSTYYNPSLRSRVTISVDTSKNQFSLKLSSVTAADTAV |
| | | YYCAREGSYPNQFDPWGQGTLVTVSS |
| SGN-B7H4V light | 92 | EIVMTQSPATLSVSPGERATLSCRASQSVSSNLAWYQQKPGQAPRL |
| chain variable region | | LIYGASTRATGIPARFSGSGSGTEFTLTISSLQSEDFAVYYCQQYHSF |
| | | PFTFGGGTKVEIK |
| SGN-B7H4V VH | 93 | GSIKSGSYYWG |
| CDR1 | | |
| SGN-B7H4V VH | 94 | NIYYSGSTYYNPSLRS |
| CDR2 | | |
| SGN-B7H4V VH | 95 | AREGSYPNQFDP |
| CDR3 | | |
| SGN-B7H4V VL | 96 | RASQSVSSNLA |
| CDR1 | | |
| SGN-B7H4V VL | 97 | GASTRAT |
| CDR2 | | |
| SGN-B7H4V VL | 98 | QQYHSFPFT |
| CDR3 | | |
| Disitamab vedotin | 99 | EVQLVQSGAEVKKPGATVKISCKVSGYTFTDYYIHWVQQAPGKGL |
| heavy chain | | EWMGRVNPDHGDSYYNQKFKDKATITADKSTDTAYMELSSLRSED |
| | | TAVYFCARNYLFDHWGQGTLVTVSSASTKGPSVFPLAPSSKSTSGG |
| | | TAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK |
| Disitamab vedotin | 100 | DIQMTQSPSSVSASVGDRVTITCKASQDVGTAVAWYQQKPGKAPK |
| light chain | | LLIYWASIRHTGVPSRFSGSGSGTDFTLTISSLQPEDFATYYCHQFAT |
| | | YTFGGGTKVEIKRTVAAPSVFIFPPSDEQLKSGTASVVCLLNNFYPR |
| | | EAKVQWKVDNALQSGNSQESVTEQDSKDSTYSLSSTLTLSKADYE |
| | | KHKVYACEVTHQGLSSPVTKSFNRGEC |
| Lifastuzumab | 101 | EVQLVESGGGLVQPGGSLRLSCAASGFSFSDFAMSWVRQAPGKGLE |
| vedotin heavy chain | | WVATIGRVAFHTYYPDSMKGRFTISRDNSKNTLYLQMNSLRAEDT |
| | | AVYYCARHRGFDVGHFDFWGQGTLVTVSSASTKGPSVFPLAPSSKS |
| | | TSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEY |
| | | KCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| Lifastuzumab | 102 | DIQMTQSPSSLSASVGDRVTITCRSSETLVHSSGNTYLEWYQQKPGK |
| vedotin light chain | | APKLLIYRVSNRFSGVPSRFSGSGSGTDFTLTISSLQPEDFATYYCFQ |
| | | GSFNPLTFGQGTKVEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSFNRGEC |
| Enfortumab Vedotin | 103 | EVQLVESGGGLVQPGGSLRLSCAASGFTFSSYNMNWVRQAPGKGL |
| (EV) heavy chain | | EWVSYISSSSSTIYYADSVKGRFTISRDNAKNSLSLQMNSLRDEDTA |
| variable region | | VYYCARAYYYGMDVWGQGTTVTVSS |
| Enfortumab Vedotin | 104 | DIQMTQSPSSVSASVGDRVTITCRASQGISGWLAWYQQKPGKAPKF |
| (EV) light chain | | LIYAASTLQSGVPSRFSGSGSGTDFTLTISSLQPEDFATYYCQQANSF |
| variable region | | PPTFGGGTKVEIK |
| Enfortumab Vedotin | 105 | SYNMN |
| (EV) VH CDR1 | | |
| Enfortumab Vedotin | 106 | YISSSSSTIYYADSVKG |
| (EV) VH CDR2 | | |
| Enfortumab Vedotin | 107 | AYYYGMDV |
| (EV) VH CDR3 | | |
| Enfortumab Vedotin | 108 | RASQGISGWLA |
| (EV) VL CDR1 | | |
| Enfortumab Vedotin | 109 | AASTLQS |
| (EV) VL CDR2 | | |
| Enfortumab Vedotin | 110 | QQANSFPPT |
| (EV) VL CDR3 | | |
| h2A2 heavy chain | 111 | QFQLVQSGAEVKKPGASVKVSCKASGYSFTDYNVNWVRQAPGQG |
| variable region | | LEWIGVINPKYGTTRYNQKFKGRATLTVDKSTSTAYMELSSLRSED |
| | | TAVYYCTRGLNAWDYWGQGTLVTVSS |
| h2A2 light chain | 112 | DIQMTQSPSSLSASVGDRVTITCGASENIYGALNWYQQKPGKAPKL |
| variable region | | LIYGATNLEDGVPSRFSGSGSGRDYTFTISSLQPEDIATYYCQNVLTT |
| | | PYTFGQGTKLEIK |
| h2A2 VH CDR1 | 113 | DYNVN |
| h2A2 VH CDR2 | 114 | VINPKYGTTRYNQKFKG |
| h2A2 VH CDR3 | 115 | GLNAWDY |
| h2A2 VL CDR1 | 116 | GASENIYGALN |
| h2A2 VL CDR2 | 117 | GATNLED |
| h2A2 VL CDR3 | 118 | QNVLTTPYT |
| h15H3 heavy chain | 119 | QVQLVQSGAEVKKPGASVKVSCKASGYSFSGYFMNWVRQAPGQG |
| variable region | | LEWMGLINPYNGDSFYNQKFKGRVTMTRQTSTSTVYMELSSLRSED |
| | | TAVYYCVRGLRRDFDYWGQGTLVTVSS |
| h15H3 light chain | 120 | DVVMTQSPLSLPVTLGQPASISCKSSQSLLDSDGKTYLNWLFQRPGQ |
| variable region | | SPRRLIYLVSELDSGVPDRFSGSGSGTDFTLKISRVEAEDVGVYYCW |
| | | QGTHFPRTFGGGTKLEIK |
| h15H3 VH CDR1 | 121 | GYFMN |
| h15H3 VH CDR2 | 122 | LINPYNGDSFYNQKFKG |
| h15H3 VH CDR3 | 123 | GLRRDFDY |
| h15H3 VL CDR1 | 124 | KSSQSLLDSDGKTYLN |
| h15H3 VL CDR2 | 125 | LVSELDS |
| h15H3 VL CDR3 | 126 | WQGTHFPRT |
| SGN-CD228A | 127 | QVQLQESGPGLVKPSETLSLTCTVSGDSITSGYWNWIRQPPGKGLEY |
| heavy chain variable | | IGYISDSGITYYNPSLKSRVTISRDTSKNQYSLKLSSVTAADTAVYYC |
| region | | ARRTLATYYAMDYWGQGTLVTVSS |
| SGN-CD228A light | 128 | DFVMTQSPLSLPVTLGQPASISCRASQSLVHSDGNTYLHWYQQRPG |
| chain variable region | | QSPRLLIYRVSNRFSGVPDRFSGSGSGTDFTLKISRVEAEDVGVYYC |
| | | SQSTHVPPTFGQGTKLEIK |
| SGN-CD228A VH | 129 | SGYWN |
| CDR1 | | |
| SGN-CD228A VH | 130 | YISDSGITYYNPSLKS |
| CDR2 | | |
| SGN-CD228A VH | 131 | RTLATYYAMDY |
| CDR3 | | |
| SGN-CD228A VL | 132 | RASQSLVHSDGNTYLH |
| CDR1 | | |
| SGN-CD228A VL | 133 | RVSNRFS |
| CDR2 | | |
| SGN-CD228A VL | 134 | SQSTHVPPT |
| CDR3 | | |
| SGN-LIV1A | 135 | QVQLVQSGAEVKKPGASVKVSCKASGLTIEDYYMHWVRQAPGQG |
| Ladiratuzumab | | LEWMGWIDPENGDTEYGPKFQGRVTMTRDTSINTAYMELSRLRSD |
| Vedotin (LV) heavy | | DTAVYYCAVHNAHYGTWFAYWGQGTLVTVSS |
| chain variable region | | |
| SGN-LIV1A | 136 | DVVMTQSPLSLPVTLGQPASISCRSSQSLLHSSGNTYLEWYQQRPGQ |
| Ladiratuzumab | | SPRPLIYKISTRFSGVPDRFSGSGSGTDFTLKISRVEAEDVGVYYCFQ |
| Vedotin (LV) light | | GSHVPYTFGGGTKVEIK |
| chain variable region | | |
| SGN-LIV1A | 137 | DYYMH |
| Ladiratuzumab | | |
| Vedotin (LV) VH | | |
| CDR1 | | |
| SGN-LIV1A | 138 | WIDPENGDTEYGPKFQG |
| Ladiratuzumab | | |
| Vedotin (LV) VH | | |
| CDR2 | | |
| SGN-LIV1A | 139 | HNAHYGTWFAY |
| Ladiratuzumab | | |
| Vedotin (LV) VH | | |
| CDR3 | | |
| SGN-LIV1A | 140 | RSSQSLLHSSGNTYLE |
| Ladiratuzumab | | |
| Vedotin (LV) VL | | |
| CDR1 | | |
| SGN-LIV1A | 141 | KISTRFS |
| Ladiratuzumab | | |
| Vedotin (LV) VL | | |
| CDR2 | | |
| SGN-LIV1A | 142 | FQGSHVPYT |
| Ladiratuzumab | | |
| Vedotin (LV) VL | | |
| CDR3 | | |
| Tisotumab Vedotin | 143 | EVQLLESGGGLVQPGGSLRLSCAASGFTFSNYAMSWVRQAPGKGL |
| (TV) heavy chain | | EWVSSISGSGDYTYYTDSVKGRFTISRDNSKNTLYLQMNSLRAEDT |
| variable region | | AVYYCARSPWGYYLDSWGQGTLVTVSS |
| Tisotumab Vedotin | 144 | DIQMTQSPPSLSASAGDRVTITCRASQGISSRLAWYQQKPEKAPKSLI |
| (TV) light chain | | YAASSLQSGVPSRFSGSGSGTDFTLTISSLQPEDFATYYCQQYNSYP |
| variable region | | YTFGQGTKLEIK |
| Tisotumab Vedotin | 145 | GFTFSNYA |
| (TV) VH CDR1 | | |
| Tisotumab Vedotin | 146 | ISGSGDYT |
| (TV) VH CDR2 | | |
| Tisotumab Vedotin | 147 | ARSPWGYYLDS |
| (TV) VH CDR3 | | |
| Tisotumab Vedotin | 148 | QGISSR |
| (TV) VL CDR1 | | |
| Tisotumab Vedotin | 149 | AAS |
| (TV) VL CDR2 | | |
| Tisotumab Vedotin | 150 | QQYNSYPYT |
| (TV) VL CDR3 | | |

What is claimed is:

1. A method of treating cancer, comprising administering to a subject with cancer (1) an antibody-drug conjugate (ADC) that comprises a first antibody that binds a tumor-associated antigen and a cytotoxic agent, wherein the cytotoxic agent is a tubulin disrupter; and (2) a second antibody that binds to an immune cell engager, wherein the second antibody comprises an Fc with enhanced binding to one or more activating FcγRs, wherein the activating FcγRs include one or more of FcγRIIIa, FcγRIIa, and/or FcγRI.

2. The method of claim 1, wherein the second antibody comprises an Fc with enhanced binding to at least FcγRIIIa.

3. The method of claim 1, wherein second antibody comprises an Fc with enhanced binding to at least FcγRIIIa and FcγRIIa.

4. The method of claim 1, wherein the second antibody comprises an Fc with enhanced binding to at least FcγRIIIa and FcγRI.

5. The method of claim 1, wherein the second antibody comprises an Fc with enhanced binding to FcγRIIIa, FcγRIIa, and FcγRI.

6. The method of any one of claims 1-5, wherein the Fc of the second antibody has reduced binding to one or more inhibitory FcγRs.

7. The method of claim 6, wherein the Fc of the second antibody has reduced binding to FcγRIIb.

8. The method of any one of claims 1-7, wherein the Fc of the second antibody has reduced fucose levels and/or has been engineered to comprise one or more mutations such that the Fc has enhanced binding to the one or more activating FcγRs.

9. The method of claim 8, wherein the second antibody is nonfucosylated.

10. The method of claim 8, wherein the second antibody comprises substitutions S293D, A330L, and 1332E in the heavy chain constant region.

11. A method of treating cancer, comprising administering to a subject with cancer an antibody-drug conjugate, wherein the antibody-drug conjugate comprises a first antibody conjugated to a cytotoxic agent, wherein the cytotoxic agent is a tubulin disrupter; and a second antibody that binds an immune cell engager, wherein the second antibody is nonfucosylated.

12. The method of any one of claims 1-11, wherein the first antibody binds a tumor-associated antigen.

13. A method of treating cancer, comprising administering to a subject with cancer (1) an antibody-drug conjugate (ADC), wherein the ADC comprises a first antibody that binds a tumor-associated antigen and a cytotoxic agent, wherein the cytotoxic agent is a tubulin disrupter, and (2) a second antibody that binds an immune cell engager, wherein the second antibody comprises an Fc with enhanced ADCC activity relative to a corresponding wild-type Fc of the same isotype.

14. The method of claim 13, wherein the second antibody comprises an Fc with enhanced ADCC and ADCP activity relative to a corresponding wild-type Fc of the same isotype.

15. The method of claim 13 or 14, wherein the second antibody is nonfucosylated.

16. The method of any one of claims 13-15, wherein the second antibody comprises an Fc with enhanced binding to one or more activating FcγRs, wherein the activating FcγRs include one or more of FcγRIIIa, FcγRIIa, and/or FcγRI.

17. The method of claim 16, wherein the second antibody comprise an Fc with enhanced binding to at least FcγRIIIa.

18. The method of claim 16, wherein second antibody comprises an Fc with enhanced binding to at least FcγRIIIa and FcγRIIa.

19. The method of claim 16, wherein the second antibody comprises an Fc with enhanced binding to at least FcγRIIIa and FcγRI.

20. The method of claim 16, wherein the second antibody comprises an Fc with enhanced binding to FcγRIIIa, FcγRIIa, and FcγRI.

21. The method of any one of claims 13-20, wherein the Fc of the second antibody has reduced binding to one or more inhibitory FcγRs.

22. The method of claim 21, wherein the Fc of the second antibody has reduced binding to FcγRIIb.

23. The method of any one of claims 1-22, wherein the first antibody binds an antigen selected from 5T4 (TPBG), ADAM-9, AG-7, ALK, ALP, AMHRII, APLP2, ASCT2, AVB6, AXL (UFO), B7-H3 (CD276), B7-H4, BCMA, C3a, C3b, C4.4a (LYPD3), C5, C5a, CA6, CA9, CanAg, carbonic anhydrase IX (CAIX), Cathepsin D, CCR7, CD1, CD10, CD100, CD101, CD102, CD103, CD104, CD105, CD106, CD107a, CD107b, CD108, CD109, CD111, CD112, CD113, CD116, CD117, CD118, CD119, CD11A, CD11b, CD11c, CD120a, CD121a, CD121b, CD122, CD123, CD124, CD125, CD126, CD127, CD13, CD130, CD131, CD132, CD133, CD135, CD136, CD137, CD138, CD14, CD140a, CD140b, CD141, CD142, CD143, CD144, CD146, CD147, CD148, CD15, CD150, CD151, CD154, CD155, CD156a, CD156b, CD156c, CD157, CD158b2, CD158e, CD158f1, CD158h, CD158i, CD159a, CD16, CD160, CD161, CD162, CD163, CD164, CD166, CD167b, CD169, CD16a, CD16b, CD170, CD171, CD172a, CD172b, CD172g, CD18, CD180, CD181, CD183, CD184, CD185, CD19, CD194, CD197, CD1a, CD1b, CD1c, CD1d, CD2, CD20, CD200, CD201, CD202b, CD203c, CD204, CD205, CD206, CD208, CD21, CD213al, CD213a2, CD217, CD218a, CD22, CD220, CD221, CD222, CD224, CD226, CD228, CD229, CD23, CD230, CD232, CD239, CD243, CD244, CD248, CD249, CD25, CD26, CD265, CD267, CD269, CD27, CD272, CD273, CD274, CD275, CD279, CD28, CD280, CD281, CD282, CD283, CD284, CD289, CD29, CD294, CD295, CD298, CD3, CD3 epsilon, CD30, CD300f, CD302, CD304, CD305, CD307, CD31, CD312, CD315, CD316, CD317, CD318, CD319, CD32, CD321, CD322, CD324, CD325, CD326, CD327, CD328, CD32b, CD33, CD331, CD332, CD333, CD334, CD337, CD339, CD34, CD340, CD344, CD35, CD352, CD36, CD37, CD38, CD39, CD3d, CD3g, CD4, CD41, CD42d, CD44, CD44v6, CD45, CD46, CD47, CD48, CD49a, CD49b, CD49c, CD49d, CD49e, CD49f, CD5, CD50, CD51, CD51 (integrin alpha-V), CD52, CD53, CD54, CD55, CD56, CD58, CD59, CD6, CD61, CD62L, CD62P, CD63, CD64, CD66a-e, CD67, CD68, CD69, CD7, CD70, CD70L, CD71, CD71 (TfR), CD72, CD73, CD74, CD79a, CD79b, CD8, CD80, CD82, CD83, CD84, CD85f, CD85i, CD85j, CD86, CD87, CD89, CD90, CD91, CD92, CD95, CD96, CD97, CD98, CDH6, CDH6 (cadherin 6), CDw210a, CDw210b, CEA, CEACAM5, CEACAM6, CFC1B, cKIT, CLDN18.2 (claudin 18.2), CLDN6, CLDN9, CLL-1, c-MET, complement factors C3, Cripto, CSP-1, CXCR5, DCLK1, DLK-1, DLL3, DPEP3, DR5 (Death receptor 5), Dysadherin, EFNA4, EGFR, EGFR wild type, EGFRviii, EGP-1 (TROP-2), EGP-2, EMP2, ENPP3, EpCAM, EphA2, EphA3, Ephrin-A4 (EFNA4), ETBR, FAP, FcRH5, FGFR2, FGFR3, FLT3, FOLR, FOLR1, FOLR-alpha, FSH, GCC, GD2, GD3, globo H, GPC1, GPC-1, GPC3, GPNMB, GPR20, HER2, HER-2, HER3, HER-3, HGFR (c-Met), HLA-DR, HM1.24, HSP90, Ia, IGF-1R, IL-13R, IL-15, IL1RAP, IL-2, IL-3, IL-4, IL7R, integrin alphaVbeta3 (integrin aVP3), integrin beta-6, Interleukin-4 Receptor (IL4R), KAAG-1, KLK2, LAMP-1, Le(y), Lewis Y antigen, LGALS3BP, LGR5, LH/hCG, LHRH, Lipid raft, LIV-1 (SLC39A6 or ZIP6), LRP-1, LRRC15, LY6E, Macrophage mannose receptor 1, MAGE, Mesothelin (MSLN), MET, MHC class I chain-related protein A and B (MICA and MICB), MN/CA IX, MRC2, MT1-MMP, MTX3, MTX5, MUC1, MUC16, MUC2, MUC3, MUC4, MUC5, MUC5ac, NaPi2b, NCA-90, NCA-95, Nectin-4, Notch3, Nucleolin, OAcGD2, OT-MUC1 (onco-tethered-MUC1), OX001L, P1GF, PAM4 antigen, p-cadherin (cadherin 3), PD-L1, Phosphatidyl Serine(PS), PRLR, Prolactin Receptor (PRLR), Pseudomonas, PSMA, PTK4, PTK7, Receptor tyrosine kinase (RTK), RNF43, ROR1, ROR2, SAIL, SEZ6, SLAMF7, SLC44A4, SLITRK6, SLMAMF7 (CS1), SLTRK6, Sortilin (SORT1), SSEA-4, SSTR2, Staphylococcus aureus (antibiotic agent), STEAP-1, STING, STn, T101, TAA, TAC, TDGF1, tenascin, TENB2, TGF-B, Thomson-Friedenreich antigens, Thy1.1, TIM-1, tissue factor (TF; CD142), TM4SF1, Tn antigen, TNF-alpha (TNFα), TRA-1-60, TRAIL receptor (R1 and R2), TROP-2, Tumor-associated glycoprotein 72 (TAG-72), uPAR, VEGFR, VEGFR-2, and xCT.

24. The method of any one of claims 1-23, wherein the first antibody does not bind Nectin-4.

25. The method of any one of claims 1-24, wherein the method does not comprise administering an antibody-drug conjugate comprising an antibody that binds Nectin-4.

26. The method of any one of claims 1-25, wherein the first antibody binds an antigen selected from CD71, Ax1, AMHRII, and LGR5, Ax1, CA9, CD142, CD20, CD22, CD228, CD248, CD30, CD33, CD37, CD48, CD7, CD71, CD79b, CLDN18.2, CLDN6, c-MET, EGFR, EphA2, ETBR, FCRH5, GCC, Globo H, gpNMB, HER-2, IL7R, Integrin beta-6, KAAG-1, LGR5, LIV-1, LRRC15, Ly6E, Mesothelin (MSLN), MET, MRC2, MUC16, NaPi2b, Nectin-4, OT-MUC1 (onco-tethered-MUC1), PSMA, ROR1, SLAMF7, SLC44A4, SLITRK6, STEAP-1, STn, TIM-1, TRA-1-60, and Tumor-associated glycoprotein 72 (TAG-72).

27. The method of any one of claims 1-25, wherein the first antibody binds an antigen selected from BCMA, GPC1, CD30, cMET, SAIL, HER3, CD70, CD46, CD48, HER2, 5T4, ENPP3, CD19, EGFR, and EphA2.

28. The method of any one of claims 1-25, wherein the first antibody binds an antigen selected from Her2, TROP2, BCMA, cMet, integrin alphVbeta6 (integrin aVP6), CD22, CD79b, CD30, CD19, CD70, CD228, CD47, and CD48.

29. The method of any one of claims 1-25, wherein the first antibody binds an antigen selected from CD142, Integrin beta-6, integrin alphaVbeta6, ENPP3, CD19, Ly6E, cMET, C4.4a, CD37, MUC16, STEAP-1, LRRC15, SLITRK6, ETBR, FCRH5, Ax1, EGFR, CD79b, BCMA, CD70, PSMA, CD79b, CD228, CD48, LIV-1, EphA2, SLC44A4, CD30, and sTn.

30. The method of any one of claims 1-29, wherein the tubulin disrupter is an auristatins, a tubulysin, a colchicine, a vinca alkaloid, a taxane, a cryptophycin, a maytansinoid, or a hemiasterlin.

31. The method of claim 30, wherein the tubulin disrupter is an auristatin.

32. The method of any one of claims 1-31, wherein the tubulin disrupter is dolostatin-10, MMAE (N-methylvaline-valine-dolaisoleuine-dolaproine-norephedrine), MMAF (N-methylvaline-valine-dolaisoleuine-dolaproine-phenylalanine), auristatin F, AEB, AEVB, or AFP (auristatin phenylalanine phenylenediamine).

33. The method of any one of claims 1-32, wherein the tubulin disrupter is MMAE.

34. The method of claim 33, wherein the MMAE is conjugated to the first antibody through a linker that comprises valine and citrulline.

35. The method of claim 34, wherein the linker-MMAE is vcMMAE.

36. The method of claim 33, wherein the MMAE is conjugated to the first antibody through a linker that comprises leucine, alanine, and glutamic acid.

37. The method of claim 36, wherein the linker-MMAE is dLAE-MMAE.

38. The method of any one of claims 1-32, wherein the tubulin disrupter is MMAF.

39. The method of any one of claims 1-32, wherein the tubulin disrupter is a tubulysin.

40. The method of claim 39, wherein the tubulysin is selected from tubulysin D, tubulysin M, tubuphenylalanine, and tubutyrosine.

41. The method of any one of claims 1-32, wherein the antibody-drug conjugate is selected from AbGn-107 (Ab1-18Hr1), AGS62P1 (ASP1235), ALT-P7 (HM2-MMAE), BA3011 (CAB-AXL-ADC), belantamab mafodotin, brentuximab vedotin, cirmtuzumab vedotin (VLS-101, UC-961ADC3), cofetuzumab pelidotin (PF-06647020, PTK7-ADC, PF-7020, ABBV-647), CX-2029 (ABBV-2029), disitamab vedotin (RC48), enapotamab vedotin (HuMax-AXL-ADC, AXL-107-MMAE), enfortumab vedotin (EV), FS-1502 (LCB14-0110), gemtuzumab ozogamicin, HTI-1066 (SHR-A1403), inotuzumab ozogamicin, PF-06804103 (NG-HER2 ADC), polatuzumab vedotin, sacituzumab govitecan, SGN-B6A, SGN-CD228A SGN-STNV, STI-6129 (CD38 ADC, LNDS1001, CD38-077 ADC), telisotuzumab vedotin (ABBV-399), tisotumab vedotin (Humax-TF-ADC, tf-011-mmae, TV), trastuzumab deruxtecan, trastuzumab emtansine, and vorsetuzumab mafodotin.

42. The method of any one of claims 1-41, wherein the first antibody is an anti-claudin-18.2 antibody that comprises a heavy chain CDR1, CDR2, and CDR3, and a light chain CDR1, CDR2, and CDR3 respectively comprising the amino acid sequences of SEQ ID NOs:61-66.

43. The method of claim 42, wherein the anti-claudin-18.2 antibody comprises a heavy chain variable region (VH) comprising the amino acid sequence of SEQ ID NO:59 and a light chain variable region (VL) comprising the amino acid sequence of SEQ ID NO:60.

44. The method of claim 43, wherein the anti-claudin-18.2 antibody is zolbetuximab (175D10).

45. The method of any one of claims 1-41, wherein the first antibody is an anti-claudin-18.2 antibody that comprises a heavy chain CDR1, CDR2, and CDR3, and a light chain CDR1, CDR2, and CDR3 respectively comprising the amino acid sequences of SEQ ID NOs: 69-74.

46. The method of claim 45, wherein the anti-claudin-18.2 antibody comprises a heavy chain variable region (VH) comprising the amino acid sequence of SEQ ID NO:67 and a light chain variable region (VL) comprising the amino acid sequence of SEQ ID NO:68.

47. The method of any one of claims 1-41, wherein the first antibody is an anti-PD-L1 antibody that comprises a heavy chain CDR1, CDR2, and CDR3, and a light chain CDR1, CDR2, and CDR3 respectively comprising the amino acid sequences of SEQ ID NOs:77-82.

48. The method of claim 47, wherein the anti-PD-L1 antibody comprises a heavy chain variable region (VH) comprising the amino acid sequence of SEQ ID NO:75 and a light chain variable region (VL) comprising the amino acid sequence of SEQ ID NO:76.

49. The method of any one of claims 1-41, wherein the first antibody is an anti-ALP antibody that comprises a heavy chain CDR1, CDR2, and CDR3, and a light chain CDR1, CDR2, and CDR3 respectively comprising the amino acid sequences of SEQ ID NOs:85-90.

50. The method of claim 49, wherein the anti-ALP antibody comprises a heavy chain variable region (VH) comprising the amino acid sequence of SEQ ID NO:83 and a light chain variable region (VL) comprising the amino acid sequence of SEQ ID NO:84.

51. The method of any one of claims 1-41, wherein the first antibody comprises an anti-B7H4 antibody that comprises a heavy chain CDR1, CDR2, and CDR3, and a light chain CDR1, CDR2, and CDR3 respectively comprising the amino acid sequences of SEQ ID NOs:93-98.

52. The method of claim 51, wherein the anti-B7H4 antibody comprises a heavy chain variable region (VH) comprising the amino acid sequence of SEQ ID NO:91 and a light chain variable region (VL) comprising the amino acid sequence of SEQ ID NO:92.

53. The method of any one of claims 1-41, wherein the first antibody is an anti-HER2 antibody that comprises a heavy chain comprising the amino acid sequence of SEQ ID NO:99 and a light chain comprising the amino acid sequence of SEQ ID NO:100.

54. The method of claim 53, wherein the antibody-drug conjugate is disitamab vedotin.

55. The method of any one of claims 1-41, wherein the first antibody is an anti-NaPi2B antibody that comprises a heavy chain comprising the amino acid sequence of SEQ ID NO:101 and a light chain comprising the amino acid sequence of SEQ ID NO:102.

56. The method of claim 55, wherein the antibody-drug conjugate is lifastuzumab vedotin.

57. The method of any one of claims 1-41, wherein the first antibody is an anti-nectin-4 antibody that comprises a heavy chain CDR1, CDR2, and CDR3, and a light chain CDR1, CDR2, and CDR3 respectively comprising the amino acid sequences of SEQ ID NOs:105-110.

58. The method of claim 57, wherein the anti-nectin-4 antibody is an antibody that comprises a heavy chain variable region (VH) comprising the amino acid sequence of SEQ ID NO:103 and a light chain variable region (VL) comprising the amino acid sequence of SEQ ID NO:104.

59. The method of claim 58, wherein the antibody-drug conjugate is enfortumab vedotin.

60. The method of any one of claims 1-41, wherein the first antibody is an anti-AVB6 antibody that comprises a heavy chain CDR1, CDR2, and CDR3, and a light chain CDR1, CDR2, and CDR3 respectively comprising the amino acid sequences of SEQ ID NOs:113-118.

61. The method of claim 60, wherein the anti-AVB6 antibody comprises a heavy chain variable region (VH) comprising the amino acid sequence of SEQ ID NO:37 and a light chain variable region (VL) comprising the amino acid sequence of SEQ ID NO:38.

62. The method of any one of claims 1-41, wherein the first antibody is an anti-AVB6 antibody that comprises a heavy chain CDR1, CDR2, and CDR3, and a light chain CDR1, CDR2, and CDR3 respectively comprising the amino acid sequences of SEQ ID NOs:121-126.

63. The method of claim 62, wherein the anti-AVB6 antibody comprises a heavy chain variable region (VH) comprising the amino acid sequence of SEQ ID NO: 119 and a light chain variable region (VL) comprising the amino acid sequence of SEQ ID NO: 120.

64. The method of any one of claims 1-41, wherein the first antibody is an anti-CD228 antibody that comprises a heavy chain CDR1, CDR2, and CDR3, and a light chain CDR1, CDR2, and CDR3 respectively comprising the amino acid sequences of SEQ ID NOs:129-134.

65. The method of claim 64, wherein the anti-CD228 antibody comprises a heavy chain variable region (VH) comprising the amino acid sequence of SEQ ID NO: 127 and a light chain variable region (VL) comprising the amino acid sequence of SEQ ID NO: 128.

66. The method of any one of claims 1-41, wherein the first antibody is an anti-LIV-1 antibody that comprises a heavy chain CDR1, CDR2, and CDR3, and a light chain CDR1, CDR2, and CDR3 respectively comprising the amino acid sequences of SEQ ID NOs:137-142.

67. The method of claim 66, wherein the anti-LIV-1 antibody comprises a heavy chain variable region (VH) comprising the amino acid sequence of SEQ ID NO:135 and a light chain variable region (VL) comprising the amino acid sequence of SEQ ID NO:136.

68. The method of any one of claims 1-41, wherein the first antibody is an anti-tissue factor antibody that comprises heavy chain CDR1, CDR2, and CDR3, and a light chain CDR1, CDR2, and CDR3 respectively comprising the amino acid sequences of SEQ ID NOs:145-150.

69. The method of claim 68, wherein the anti-tissue factor antibody comprises a heavy chain variable region (VH) comprising the amino acid sequence of SEQ ID NO:143 and a light chain variable region (VL) comprising the amino acid sequence of SEQ ID NO:144.

70. The method of claim 69, wherein the antibody-drug conjugate is tisotumab vedotin.

71. The method of any one of claims 1-70, wherein the second antibody binds an immune cell engager selected from anti-Mullerian Hormone Receptor II (AMHR2), B7, B7H1, B7H2, B7H3, B7H4, BAFF-R, BCMA (B-cell maturation antigen), Bst1/CD157, C5 complement, CC chemokine receptor 4 (CCR4), CD123, CD137, CD19, CD20, CD25 (IL2RA), CD276, CD278, CD3, CD32, CD33, CD37, CD38, CD4 and HIV-1 gp120-binding sites, CD40, CD70, CD70 (a member of the TNF receptor ligand family), CD80, CD86, Claudin 18.2, c-MET, CSF1R, CTLA-4, EGFR, EGFR MET proto-oncogene, EPHA3, ERBB2, ERBB3, FGFR2b, FLT3, GITR, glucocorticoid-induced TNF receptor (GITR), HER2, HER3, HLA, ICOS, IDO1, IFNAR1, IFNAR2, IGF-1R, IL-3Ralpha (CD123), IL-5R, IL-5Ralpha, LAG-3, MET proto-oncogene, OX40 (CD134), PD-1, PD-L1, PD-L2, PVRIG, respiratory syncytial virus (RSV) heavily glycosylated mucin-like domain of EBOV glycoprotein (GP), Rhesus (Rh) D, sialic acid immunoglobulin-like lectins 8 (Siglec-8), signaling lymphocyte activation molecule (SLAMF7/CS1), T-cell receptor cytotoxic T-lymphocyte-associated antigen 4 (CTLA4), TIGIT, TIM3 (HAVCR2), tumor specific glycoepitope of Muc1 (TA-Mucd), VSIR (VISTA), and VTCN1.

72. The method of any one of claims 1-71, wherein the second antibody binds TIGIT.

73. The method of claim 72, wherein the second antibody comprises:
(a) a heavy chain CDR1 comprising an amino acid sequence selected from SEQ ID NOs: 7-9;
(b) a heavy chain CDR2 comprising an amino acid sequence selected from SEQ ID NOs: 10-13;
(c) a heavy chain CDR3 comprising an amino acid sequence selected from SEQ ID NOs: 14-16;
(d) a light chain CDR1 comprising the amino acid sequence of SEQ ID NO: 17;
(e) a light chain CDR2 comprising the amino acid sequence of SEQ ID NO: 18; and
(f) a light chain CDR3 comprising the amino acid sequence of SEQ ID NO: 19.

74. The method of claim 72, wherein the second antibody comprises a heavy chain CDR1, CDR2, and CDR3 and a light chain CDR1, CDR, and CDR3 comprising the sequences of:
(a) SEQ ID NOs: 7, 10, 14, 17, 18, and 19, respectively; or
(b) SEQ ID NOs: 8, 11, 14, 17, 18, and 19, respectively; or
(c) SEQ ID NOs: 9, 12, 15, 17, 18, and 19, respectively; or
(d) SEQ ID NOs: 8, 13, 16, 17, 18, and 19, respectively; or
(e) SEQ ID NOs: 8, 12, 16, 17, 18, and 19, respectively.

75. The method of claim 72, wherein the second antibody comprises a heavy chain variable region comprising an amino acid sequence selected from SEQ ID NOs: 1-5 and a light chain variable region comprising the amino acid sequence of SEQ ID NO: 6.

76. The method of claim 72, wherein the second antibody comprises a heavy chain comprising an amino acid sequence selected from SEQ ID NOs: 20-24 and a light chain comprising the amino acid sequence of SEQ ID NO: 25.

77. The method of any one of claims 1-71, wherein the second antibody binds CD40.

78. The method of claim 77, wherein the second antibody comprises a heavy chain CDR1, CDR2, and CDR3 and a light chain CDR1, CDR, and CDR3 comprising the sequences of: (a) SEQ ID NOs: 30, 31, 32, 33, 34, and 35, respectively; or (b) SEQ ID NOs: 30, 36, 32, 33, 34, and 35, respectively.

79. The method of claim 77, wherein the second antibody comprises a heavy chain variable region comprising the amino acid sequence of SEQ ID NO: 28 and a light chain variable region comprising the amino acid sequence of SEQ ID NO: 29.

80. The method of claim 77, wherein the second antibody comprises a heavy chain comprising the amino acid sequence of SEQ ID NO: 26 and a light chain comprising the amino acid sequence of SEQ ID NO: 27.

81. The method of any one of claims 1-71, wherein the second antibody binds CD70.

82. The method of claim 81, wherein the second antibody comprises a heavy chain CDR1, CDR2, and CDR3 and a light chain CDR1, CDR, and CDR3 comprising the sequences of SEQ ID NOs: 53-58, respectively.

83. The method of claim 81, wherein the second antibody comprises a heavy chain variable region comprising the amino acid sequence of SEQ ID NO: 41 and a light chain variable region comprising the amino acid sequence of SEQ ID NO: 42.

84. The method of any one of claims 1-71, wherein the second antibody binds BCMA.

85. The method of claim 84, wherein the second antibody comprises a heavy chain CDR1, CDR2, and CDR3 and a light chain CDR1, CDR, and CDR3 comprising the sequences of SEQ ID NOs: 47-52, respectively.

86. The method of claim 84, wherein the second antibody comprises a heavy chain variable region comprising the amino acid sequence of SEQ ID NO: 45 and a light chain variable region comprising the amino acid sequence of SEQ ID NO: 46.

87. The method of any one of claims 1-86, wherein the second antibody is an IgG1 or IgG3 antibody.

88. The method of any one of claims 1-87, wherein the second antibody is comprised in a composition of antibodies, wherein at least 90%, at least 91%, at least 92%, at least 93%, at least 94%, at least 95%, at least 96%, at least 97%, at least 98%, or at least 99% of the antibodies in the composition are nonfucosylated.

89. The method of claim 88, wherein each antibody in the composition comprises the same heavy chain and light chain amino acid sequences as the second antibody.

90. The method of any one of claims 1-89, wherein the Fc of the second antibody has enhanced binding to one or more activating FcγRs as compared to a corresponding wild-type Fc of the same isotype, wherein the activating FcγRs include one or more of FcγRIIIa, FcγRIIa, and/or FcγRI.

91. The method of claim 90, wherein the Fc of the second antibody has enhanced binding to FcγRIIIa.

92. The method of any one of claims 1-91, wherein the Fc of the second antibody has reduced binding to one or more inhibitory FcγRs as compared to a corresponding wild-type Fc of the same isotype.

93. The method of claim 92, wherein the Fc of the second antibody has reduced binding to FcγRIIb.

94. The method of any one of claims 1-93, wherein the Fc of the second antibody has enhanced binding to FcγRIIIa and reduced binding to FcγRIIb.

95. The method of any one of claims 1-94, wherein the second antibody is a monoclonal antibody.

96. The method of any one of claims 1-95, wherein the second antibody is a humanized antibody or a human antibody.

97. The method of any one of claims 1-96, wherein the cancer is bladder cancer, breast cancer, uterine cancer, cervical cancer, ovarian cancer, prostate cancer, testicular cancer, esophageal cancer, gastrointestinal cancer, gastric cancer, pancreatic cancer, colorectal cancer, colon cancer, kidney cancer, clear cell renal carcinoma, head and neck cancer, lung cancer, lung adenocarcinoma, stomach cancer, germ cell cancer, bone cancer, liver cancer, thyroid cancer, skin cancer, melanoma, neoplasm of the central nervous system, mesothelioma, lymphoma, leukemia, chronic lymphocytic leukemia, diffuse large B cell lymphoma, follicular lymphoma, Hodgkin lymphoma, myeloma, or sarcoma.

98. The method of any one of claims 1-97, wherein the cancer is lymphoma, leukemia, chronic lymphocytic leukemia, diffuse large B cell lymphoma, follicular lymphoma, or Hodgkin lymphoma.

99. The method of any one of claims 1-98, wherein the antibody-drug conjugate and the second antibody are administered concurrently.

100. The method of claim 99, wherein the antibody-drug conjugate and the second antibody are administered in a single pharmaceutical composition.

101. The method of any one of claims 1-98, wherein the antibody-drug conjugate and the second antibody are administered sequentially.

102. The method of claim 101, wherein at least a first dose of the antibody-drug conjugate is administered prior to a first dose of the second antibody; or wherein at least a first dose of the second antibody is administered prior to a first dose of the antibody-drug conjugate.

103. The method of any one of claims 1-102, wherein the second antibody depletes T regulatory cells (Tregs).

104. The method of any one of claims 1-103, wherein the antibody-drug conjugate induces immune memory against cells expressing the antigen bound by the antibody-drug conjugate.

105. The method of claim 104, wherein the induction of immune memory comprises induction of memory T cells.

106. The method of any one of claims 1-105, wherein the second antibody activates antigen presenting cells (APCs).

107. The method of any one of claims 1-106, wherein the second antibody enhances CD8 T cell responses.

108. The method of any one of claims 1-107, wherein the second antibody upregulates co-stimulatory receptors.

109. The method of any one of claims 1-108, wherein administration of the ADC and the second antibody promotes release of an immune activating cytokine.

110. The method of claim 109, wherein the immune activating cytokine is CXCL10 or IFNγ.

111. The method of any one of claims 1-110, wherein the ADC and the second antibody act synergistically.

112. The method of any one of claims 1-111, wherein administration of the ADC and the second antibody in combination has a toxicity profile comparable to that of the ADC or the second antibody when either is administered as monotherapy.

113. The method of any one of claims 1-112, wherein the effective dose of the ADC and/or the second antibody when dosed in combination is less than when administered as monotherapy.

114. The method of any one of claims 1-113, wherein the cancer has high tumor mutation burden.

115. The method of any one of claims 1-114, wherein the cancer has microsatellite instability.
